SEQUENCE LISTING (28 sequences)

SEQ ID NO: 1 - protein, 901 aa, Actinomyces viscosus
MTSHSPFSRRRLPALLGSLPLAATGLIAAAPPAHAVPTSDGLADVTITQVNAPADGLYSVGDVMTFNITLTNTSGEAHSYAPASTNLSGNVSKCRWRNVPAGTTKTDCTGLATHTVTAEDLKAGGFTPQIAYEVKAVEYAGKALSTPETIKGATSPVKANSLRVESITPSSSQENYKLGDTVSYTVRVRSVSDKTINVAATESSFDDLGRQCHWGGLKPGKGAVYNCKPLTHTITQADVDAGRWTPSITLTATGTDGATLQTLTATGNPINVVGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAEPSPAPSPTAAPSAAPTEKPAPSAAPSAEPTQAPAPSSAPEPSAAPEPSSAPAPEPTTAPSTEPTPAPAPSSAPEQTDGPTAAPAPETSSAPAAEPTQAPTVAPSVEPTQAPGAQPSSAPKPGATGRAPSVVNPKATGAATEPGTPSSSASPAPSRNAAPTPKPGMEPDEIDRPSDGTMAQPTGGASAPSAAPTQAAKAGSRLSRTGTNALLILGLAGVAVVGGYLLLRARRSKN

SEQ ID NO: 2 - protein, 394 aa, Actinomyces viscosus
GDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAE

SEQ ID NO: 3 - protein, 24 aa, Homo sapiens
NGRRICLDLQAPLYKKIIKKLLES

SEQ ID NO: 4 - protein, 27 aa, Homo sapiens
GRELCLDPKENWVQRVVEKFLKRAENS

SEQ ID NO: 5 - protein, 34 aa, Homo sapiens
QIHFFFAKLNCRLYRKANKSSKLVSANRLFGDKS

SEQ ID NO: 6 - protein, 34 aa, Homo sapiens
ELRVRLASHLRKLRKRLLRDADDLQKRLAVYQAG

SEQ ID NO: 7 - protein, 12 aa, Homo sapiens
RRLRRMESESES

SEQ ID NO: 8 - protein, 21 aa, Homo sapiens
KRKKKGGKNGKNRRNRKKKNP

SEQ ID NO: 9 - protein, 400 aa, Artificial Sequence
  Synthetic construct
MGELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

SEQ ID NO: 10 - protein, 422 aa, Artificial Sequence
  Synthetic construct
MVKRKKKGGKNGKNRRNRKKKNPGGGGSGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAE

SEQ ID NO: 11 - protein, 415 aa, Artificial Sequence
  Synthetic construct
MGELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAEPSPAPSPTAAPSAAKRKKKGGKNGKNRRNRKKKNP

SEQ ID NO: 12 - protein, 404 aa, Artificial Sequence
  Synthetic construct
MGHHHHHHLEGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAE

SEQ ID NO: 13 - protein, 416 aa, Artificial Sequence
  Synthetic construct
MKRKKKGGKNGKNRRNRKKKNPGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAE

SEQ ID NO: 14 - protein, 10 aa, Artificial Sequence
  Random primer sequence for N-terminal optimization (aa)
MXXKRKKKGG

SEQ ID NO: 15 - protein, 374 aa, Homo sapiens
  protease inhibitor 8 (PI8) (Serpin B8)
MDDLCEANGTFAISLFKILGEEDNSRNVFFSPMSISSALAMVFMGAKGSTAAQMSQALCLYKDGDIHRGFQSLLSEVNRTGTQYLLRTANRLFGEKTCDFLPDFKEYCQKFYQAELEELSFAEDTEECRKHINDWVAEKTEGKISEVLDAGTVDPLTKLVLVNAIYFKGKWNEQFDRKYTRGMLFKTNEEKKTVQMMFKEAKFKMGYADEVHTQVLELPYVEEELSMVILLPDDNTDLAVVEKALTYEKFKAWTNSEKLTKSKVQVFLPRLKLEESYDLEPFLRRLGMIDAFDEAKADFSGMSTEKNVPLSKVAHKCFVEVNEEGTEAAAATAVVRNSRCSRMEPRFCADHPFLFFIRRHKTNCILFCGRFSSP

SEQ ID NO: 16 - DNA, 4176 bp, Artificial Sequence
  Plasmid vector PTrc99A
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 17 - protein, 415 aa, Artificial Sequence
  DAS181
MGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

SEQ ID NO: 18 - protein, 116 aa, Homo sapiens
  Preprosomatostatin
MLSCRLQCALAALSIVLALGCVTGAPSDPRLRQFLQKSLAAAAGKQELAKYFLAELLSEPNQTENDALEPEDLSQAAEQDEMRLELQRSANSNPAMAPRERKAGCKNFFWKTFTSC

SEQ ID NO: 19 - protein, 92 aa, Homo sapiens
  Prosomatostatin
APSDPRLRQFLQKSLAAAAGKQELAKYFLAELLSEPNQTENDALEPEDLSQAAEQDEMRLELQRSANSNPAMAPRERKAGCKNFFWKTFTSC

SEQ ID NO: 20 - protein, 28 aa, Homo sapiens
  somatostatin 28 (SS-28)
SANSNPAMAPRERKAGCKNFFWKTFTSC

SEQ ID NO: 21 - protein, 14 aa, Homo sapiens
  somatostatin 14 (SS-14)
AGCKNFFWKTFTSC

SEQ ID NO: 22 - protein, 9 aa, Artificial Sequence
  Leuprolide
EHWSYLLRP

SEQ ID NO: 23 - DNA, 21 bp, Artificial Sequence
  Synthetic siRNA NP1496 sense strand
ggaucuuauuucuucggagnn

SEQ ID NO: 24 - DNA, 21 bp, Artificial Sequence
  Synthetic siRNA NP-1496 antisense strand
cuccgaagaaauaagauccnn

SEQ ID NO: 25 - DNA, 21 bp, Artificial Sequence
  Synthetic siRNA PA-2087 sense strand
gcaauugaggagugccugann

SEQ ID NO: 26 - DNA, 21 bp, Artificial Sequence
  Synthetic siRNA PA-2087 antisense strand
ucaggcacuccucaauugcnn

SEQ ID NO: 27 - RNA, 21 nt, Artificial Sequence
  Synthetic siRNA GAPDH sense strand
ugguuuacauguuccaauauu

SEQ ID NO: 28 - RNA, 21 nt, Artificial Sequence
  Synthetic siRNA GAPDH antisense strand
uauuggaacauguaaaccauu

(19) United States
(12) Patent Application Publication
(10) Pub. No.: US 2012/0141590 A1
(43) Pub. Date: Jun. 7, 2012

(54) Technology for the Preparation of Microparticles

(76) Inventors: Michael Malakhov, San Francisco, CA (US); Fang Fang, San Diego, CA (US)

(21) Appl. No.: 13/250,653

(22) Filed: Sep. 30, 2011

Related U.S. Application Data
(63) Continuation of application No. 12/179,520, filed on Jul. 24, 2008, now abandoned.
(60) Provisional application No. 60/961,872, filed on Jul. 24, 2007.

(51) Int. Cl. A61K 9/50; C07K 14/435; C07K 14/81; C07K 14/77; C07K 9/00; C07H 15/234; C07D 499/68; C07H 21/02; A61K 38/08; A61K 38/31; C07D 305/14; C07D 307/937; C08B 37/02; C08B 37/16; C07C 227/00; C07C 323/58; A61K 31/7088; A61P 3/02; A61P 37/02; A61K 47/36; C12N 9/24; C12N 9/76; C12N 9/36; C12N 9/22; C12N 7/00; C12N 7/02; C07K 14/765

(52) U.S. Cl. 424/490; 530/363; 530/385; 530/350; 530/367; 530/322; 536/16.8; 536/13.7; 540/336; 536/24.5; 530/328; 530/311; 549/510; 549/465; 530/354; 536/112; 536/103; 562/575; 562/573; 548/497; 562/559; 562/443; 514/44 R; 514/44 A; 514/777; 435/200; 435/213; 435/206; 435/199; 435/235.1; 435/239

(57) ABSTRACT

Microspheres are produced by contacting a solution of a macromolecule or small molecule in a solvent with an antisolvent and a counterion, and chilling the solution. The microspheres are useful for preparing pharmaceuticals, nutraceuticals, cosmetic products and the like of defined dimensions.

### RELATED APPLICATIONS

This application claims priority under 35 U.S.C. §119(e) to U.S. provisional application Ser. No. 60/961,872, entitled “TECHNOLOGY FOR THE PREPARATION OF MICROPARTICLES” to Fang et al. filed Jul. 24, 2007. The subject matter of the provisional application is incorporated in its entirety by reference herein. This application also is related to International PCT Application No. (Attorney Dkt. No. 21865-005WO1/6505PC) filed on the same day herewith. The subject matter of the PCT application is incorporated by reference herein.

This application is related to International PCT Application Serial No. (Attorney Docket No. 21865-004W01/6504PC, filed Jan. 24, 2007), and to U.S. application Ser. No. 11/657,812, filed Jan. 24, 2007 (Attorney Docket No. 21865-004001/6504). This application also is related to published U.S. applications Serial Nos. US20050004020 A1 and US20050112751 A1. Each of these applications is incorporated by reference herein in its entirety.

### INCORPORATION BY REFERENCE OF SEQUENCE LISTING FILED ELECTRONICALLY

An electronic version of the Sequence Listing is filed herewith, the contents of which are incorporated by reference in their entirety. The computer-readable file, created on Jul. 24, 2008, is 46 kilobytes in size and titled 21865005001 SeqList.txt.

### BACKGROUND

The preparation and delivery of compounds of interest in powder or particle form is an area of concentrated research and development activity in a variety of industries, including the pharmaceutical, nutraceutical and cosmetic industries. For optimal efficacy, it is desirable to have a uniform formulation of the compound, whether it is a small molecule, such as a steroid hormone or penicillin antibiotic, or a macromolecule, such as a protein or nucleic acid. For example, for pulmonary administration of a compound, such as a therapeutic protein, antibiotic or chemotherapeutic agent, the compound ideally should be prepared in the form of discrete microspheres, which are solid or semi-solid particles having a diameter of between 0.5 and 5.0 microns. It also is desirable for the microparticles to have as high a content of the compound as possible, in a form that maintains its activity for concentrated delivery and therapeutic efficacy.

Previous methods of producing microparticles or nanoparticles of compounds have involved complex steps, such as blending with organic polymers and/or forming a lattice array with polymers; spray drying, spray freeze-drying or supercritical fluid antisolvent techniques that use specialized and complex equipment; or lyophilization followed by pulverization or milling that often results in non-uniform particles that must further be sorted. Often such methods include processing steps, such as heating, that inactivate the compounds and compromise their activity (e.g., denaturation of a protein). In addition, some methods do not provide a quantitative recovery of the compound from solution into the solid microparticle formulation. Other methods, such as directly precipitating a compound out of solution by adding an antisolvent, can generate microparticles in an uncontrolled manner that results in uneven-sized and/or aggregated microparticles.

Accordingly, there is a need for a method for producing protein and other macromolecular microparticles, and small-molecule microparticles, which does not require complex or specialized equipment and that produces uniform-sized microparticles for delivery. There further is a need for a method of producing microparticles of a compound that contain high concentrations of the compound relative to other components of the microparticles, that are stable and maintain their activity for long periods of time when stored at ambient temperature, and that do not contain a significant amount of inactive compound. There also is a need for a method of producing microparticles of compounds where substantially all of the compound present in the starting material (e.g., a solution of the compound) is recovered in the microparticle formulation, with minimal loss. There also is a need for microparticles containing these properties for administration, for example, as a therapeutic or nutritional supplement, or in a cosmetic product.

### SUMMARY

The methods of making a microparticle, the microparticles themselves, combinations, and articles of manufacture provided below are characterized by a variety of component ingredients, steps of preparation, and biophysical, physical, biochemical and chemical parameters. As would be apparent to one of skill in the art, the compositions and methods provided herein include any and all permutations and combinations of the ingredients, steps and/or parameters described below.

Provided herein are methods for producing microparticles of a compound, which do not require complex or specialized equipment and that produce uniform-sized microparticles for delivery. Also provided herein are methods of producing microparticles of a compound that contain high concentrations of the compound relative to other components of the microparticles, that are stable and maintain their activity for long periods of time when stored at ambient temperature, and that do not contain a significant amount of inactive compound. Also provided are methods of producing microparticles of compounds where substantially all of the compound present in the starting material is recovered in the microparticle formulation, with minimal loss. Also provided are methods of producing microparticle containing a carrier that facilitates the formation of microspheres containing the molecule that is the active agent or therapeutic agent of interest, or promotes stability of the resulting microspheres, or facilitates transportation of the resulting microsphere to the target (cells, tissues, etc.) of interest. In some embodiments, the carrier can be a material, such as gelatin or dextran, which is capable of forming a hydrogel. Further, provided herein are microparticles containing these properties for administration, for example, as a therapeutic or nutritional supplement, as a diagnostic or in a cosmetic product.

The methods of making the microparticles of the compounds, including macromolecular microparticles and small-molecule microparticles, the compositions themselves, combinations and articles of manufacture provided below are characterized by a variety of component ingredients, steps of preparation, and biophysical, physical, biochemical and chemical parameters. As would be apparent to one of skill in the art, the compositions and methods provided herein include any and all permutations and combinations of the ingredients, steps and/or parameters described below.

The methods provided herein can include the steps of:

a) adding a counterion to a solution containing the compound in a solvent;

b) adding an antisolvent to the solution; and

c) gradually cooling the solution to a temperature below about 25° C., whereby a composition containing microparticles of the compound is formed, In the method, steps a), b) and c) can be performed simultaneously, sequentially, intermittently, or in any order.

In some examples, the counterion is not a polymer. In further examples, the antisolvent is not a polymer. The temperature at which the steps are performed also can be altered. In some embodiments, the compound is dissolved in the solvent at a temperature of about or at 30° C. or below prior to step a). In other embodiments, the compound is dissolved in the solvent at a temperature of about or at 25° C. or below. In one aspect, none of the solutions of steps a)-c) are heated and/or maintained at a temperature above about or at 30° C. In some examples, the compound in these methods is not a protein or polypeptide.

In some embodiments, the compounds can be heated to temperatures of above ambient temperature to dissolve the compound in the solvent/antisolvent system, then cooled to a temperature at which microspheres are formed. For example, for some macromolecules and small molecules, the compound can be heated in solution to about or at 35° C., 37° C., 40° C., 45° C., 50° C., 60° C., 65° C., 70° C., 75° C., 80° C., 85° C., 90° C., 95° C., 100° C., 125° C., 150° C., 175° C., 200° C. or greater, then cooled to a temperature of, for example, about or at 190° C., 170° C., 150° C., 125° C., 100° C., 80° C., 75° C., 60° C., 50° C., 40° C., 30° C., 20° C., 15° C. or lower, at which the microspheres are formed.

The order in which the steps are performed can be varied. For example, steps a) and b) can be performed simultaneously, sequentially, intermittently, or in any order, followed by step c). In other examples, steps b) and c) are performed simultaneously, sequentially, intermittently, or in any order, preceded by step a). In a further example, steps a) and c) are performed simultaneously. In other embodiments, steps a), b) and c) are performed sequentially in the order: a), then b), then c). In some embodiments, the counterion and the compound are identical to one another. In other embodiments, the compound and the counterion are different from one another. In other examples, the counterion and the antisolvent are identical to one another.

The compound can be a small molecule or a macromolecule. In instances where the compound is a macromolecule, the macromolecule can have a molecular weight of about or at 1000 or 1000 to about or at five billion or five billion Daltons; about or at 1000 or 1000 to about or at one billion or one billion Daltons; about or at 1000 or 1000 to about or at 50 million or 50 million Daltons; about or at 1000 or 1000 to about or at 20 million or 20 million Daltons; about or at 1000 or 1000 to about or at 15 million or 15 million Daltons; about or at 1000 or 1000 to about or at 10 million or 10 million Daltons; about or at 1000 or 1000 to about or at 5 million or 5 million Daltons; about or at 1000 or 1000 to about or at one million or one million Daltons; about or at 1000 or 1000 to about or at 500,000 or 500,000 Daltons; about or at 1000 or 1000 to about or at 300,000 or 300,000 Daltons; about or at 1000 or 1000 to about or at 200,000 or 200,000 Daltons; about or at 1000 or 1000 to about or at 100,000 or 100,000 Daltons; about or at 1000 or 1000 to about or at 50,000 or 50,000 Daltons; about or at 1000 or 1000 to about or at 25,000 or 25,000 Daltons; about or at 1000 or 1000 to about or at 15,000 or 15,000 Daltons; about or at 1000 or 1000 to about or at 10,000 or 10,000 Daltons; about or at 1000 or 1000 to about or at 5,000 or 5,000 Daltons; about or at 1000 or 1000 to about or at 3,000 or 3000 Daltons; or about or at 1000 or 1000 to about or at 2,000 or 2000 Daltons.

The macromolecule can be a polynucleotide, a nucleic acid, a polypeptide, a glycopeptide, a protein, a carbohydrate, a lipid, a fatty acid, a polysaccharide, carbohydrate- or polysaccharide-protein conjugate, virus, virus particle, viroid, prion or mixture thereof. In other examples, the macromolecule is a hormone, prostaglandin, antibiotic, chemotherapeutic agent, hematopoietic, anti-infective agent, antiulcer agent, antiallergic agent, antipyretic, analgesic, anti-inflammatory agent, antidementia agent, antiviral agent, antitumor agent, antidepressant, psychotropic agents, cardiotonics, diuretic, antiarrhythmic agent, vasodilator, antihypertensive agent, antidiabetic agent, anticoagulant, or cholesterol lowering agent.

In one embodiment, the macromolecule is conjugated to a small molecule. In some embodiments, the small molecule has a molecular weight of about or at 50 to about or at 1000 Daltons. The small molecule can be selected from among haptens, hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, and cholesterol lowering agents. For example, the small molecule can be an antibiotic, and can be selected from among aminoglycosides, ansamycins, carbacephem, carbapenems, cephalosporins, macrolides, penicillins, quinolones, sulfonamides and tetracyclines. In instances where the antibiotic is an aminoglycoside, the aminoglycoside can be kanamycin or tobramycin. In instances where the small molecule is an antiviral agent, the antiviral agent can be for treatment of influenza, parainfluenza or respiratory syncytial virus-mediated infections. In some examples, the antiviral agent is zanamivir or oseltamivir phosphate. In embodiments where the small molecule is a chemotherapeutic agent, the chemotherapeutic agent can be selected from among alkylating agents, anthracyclines, cytoskeletal disruptors, epothilones, inhibitors of topoisomerase II, nucleotide analogs, platinum-based agents, retinoids and vinca alkaloids. In some examples, the chemotherapeutic agent is a cytoskeletal disruptor, and the cytoskeletal disruptor is paclitaxel. I other examples, the small molecule is a prostaglandin.

In some embodiments, the macromolecule in the methods presented herein is a nucleic acid. The nucleic acid can be selected from among DNA, RNA and PNA. In instances where the nucleic acid is RNA, the RNA can be siRNA, snRNA, tRNA or a ribozyme. In some examples, the macromolecule is a virus, and the virus is tobacco mosaic virus. In other embodiments, the macromolecule is a glycopeptides, and the glycopeptide is vancomycin. In further embodiments, the macromolecule is a peptide. For example, the peptide can be leuprolide or somatostatin.

The solvent used in the methods provided herein can be miscible or partially miscible with the antisolvent. The methods provided herein also can contain a further process of separating the microparticles from the solution to remove components other than the microparticles after step c). In one aspect, the composition of this method can consist essentially of the microparticles containing the compound. In one embodiment, the separation is effected by sedimentation or by filtration. In another embodiment, the separation is effected by freeze-drying.

The antisolvent used in the methods provided herein can be selected from among water, buffered solutions, aliphatic alcohols, aromatic alcohols, chloroform, polyhydric sugar alcohols, aromatic hydrocarbons, aldehydes, ketones, esters, ethers, dioxanes, alkanes, alkenes, conjugated dienes, dichloromethane, carbon tetrachloride, dimethylformamide (DMF), dimethyl sulfoxide (DMSO), acetonitrile, ethyl acetate, polyols, polyimides, polyimines, polyesters, polyaldehydes and mixtures thereof. For example, the antisolvent is an aliphatic alcohol or an aromatic alcohol. In instances where the antisolvent is an aliphatic alcohol, the aliphatic alcohol can be isopropanol.

In examples, the counterion used in the methods provided herein is selected from among an anionic compound, a cationic compound and a zwitterionic compound. In examples, where the counterion is an anionic compound, the anionic compound can be sodium citrate, sodium sulfate, zinc sulfate, magnesium sulfate, potassium sulfate or calcium sulfate. In one aspect, the anionic compound is sodium sulfate. In other examples, the counterion is selected from among citric acid, itaconic acid and pivalic acid. In further examples, the counterion is an amino acid, such as, for example, glycine or arginine.

In some embodiments of the methods of making microparticles provided herein, the counterion is a polymer, and the macromolecule is selected from among a polynucleotide, a nucleic acid, a carbohydrate, a lipid, a fatty acid, a polysaccharide, carbohydrate- or polysaccharide-protein conjugates, a virus, virus particles, viroids, prions and mixtures thereof. In one aspect, the polymer is the counterion and the antisolvent. The polymer can be, for example, polyethylene glycol (PEG) or polyethyleneimine (PEI). The counterion used in the methods provided herein also can be a polymer. In one example, the polymer is the counterion and the antisolvent. The polymer can be, for example, polyethylene glycol (PEG) or polyethyleneimine (PEI).

The pH of the solution used in the methods provided herein can be from about 4.0 or 4.0 to about 9.0 or 9.0; from about 4.0 or 4.0 to about 8.0 or 8.0; from about 4.5 or 4.5 to about 7.5 or 7.5; or from about 5.0 or 5.0 to about 7.0 or 7.0.

The microparticles formed in the methods provided herein can be obtained by precipitation, by phase separation or by colloid formation. The resulting microparticle composition can further contain acid-resistant coating agents, protease-resistant coating agents, enteric coating agents, bulking agents, excipients, inactive ingredients, stability enhancers, taste and/or odor modifiers or masking agents, vitamins, sugars, therapeutic agents, anti-oxidants, immuno-modulators, trans-membrane transport modifiers, anti-caking agents, chitosans or flowability enhancers. In some examples, the amount of compound in the microparticles relative to the total amount of compound in the solution of step a) is about 5% or 5% to greater than about 99% or 99%, w/w; is about 5% or 5% to about 20% or 20%, w/w; about 10% or 10% to about 85% or 85%, w/w; about 20% or 20% to about 60% or 60%, w/w; about 25% or 25% to about 55% or 55%, w/w; about 30% or 30% to about 50% or 50%, w/w; or about 80% or 80% to greater than about 99% or 99%, w/w.

The temperature at which the solution is gradually cooled to can be between about or at 4° C. to about or at −200° C.; between about or at 2° C. to about or at −180° C.; between about or at 2° C. to about or at −170° C.; or between about 0° C. or 0° C. to about −2° C. or −2° C. to from about −150° C. or −150° C. to about −165° C. or −165° C.

In some aspects, the resulting composition has a shelf life of from about or at one week to about or at 1 month, from about or at 1 month to about or at six months, from about or at six months to about or at one year, from about or at 1 year to about or at 2 years, or from about or at 2 years to about or at 5 years at a temperature of about or at 55° C., 50° C., 45° C., 44° C., 42° C., 40° C., 39° C., 38° C., 37° C. or below.

In some embodiments, the solution and/or the resulting composition further includes an active agent. In embodiments where the resulting composition further includes an active agent, the active agent can be selected from among antibiotics, chemotherapeutic agents, antidiabetics, anticonvulsants, analgesics, antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids, sympathomimetics, parasympathomimetics, tranquilizers, urinary tract drugs, vaccines, vaginal drugs, nonsteroidal anti-inflammatory drugs, angiotensin converting enzymes, polynucleotides, polypeptides, polysaccharides, enzymes, hormones, vitamins, minerals, and nutritional supplements.

The moisture content of the microparticles formed in the methods provided herein can be adjusted whereby at least about 90% or 90% of the activity of the compound is retained after storage for about six months to about 1 year at a temperature of about 25° C. In other examples, the moisture content of the microparticles is adjusted whereby at least about 90% of the microparticles are not aggregated after storage for about six months to about 1 year at a temperature of about 25° C. In some aspects, the moisture content of the microparticles is from about or at 0.01% to about or at 20%; from about or at 0.05% to about or at 15%; from about or at 0.1% to about or at 10%; from about or at 0.2% to about or at 5%; from about or at 6% to about or at 12%; or from about or at 7% to about or at 10.5%.

In some embodiments of the methods provided herein, the concentration of counterion added to the solution is from about or at 0 mM or 0 mM to about or at 100 mM or 100 mM; from about or at 0 mM or 0 mM to about or at 50 mM or 50 mM; from about or at 0 mM or 0 mM to about or at 20 mM or 20 mM; from about or at 0 mM or 0 mM to about or at 10 mM or 10 mM; about or at 1 mM or 1 mM to about or at 5 mM or 5 mM; or is about or at 2 mM.

The gradual cooling of the solution in the methods provided herein can be effected by chilling. In other embodiments, the gradual cooling is by an endothermic reaction. In some aspects, the gradual cooling is at a rate of from about or at 0.01° C./min or 0.01° C./min to about or at 20° C./min or 20° C./min; from about or at 0.05° C./min or about or at 0.1° C./min to about or at 10° C./min or about or at 15° C./min; about or at 0.2° C./min to about or at 5° C./min; about or at 0.5° C./min to about or at 2° C./min; or at a rate of about or at 1° C./min.

In one embodiment, the size of the size of the microparticles is from about or at 0.001 μm or 0.001 μm to about or at 50 μm or 50 μm; about or at 0.3 μm or 0.3 μm to about or at 30 μm or 30 μm; about or at 0.5 μm or 0.5 μm to about or at 10 μm or 10 μm; about or at 0.5 μm or 0.5 μm to about or at 5.0 μm or 5.0 μm; about or at 1.0 μm or 1.0 μm to about or at 5.0 μm or 5.0 μm; or from about or at 1.0 μm to about or at 2.0, 3.0, 4.0 or 5.0 μm.

Also provided herein are compositions containing microparticles of a compound and a counterion, wherein the compound and the counterion are different from one another. In some embodiments, the compound is a macromolecule with a molecular weight of about or at 1000 or 1000 to about or at five billion or five billion Daltons; about or at 1000 or 1000 to about or at one billion or one billion Daltons; about or at 1000 or 1000 to about or at 50 million or 50 million Daltons; about or at 1000 or 1000 to about or at 20 million or 20 million Daltons; about or at 1000 or 1000 to about or at 15 million or 15 million Daltons; about or at 1000 or 1000 to about or at 10 million or 10 million Daltons; about or at 1000 or 1000 to about or at 5 million or 5 million Daltons; about or at 1000 or 1000 to about or at one million or one million Daltons; about or at 1000 or 1000 to about or at 500,000 or 500,000 Daltons; about or at 1000 or 1000 to about or at 300,000 or 300,000 Daltons; about or at 1000 or 1000 to about or at 200,000 or 200,000 Daltons; about or at 1000 or 1000 to about or at 100,000 or 100,000 Daltons; about or at 1000 or 1000 to about or at 50,000 or 50,000 Daltons; about or at 1000 or 1000 to about or at 25,000 or 25,000 Daltons; about or at 1000 or 1000 to about or at 15,000 or 15,000 Daltons; about or at 1000 or 1000 to about or at 10,000 or 10,000 Daltons; about or at 1000 or 1000 to about or at 5,000 or 5,000 Daltons; about or at 1000 or 1000 to about or at 3,000 or 3000 Daltons; or about or at 1000 or 1000 to about or at 2,000 or 2000 Daltons.

In some examples, the compound in the composition is a small molecule. The small molecule can have molecular weight of about or at 50 to about or at 1000 Daltons. In examples where the compound in the composition is a macromolecule, the macromolecule can selected from among a polynucleotide, a nucleic acid, a polypeptide, a glycopeptide, a protein, a carbohydrate, a lipid, a fatty acid, a polysaccharide, carbohydrate- or polysaccharide-protein conjugates, virus, virus particles, viroids, prions and mixtures thereof. In some embodiments, the macromolecule is selected from among hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, and cholesterol lowering agents.

In some embodiments, the macromolecule in the composition is conjugated to a small molecule. In such instances, the small molecule is selected from among haptens, hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, and cholesterol lowering agents.

In one aspect, the small compound is selected from among hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, and cholesterol lowering agents. In embodiments where the small molecule is an antibiotic, the antibiotic can be selected from among aminoglycosides, ansamycins, carbacephem, carbapenems, cephalosporins, macrolides, penicillins, quinolones, sulfonamides and tetracyclines. For example, the antibiotic is a penicillin or a tetracycline. In other examples, the antibiotic is an aminoglycoside, such as, for example, kanamycin or tobramycin. In other aspects, the compound is an antiviral agent. In such instances, the antiviral agent can be for treatment of influenza, parainfluenza, or respiratory syncytial virus-mediated infections. For example, the antiviral agent can be zanamivir or oseltamivir phosphate. In further embodiments, the compound is a chemotherapeutic agent. Where the compound is a chemotherapeutic agent, the chemotherapeutic agent can be selected from among alkylating agents, anthracyclines, cytoskeletal disruptors, epothilones, inhibitors of topoisomerase II, nucleotide analogs, platinum-based agents, retinoids and vinca alkaloids. In some examples, the chemotherapeutic agent is a cytoskeletal disruptor, such as, for example, paclitaxel. In still further examples, the compound is a prostaglandin.

In some embodiments, the macromolecule in the compositions provided herein is a nucleic acid. The nucleic acid can be selected from, for example, among DNA, RNA and PNA. In instances where the nucleic acid is RNA, the RNA can be selected from among siRNA, snRNA, tRNA and ribozymes. In some examples, the RNA is siRNA. In other embodiments, the macromolecule in the compositions provided herein is a virus. For example, the macromolecules can be a tobacco mosaic virus. In other aspects, the macromolecule is a glycopeptides, such as, for example, vancomycin. In further aspects, the macromolecule is a peptide. The peptide can be, for example, leuprolide or somatostatin.

The compound in the compositions provided herein can be water-insoluble. The counterion can be selected from among an anionic compound, a cationic compound and a zwitterionic compound. In instances where the counterion is an anionic compound, the anionic compound can be sodium citrate, sodium sulfate, zinc sulfate, magnesium sulfate, potassium sulfate and calcium sulfate. In some examples, the anionic compound is sodium sulfate. In other examples, the counterion is selected from among citric acid, itaconic acid and pivalic acid. In a further aspect, the counterion is an amino acid, such as, for example, glycine or arginine. In other aspects, the counterion is polyethylene glycol (PEG) or polyethyleneimine (PEI).

The resulting microparticle compositions provided herein can further contain acid-resistant coating agents, protease-resistant coating agents, enteric coating agents, bulking agents, excipients, inactive ingredients, stability enhancers, taste and/or odor modifiers or masking agents, vitamins, sugars, therapeutic agents, anti-oxidants, immuno-modulators, trans-membrane transport modifiers, anti-caking agents, chitosans or flowability enhancers. In some aspects, the composition has a shelf life of from about or at one week to about or at 1 month, from about or at 1 month to about or at six months, from about or at six months to about or at one year, from about or at 1 year to about or at 2 years, or from about or at 2 years to about or at 5 years at a temperature of about or at 55° C., 50° C., 45° C., 44° C., 42° C., 40° C., 39° C., 38° C., 37° C. or below.

The compositions provided herein also can contain an active agent. The active agent can be selected from among antibiotics, chemotherapeutic agents, antidiabetics, anticonvulsants, analgesics, antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids, sympathomimetics, parasympathomimetics, tranquilizers, urinary tract drugs, vaccines, vaginal drugs, nonsteroidal anti-inflammatory drugs, angiotensin converting enzymes, polynucleotides, polypeptides, polysaccharides, enzymes, hormones, vitamins, minerals, and nutritional supplements.

The amount of compound in the microparticles of the compositions provided herein can be from about or at 0.1% to about or at 99% or greater, w/w; from about or at 0.2% to about or at 95% or greater, w/w; from about or at 0.5% to about or at 90% or greater, w/w; from about or at 1% to about or at 85% or greater, w/w; from about or at 2% to about or at 80% or greater, w/w; from about or at 5% to about or at 75% or greater, w/w; from about 65% to about 90% w/w; from about 70% to about 85%, 86%, 87%, 88%, 89% or 90% w/w; or from about 90% to about 99% w/w.

In some aspects, the moisture content of the microparticles is adjusted whereby at least about 90% or 90% of the activity of the compound is retained after storage for about or at six months to about or at 1 year at a temperature of about 25° C. In some embodiments, the amount of counterion in the microparticles is from about 0.01% or 0.01% to about 60% or 60% w/w; from about 0.5% or 0.5% to about 50% or 50% w/w; from about 1% or 1% to about 2% or 2% w/w; from about 0.01% or 0.01% to about 20% or 20% w/w; from about 0.05% or 0.05% to about 15% or 15% w/w; from about 0.1% or 0.1% to about 10% or 10% w/w; or from about 0.2% or 0.2% to about 5% or 5% w/w.

In one aspect, the moisture content of the microparticles is from about 6% or 6% to about 12% or 12%. In another aspect, the moisture content of the microparticles is from about 7% or 7% to about 10.5% or 10.5%.

The compositions provided herein can be for ingestion, inhalation, oral administration, intravenous, intranasal, parenteral, pulmonary, subcutaneous, ophthalmic or intramuscular administration. In one aspect, the size of the microparticles of the compositions provided herein is from about 0.001 μm or 0.001 μm to about 50 μm or 50 μm; from about 0.3 μm or 0.3 μm to about 30 μm or 30 μm; from about 0.5 μm or 0.5 μm to about 10 μm or 10 μm; from about 0.5 μm or 0.5 μm to about 5.0 μm or 5.0 μm; from about 1.0 μm or 1.0 μm to about 5.0 μm or 5.0 μm; or from about 1.0 μm to about 2.0, 3.0, 4.0 or 5.0 μm.

Also provided herein are articles of manufacture containing the composition provided herein, a packaging material for the composition and a label that indicates that the composition is for a therapeutic, nutraceutical or cosmetic indication. In some examples, the composition used in the article is for a therapeutic indication, such as, for example, cancer, influenza, parainfluenza or respiratory disorders. The article of can further contain an inhaler for pulmonary administration of the composition. In some embodiments, the inhaler is a dry powder inhaler, a metered dose inhaler or an electrostatic delivery device.

Provided herein are methods of preventing or treating an infectious disease, by administering a therapeutically effective amount of the composition provided herein to a subject. In some aspects, the infectious disease is selected from among arboviral infections, botulism, brucellosis, candidiasis, campylobacteriosis, chickenpox, chlamydia, cholera, coronovirus infections, staphylococcus infections, coxsackie virus infections, Creutzfeldt-Jakob disease, cryptosporidiosis, cyclospora infection, cytomegalovirus infections, Epstein-Barr virus infection, dengue fever, diphtheria, ear infections, encephalitis, influenza virus infections, parainfluenza virus infections giardiasis, gonorrhea, Haemophilus influenzae infections, hantavirus infections, viral hepatitis, herpes simplex virus infections, HIV/AIDS, helicobacter infection, human papillomavirus (HPV) infections, infectious mononucleosis, legionellosis, leprosy, leptospirosis, listeriosis, lyme disease, lymphocytic choriomeningitis, malaria, measles, marburg hemorrhagic fever, meningitis, monkeypox, mumps, mycobacteria infection, mycoplasma infection, norwalk virus infection, pertussis, pinworm infection, pneumococcal disease, Streptococcus pneumonia infection, Mycoplasma pneumoniae infection, Moraxella catarrhalis infection, Pseudomonas aeruginosa infection, rotavirus infection, psittacosis, rabies, respiratory syncytial virus infection, (RSV), ringworm, rocky mountain spotted fever, rubella, salmonellosis, SARS, scabies, sexually transmitted diseases, shigellosis, shingles, sporotrichosis, streptococcal infections, syphilis, tetanus, trichinosis, tuberculosis, tularemia, typhoid fever, viral meningitis, bacterial meningitis, west nile virus infection, yellow fever, adenovirus-mediated infections and diseases, retrovirus-mediated infectious diseases and yersiniosis zoonoses. For example, the infectious disease can be influenza, parainfluenza, respiratory syncytial virus.

The methods of treatment can be administered by oral, intravenous, intranasal, parenteral, subcutaneous, transdermal, topical, intraarticular, intramuscular or inhalation administration of the composition.

Provided herein also are methods making microparticles of siRNA, which includes the steps of:

(a) adding an antisolvent to a solution of siRNA in an aqueous solvent; and

(b) gradually cooling the solution to a temperature below about 25° C., whereby a composition containing microparticles of siRNA is formed, and steps (a) and (b) are performed simultaneously, sequentially, intermittently, or in any order.

The method can further include a step (c), adding a counterion, where steps (a), (b) and (c) are performed simultaneously, sequentially, intermittently, or in any order.

In some examples, the antisolvent used in the methods making microparticles of siRNA is isopropanol. In some examples, the solvent is water.

Also provided herein are compositions that include microparticles of siRNA. In some examples, the composition also contains a counterion.

Also provided herein are methods of making microparticles of a virus, which includes the steps of:

(a) adding an antisolvent to a solution of virus in an aqueous solvent; and

(b) gradually cooling the solution to a temperature below about 25° C., whereby a composition containing microparticles of a virus is formed, where steps (a) and (b) are performed simultaneously, sequentially, intermittently, or in any order.

The method also can include a step (c), adding a counterion, where steps (a), (b) and (c) are performed simultaneously, sequentially, intermittently, or in any order. In some examples, the antisolvent used in the methods making microparticles of a virus is isopropanol.

Also provided herein are methods of making microparticles of a virus, which includes the steps of:

(a) adding a counterion to a solution of virus in an aqueous solvent; and

(b) gradually cooling the solution to a temperature below about 25° C.,

whereby a composition containing microparticles of a virus is formed, where steps (a) and (b) are performed simultaneously, sequentially, intermittently, or in any order.

The method also can include a step (c), adding an antisolvent, wherein steps (a), (b) and (c) are performed simultaneously, sequentially, intermittently, or in any order. In some embodiments, the antisolvent is isopropanol. In other embodiments, the solvent is water.

Also provided herein are compositions containing microparticles of a virus. Such compositions also can contain a counterion. In some aspects, the virus is tobacco mosaic virus.

### DETAILED DESCRIPTION

### A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

The term “molecule” is used interchangeably herein with “compound,” and refers to a naturally occurring or chemically synthesized entity containing two or more atoms or ions linked by a covalent bond. The atoms or ions can belong to the same chemical element, or they can belong to different elements. A molecule or compound as used herein contains the composite elements in definite, unvarying proportions by weight, and is characterized by a chemical formula. The compound can be an inorganic compound, which as used herein is a compound that generally does not contain carbon-carbon bonds, or the compound can be an organic compound, which generally is characterized by the presence of carbon and hydrogen, and can additionally contain hetero atoms, such as nitrogen, oxygen, halogens and other such atoms. Examples of inorganic compounds, discussed elsewhere herein, include alkali and alkaline earth metal compounds and salts and other derivatives thereof, transition metal compounds, including coordination compounds and salts and other derivatives thereof, inorganic polymers, such as polysiloxanes, and other such compounds known to those of skill in the art. Examples of organic compounds, discussed elsewhere herein, include aliphatic, aromatic and alicyclic alcohols, aldehydes, carboxylic acids, esters, ketones, ethers, amines, amides, lactams, polymers thereof, and other such compounds known to those of skill in the art.

The term compound, as used herein, also refers to assemblies of inorganic and/or organic compounds, including macromolecular assemblies such as phages and viruses.

A compound as used herein, whether inorganic or organic, can be a macromolecule or a small molecule. The term “macromolecule” is used herein in the sense that is understood by those of skill in the art, and generally refers to a naturally occurring or chemically synthesized organic or inorganic molecule that is greater than or equal to about a 1000 Daltons to about or greater than 1, 2, 3, 5, 7, 10 or more trillion Daltons. A “macromolecule” as used herein includes a molecule containing two or more monomeric subunits, or derivatives thereof, which are linked by a covalent bond, an ionic bond, or other chemical interactions, such as hydrogen bonding, ionic pairing, base pairing or pairing between charges formed by charge polarization. The monomeric subunits can be different from one another, or identical to one another, and, in some embodiments, can form a polymer. The polymers can be inorganic polymers, such as silicones, polysilanes, polygermanes, polystannanes or polyphospahazenes, organic polymers, such as polyethylene or polythene, polypropylene, nylon, teflon, polystyrene, polyesters, polymethylmethacrylate, polyvinylchloride or polyisobutylene, or biological polymers, such as polysaccharides, polynucleotides and polypeptides. A macromolecule also refers to a molecule that, regardless of whether it has more than one subunit and/or is a polymer, can form tertiary and/or quaternary structure. Examples of macromolecules include a polynucleotide, a nucleic acid molecule including DNA, RNA, including siRNA, snRNA, tRNA, antisense RNA, and ribozymes, peptide nucleic acid (PNA), a polypeptide, such as leuprolide and somatostatin, glycopeptides, such as vancomycin, a protein, a carbohydrate, or a lipid, or derivatives or combinations thereof, for example, a nucleic acid molecule containing a peptide nucleic acid portion or a glycoprotein, respectively. Examples of macromolecules further include macromolecular assemblies, for examples, viruses, virus particles, phages, viroids, prions and combinations and conjugates thereof.

The term “macromolecule” as used herein also is intended to encompass all molecules that are within the scope of the description above and have a function, including macromolecules having a biological function, such as a nucleic acid, peptide, protein, hormone, cytokine, chemokine, etc., macromolecules having a therapeutic function, such as a drug, macromolecules having a nutraceutical function, such as a nutritional supplement, and macromolecules having a cosmetic formulation, such as a soap or a skin cream. For example, a compound can be a macromolecule and also can belong to one or more of the classes of compounds selected from among hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, cholesterol lowering agents and nutritional supplements. The methods, compositions, combinations, kits and articles of manufacture provided herein, described with reference to some macromolecules, such as proteins, peptides, nucleic acids and viruses, can be adapted for use with other macromolecules as defined and/or provided herein.

The term “polymer” as used herein includes any of numerous natural and synthetic compounds containing two or more repeat units of molecules linked together, generally about or at 5, 10, 15, 20, hundreds, thousands, up to millions of repeating units. Each repeating unit generally is understood by those of skill in the art as a monomer. A polymer can have identical repeating units, or more than one type of repeating unit. Exemplary repeating monomeric units include, for example, nucleotides or nucleotide derivatives such as those found in deoxyribonucleic acid (DNA), ribonucleic acid (RNA), and mixed DNA or RNA derivatives, or peptide nucleic acids (PNA). Other monomer units can include, such as those found in synthetic organic polymers, include, but are not limited to, acrylamides, styrenes, alkyl-substituted styrenes, acrylates, methacrylates, acrylic acid, methacrylic acid, vinyl chloride, vinyl acetate, butadiene, isoprene, ethylene glycol and ethyleneimine. Exemplary organic or inorganic polymers, natural and synthetic polymers, include, but are not limited to, agarose, cellulose, nitrocellulose, cellulose acetate, other cellulose derivatives, dextran, dextran-derivatives and dextran co-polymers, other polysaccharides, glass, silica gels, gelatin, polyethylene glycols, polyethyleneimines, polyethyleneimides, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and the like.

The term “small molecule” is used herein in the sense that is understood by those of skill in the art, and generally refers to a naturally occurring or chemically synthesized organic or inorganic molecule that is less than about 1000 Daltons, from about or at 1000 Daltons to about or at 950, 900, 850, 800, 750, 700, 650, 600, 550, 500, 450, 400, 375, 350, 325, 300, 275, 250, 225, 200, 175, 150, 125, 100, 75, 70, 65, 60, 55, 50, 45, 40, 35, 30, 25, 20, 15, 10, 5 or less Daltons. A small molecule as understood by those of skill in the art and used herein is a term that evolved to differentiate traditional drugs, such as the penicillin antibiotics, from the new class of drugs based on developments in genetic engineering and biotechnology, such as proteins, nucleic acids and the like. A small molecule is understood to mean any molecule that is not a macromolecule, such as a protein or nucleic acid. A “small molecule” as used herein can include a molecule containing two or more monomeric subunits, such as a dipeptide or dinucleotide, and generally is understood to refer to molecules that are about or at 1000 Daltons or below in molecular weight.

Examples of small molecules include, but are not limited to, inorganic molecules such as, but not limited to, carbon monoxide, carbon dioxide, metal (alkali metal, alkaline earth metal, transition metal, e.g.) carbonates, cyanides, cyanates, carbides, halides, thiocyanates, oxides, hydroxides, sulfides and hydrozide, coordination compounds, e.g., the cobalt salt [Co(NH₃)₆]Cl₃, and organometallic compounds, e.g. Fe(C₅H₅)₂. Small molecules that are organic compounds include, for example, nucleotides, amino acids, pteridines such as Furterene and Triamterene; purines such as Acefylline, 7-Morpholinomethyltheophylline, Pamabrom, Protheobromine and Theobromine; sterols such as cholesterol and lanosterol, steroids such as estrogen, testosterone, canrenone, oleandrin and spironolactone; penicillins, tetracyclines, sulfonamide derivatives such as Acetazolamide, Ambuside, Azosemide, Bumetanide, Butazolamide, Diphenylmethane-4.4′-disulfonamide, Disulfamide, Furosemide, uracils such as Aminometradine and Amisometradine, and the like, and prostaglandins.

The term “small molecule” as used herein also is intended to encompass all molecules that are within the scope of the description above and have a function, including a biological function, such as a hormone, a therapeutic function, such as a drug, a nutraceutical function, such as a nutritional supplement, and a cosmetic formulation, such as a soap or a skin cream. For example, a compound can be a small molecule and also belong to one or more of the classes of compounds selected from among hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, cholesterol lowering agents and nutritional supplements. The methods, compositions, combinations, kits and articles of manufacture provided herein, exemplified for some types of small molecules such as aminoglycosides, penicillins, ampicillins and prostaglandins, can be adapted for use with other small molecules as defined and/or provided herein.

The term “conjugate” as used herein refers to a chemical linkage or interaction. A conjugate can be a covalent or ionic chemical linkage between two or more atoms, ions, or compounds, or can be formed by other chemical interactions, such as hydrogen bonding, ionic pairing, base pairing or pairing between charges formed by charge polarization. Exemplary conjugation means include streptavidin- or avidin- to biotin interaction; hydrophobic interaction; magnetic interaction (e.g., using functionalized magnetic beads), polar interactions, such as wetting associations between two polar surfaces or between oligo/polyethylene glycol; formation of a covalent bond, such as an amide bond, disulfide bond, thioether bond, or via crosslinking agents, or via acid-labile or photocleavable linkers.

The term “substantially” or “substantial” as used herein generally means at least about 60% or 60%, about 70% or 70%, or about or at 75%, 80%, 85%, 90%, 95%, 96%, 97%, 98%, 99% or higher relative to a reference such as, for example, a nucleic acid or protein sequence or the original composition of an entity. Thus, a composition containing microparticles separated from “substantially” all other contaminants and/or ingredients including counterions, salts and solvents from the cocktail solution means that at least about 60% or 60%, about 70% or 70%, or about or at 75%, 80%, 85%, 90%, 95%, 96%, 97%, 98%, 99% or higher amounts of contaminants and/or reagents have been removed from the cocktail solution in which the microparticles are formed. The term “substantially identical” or “substantially homologous” or similar varies with the context as understood by those skilled in the relevant art and generally means at least about 60% or 60%, about 70% or 70%, or about or at 75%, 80%, 85%, 90%, 95%, 96%, 97%, 98%, 99% or higher identity.

The term “consists essentially of” or “consisting essentially of” as used herein refers to an entity from which substantially all other components/ingredients that are not associated with the entity or its properties have been removed or separated from the entity. Thus, a composition “consisting essentially of” microparticles means that all other ingredients such as contaminants and solvents have substantially been removed from the solution/suspension containing the microparticles.

The term “microparticle” as used herein is interchangeable with “microsphere” and refers to particles in the size range (average length, width or diameter) of about or at 0.001 micron (μm) to about or at 500 microns that contain a compound of interest. The compound of interest can be a macromolecule or a small molecule, an organic compound or an inorganic compound. The compound of interest can be an active agent, or the microparticle can in addition contain an active agent. The compound of interest that forms the microparticle, e.g., a macromolecule including a protein, nucleic acid, lipid or polysaccharide, or a small molecule including a sterol or steroid hormone, can be a carrier for the active agent, such as a drug or a nutritional supplement. The microparticles also can contain synthetic macromolecules including polymers, such as polyethylene glycol (PEG), polylactic acid (PLA), polylactic-co-glycolic acid (PLGA), and natural polymers such as albumin, gelatin, chitosan and dextran. The “microparticles” as described herein can contain and can be made from a particular natural or synthetic compound alone, or from more than one type of the same natural or synthetic compound (e.g., more than one type of protein), or from combinations of more than one different type of natural or synthetic compound (e.g., an antibiotic and a leuprolide peptide).

The term “microparticle” as used herein also generally refers to a particle that is not a solid form of the entire solution from which it is produced, although frozen and/or dried particles of a solution containing macromolecules also are contemplated herein. Rather, the microparticle as used herein generally is an assembly of a fraction of the components of a solution, including salts, counterions, solvents and other ingredients, that is formed by a process including, but not limited to, precipitation, sedimentation, phase separation and colloid formation.

The term “precipitation” as used herein refers to a process whereby a solute or solutes of interest in a solution, such as the components of a microparticle, no longer stay in solution and form a phase that is distinct from the solvent or solvents that were used to form the solution. Precipitation of a microparticle and controlling the size of the precipitated microparticle can be accomplished by a variety of means including, but not limited to, adjusting temperature, ionic strength, pH, dielectric constant, counterion concentration, organic solvent concentration, the addition of polyelectrolytes or polymers, surfactants, detergents, or a combination thereof.

The term “phase separation” as used herein refers to the transformation of a single homogeneous phase, such as a solution, into two or more phases, such as a suspension of a solid particle in a solvent or solution.

The term “sedimentation” as used herein refers to the motion of particles, such as microparticles, which are in a suspension in a liquid or which are formed in a solution in response to an external force such as gravity, centrifugal force or electric force.

The term “solution” is used interchangeably with “cocktail solution” herein and refers to a homogeneous mixture of two or more ingredients in a single phase, solid, liquid, or gas, where the distinct ingredients only are recognizable at the molecular level. The solution can be a liquid in which one or more solutes, such as salts, are dissolved in a solvent, such as water or alcohol, or dissolved in a mixture of miscible solvents, such as a mixture of water and ethyl alcohol. The solution also can be a frozen form of a liquid solution.

The term “miscible” as used herein refers to the ability of one or more components, such as liquids, solids and gases, to mix together to form a single, homogeneous phase. Thus, two liquids are miscible if they can be mixed to form a single, homogenous liquid whose distinct components are recognized only at the molecular level. When components are “partially miscible,” it means that they can be mixed to form a single homogenous phase in a certain concentration range, but not at other concentration ranges. As used herein, when a solvent is “partially miscible” with another solvent, it means that it is miscible at a concentration of about or at 50%, 45%, 40%, 35%, 30%, 25%, 20%, 15%, 10%, 9%, 8%, 7%, 6%, 5%, 4%, 3%, 2%, 1%, 0.5% or below volume/volume (v/v), when mixed with the other solvent.

As used herein, “immiscible” means that when two or more components, such as liquids, solids or gases are mixed, they form more than one phase. For example, when an organic solvent is immiscible with an aqueous solvent (e.g., hexane and water), the organic solvent is visible as a distinct layer that does not mix with the layer of aqueous solvent.

As used herein, the term “polypeptide,” means at least two amino acids, or amino acid derivatives, including mass modified amino acids and amino acid analogs, that are linked by a peptide bond, which can be a modified peptide bond. The terms “polypeptide,” “peptide” and “protein” are used essentially synonymously herein, although the skilled artisan will recognize that peptides generally contain fewer than about fifty to about one hundred amino acid residues, and that proteins often are obtained from a natural source and can contain, for example, post-translational modifications.

A polypeptide or protein can be translated from a polynucleotide, which can include at least a portion of a coding sequence, or a portion of a nucleotide sequence that is not naturally translated due, for example, to it being located in a reading frame other than a coding frame, or it being an intron sequence, a 3′ or 5′ untranslated sequence, a regulatory sequence such as a promoter, or the like. A polypeptide also can be chemically synthesized and can be modified by chemical or enzymatic methods following translation or chemical synthesis. A polypeptide can be post-translationally modified by phosphorylation (phosphoproteins), glycosylation (glycoproteins, proteoglycans), and the like, which can be performed in a cell or in a reaction in vitro.

As used herein, the term “fusion protein” refers to a protein that is a conjugate of domains obtained from more than one protein or polypeptide. A domain can be a polypeptide tag, such as a His₆ tag. The conjugates can be prepared by linking the domains by chemical conjugation, recombinant DNA technology, or combinations of recombinant expression and chemical conjugation.

A variety of chemical linkers are known to those of skill in the art and include, but are not limited to, amino acid and peptide linkages, typically containing between one and about 60 amino acids, more generally between about 10 and 30 amino acids, heterobifunctional cleavable cross-linkers, including but are not limited to, N-succinimidyl (4-iodoacetyl)-aminobenzoate, sulfosuccinimidyl (4-iodoacetyl)-aminobenzoate, 4-succinimidyl-oxycarbonyl-a-(2-pyridyldithio)toluene, sulfosuccinimidyl-6-[a-methyl-a-(pyridyldithiol)-toluamido]hexanoate, N-succinimidyl-3-(-2-pyridyldithio)-propionate, succinimidyl 6[3(-(-2-pyridyldithio)-propionamido]hexanoate, sulfosuccinimidyl 6[3(-(-2-pyridyldithio)-propionamido]hexanoate, 3-(2-pyridyldithio)-propionyl hydrazide, Ellman's reagent, dichlorotriazinic acid, and S-(2-thiopyridyl)-L-cysteine.

The term “sialidase fusion protein” as used herein refers to a fusion protein in which one or more domains is a sialidase or a portion thereof that retains at least about 60% or 60%, about 70% or 70%, or about or at 75%, 80%, 85%, 90%, 95%, 96%, 97%, 98%, 99% or more of its catalytic activity. A sialidase fusion protein as used herein also can refer to a fusion protein that contains a protein or polypeptide that is substantially homologous to a sialidase and possesses the enzymatic activity of a sialidase.

The term “catalytic domain” of a protein as used herein refers to a protein or polypeptide in which the only portion of the sequence that is substantially homologous to a sialidase is a sequence of amino acid residues that includes the domain responsible for the catalytic activity of the protein (e.g., residues 274-666 of SEQ ID NO: 1 are identified as the catalytic domain of Actinomyces viscosus sialidase) or catalytically active fragments thereof. The catalytic domain or catalytically active fragment thereof retains at least about 60% or 60%, about 70% or 70%, or about or at 75%, 80%, 85%, 90%, 95%, 96%, 97%, 98%, 99% or more of the catalytic activity of the protein.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, siRNA, snRNA, tRNA, ribozymes and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “oligonucleotide” or “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). The term “oligonucleotide” also is used herein essentially synonymously with “polynucleotide,” although those in the art will recognize that oligonucleotides, for example, PCR primers, generally are less than about fifty to one hundred nucleotides in length.

As used herein, the term “flowability characteristic” refers to a property that renders the ability to “flow,” where “flow” is a property that can permit a substance to be poured and to assume the shape of a container that it is poured into, without hindrance due to, for example, aggregation. Fluids generally have the property of “flow,” which generally renders them deformable, i.e., they can change their shape. The term “fluid” as used herein encompasses colloids containing liquids, including emulsions, aerosols and gases. Liquids, aerosols and gases with suspensions of solid particles, such as microparticles, also are considered “fluid” as defined herein.

As used herein, an emulsion is defined as a colloid of two immiscible liquids, a first liquid and a second liquid, where the first liquid is dispersed in the second liquid.

As used herein, surfactants (or “surface-active agents”) are chemical or naturally occurring entities which, when dissolved in an aqueous solution, reduce the surface tension of the solution or the interfacial tension between two or more phases in solution. The surfactant molecules generally are amphiphilic and contain hydrophilic head groups and hydrophobic tails. The surfactant molecules can act as stabilizers and/or improve flowability characteristics of the microparticles provided herein.

As used herein, a combination refers to any association between two or among more items for a purpose. For example, a combination of microparticles and an inhaler can be used for pulmonary delivery of a therapeutic agent.

As used herein, a composition refers to any mixture. It can be a solution, a suspension, liquid, powder, a paste, aqueous, non-aqueous or any combination thereof.

As used herein, a kit refers to a combination in which components are packaged optionally with instructions for use and/or reagents and apparatus for use with the combination.

As used herein, the term “enzyme” means a protein that catalyzes a chemical reaction or biological process. Enzymes generally facilitate and/or speed up such reactions and processes. In addition, enzymes generally are specific for a particular reaction or process, converting a specific set of reactants into specific products.

As used herein, the term “colloid” refers to a dispersion of solid particles, such as microparticles, in a liquid, such as the solution in which the microparticles are formed. The term “colloidal stability” refers to a colloid in which the particles are not substantially aggregated. For example, a stable colloid is one in which about 30%, 25%, 20%, 15%, 10%, 5%, 4%, 3%, 2%, 1%, 0.5% or less of the solid particles, such as microparticles, have formed aggregates.

The term “agglomerates” refers to the association of one or more particles, such as microspheres, loosely held together by van der Waals forces or surface tension or electrostatic or combinations thereof. In some instances, associations held by electrostatic forces can be defined as “Flocculates.” For the purposes herein, “Agglomerates” also encompass “Flocculates”. Agglomerates can generally readily be broken apart by shear forces within the air or liquid. The term “disperse” or “dispersivity” refers to the ability of the particles to “flow,” i.e., the extent to which the movement is not impeded by the presence of, for example, aggregates.

The term “aggregates” or “clumps” refers to the association of one or more particles, such as microspheres, amorphous precipitates, crystal- or glass-like particles or combinations thereof. Aggregates generally are not easily broken apart which inhibits their ability to disperse or form homogeneous suspensions or to form aerosols with desirable properties.

The term “non-denatured” as used herein is in reference to proteins and means a conformation of a protein, i.e., its secondary structure, tertiary structure, quaternary structure or combinations thereof, which essentially is unaltered from the protein in its naturally occurring state. The terms “non-denatured” and “native” are used interchangeably herein and mean a protein that retains all or at least about 50%, 60%, 70%, 80%, 85%, 90° k 91%, 92%, 93%, 94%, 95%, 96%, 97%, 98% or 99% of its length and/or natural conformation. The terms “non-denatured” or “native” as used interchangeably herein include the natural state of a protein in a cell, such as it's length and conformation including secondary, tertiary and quaternary structures. As defined herein, the “non-denatured” or “native” proteins including those in the compositions provided herein generally retain all or at least about 50%, 60%, 70%, 80%, 85%, 90%, 91%, 92%, 93%, 94%, 95%, 96%, 97%, 98% or 99% of the normal activity or function of the proteins in their natural state, e.g., as a nutrient to provide amino acid building blocks, an antioxidant, an enzyme, an antibody, a regulator of gene expression, a scaffold, etc.

As used herein, the terms “activity” or “function” are interchangeable with “biological activity” and refer to the in vivo activities of a compound, such as a protein, vitamin, mineral or drug, or physiological responses that result upon in vivo administration of a compound, composition or other mixture. Activity, thus, encompasses therapeutic effects and pharmaceutical activity of compounds, compositions and mixtures. Biological activities also can be observed in in vitro systems designed to test or use such activities.

As used herein, “functional activity” also is interchangeable with “activity,” “biological activity” or “function” and refers to a compound that displays one or more activities associated with its natural state, or with the class of compounds to which it belongs. For example, an aminoglycoside that is an antibiotic is exhibiting the functional activity of several compounds of its class. Similarly, a polypeptide or portion thereof that displays one or more activities associated with the native or non-denatured protein is functionally active. Functional activities include, but are not limited to, therapeutic efficacy, biological in vivo activity, catalytic or enzymatic activity, antigenicity (ability to bind to or compete with a polypeptide for binding to an anti-polypeptide antibody), immunogenicity, ability to form multimers, and the ability to specifically bind to a receptor or ligand for the polypeptide.

The term “denatured” as used herein refers to a protein that is altered from its native or non-denatured conformation, i.e., its secondary, tertiary or quaternary structure or combinations thereof. The altered conformation generally occurs by processing steps that include pasteurization, radiation, heat, chemicals, enzyme action, exposure to acids or alkalis, and ion-exchange and any combinations thereof. Denaturation of a protein generally results in diminishing all or some, generally more than 50% and at least about 70%, 80%, 85%, 90%, 91%, 92%, 93%, 94%, 95%, 96%, 97%, 98% or 99%, of the original properties including activity and function of the protein in its native or non-denatured state.

As used herein, the term “nutritional supplement” means a substance or composition that provides nutrients, including vitamins, minerals, fatty acids, amino acids, carbohydrates, enzymes, proteins, biochemicals and their metabolites, herbs and plants, to a host, such as an animal, including a human being. Nutrients that are supplied to the host through nutritional supplements can include nutrients essential for survival, good health, curing disease or preventing disease that are missing or deficient in a host's diet, and nutrients that are believed to augment good health, prevent disease or cure disease but are not considered essential for survival or good health.

As used herein, “hydrophobic” refers to a substance that is not charged or charge-polarized, or is not sufficiently charged or charge-polarized to bond with water or other polar solvents, as understood by those of skill in the art. Hydrophobic ligands can associate with each other or with other non-polar molecules or solvents in the presence of water or a polar solvent, through hydrophobic interactions. A hydrophobic ligand generally also is more soluble in non-polar solvents than in polar solvents. Examples of non-polar solvents include alkanes such as hexane, alkyl ethers such as diethyl ether, aromatic hydrocarbons such as benzene and alkyl halides such as methylene chloride and carbon tetrachloride, mono-, di- and triglycerides, fatty acids, such as oleic, linoleic, palmitic, stearic, conjugated forms thereof and their esters.

The term “water-insoluble” compound is used interchangeably herein with “hydrophobic” compound and refers to a compound that has a greater solubility in non-aqueous solvents than in aqueous solvents. For example, a “water-insoluble” compound is a compound that is fully or partly—about or equal to 20, 25, 30, 35, 40, 45, 50, 55, 60, 65, 70, 75, 80, 85, 90, 95, 96, 97, 98, 99 or 100%, insoluble in solutions that contain about or equal to 20, 25, 30, 35, 40, 45, 50, 55, 60, 65, 70, 75, 80, 85, 90, 95, 96, 97, 98, 99 or 100% by volume of water or aqueous solution, such as a buffer.

As used herein, a “hydrophilic” or “polar” ligand is a ligand that has a charge or is charge-polarized. A hydrophilic ligand as used herein has either a charged functional group, such as a carboxylate or ammonium, or a charge-polarized bond, such as hydroxyl or sulfhydryl that provides a charge to the ligand. Hydrophilic ligands can bond with water and other polar solvents including alcohols, amines, amides, acids, carboxylic acids, esters, nitriles, ketones, glycols and glycol ethers, through hydrogen bonds or ionic interactions. A hydrophilic ligand also has greater solubility in polar solvents than in non-polar solvents.

As used herein, the term “therapeutic agent” means an agent which, upon administration to a host, including humans, effectively ameliorates or eliminates symptoms or manifestations of an inherited or acquired disease or that cures said disease. Exemplary therapeutic agents include, for example, chemical compounds for cancer therapy, e.g., chemotherapeutic agents, chemical compounds directed against bacterial infections, e.g., antibiotics, antiviral compounds and the like, as understood by those of skill in the art.

As used herein, the term “carrier” or “micro-carrier” refers to a molecule that facilitates the formation of microspheres containing the molecule that is the active agent or therapeutic agent of interest, or promotes stability of the resulting microspheres, or facilitates transportation of the resulting microsphere to the target (cells, tissues, etc.) of interest. In some embodiments, carriers can be employed to impart stability to the microspheres. In embodiments where the therapeutic agent or active agent of interest contained in the microspheres has a high potency and is incorporated at a relatively low concentration (generally, about or at 0.001%, 0.005%, 0.01%, 0.02%, 0.05, 0.1%, 0.2%, 0.5%, 1%, 2%, 3%, 5%, 10%, 15%, 20%, 25%, 30%, 35%, 40%, 45% or 50%, or within a range of about or at 0.001% to about or at 50%), carriers can stabilize microsphere formulations that might otherwise be readily degraded. Examples of high potency compounds can include cytotoxic anti-cancer agents or nucleic acids such as siRNA. Exemplary carriers include amino acids, carboxylic acids (e.g. citric acid, maleic acid), polymers including proteins and nucleic acids, materials capable of forming hydrogels including gelatin and various polysaccharides, and their combinations. In some embodiments, active agents that are proteins or nucleic acids such as tRNA and siRNA are incorporated into microspheres that are stabilized using polysaccharides such as dextran or proteins such as gelatin as micro-carriers.

Molecules used as carriers generally have demonstrated safety and stability. For a given active agent or therapeutic agent, carrier systems can be optimized in a high-throughput manner.

As used herein, “shelf life” or “stability” refers to the time after preparation of the microparticle composition that the composition retains at least about or 70%, 80%, 85%, 90%, 91%, 92%, 93%, 94%, 95%, 96%, 97%, 98% or 99% of the initial protein activity that is present in the composition and other general physical characteristics of microspheres such as size, shape, and aerodynamic particle size distribution. Thus, for example, a composition that is stable for or has a shelf life of 30 days at room temperature, defined herein as range of between about 18° C. to about 25° C., 26° C., 27° C. or 28° C., would have at least about 70%, 80%, 85%, 90% 91%, 92%, 93%, 94%, 95%, 96%, 97%, 98% or 99% of the initial amount of the activity of protein present in the composition at 30 days following storage at 18° C. to about 25° C., 26° C., 27° C. or 28° C. The shelf life of the microparticle compositions provided herein generally is at least about 10 days at 55° C., at least about 2-3 weeks at 42° C., and at least about eight months or greater at 25° C., however, microparticles compositions of any length of shelf life at any temperature that are produced by the methods provided herein are contemplated herein.

As used herein, “a biologically active agent, “an active agent,” “a biological agent,” or “an agent,” is any substance which when introduced into the body causes a desired biological response, such as altering body function at the cellular, tissue or organ level and/or altering cosmetic appearance, such as body weight and shape. Such substance can be any synthetic or natural element or compound, protein, cell, or tissue including a pharmaceutical, drug, therapeutic, nutritional supplement, herb, hormone, or the like, or any combinations thereof. The terms also encompass pharmaceutically acceptable, pharmacologically active derivatives of those active agents specifically mentioned herein, including, but not limited to, salts, esters, amides, prodrugs, active metabolites, isomers, fragments, analogs, and the like. When the terms “biologically active agent,” “biological agent” and “agent” are used, then, or when a particular active agent is specifically identified, it is intended to include the active agent per se as well as pharmaceutically acceptable, pharmacologically active salts, esters, amides, prodrugs, active metabolites, isomers, fragments and analogs.

As used herein, a “subject” is defined as an animal, including a mammal, typically a human.

As used herein, “therapeutically effective amount” refers to an amount of the active agent for a desired therapeutic, prophylactic, or other biological effect or response when a composition is administered to a subject in a single dosage form. The particular amount of active agent in a dosage will vary widely according to conditions such as the nature of the active agent, the nature of the condition being treated, the age and size of the subject.

As used herein, “pharmaceutically acceptable derivatives” of a compound include salts, esters, enol ethers, enol esters, acids, bases, solvates, hydrates or prodrugs thereof. Such derivatives can be readily prepared by those of skill in this art using known methods for such derivatization. The compounds produced can be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

As used herein, “treatment” means any manner in which one or more of the symptoms of a condition, disorder or disease are ameliorated or otherwise beneficially altered. Treatment also encompasses any pharmaceutical use of the compositions herein, such as use for treating influenza.

As used herein, “organic solvent” refers to a solvent that is an organic compound, which is any member of a large class of chemical compounds whose molecules contain carbon and hydrogen. Such solvents can include, for example, compounds from the following classes: aliphatic or aromatic alcohols, polyols, aldehydes, alkanes, alkenes, alkynes, amides, amines, aromatics, azo compounds, carboxylic acids, esters, dioxanes, ethers, haloalkanes, imines, imides, ketones, nitriles, phenols and thiols.

As used herein, an “aqueous solvent” refers to water, or a mixture of solvents that contains at least about 50% or 50%, at least about 60% or 60%, at least about 70% or 70%, or about or at 75%, 80%, 85%, 90%, 95%, 96%, 97%, 98%, 99% or higher amounts of water. The term “aqueous solvent” as used herein also refers to solutions containing water as a solvent, such as buffers, salt solutions, solutions containing counterions, and other solutes that are soluble in water.

As used herein, “antisolvent” means a solvent which, when added to a solution of the microparticle-forming compound of interest, lowers the solubility of the compound in the resulting mixture (i.e., the “cocktail solution” from which the microparticles are eventually obtained). The antisolvent generally is added in an amount that retains the compound in solution until the microparticles are formed by a step of gradual chilling followed by microparticle recovery, e.g., by lyophilization. Thus, the antisolvent is added to the solution of the compound in an amount that is insufficient to precipitate the compound out of solution at the temperature (generally, ambient temperature) used to prepare the cocktail solution. The antisolvent can be miscible or partially miscible with the solvent in which the compound is dissolved, or the solvent/counterion solution, or the solvent/counterion/compound solution. For example, an organic solvent such as isopropanol can be an antisolvent for compounds that are water-soluble, and water or an aqueous buffer can be an antisolvent for compounds that are water-insoluble. Both solvent and antisolvent, however, can be organic solvents. Some antisolvents and solvents can also serve as counterions. For example, aqueous buffered solutions can be a counterion and a solvent or antisolvent. Similarly, a polymer, such as polyethylene glycol (PEG) or polyethyleneimine (PEI), can be an antisolvent and a counterion.

As used herein, the term “solvent/antisolvent system” means a mixture of solvents in which a compound that can form a microsphere is soluble at ambient temperature, but forms microspheres upon chilling of the mixture, generally in the presence of a counterion, to temperatures below ambient temperature. As noted above, a solvent and/or an antisolvent can also be a counterion and eliminate the need for an additional counterion. The solvent and the antisolvent generally are miscible or partially miscible with one another, although solvent/antisolvent systems in which the solvent and antisolvent are immiscible also can be used.

As used herein, the term “pl” or “isoelectric point” refers to the pH at which there is no net charge on a protein or polypeptide.

As used herein, the term “counterion” refers to a charged or charge-polarizable molecule that can initiate formation of a microparticle from a macromolecule, such as a protein, nucleic acid, lipid or oligosaccharide, or from a small molecule, such as a tetracycline or prostaglandin. A counterion can be a polymer, such as polyethylene glycol (PEG) or polyethyleneimine (PEI).

The choice of counterion can empirically be determined for each compound (macromolecule or small molecule) of interest. For example, in the case of the DAS181 fusion protein (SEQ ID NO:17), sodium sulfate is a counterion because it can initiate the formation of microparticles in the methods provided herein, whereas glycine, sodium chloride or sodium acetate generally are not suitable as counterions for DAS181. For kanamycin, itaconic and citric acids can serve as suitable counterions because they can initiate the formation of microparticles of kanamycin in the methods provided herein, whereas arginine generally is not suitable as a counterion for kanamycin.

Whether a charged molecule is a counterion can be determined empirically based on parameters including, but not limited to, the type of molecule to be formulated into a microsphere, the pH, the ionic strength, the type of solvent/antisolvent system used, and the presence of salts and additional ingredients such as active agents. As provided and described herein, counterions can be anionic or having a net negative charge or charge-polarizable group(s), cationic or having a net positive charge or charge-polarizable group(s), or zwitterionic and possessing both negative and positive charged or charge-polarizable groups.

A compound can sometimes be its own counterion, facilitating the formation of microparticles in the absence of any additional counterion. For example, under certain conditions, small molecule compounds such as tetracycline, kanamycin and ampicillin, and macromolecules such as siRNA and tobacco mosaic virus, can form microparticles in the absence of added counterion. Other counterions, such as polyethyleneimine (PEI) and Na-acetate/Na-sulfate buffer, which are capable of forming microparticles on their own, in the absence of a compound of interest, can facilitate formation and/or nucleation of microparticles of the compound of interest by acting as “carriers” or “seeds.”

As used herein, the term “cooling” refers to a lowering of temperature to a desired temperature for obtaining microparticles or, once the microparticles of desired dimensions are obtained, further lowering the temperature to a desired temperature for obtaining dry preparations of the microparticles by volatilizing solvents (e.g., for freeze-drying). The term “gradual cooling” or “gradually cooling” or “gradually cooled” as used herein means that the lowering of temperature to a desired temperature from ambient temperature (about or at 15° C. to about or at 50° C., generally about or at 18° C. to about or at 30° C.) for microparticle formation occurs at a rate or for an amount of time that is suitable for generating microparticles in a solution before the solution becomes frozen. Thus gradual cooling is different from, for example, snap freezing, spray drying or spray freeze-drying, whereby the entire solution is converted to a solid form without the generation of distinct microparticles.

The rate of gradual cooling is empirically determined based on the type of macromolecule, solvents, counterions and other ingredients as well as the method of cooling (e.g., an endothermic reaction, a heat exchanger, refrigerator or freezer or freeze-dryer) and can vary, for example, for an amount of time for microparticle formation of between about or at 1 min, 2 min, 3 min, 5 min, 7 min, 10 min, 15 min, 20 min, 25 min, 30 min, 1 h, 2 h, 5 h or 10 h to about or at 1.5 min, 2 min, 3 min, 5 min, 7 min, 10 min, 15 min, 20 min, 25 min, 30 min, 1 h, 2 h, 5 h, 10 h or 15 h.

Microparticles of desired size also can be formed, for example, by rapidly chilling the cocktail (e.g. using a heat exchanger) and allowing the suspension of microparticles to be maintained for a certain period of time without significant temperature changes, then snap freezing the cocktail.

The temperature at which microparticles are formed also is empirically determined based on the type of macromolecule or small molecule, solvents, counterions and other ingredients as well as the method and uniformity of cooling and can vary from about or at 15° C., 10° C., 8° C., 5° C., 4 C, 3° C., 2° C., 1° C., −2° C., −5° C., −7.5° C., −10° C., −15° C., −20° C., −25° C., −30° C., −35° C., −40° C., −45° C., −50° C., −55° C., −60° C., −70° C., −80° C., −85° C., −90° C., −100° C., −110° C., −115° C., −120° C., −125° C., −135° C., −145° C., −150° C., −160° C., −165° C., −170° C., −175° C., −180° C., −185° C., −190° C., −195° C., or −200° C.

The term “ambient temperature” is sometimes used interchangeably herein with “room temperature” and refers to the temperature of air or other media in the environment of the designated area in which the cocktail reactions are mixed and/or are maintained prior to the initiation of microsphere formation. Ambient temperature as used herein can be from about or at 15° C. to about or at 50° C., generally about or at 18° C. to about or at 30° C., or about or at 25° C. to about or at 30° C.

As used herein, an “endothermic reaction” is any chemical reaction that absorbs heat from its environment, e.g., in solution, thus cooling the surrounding environment or solution. For example, the addition of ammonium sulfate or acetonitrile to water results in an endothermic reaction; these compounds, therefore, can serve as counterion and antisolvent, respectively, and also facilitate chilling to form microparticles. Other examples of endothermic reactions include, but are not limited to, dissolving ammonium chloride in water, mixing water and ammonium nitrate, mixing water with potassium chloride, and reacting ethanoic acid with sodium carbonate.

As used herein, the term “spray drying” refers to a process wherein a solution containing a molecule, such as a protein or small molecule, is transformed into a dry particulate form by atomizing into a hot drying medium, generally for a period of about a few milliseconds to 1-2 seconds to a few tens of seconds. The term “spray freeze-drying” as used herein refers to a process wherein a solution containing a macromolecule, such as a protein, is atomized into a cryogenic medium, such as liquid nitrogen, to obtain frozen droplets of solution that can then be dried by lyophilization. The term “snap freezing” or “rapid freezing” or “quick freezing” or “flash freezing” as used interchangeably herein refers to freezing a solvent or solution, including solutions containing macromolecules, such as proteins, by immersing the container with good heat transfer properties (e.g. thin-wall glass or plastic or metal test tube) holding the solvent or solution in liquid nitrogen or pouring the solution directly into liquid nitrogen. “Snap freezing” and “rapid freezing” generally occur within a period of about a few milliseconds to 1-2 seconds to a few tens of seconds.

The term “lyophilize” or “lyophilization” as used herein is synonymous with “freeze drying” and refers to a process wherein a solution, including an emulsion, colloid or suspension, is frozen and the solvents are volatilized (sublimated) directly into the vapor state, leaving behind the solid components.

### B. Methods for Preparing Microparticle Compositions

Provided herein are methods of making microspheres having a high content of a compound. The compound can be macromolecule, such as a protein, or a small molecule, such as a prostaglandin. The microspheres provided herein are prepared by controlled precipitation in the presence of a counterion and an antisolvent. The microspheres are suitable for preparing pharmaceutical, diagnostic, nutraceutical or cosmetic compositions that can be delivered to subjects by a variety of delivery routes, including pulmonary, subcutaneous, transdermal, intramuscular, parenteral and oral administration routes. The method also can be performed in a batch or continuous mode, for increased efficiency and production.

The microspheres obtained by the methods provided herein are useful as prophylactic, therapeutic or diagnostic agents for treating or diagnosing disease states in a subject in vivo or in vitro. The sizes of the microspheres obtained by the methods provided herein can be controlled by adjusting parameters including type and concentration of antisolvent, types and relative concentrations of solvent and antisolvent in the solvent/antisolvent system, macromolecule or small molecule concentration, ionic strength, counterion type and concentration, rate and time of cooling, to provide microspheres in a wide range of sizes, from 0.001 micron to 50 microns or greater, that can deliver therapeutic agents via a desired route including pulmonary (exemplary sizes can include, but are not limited to, 1 micron to 5 micron particles for delivery to the throat, trachea and bronchi for treatment of influenza and other respiratory infections), subcutaneous, intramuscular, intravenous and other routes (using particles that can include, but are not limited to, particles that are tens of microns in size).

The compositions provided herein can be formulated for a variety of modes of administration. For example, the compositions can be orally e.g. by ingestion, intravenously, intranasally, parenterally, subcutaneously, transdermally, topically, cutaneously, intraarticularly or intramuscularly administered. The compositions also can be formulated for pulmonary or ophthalmic administration. In a certain aspect, the composition provided herein is for inhalation.

The compositions provided herein can be formulated as tablets, caplets, capsules, gels, vials, pre-filled syringes, inhalers, electrostatic devices and other devices for delivery. The delivery dosage of the compositions can be from between about or at 0.01 mg to about or at 0.1 mg; about or at 0.1 mg compound per dose to about or at 1000 mg compound per dose, or about or at 0.2 mg, 0.3 mg, 0.5 mg, 0.6 mg, 0.75 mg, 1 mg, 1.5 mg, 2 mg, 3 mg, 5 mg, 10 mg, 15 mg, 20 mg, 30 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 600 mg, 700 mg, 800 mg, 900 mg or about or at 1000 mg compound per dose. The frequency of administration of a dose, for example, for the treatment or prophylaxis of influenza, can be from three or more times a day, to two times a day, to once a day, to two times a week, to once a week, to once every two weeks or less frequent than once every two weeks. For prophylaxis, the administration generally can be of the order of about once every two weeks or less frequent, such as once every three weeks or once every four weeks or longer.

The compositions formulated according to the methods provided herein can be used for the prevention, prophylaxis and/or treatment of diseases and disorders. Accordingly, provided herein are methods of prevention, prophylaxis or treatment of a disease by administering a therapeutically effective amount of microspheres of a compound of interest. The diseases and disorders can include, but are not limited to neural disorders, respiratory disorders, immune system disorders, muscular disorders, reproductive disorders, gastrointestinal disorders, pulmonary disorders, digestive disorders, metabolic disorders, cardiovascular disorders, renal disorders, proliferative disorders, cancerous diseases and inflammation.

For example, the microparticles provided herein can be used in methods of treating Infectious diseases, such as arboviral infections, botulism, brucellosis, candidiasis, campylobacteriosis, chickenpox, chlamydia, cholera, coronovirus infections, staphylococcus infections, coxsackie virus infections, Creutzfeldt-Jakob disease, cryptosporidiosis, cyclospora infection, cytomegalovirus infections, Epstein-Barr virus infection, dengue fever, diphtheria, ear infections, encephalitis, influenza virus infections, parainfluenza virus infections giardiasis, gonorrhea, Haemophilus influenzae infections, hantavirus infections, viral hepatitis, herpes simplex virus infections, HIV/AIDS, helicobacter infection, human papillomavirus (HPV) infections, infectious mononucleosis, legionellosis, leprosy, leptospirosis, listeriosis, lyme disease, lymphocytic choriomeningitis, malaria, measles, marburg hemorrhagic fever, meningitis, monkeypox, mumps, mycobacteria infection, mycoplasma infection, norwalk virus infection, pertussis, pinworm infection, pneumococcal disease, Streptococcus pneumonia infection, Mycoplasma pneumoniae infection, Moraxella catarrhalis infection, Pseudomonas aeruginosa infection, rotavirus infection, psittacosis, rabies, respiratory syncytial virus infection, (RSV), ringworm, rocky mountain spotted fever, rubella, salmonellosis, SARS, scabies, sexually transmitted diseases, shigellosis, shingles, sporotrichosis, streptococcal infections, syphilis, tetanus, trichinosis, tuberculosis, tularemia, typhoid fever, viral meningitis, bacterial meningitis, west nile virus infection, yellow fever, adenovirus-mediated infections and diseases, retrovirus-mediated infectious diseases, yersiniosis zoonoses, and any other infectious respiratory, pulmonary, dermatological, gastrointestinal and urinary tract diseases.

Other diseases and conditions that can be treated by administering a therapeutically effective amount of microspheres of a compound of interest can include arthritis, asthma, allergic conditions, Alzheimer's disease, cancers, cardiovascular disease, multiple sclerosis (MS), Parkinson's disease, cystic fibrosis (CF), diabetes, non-viral hepatitis, hemophilia, bleeding disorders, blood disorders, genetic disorders, hormonal disorders, drug addictions and dependencies, pain, kidney disease, liver disease, angiogenesis, pulmonary arterial hypertension, neurological disorders, metabolic diseases, skin conditions, thyroid disease, osteoporosis, obesity, stroke, anemia, inflammatory diseases and autoimmune diseases.

The steps of the method provided herein include: combining a solution containing the compound with a counterion and an antisolvent, and gradually cooling the resulting solution to a temperature whereby microparticles are formed. In one embodiment, the steps can be described as follows:

1) To a solution containing a compound dissolved in a suitable solvent, adding a counterion and an antisolvent at concentrations that do not cause precipitation of the compound at ambient temperature;

2) Precipitation: chilling the compound/counterion/antisolvent cocktail solution, via methods including chilling (heat-exchange) and endothermic reactions, to initiate formation of microspheres; and

3) Dehydration: freezing of the microsphere suspension and removal of antisolvent and water by sublimation (freeze-drying, e.g., at a temperature of about or at −5° C. to about or at −200° C.; or to about or at −20° C. to about or at −200° C., or about or at −30° C. to about or at −200° C., or about or at −40° C. to about or at −180° C., or about or at −45° C. to about or at −180° C., or about −65° C. to about −175° C., or about −80° C. to about or at −120° C., or about or at −65° C. to about or at −100° C.).

The above steps of the method can be performed sequentially, intermittently or simultaneously in any order, although one of the skill in the art would understand that the step of dehydration to separate the solvent from the microspheres can occur simultaneously with, or following, microsphere formation, but not prior to microsphere initiation and/or formation. In one embodiment, the counterion and the antisolvent are added simultaneously or sequentially in any order to the solution containing the compound, followed by chilling. In other embodiments, the same substance serves as the counterion and the antisolvent (for example, a polymer such as polyethylene glycol or polyethyleneimine). In yet other embodiments, the solution containing the compound can be pre-chilled to a temperature suitable for microsphere formation, prior to adding the counterion and antisolvent. Pre-chilling can be performed using a device, such as a refrigerator or freezer, or by endothermic reaction. For example, a pre-chilled aqueous solution of a compound can be formed by adding ammonium sulfate and acetonitrile, whose dissolution proceeds via an endothermic reaction, prior to or simultaneously with forming microspheres.

The resulting suspension of microparticles can be converted into a dry powder by further cooling to a temperature below freezing point and subsequent removal of volatiles (solvent, antisolvent and, where desired, the counterion) by, for example, sublimation using a standard freeze dryer.

In some embodiments, the addition of a counterion is not necessary. For example, under certain conditions, some molecules in solution with a suitable solvent can form microparticles in the presence of an antisolvent and no added counterion. Without being bound by any theory, it is possible that the molecules can act as counterions to themselves, or other components in the resulting cocktail solution or combinations thereof, such as the solvent, antisolvent. Several such molecules are exemplified herein, including siRNA, tobacco mosaic virus, tetracycline, kanamycin and ampicillin. Thus, also provided herein is a method of making microparticles by:

(a) adding an antisolvent to a solution of a compound in an a solvent; and
(b) gradually cooling the solution to a temperature below about 25° C., whereby a composition containing microparticles of the compound is formed, wherein steps (a) and (b) are performed simultaneously, sequentially, intermittently, or in any order.

In other embodiments, the microparticles can be formed in the absence of antisolvent. Thus, also provided herein is a method of making microparticles, by: (a) adding a counterion to a solution of a compound in a solvent; and (b) gradually cooling the solution to a temperature below about 25° C., whereby a composition containing microparticles of the compound is formed, wherein steps (a) and (b) are performed simultaneously, sequentially, intermittently, or in any order.

In one embodiment, the microspheres formed by contacting the compound with a counterion and antisolvent and exposed to low temperature, are separated from the suspension by methods including sedimentation or filtration techniques. After separation from the original precipitation mix, the microspheres can be washed and/or combined with other materials that improve and/or modify characteristics of the compounds and/or the microspheres.

In another embodiment, the microspheres prepared by the methods provided herein do not have a direct therapeutic effect, but serve as micro-carriers for other therapeutic agent(s) or active agent(s), including diagnostic markers and nutritional supplements. The additional agents can be added at the time of precipitation or can be added to the suspension of formed microspheres prior to lyophilization. Alternatively, the additional agents can be blended into dry powder containing microspheres.

Without being bound by any theory, in one aspect, the methods provided herein can permit the formation of microspheres by: (1) neutralization of charges on the surface of the compound by the counterion and (2) decreased solubility of the compound in the solvent, caused by the combined effects of added antisolvent and gradual cooling.

By choosing a suitable pH that is empirically determined and can be in the range of, for example, about or at 1.0 to about or at 14.0, generally about or at pH 2.0 to about or at 10.5 or greater, depending on the compound, counterion, and antisolvent, in the presence of a suitable amount of the counterion, a substantial number of the charged groups, in some embodiments all charged groups, on the surface of the compound can become neutralized. A decrease in the polarity of the solution by adding a suitable antisolvent can then initiate the formation of microspheres by precipitation, phase separation, colloid formation, or other such method.

Alternatively, without being bound by any theory, in some embodiments, the observed phenomenon of the precipitation of microspheres also can be explained by the kosmotropic (structure forming) effect of counterions and antisolvents due to interactions with the solvent containing the compound at low temperatures. Regardless of the underlying mechanism, in the methods provided herein, the addition of relatively small amounts of antisolvent and counterion to a solution containing a compound of interest (aqueous or polar solvent for polar compounds; non-polar or organic solvent for water-insoluble compounds) and cooling of the resulting cocktail solution results in the production of compositions containing microspheres of the compounds.

In one embodiment, gradual cooling chilling of the cocktail solution can be performed by passing the cocktail solution through a heat exchanger. The temperature of the heat exchanger and the flow rate of the cocktail through the heat exchanger can be adjusted so that the cocktail is either pre-chilled prior to formation of the microspheres, or is chilled to a temperature whereby microspheres are formed.

In another embodiment, the microspheres formed by the methods provided herein are concentrated or separated from the suspension by methods such as sedimentation or filtration techniques. Upon formation of the microspheres, their growth (size) can be controlled by adjusting the ionic strength, polarity, pH, or other parameters of the suspension. The separation of microspheres from the liquid phase of the cocktail solution can be performed by centrifugation, filtration (hollow fiber, tangential flow, etc.), or other techniques. The resulting microspheres or concentrated suspensions thereof can be lyophilized or air dried.

In some embodiments, the microspheres separated from the original precipitation mix or the dried microspheres can be reconstituted prior to administration as a therapeutic agent or a carrier, or can be suspended in solutions that contain agents that modify characteristics of the microspheres. The modifying agents can include but are not limited to bulking agents, excipients, inactive ingredients, stability enhancers, taste and/or odor modifiers or masking agents, vitamins, sugars, therapeutic agents, anti-oxidants, immuno-modulators, trans-membrane transport modifiers, anti-caking agents, enteric coating agents, agents that confer acid resistance, such as against the acids of the digestive system, agents that confer protease resistance, chitosans, polymers, and flowability enhancers.

The formation and characteristics of the microspheres produced by the methods provided herein can empirically be determined by varying parameters, including: nature and concentration of the compound, pH of the cocktail solution, nature and concentration of the counterion, nature and concentration of the antisolvent, ionic strength and the cooling rate by which gradual cooling is effected. The steps of the methods provided herein render the method amenable to high-throughput screening, such as in a microplate format, for determining suitable combinations of compound, antisolvent, counterion, pH, ionic strength and cooling ramp for the generation of microspheres.

Molecules

Any naturally occurring or synthetic molecule or compound that can form microparticles when in solution in the presence of one or more of a counterion and an antisolvent, is contemplated for use in the methods provided herein. The compound can be an inorganic compound, including alkali and alkaline earth metal compounds and salts and other derivatives thereof, transition metal compounds, including coordination compounds and salts and other derivatives thereof, inorganic polymers, such as polysiloxanes, and other such compounds known to those of skill in the art. Examples of inorganic compounds include some compounds that contain carbon, but generally no carbon-carbon bonds; for example, carbon monoxide, carbon dioxide, carbonates, cyanides, cyanates, carbides, and thiocyanates. Other inorganic compounds include compounds formed from elements of the periodic table other than carbon. For example, any metal (alkali metal, alkaline earth metal, transition metal, e.g.) carbonates, cyanides, cyanates, carbides, halides (F, Cl, Br, I), thiocyanates, selenocyanate, azides, oxides, hydroxides, sulfides and hydrozides, coordination compounds, organometallic compounds, and other such compounds as understood by those of skill in the art.

Other classes of inorganic compounds are studied and developed by chemists trained in materials science, for example, polymeric and/or refractory materials such as silicon and gallium arsenide, yttrium barium copper oxide, polymers such silicones, polysilanes, polygermanes, polystannanes and polyphospahazenes.

The compound can be an organic compound, including aliphatic, aromatic and alicyclic alcohols, aldehydes, carboxylic acids, esters, ketones, ethers, amines, amides, lactams, polymers thereof, and other such compounds known to those of skill in the art. Examples of organic compounds, which can be aliphatic, aromatic or alicyclic, can be any of the following, and similar classes of compounds known and understood by those of skill in the art:

“Alkyl” refers to straight or branched chain substituted or unsubstituted hydrocarbon groups, generally from about 1 to 40 carbon atoms, 1 to 20 carbon atoms, or 1 to 10 carbon atoms. “Lower alkyl” generally is an alkyl group of 1 to 6 carbon atoms. An alkyl group can be a “saturated alkyl,” meaning that it does not contain any alkene or alkyne groups, or an alkyl group can be an “unsaturated alkyl,” meaning that it contains at least one alkene or alkyne group. An alkyl group that includes at least one carbon-carbon double bond (C═C) is referred to by the term “alkenyl,” and an alkyl group that includes at least one carbon-carbon triple bond (C≡C) is referred to by the term “alkynyl.” and in certain embodiments, alkynyl groups are optionally substituted. Alkyls include, but are not limited to, methyl, ethyl, propyl, isopropyl, butyl, isobutyl, tertiary butyl, pentyl, hexyl, ethenyl, propenyl, butenyl, hexenyl, ethynyl, propynyl, butynyl, hexynyl, haloalkyl and heteroalkyl.

“Cycloalkyl,” i.e. a saturated mono- or multicyclic ring system where each of the atoms forming a ring is a carbon atom. Cycloalkyls can be formed by three, four, five, six, seven, eight, nine, or more than nine carbon atoms. The ring system generally includes about 3 to about 12 carbon atoms. The term “cycloalkyl” includes rings that contain one or more unsaturated bonds, and those that are substituted. Examples of cycloalkyls include, but are not limited to, cyclopropane, cyclobutane, cyclopentane, cyclopentene, cyclopentadiene, cyclohexane, cyclohexene, 1,3-cyclohexadiene, 1,4-cyclohexadiene, cycloheptane and cycloheptene.

“Heterocyclic” compounds, which are rings where at least one atom forming the ring is a carbon atom and at least one atom forming the ring is a heteroatom.

“Bicyclic ring,” which refers to two rings that are fused. Bicyclic rings include, for example, decaline, pentalene, naphthalene, azulene, heptalene, isobenzofuran, chromene, indolizine, isoindole, indole, purine, indoline, indene, quinolizine, isoquinoline, quinoline, phthalazine, naphthyrididine, quinoxaline, cinnoline, pteridine, isochroman, chroman and various hydrogenated derivatives thereof. Bicyclic rings can be optionally substituted. Each ring is independently aromatic or non-aromatic.

“Aromatic” compounds, such as phenyl, naphthalenyl, phenanthrenyl, anthracenyl, tetralinyl, fluorenyl, indenyl and indanyl. Aromatic compounds include benzenoid groups, connected via one of the ring-forming carbon atoms, and optionally carrying one or more substituents selected from an aryl, a heteroaryl, a cycloalkyl, a non-aromatic heterocycle, a halo, a hydroxy, an amino, a cyano, a nitro, an alkylamido, an acyl, a C₁₋₆ alkoxy, a C₁₋₆ alkyl, a C₁₋₆ hydroxyalkyl, a C₁₋₆ aminoalkyl, a C₁₋₆ alkylamino, an alkylsulfenyl, an alkylsulfinyl, an alkylsulfonyl, an sulfamoyl, or a trifluoromethyl. An aromatic group can be substituted at one or more of the para, meta, and/or ortho positions. Examples of aromatic groups containing substitutions include, but are not limited to, phenyl, 3-halophenyl, 4-halophenyl, 3-hydroxyphenyl, 4-hydroxy-phenyl, 3-aminophenyl, 4-aminophenyl, 3-methylphenyl, 4-methylphenyl, 3-methoxyphenyl, 4-methoxyphenyl, 4-trifluoromethoxyphenyl, 3-cyano-phenyl, 4-cyanophenyl, dimethylphenyl, naphthyl, hydroxynaphthyl, hydroxymethyl-phenyl, (trifluoromethyl)phenyl, alkoxyphenyl, 4-morpholin-4-ylphenyl, 4-pyrrolidin-1-ylphenyl, 4-pyrazolylphenyl, 4 triazolylphenyl and 4-(2-oxopyrrolidin-1-yl)phenyl.

“Aryl” compounds, which are monocyclic, bicyclic or tricyclic aromatic systems that contain no ring heteroatoms. Examples of aryl include phenyl, naphthyl, anthracyl, indanyl, 1,2-dihydro-naphthyl, 1,4-dihydronaphthyl, indenyl, 1,4-naphthoquinonyl and 1,2,3,4-tetrahydronaphthyl.

“Heteroaryl” compounds, which refer to an aromatic ring in which at least one atom forming the aromatic ring is a heteroatom. Such groups include oxazolyl, isoxazolyl, oxadiazolyl, thiazolyl, isothiazolyl, pyridinyl, pyridazinyl, pyrimidinyl, pyrazinyl, indolyl, benzimidazolyl, quinolinyl, isoquinolinyl, quinazolinyl, quinoxalinyl, pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. The heteroaryl compounds can be in the form of bicyclic radicals, and/or can optionally be substituted. Examples of substituents include halo, hydroxy, amino, cyano, nitro, alkylamido, acyl, C₁₋₆-alkoxy, C₁₋₆-hydroxy-alkyl, C₁₋₆-aminoalkyl, C₁₋₆-alkylamino, alkylsulfenyl, alkylsulfinyl, alkylsulfonyl, sulfamoyl, or trifluoromethyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. Substituents can be, for example, halo, hydroxy, cyano, O—C₁₋₆-alkyl, C₁₋₆-alkyl, hydroxy-C₁₋₆-alkyl and amino C₁₋₆-alkyl.

“Non-aromatic heterocycle”, i.e., a non-aromatic ring wherein one or more atoms forming the ring is a heteroatom. Non-aromatic heterocyclic rings can be formed by three, four, five, six, seven, eight, nine, or more than nine atoms. Non-aromatic heterocycles can be optionally substituted. Examples of non-aromatic heterocycles include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine and 1,3-oxathiolane.

“Arylalkyl” compounds refer to an alkyl substituted with an aryl that can be optionally substituted.

“Heteroarylalkyl” compounds to an alkyl substituted with a heteroaryl that can be optionally substituted.

The substituent groups on organic compounds can be one of several, including: “Amino” compounds refer to those containing a group of —NH₂; “Hydroxy” refers to a group of —OH; “Nitro” refers to a group of —NO₂; “O carboxy” refers to a group of formula RC(═O)O—; “C carboxy” refers to a group of formula C(═O)OR; “alkoxy” refers to a group of formula —OR; “acetyl” or “acyl” refers to a group of formula C(═O)CH₃; “cyano” refers to a group of formula CN; “nitrile” refers to a compound having the structure RC≡N; “isocyanato” refers to a group of formula NCO; “thiocyanato” refers to a group of formula CNS; “isothiocyanato” refers to a group of formula NCS; “C amido” refers to a group of formula C(═O)NR₂; “N amido” refers to a group of formula RC(═O)NR; “sulfenyl” refers to a group of formula —SR; “sulfinyl” refers to a group of formula —S(═O)R; “sulfonyl” refers to a group of formula —S(═O)₂R; “sulfamoyl” refers to a group of formula —S(═O)₂NR₂; “sulfonyl halide” refers to compound of formula X—S(═O)₂R, where X is halo; “ester” refers to a group of formula RC(═O)OR′, where R′≠H; “amide” refers to a group of formula RC(═O)NR′₂.

Macromolecules and Small Molecules

The compounds used to form microparticles according to the methods provided herein can be macromolecules, or small molecules. The term “macromolecule” is understood by those of skill in the art, and generally refers to a naturally occurring or chemically synthetized organic or inorganic molecule whose molecular weight is greater than or equal to about a 1000 Daltons to about or greater than 1, 2, 3, 5, 7, 10 or more trillion Daltons, about 1000 or 1000 to about five billion or five billion, about 1000 or 1000 to about one billion or one billion, about 1000 or 1000 to about 50 million or 50 million, about 1000 or 1000 to about 20 million or 20 million, about 1000 or 1000 to about 15 million or 15 million, about 1000 or 1000 to about 10 million or 10 million, about 1000 or 1000 to about 5 million or 5 million, about 1000 or 1000 to about one million or one million, about 1000 or 1000 to about 500,000 or 500,000, about 1000 or 1000 to about 300,000 or 300,000, about 1000 or 1000 to about 200,000 or 200,000, about 1000 or 1000 to about 100,000 or 100,000, about 1000 or 1000 to about 50,000 or 50,000, about 1000 or 1000 to about 25,000 or 25,000, about 1000 or 1000 to about 15,000 or 15,000, about 1000 or 1000 to about 10,000 or 10,000, about 1000 or 1000 to about 5,000 or 5,000, about 1000 or 1000 to about 3,000 or 3000, or about 1000 or 1000 to about 2,000 or 2000 Daltons. Examples of macromolecules include proteins, peptides, nucleic acids, including DNA, RNA, siRNA, snRNA, antisense RNA, and ribozymes, carbohydrates, lipids, fatty acids, polysaccharides, protein conjugates, viruses, virus particles, hormones, carbohydrate- or polysaccharide-protein conjugates, viroids, prions and mixtures thereof.

The term “small molecule” is used herein in the sense that is understood by those of skill in the art, and generally refers to a naturally occurring or chemically synthetized organic or inorganic molecule that is less than about 1000 Daltons, from about or at 1000 Daltons to about or at 950, 900, 850, 800, 750, 700, 650, 600, 550, 500, 450, 400, 375, 350, 325, 300, 275, 250, 225, 200, 175, 150, 125, 100, 75, 70, 65, 60, 55, 50, 45, 40, 35, 30, 25, 20, 15, 10, 5 or less Daltons. A small molecule is understood to mean any molecule that is not a macromolecule, such as a protein or nucleic acid, nor a macrmolecular assembly, such as a virus. A “small molecule” as used herein can include a molecule containing two or more monomeric subunits, such as a dipeptide or dinucleotide, and generally is understood to refer to molecules that are about or at 1000 Daltons or below in molecular weight. Examples of small molecules include, but are not limited to, inorganic molecules such as, but not limited to, carbon monoxide, carbon dioxide, metal (alkali metal, alkaline earth metal, transition metal, e.g.) carbonates, cyanides, cyanates, carbides, halides, thiocyanates, oxides, hydroxides, sulfides and hydrozide, coordination compounds, e.g., the cobalt salt [Co(NH₃)₆]Cl₃, and organometallic compounds, e.g. Fe(C₅H₅)₂. Small molecules that are organic compounds include, for example, nucleotides, amino acids, pteridines such as Furterene and Triamterene; purines such as Acefylline, 7-Morpholinomethyltheophylline, Pamabrom, Protheobromine and Theobromine; sterols such as cholesterol and lanosterol, steroids such as estrogen, testosterone, canrenone, oleandrin and spironolactone; penicillins, tetracyclines, sulfonamide derivatives such as Acetazolamide, Ambuside, Azosemide, Bumetanide, Butazolamide, Diphenylmethane-4.4′-disulfonamide, Disulfamide, Furosemide, uracils such as Aminometradine and Amisometradine, and the like, and prostaglandins.

The macromolecules and small molecules can further be inorganic compounds or organic compounds, as discussed above, or combinations thereof. In addition, the macromolecules and small molecules can have a variety of functional applications, such as therapeutic agents, diagnostic agents, nutritional supplements and other active agents. Macromolecule and small molecule agents that can be formulated into microparticles according to the methods provided herein include, for example, antibiotics, chemotherapeutic agents, vaccines, hematopoietics, anti-infective agents, antiulcer agents, antiallergic agents, antipyretics, analgesics, anti-inflammatory agents, antidementia agents, antiviral agents, antitumoral agents, antidepressants, psychotropic agents, cardiotonics, antiarrhythmic agents, vasodilators, antihypertensive agents, antidiabetic agents, anticoagulants, cholesterol lowering agents, diagnostic markers, and nutritional supplements, including herbal supplements.

The macromolecule and small molecule agents additionally can be selected from inorganic and organic drugs including, but not limited to drugs that act on the peripheral nerves, adrenergic receptors, cholinergic receptors, nervous system, skeletal muscles, cardiovascular system, smooth muscles, blood circulatory system, synaptic sites, neuro-effector junctional sites, endocrine system, hormone systems, immunological system, reproductive system, skeletal system, autocoid systems, alimentary and excretory systems, histamine systems, and the like. The active agents that can be delivered using the compositions provided herein include, but are not limited to, anticonvulsants, analgesics, antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids, sympathomimetics, parasympathomimetics, tranquilizers, urinary tract drugs, vaccines, vaginal drugs, vitamins, minerals, nonsteroidal anti-inflammatory drugs, angiotensin converting enzymes, polynucleotides, polypeptides and polysaccharides.

Exemplary agents that are macromolecules or small molecules that can be used to form microparticles according to the methods provided herein include:

### Exemplary Active Agent Categories for Macromolecules and Small Molecules

α-Adrenergic agonists such as Adrafinil, Adrenolone, Amidephrine, Apraclonidine, Budralazine, Clonidine, Cyclopentamine, Detomidine, Dimetofrine, Dipivefrin, Ephedrine, Epinephrine, Fenoxazoline, Guanabenz, Guanfacine, Hydroxyamphetamine, Ibopamine, Indanazoline, Isometheptene, Mephentermine, Metaraminol, Methoxamine Hydrochloride, Methylhexaneamine, Metizolene, Midodrine, Naphazoline, Norepinephrine, Norfenefrine, Octodrine, Octopamine, Oxymetazoline, Phenylephrine Hydrochloride, Phenylpropanolamine Hydrochloride, Phenylpropylmethylamine, Pholedrine, Propylhexedrine, Pseudoephedrine, Rilmenidine, Synephrine, Tetrahydrozoline, Tiamenidine, Tramazoline, Tuaminoheptane, Tymazoline, Tyramine and Xylometazoline;

β-Adrenergic agonists such as Albuterol, Bambuterol, Bitolterol, Carbuterol, Clenbuterol, Clorprenaline, Denopamine, Dioxethedrine, Dopexamine, Ephedrine, Epinephrine, Etafedrine, Ethylnorepinephrine, Fenoterol, Formoterol, Hexoprenaline, Ibopamine, Isoetharine, Isoproterenal, Mabuterol, Metaproterenol, Methoxyphenamine, Oxyfedrine, Pirbuterol, Prenalterol, Procaterol, Protokylol, Reproterol, Rimiterol, Ritodrine, Soterenol, Terbuterol and Xamoterol;

α-Adrenergic blockers such as Amosulalol, Arotinolol, Dapiprazole, Doxazosin, Ergoloid Mesylates, Fenspiride, Indoramin, Labetalol, Nicergoline, Prazosin, Terazosin, Tolazoline, Trimazosin and Yohimbine;

β-Adrenergic blockers such as Acebutolol, Alprenolol, Amosulalol, Arotinolol, Atenolol, Befunolol, Betaxolol, Bevantolol, Bisoprolol, Bopindolol, Bucumolol, Befetolol, Bufuralol, Bunitrolol, Bupranolol, Butidrine Hydrochloride, Butofilolol, Carazolol, Carteolol, Carvedilol, Celiprolol, Cetamolol, Cloranolol, Dilevalol, Epanolol, Esmolol, Indenolol, Labetalol, Levobunolol, Mepindolol, Metipranalol, Metoprolol, Moprolol, Nadoxolol, Nifenalol, Nipradilol, Oxprenolol, Penbutolol, Pindolol, Practolol, Pronethalol, Propranolol, Sotalol, Sulfinalol, Talinolol, Tertatolol, Timolol, Toliprolol and Xibenolol;

Alcohol deterrents such as Calcium Cyanamide Citrated, Disulfuram, Nadide and Nitrefazole;

Aldose reductase inhibitors such as Epalrestat, Ponalrestat, Sorbinil and Tolrestat;

Anabolics such as Androisoxazole, Androstenediol, Bolandiol, Bolasterone, Clostebol, Ethylestrenol; Formyldienolone, 4-Hydroxy-19-nortestosterone, Methandriol, Methenolone, Methyltrienolone, Nandrolone, Nandrolone Decanoate, Nandrolone p-Hexyloxyphenylpropionate, Nandrolone Phenpropionate, Norbolethone, Oxymesterone, Pizotyline, Quinbolone, Stenbolone and Trenbolone;

Analgesics (dental) such as Chlorobutanol, Clove and Eugenol;

Analgesics (narcotic) such as Alfentanil, Allylprodine, Alphaprodine, Anileridine, Benzylmorphine, Bezitramide, Buprenorphine, Butorphanol, Clonitazene, Codeine, Codeine Methyl Bromide, Codeine Phosphate, Codeine Sulfate, Desomorphine, Dextromoramide, Dezocine, Diampromide, Dihydrocodeine, Dihydrocodeinone Enol Acetate, Dihydromorphine, Dimenoxadol, Dimepheptanol, Dimethylthiambutene, Dioxaphetyl Butyrate, Dipipanone, Eptazocine, Ethoheptazine, Ethylmethlythiambutene, Ethylmorphine, Etonitazene, Fentanyl, Hydrocodone, Hydrocodone Bitartrate, Hydromorphone, Hydroxypethidine, Isomethadone, Ketobemidone, Levorphanol, Lofentanil, Meperidine, Meptazinol, Metazocine, Methadone Hydrochloride, Metopon, Morphine, Morphine Derivatives, Myrophine, Nalbuphine, Narceine, Nicomorphine, Norlevorphanol, Normethadone, Normorphine, Norpipanone, Opium, Oxycodone, Oxymorphone, Papavereturn, Pentazocine, Phenadoxone, Phenazocine, Pheoperidine, Piminodine, Piritramide, Proheptazine, Promedol, Properidine, Propiram, Propoxyphene, Sufentanil and Tilidine;

Analgesics (non-narcotic) such as Acetaminophen, Acetaminosalol, Acetanilide, Acetylsalicylsalicylic Acid, Alclofenac, Alminoprofen, Aloxiprin, Aluminum Bis(acetylsalicylate), Aminochlorthenoxazin, 2-Amino-4-picoline, Aminopropylori, Aminopyrine, Ammonium Salicylate, Antipyrine, Antipyrine Salicylate, Antrafenine, Apazone, Aspirin, Benorylate, Benoxaprofen, Benzpiperylon, Benzydamine, p-Bromoacetanilide, 5-Bromosalicylic Acid Acetate, Bucetin, Bufexamac, Bumadizon, Butacetin, Calcium Acetylsalicylate, Carbamazepine, Carbetidine, Carbiphene, Carsalam, Chloralantipyrine, Chlorthenoxazin(e), Choline Salicylate, Cinchophen, Ciramadol, Clometacin, Cropropamide, Crotethamide, Dexoxadrol, Difenamizole, Diflunisal, Dihydroxyaluminum Acetylsalicylate, Dipyrocetyl, Dipyrone, Emorfazone, Enfenamic Acid, Epirizole, Etersalate, Ethenzamide, Ethoxazene, Etodolac, Felbinac, Fenoprofen, Floctafenine, Flufenamic Acid, Fluoresone, Flupirtine, Fluproquazone, Flurbiprofen, Fosfosal, Gentisic Acid, Glafenine, Ibufenac, Imidazole Salicylate, Indomethacin, Indoprofen, Isofezolac, Isoladol, Isonixin, Ketoprofen, Ketorolac, p-Lactophenetide, Lefetamine, Loxoprofen, Lysine Acetylsalicylate, Magnesium Acetylsalicylate, Methotrimeprazine, Metofoline, Miroprofen, Morazone, Morpholine Salicylate, Naproxen, Nefopam, Nifenazone, 5′ Nitro-2′ propoxyacetanilide, Parsalmide, Perisoxal, Phenacetin, Phenazopyridine Hydrochloride, Phenocoll, Phenopyrazone, Phenyl Acetylsalicylate, Phenyl Salicylate, Phenyramidol, Pipebuzone, Piperylone, Prodilidine, Propacetamol, Propyphenazone, Proxazole, Quinine Salicylate, Ramifenazone, Rimazolium Metilsulfate, Salacetamide, Salicin, Salicylamide, Salicylamide O-Acetic Acid, Salicylsulfuric Acid, Salsalte, Salverine, Simetride, Sodium Salicylate, Sulfamipyrine, Suprofen, Talniflumate, Tenoxicam, Terofenamate, Tetradrine, Tinoridine, Tolfenamic Acid, Tolpronine, Tramadol, Viminol, Xenbucin and Zomepirac;

Androgens such as Androsterone, Boldenone, Dehydroepiandrosterone, Fluoxymesterone, Mestanolone, Mesterolone, Methandrostenolone, 17-Methyltestosterone, 17α-Methyltestosterone 3-Cyclopentyl Enol Ether, Norethandrolone, Normethandrone, Oxandrolone, Oxymesterone, Oxymetholone, Prasterone, Stanlolone, Stanozolol, Testosterone, Testosterone 17-Chloral Hemiacetal, Testosterone 17β-Cypionate, Testosterone Enanthate, Testosterone Nicotinate, Testosterone Pheynylacetate, Testosterone Propionate and Tiomesterone;

Anesthetics such as Acetamidoeugenol, Alfadolone Acetate, Alfaxalone, Amucaine, Amolanone, Amylocalne Hydrochloride, Benoxinate, Benzocaine, Betoxycaine, Biphenamine, Bupivacaine, Butacaine, Butaben, Butanilicaine, Burethamine, Buthalital Sodium, Butoxycaine, Carticaine, 2-Chloroprocaine Hydrochloride, Cocaethylene, Cocaine, Cyclomethycaine, Dibucaine Hydrochloride, Dimethisoquin, Dimethocaine, Diperadon Hydrochloride, Dyclonine, Ecgonidine, Ecgonine, Ethyl Aminobenzoate, Ethyl Chloride, Etidocaine, Etoxadrol, β-Eucaine, Euprocin, Fenalcomine, Fomocaine, Hexobarbital, Hexylcaine Hydrochloride, Hydroxydione Sodium, Hydroxyprocaine, Hydroxytetracaine, Isobutyl p-Aminobenzoate, Kentamine, Leucinocaine Mesylate, Levoxadrol, Lidocaine, Mepivacaine, Meprylcaine Hydrochloride, Metabutoxycaine Hydrochloride, Methohexital Sodium, Methyl Chloride, Midazolam, Myrtecaine, Naepaine, Octacaine, Orthocaine, Oxethazaine, Parethoxycaine, Phenacaine Hydrochloride, Phencyclidine, Phenol, Piperocaine, Piridocaine, Polidocanol, Pramoxine, Prilocalne, Procaine, Propanidid, Propanocaine, Proparacaine, Propipocaine, Propofol, Propoxycaine Hydrochloride, Pseudococaine, Pyrrocaine, Quinine Urea Hydrochloride, Risocaine, Salicyl Alcohol, Tetracaine Hydrochloride, Thialbarbital, Thimylal, Thiobutabarbital, Thiopental Sodium, Tolycaine, Trimecaine and Zolamine;

Anorexics such as Aminorex, Amphecloral, Amphetamine, Benzaphetamine, Chlorphentermine, Clobenzorex, Cloforex, Clortermine, Cyclexedrine, Destroamphetamine Sulfate, Diethylpropion, Diphemethoxidine, N-Ethylamphetamine, Fenbutrazate, Fenfluramine, Fenproporex, Furfurylmethylamphetamine, Levophacetoperate, Mazindol, Mefenorex, Metamfeproamone, Methamphetamine, Norpseudoephedrine, Phendimetrazine, Phendimetrazine Tartrate, Phenmetrazine, Phenpentermine, Phenylpropanolamine Hydrochloride and Picilorex;

Anthelmintics (Cestodes) such as Arecoline, Aspidin, Aspidinol, Dichlorophen(e), Embelin, Kosin, Napthalene, Niclosamide, Pellertierine, Pellertierine Tannate and Quinacrine;

Anthelmintics (Nematodes) such as Alantolactone, Amoscanate, Ascaridole, Bephenium, Bitoscanate, Carbon Tetrachloride, Carvacrol, Cyclobendazole, Diethylcarbamazine, Diphenane, Dithiazanine Iodide, Dymanthine, Gentian Violet, 4-Hexylresorcinol, Kainic Acid, Mebendazole, 2-Napthol, Oxantel, Papain, piperazine, piperazine Adipate, piperazine Citrate, piperazine Edetate Calcium, piperazine Tartrate, Pyrantel, Pyrvinium Pamoate, α-Santonin, Stilbazium Iodide, Tetrachloroethylene, Tetramisole, thiabendazole, Thymol, Thymyl N-Isoamylcarbamate, Triclofenol piperazine and Urea Stibamine;

Anthelmintics (Onchocerca) such as Ivermectin and Suramin Sodium;

Anthelmintics (Schistosoma) such as Amoscanate, Amphotalide, Antimony Potassium Tartrate, Antimony Sodium Gluconate, Antimony Sodium Tartrate, Antimony Sodium Thioglycollate, Antimony Thioglycollamide, Becanthone, Hycanthone, Lucanthone Hydrochloride, Niridazole, Oxamniquine, Praziquantel, Stibocaptate, Stibophen and Urea Stibamine;

Anthelmintic (Trematodes) such as Anthiol imine and Tetrachloroethylene;

Antiacne drugs such as Adapelene, Algestone Acetophenide, Azelaic Acid, Benzoyl Peroxide, Cyoctol, Cyproterone, Motretinide, Resorcinol, Retinoic Acid, Tetroquinone and Tretinonine;

Antiallergics such as Amlexanox, Astemizole, Azelastine, Cromolyn, Fenpiprane, Histamine, Ibudilast, Nedocromil, Oxatomide, Pentigetide, Poison Ivy Extract, Poison Oak Extract, Poison Sumac Extract, Repirinast, Tranilast, Traxanox and Urushiol;

Antiamebics such as Arsthinol, Bialamicol, Carbarsone, Cephaeline, Chlorbetamide, Chloroquine, Chlorphenoxamide, Chlortetracycline, Dehydroemetine, Dibromopropamidine, Diloxanide, Dephetarsone, Emetine, Fumagillin, Glaucarubin, Glycobiarsol, 8-Hydroxy-7-iodo-5-quinolinesulfonic Acid, Iodochlorhydroxyquin, Iodoquinol, Paromomycin, Phanquinone, Phearsone Sulfoxylate, Polybenzarsol, Propamidine, Quinfamide, Secnidazole, Sulfarside, Teclozan, Tetracycline, Thiocarbamizine, Thiocarbarsone and Timidazole;

Antiandrogens such as Bifluranol, Cyoctol, Cyproterone, Delmadinone Acetate, Flutimide, Nilutamide and Oxendolone;

Antianginals such as Acebutolol, Alprenolol, Amiodarone, Amlodipine, Arotinolol, Atenolol, Bepridil, Bevantolol, Bucumolol, Bufetolol, Bufuralol, Bunitrolol, Bupranolol, Carozolol, Carteolol, Carvedilol, Celiprolol, Cinepazet Maleate, Diltiazem, Epanolol, Felodipine, Gallopamil, Imolamine, Indenolol, Isosorbide Dinitrate, Isradipine, Limaprost, Mepindolol, Metoprolol, Molsidomine, Nadolol, Nicardipine, Nifedipine, Nifenalol, Nilvadipine, Nipradilol, Nisoldipine, Nitroglycerin, Oxprenolol, Oxyfedrine, Ozagrel, Penbutolol, Pentaerythritol Tetranitrate, Pindolol, Pronethalol, Propranolol, Sotalol, Terodiline, Timolol, Toliprolol and Verapamil;

Antiarrhythmics such as Acebutol, Acecaine, Adenosine, Ajmaline, Alprenolol, Amiodarone, Amoproxan, Aprindine, Arotinolol, Atenolol, Bevantolol, Bretylium Tosylate, Bubumolol, Bufetolol, Bunaftine, Bunitrolol, Bupranolol, Butidrine Hydrochloride, Butobendine, Capobenic Acid, Carazolol, Carteolol, Cifenline, Cloranolol, Disopyramide, Encamide, Esmolol, Flecamide, Gallopamil, Hydroquinidine, Indecamide, Indenolol, Ipratropium Bromide, Lidocaine, Lorajmine, Lorcamide, Meobentine, Metipranolol, Mexiletine, Moricizine, Nadoxolol, Nifenalol, Oxprenolol, Penbutolol, Pindolol, Pirmenol, Practolol, Prajmaline, Procainamide Hydrochloride, Pronethalol, Propafenone, Propranolol, Pyrinoline, Quinidine Sulfate, Quinidine, Sotalol, Talinolol, Timolol, Tocamide, Verapamil, Viquidil and Xibenolol;

Antiarteriosclerotics such as Pyridinol Carbamate;

Antiarthritic/Antirheumatics such as Allocupreide Sodium, Auranofin, Aurothioglucose, Aurothioglycanide, Azathioprine, Calcium 3-Aurothio-2-propanol-1-sulfonate, Celecoxib, Chloroquine, Clobuzarit, Cuproxoline, Diacerein, Glucosamine, Gold Sodium Thiomalate, Gold Sodium Thiosulfate, Hydroxychloroquine, Kebuzone, Lobenzarit, Melittin, Methotrexate, Myoral and Penicillamine;

Antibacterial (antibiotic) drugs including: Aminoglycosides such as Amikacin, Apramycin, Arbekacin, Bambermycins, Butirosin, Dibekacin, Dihdrostreptomycin, Fortimicin(s), Gentamicin, Ispamicin, Kanamycin, Micronomicin, Neomycin, Neomycin Undecylenate, Netilmicin, Paromomycin, Ribostamycin, Sisomicin, Spectinomycin, Streptomycin, Streptonicozid, Vancomycin (also considered a glycopeptide) and Tobramycin;

Amphenicols such as Azidamfenicol, Chloramphenicol, Chloramphenicol Palmitate, Chloramphenicol Pantothenate, Florfenicol and Thiamphenicol;

Ansamycins such as Rifamide, Rifampin, Rifamycin and Rifaximin;

β-Lactams, including: Carbapenems such as Imipenem;

Cephalosporins such as Cefactor, Cefadroxil, Cefamandole, Cefatrizine, Cefazedone, Cefazolin, Cefixime, Cefinenoxime, Cefodizime, Cefonicid, Cefoperazone, Ceforanide, Cefotaxime, Cefotiam, Cefpimizole, Cefpirimide, Cefpodoxime Proxetil, Cefroxadine, Cefsulodin, Ceftazidime, Cefteram, Ceftezole, Ceftibuten, Ceftizoxime, Ceftriaxone, Cefuroxime, Cefuzonam, Cephacetrile Sodium, Cephalexin, Cephaloglycin, Cephaloridine,

Cephalosporin, Cephalothin, Cephapirin Sodium, Cephradine and Pivcefalexin;

Cephamycins such as Cefbuperazone, Cefinetazole, Cefminox, Cefetan and Cefoxitin;

Monobactams such as Aztreonam, Carumonam and Tigemonam;

Oxacephems such as Flomoxef and Moxolactam;

Penicillins such as Amidinocillin, Amdinocillin Pivoxil, Amoxicillin, Ampicillin, Apalcillin, Aspoxicillin, Azidocillan, Azlocillan, Bacampicillin, Benzylpenicillinic Acid, Benzylpenicillin Sodium, Carbenicillin, Carfecillin Sodium, Carindacillin, Clometocill in, Cloxacill in, Cyclacillin, Dicloxacillin, Diphenicillin Sodium, Epicillin, Fenbenicillin, Floxicillin, Hetacillin, Lenampicillin, Metampicillin, Methicillin Sodium, Mezlocillin, Nafcillin Sodium, Oxacillin, Penamecillin, Penethamate Hydriodide, Penicillin G Benethamine, Penicillin G Benzathine, Penicillin G Benzhydrylamine, Penicillin G Calcium, Penicillin G Hydrabamine, Penicillin G Potassium, Penicillin G Procaine, Penicillen N, Penicillin O, Penicillin V, Penicillin V Benzathine, Penicillin V Hydrabamine, Penimepicycline, Phenethicillin Potassium, Piperacillin, Pivapicillin, Propicillin, Quinacillin, Sulbenicillin, Talampicillin, Temocillin and Ticarcillin;

Lincosamides such as Clindamycin and Lincomycin;

Macrolides such as Azithroimycin, Carbomycin, Clarithromycin, Erythromycin, Erythromycin Acistrate, Erythromycin Estolate, Erythromycin Glucoheptonate, Erythromycin Lactobionate, Erythromycin Propionate, Erythromycin Stearate, Josamycin, Leucomycins, Midecamycins, Miokamycin, Oleandomycin, Primycin, Rokitamycin, Rosaramicin, Roxithromycin, Spiramycin and Troleandomycin;

Polypeptides such as Amphomycin, Bacitracin, Capreomycin, Colistin, Enduracidin, Enviomycin, Fusafungine, Gramicidin(s), Gramicidin S, Mikamycin, Polymyxin, Polymyxin B-Methanesulfonic Acid, Pristinamycin, Ristocetin, Teicoplanin, Thiostrepton, Tuberactinomycin, Tyrocidine, Tyrothricin, Vancomycin, Viomycin, Viomycin Pantothenate, Virginiamycin and Zinc Bacitracin;

Tetracyclines such as Apicycline, Chlortetracycline, Clomocycline, Demeclocycline, Doxycycline, Guamecycline, Lymecycline, Meclocycline, Methacycline, Minocycline, Oxytetracycline, Penimepicycline, Pipacycline, Rolitetracycline, Sancycline, Senociclin and Tetracycline; and

other antibiotics such as Cycloserine, Mupirocin and Tuberin;

Antibacterial drugs (synthetic), including: 2,4-Diaminopyrimidines such as Brodimoprim, Tetroxoprim and Trimethoprim;

Nitrofurans such as Furaltadone, Furazolium Chloride, Nifuradene, Nifuratel, Nifurfoline, Nifurpirinol, Nifurprazine, Nifurtoinol and Nitrofurantoin;

Quinolones and Analogs such as Amifloxacin, Cinoxacin, Ciprofloxacin, Difloxacin, Enoxacin, Fleroxacin, Flumequine, Lomefloxacin, Miloxacin, Nalidixic Acid, Norfloxacin, Ofloxacin, Oxolinic Acid, Pefloxacin, Pipemidic Acid, Piromidic Acid, Rosoxacin, Temafloxacin and Tosufloxacin;

Sulfonamides such as Acetyl Sulfamethoxypyrazine, Acetyl Sulfisoxazole, Azosulfamide, Benzylsulfamide, Chloramine-B, Chloramine-T, Dichloramine T, Formosulfathiazole, N₂ Formylsulfisomidine, N²-β-D-Glucosylsulfanilamide, Mafenide, 4′-(Methylsulfamoyl)sulfanilanilide, p-Nitrosulfathiazole, Noprylsulfamide, Phthalylsulfacetamide, Phthalylsulfathiazole, Salazosulfadimidine, Succinylsulfathiazole, Sulfabenzamide, Sulfacetamide, Sulfachlorpyridazine, Sulfachrysoidine, Sulfacytine, Sulfadiazine, Sulfadicramide, Sulfadimethoxine, Sulfadoxine, Sulfaethidole, Sulfaguanidine, Sulfaguanol, Sulfalene, Sulfaloxic Acid, Sulfamerazine, Sulfameter, Sulfamethazine, Sulfamethizole, Sulfamethomidine, Sulfamethoxazole, Sulfamethoxypyridazine, Sulfametrole, Sulfamidochrysoidine, Sulfamoxole, Sulfanilamide, Sulfanilamidomethanesulfonic Acid Triethanolamine Salt, 4-Sulfanilamidosalicylic Acid, N-Sulfanilylsulfanilamide, Sulfanilylurea, N-Sulfanilyl-3,4-xylamide, Sulfanitran, Sulfaperine, Sulfaphenazole, Sulfaproxyline, Sulfapyrazine, Sulfapyridine, Sulfasomizole, Sulfasymazine, Sulfathiazole, Sulfathiourea, Sulfatolamide, Sulfisomidine and Sulfisoxazole;

Sulfones such as Acedapsone, Acediasulfone, Acetosulfone Sodium, Dapsone, Diathymosulfone, Glucosulfone Sodium, Solasulfone, Succisulfone, Sulfanilic Acid, p-Sulfanilylbenzylamine, p,p′-Sulfonyldianiline-N,N′ digalactoside, Sulfoxone Sodium and Thiazolsulfone; and

others such as Clofoctol, Hexedine, Methenamine, Methenamine Anhydromethylene-citrate, Methenamine Hippurate, Methenamine Mandelate, Methenamine Sulfosalicylate, Nitroxoline and Xibornol;

Anticholinergics such as Adiphenine Hydrochloride, Alverine, Ambutonomium Bromide, Aminopentamide, Amixetrine, Amprotropine Phosphate, Anisotropine Methylbromide, Apoatropine, Atropine, Atropine N-Oxide, Benactyzine, Benapryzine, Benzetimide, Benzilonium Bromide, Benztropine Mesylate, Bevonium Methyl Sulfate, Biperiden, Butropium Bromide, N-Butylscopolammonium Bromide, Buzepide, Camylofine, Caramiphen Hydrochloride, Chlorbenzoxamine, Chlorphenoxamine, Cimetropium Bromide, Clidinium Bromide, Cyclodrine, Cyclonium Iodide, Cycrimine Hydrochloride, Deptropine, Dexetimide, Dibutoline Sulfate, Dicyclomine Hydrochloride, Diethazine, Difemerine, Dihexyverine, Diphemanil Methylsulfate, N-(1,2-Diphenylethyl)nicotinamide, Dipiproverine, Diponium Bromide, Emepronium Bromide, Endobenzyline Bromide, Ethopropazine, Ethybenztropine, Ethylbenzhydramine, Etomidoline, Eucatropine, Fenpiverinium Bromide, Fentonium Bromide, Flutropium Bromide, Glycopyrrolate, Heteronium Bromide, Hexocyclium Methyl Sulfate, Homatropine, Hyoscyamine, Ipratropium Bromide, Isopropamide, Levomepate, Mecloxamine, Mepenzolate Bromide, Metcaraphen, Methantheline Bromide, Methixene, Methscopolamine Bromide, Octamylamine, Oxybutynin Chloride, Oxyphencyclimine, Oxyphenonium Bromide, Pentapiperide, Penthienate Bromide, Phencarbamide, Phenglutarimide, Pipenzolate Bromide, Piperidolate, Piperilate, Poldine Methysulfate, Pridinol, Prifinium Bromide, Procyclidine, Propantheline Bromide, Propenzolate, Propyromazine, Scopolamine, Scopolamine N-Oxide, Stilonium Iodide, Stramonium, Sultroponium, Thihexinol, Thiphenamil, Tiemonium Iodide, Timepidium Bromide, Tiquizium Bromide, Tridihexethyl Iodide, Trihexyphenidyl Hydrochloride, Tropacine, Tropenzile, Tropicamide, Trospium Chloride, Valethamate Bromide and Xenylropium Bromide;

Anticonvulsants such as Acetylpheneturide, Albutoin, Aloxidone, Aminoglutethimide, 4-Amino-3-hydroxybutyric Acid, Atrolactamide, Beclamide, Buramate, Calcium Bromide, Carbamazepine, Cinromide, Clomethiazole, Clonazepam, Decimemide, Diethadione, Dimethadione, Doxenitoin, Eterobarb, Ethadione, Ethosuximide, Ethotoin, Fluoresone, Garbapentin, 5-Hydroxytryptophan, Lamotrigine, Lomactil, Magnesium Bromide, Magnesium Sulfate, Mephenyloin, Mephobarbital, Metharbital, Methetoin, Methsuximide, 5-Methyl-5-(3-phenanthryl)hydantoin, 3-Methyl-5-phenylhydantoin, Narcobarbital, Nimetazepam, Nitrazepam, Paramethadione, Phenacemide, Phenetharbital, Pheneturide, Phenobarbital, Phenobarbital Sodium, Phensuximide, Phenylmethylbarbituric Acid, Phenyloin, Phethenylate Sodium, Potassium Bromide, Pregabatin, Primidone, Progabide, Sodium Bromide, Sodium Valproate, Solanum, Strontium Bromide, Suclofenide, Sulthiame, Tetrantoin, Tiagabine, Trimethadione, Valproic Acid, Valpromide, Vigabatrin and Zonisamide;

Antidepressants, including: Bicyclics such as Binedaline, Caroxazone, Citalopram, Dimethazan, Indalpine, Fencamine, Fluvoxamine Maleate, Indeloxazine Hydrochcloride, Nefopam, Nomifensine, Oxitriptan, Oxypertine, Paroxetine, Sertraline, Thiazesim, Trazodone, Venlafaxine and Zometapine;

Hydrazides/Hydrazines such as Benmoxine, Iproclozide, Iproniazid, Isocarboxazid, Nialamide, Octamoxin and Phenelzine;

Pyrrolidones such as Cotinine, Rolicyprine and Rolipram;

Tetracyclics such as Maprotiline, Metralindole, Mianserin and Oxaprotiline;

Tricyclics such as Adinazolam, Amitriptyline, Amitriptylinoxide, Amoxapine, Butriptyline, Clomipramine, Demexiptiline, Desipramine, Dibenzepin, Dimetracrine, Dothiepin, Doxepin, Fluacizine, Imipramine, Imipramine N-Oxide, Iprindole, Lofepramine, Melitracen, Metapramine, Nortriptyline, Noxiptilin, Opipramol, Pizotyline, Propizepine, Protriptyline, Quinupramine, Tianeptine and Trimipramine; and

others such as Adrafinil, Benactyzine, Bupropion, Butacetin, Deanol, Deanol Aceglumate, Deanol Acetamidobenzoate, Dioxadrol, Etoperidone, Febarbamate, Femoxetine, Fenpentadiol, Fluoxetine, Fluvoxamine, Hematoporphyrin, Hypercinin, Levophacetoperane, Medifoxamine, Minaprine, Moclobemide, Oxaflozane, Piberaline, Prolintane, Pyrisuccideanol, Rubidium Chloride, Sulpiride, Sultopride, Teniloxazine, Thozalinone, Tofenacin, Toloxatone, Tranylcypromine, L-Tryptophan, Viloxazine and Zimeldine;

Antidiabetics, including: Biguanides such as Buformin, Metformin and Phenformin;

Hormones such as Glucagon, Insulin, Insulin Injection, Insulin Zinc Suspension, Isophane Insulin Suspension, Protamine Zinc Insulin Suspension and Zinc Insulin Crystals;

Sulfonylurea derivatives such as Acetohexamide, 1-Butyl-3-metanilylurea, Carbutamide, Chlorpropamide, Glibornuride, Gliclazide, Glipizide, Gliquidone, Glisoxepid, Glyburide, Glybuthiazol(e), Glybuzole, Glyhexamide, Glymidine, Glypinamide, Phenbutamide, Tolazamide, Tolbutamide and Tolcyclamide; and

others such as Acarbose, Calcium Mesoxalate and Miglitol;

Antidiarrheal drugs such as Acetyltannic Acid, Albumin Tannate, Alkofanone, Aluminum Salicylates—Basic, Catechin, Difenoxin, Diphenoxylate, Lidamidine, Loperamide, Mebiquine, Trillium and Uzarin;

Antidiuretics such as Desmopressin, Felypressin, Lypressin, Ornipressin, Oxycinchophen, Pituitary—Posterior, Terlipressin and Vasopressin;

Antiestrogens such as Delmadinone Acetate, Ethamoxytriphetol, Tamoxifen and Toremifene;

Antifungal drugs (antibiotics), including: Polyenes such as Amphotericin-B, Candicidin, Dermostatin, Filipin, Fungichromin, Hachimycin, Hamycin, Lucensomycin, Mepartricin, Natamycin, Nystatin, Pecilocin and Perimycin; and others such as Azaserine, Griseofulvin, Oligomycins, Neomycin Undecylenate, PyrroInitrin, Siccanin, Tubercidin and Viridin;

Antifungal drugs (synthetic), including: Allylamines such as Naftifine and Terbinafine;

Imidazoles such as Bifonazole, Butoconazole, Chlordantoin, Chlormidazole, Cloconazole, Clotrimazole, Econazole, Enilconazole, Fenticonazole, Isoconazole, Ketoconazole, Miconazole, Omoconazole, Oxiconazole, Nitrate, Sulconazole and Tioconazole;

Triazoles such as Fluconazole, Itraconazole and Terconazole; and

others such as Acrisorcin, Amorolfine, Biphenamine, Bromosalicylchloranilide, Buclosamide, Calcium Propionate, Chlophenesin, Ciclopirox, Cloxyquin, Coparaffinate, Diamthazole, Dihydrochloride, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Tolciclate, Tolindate, Tolnaftate, Tricetin, Ujothion, Undecylenic Acid and Zinc Propionate;

Antiglaucoma drugs such as Acetazolamide, Befunolol, Betaxolol, Bupranolol, Carteolol, Dapiprazoke, Dichlorphenamide, Dipivefrin, Epinephrine, Levobunolol, Methazolamide, Metipranolol, Pilocarpine, Pindolol and Timolol;

Antigonadotropins such as Danazol, Gestrinone and Paroxypropione;

Antigout drugs such as Allopurinol, Carprofen, Colchicine, Probenecid and Sulfinpyrazone;

Antihistamines, including: Alkylamine derivatives such as Acrivastine, Bamipine, Brompheniramine, Chlorpheniramine, Dimethindene, Metron S, Pheniramine, Pyrrobutamine, Thenaldine, Tolpropamine and Triprolidine;

Aminoalkyl ethers such as Bietanautine, Bromodiphenhydramine, Carbinoxamine, Clemastine, Diphenlypyraline, Doxylamine, Embrammine, Medrylamine, Mephenphydramine, p-Methyldiphenhydramine, Orphenadrine, Phenyltoloxamine, Piprinhydrinate and Setasine;

Ethylenediamine derivatives such as Alloclamide, p-Bromtripelennamine, Chloropyramine, Chlorothen, Histapyrrodine, Methafurylene, Methaphenilene, Methapyrilene, Phenbenzamine, Pyrilamine, Talastine, Thenyldiamine, Thonzylamine Hydrochloride, Tripelennamine and Zolamine;

Piperazines such as Cetirizine, Chlorcyclizine, Cinnarizine, Clocinizine and Hydroxyzine;

Tricyclics, including: Phenothiazines such as Ahistan, Etymemazine, Fenethazine, N-Hydroxyethylpromethazine Chloride, Isopromethazine, Mequitazine, Promethazine, Pyrathiazine and Thiazinamium Methyl Sulfate; and

others such as Azatadine, Clobenzepam, Cyproheptadine, Deptropine, Isothipendyl, Loratadine and Prothipendyl; and

other antihistamines such as Antazoline, Astemizole, Azelastine, Cetoxime, Clemizole, Clobenztropine, Diphenazoline, Diphenhydramine, Fluticasone Propionate, Mebhydroline, Phenindamine, Terfenadine and Tritoqualine;

Antihyperlipoproteinemics, including: Aryloxyalkanoic acid derivatives such as Beclorbrate, Bazafibrate, Binifibrate, Ciprofibrate, Clinofibrate, Clofibrate, Clofibric Acid, Etonfibrate, Fenofibrate, Gemfibrozil, Nicofibrate, Pirifibrate, Ronifibrate, Simfibrate and Theofibrate;

Bile acid sequesterants such as Cholestyramine Resin, Colestipol and Polidexide;

HMG CoA reductase inhibitors such as Fluvastatin, Lovastatin, Pravastatin Sodium and Simvastatin;

Nicotinic acid derivatives Aluminum Nicotinate, Acipimox, Niceritrol, Nicoclonate, Nicomol and Oxiniacic Acid;

Thyroid hormones and analogs such as Etiroxate, Thyropropic Acid and Thyroxine; and

others such as Acifran, Azacosterol, Benfluorex, β-Benzalbutyramide, Carnitine, Chondroitin Sulfate, Clomestone, Detaxtran, Dextran Sulfate Sodium, 5,8,11,14,17-Eicosapentaenoic Acid, Eritadenine, Furazbol, Meglutol, Melinamide, Mytatrienediol, Ornithine, γ-Oryzanol, Pantethine, Penataerythritol Tetraacetate, α-Phenylbutyramide, Pirozadil, Probucol, α-Sitosterol, Sultosilic Acid, piperazine Salt, Tiadenol, Triparanol and Xenbucin;

Antihypertensive drugs, including: Arylethanolamine derivatives such as Amosulalol, Bufuralol, Dilevalol, Labetalol, Pronethalol, Sotalol and Sulfinalol;

Aryloxypropanolamine derivatives such as Acebutolol, Alprenolol, Arotinolol, Atenolol, Betaxolol, Bevantolol, Bisoprolol, Bopindolol, Bunitrolol, Bupranolol, Butofilolol, Carazolol, Cartezolol, Carvedilol, Celiprolol, Cetamolol, Epanolol, Indenolol, Mepindolol, Metipranolol, Metoprolol, Moprolol, Nadolol, Nipradilol, Oxprenolol, Penbutolol, Pindolol, Propranolol, Talinolol, Tetraolol, Timolol and Toliprolol;

Benzothiadiazine derivatives such as Althiazide, Bendroflumethiazide, Benzthiazide, Benzylhydrochlorothiazide, Buthiazide, Chlorothiazide, Chlorthalidone, Cyclopenthiazide, Cyclothiazide, Diazoxide, Epithiazide, Ethiazide, Fenquizone, Hydrochlorothiazide, Hydroflumethiazide, Methyclothiazide, Meticrane, Metolazone, Paraflutizide, Polythiazide, Tetrachlormethiazide and Trichlormethiazide;

N-Carboxyalkyl (peptide/lactam) derivatives such as Alacepril, Captopril, Cilazapril, Delapril, Enalapril, Enalaprilat, Fosinopril, Lisinopril, Moveltipril, Perindopril, Quinapril and Ramipril;

Dihydropyridine derivatives such as Amlodipine, Felodipine, Isradipine, Nicardipine, Nifedipine, Nilvadipine, Nisoldipine and Nitrendipirne;

Guanidine derivatives such as Bethanidine, Debrisoquin, Guanabenz, Guanacline, Guanadrel, Guanazodine, Guanethidine, Guanfacine, Guanochlor, Guanoxabenz and Guanoxan;

Hydrazines and phthalazines such as Budralazine, Cadralazine, Dihydralazine, Endralazine, Hydracarbazine, Hydralazine, Pheniprazine, Pildralazine and Todralazine;

Imidazole derivatives such as Clonidine, Lofexidine, Phentolamine, Phentolamine Mesylate, Tiamenidine and Tolonidine;

Quaternary ammonium compounds Azamethonium Bromide, Chlorisondamine Chloride, Hexamethonium, Pentacynium Bis(methyl sulfate), Pentamethonium Bromide, Pentolinium Tartate, Phenactopinium Chloride and Trimethidiunum Methosulfate;

Quinazoline derivatives such as Alfuzosin, Bunazosin, Doxazosin, Prasosin, Terazosin and Trimazosin;

Reserpine derivatives such as Bietaserpine, Deserpidine, Rescinnamine, Reserpine and Syrosingopine;

Sulfonamide derivatives such as Ambuside, Clopamide, Furosemide, Indapamide, Quinethazone, Tripamide and Xipamide; and

others such as Ajmaline, γ-Aminobutyric Acid, Bufeniode, Candesartan, Chlorthalidone, C₁₋cletaine, C₁₋closidomine, Cryptenamine Tannates, Eprosartan, Fenoldopam, Flosequinan, Indoramin, Irbesartan, Ketanserin, Losartan, Metbutamate, Mecamylamine, Methyldopa, Methyl 4-Pyridyl Ketone Thiosemicarbarzone, Metolazone, Minoxidil, Muzolimine, Pargyline, Pempidine, Pinacidil, Piperoxan, Primaperone, Protoveratrines, Raubasine, Rescimetol, Rilmenidene, Saralasin, Sodium Nitroprusside, Ticrynafen, Trimethaphan Camsylate, Tyrosinase, Urapidil and Valsartan;

Antihyperthyroids such as 2-Amino-4-methylthiazole, 2-Aminothiazole, Carbimazole, 3,5-Dibromo-L-tyrosine, 3,5-Diiodotyrosine, Hinderin, Iodine, lothiouracil, Methimazole, Methylthiouracil, Propylthiouracil, Sodium Perchlorate, Thibenzazoline, Thiobarbital and 2-Thiouracil;

Antihypotensive drugs such as Amezinium Methyl Sulfate, Angiotensin Amide, Dimetofrine, Dopamine, Etifelmin, Etilefrin, Gepefrine, Metaraminol, Midodrine, Norepinephrine, Pholedrinead and Synephrine;

Antihypothyroid drugs such as Levothyroxine Sodium, Liothyronine, Thyroid, Thyroidin, Thyroxine, Tiratricol and TSH;

Anti-Inflammatory (non-steroidal) drugs, including: Aminoarylcarboxylic acid derivatives such as Enfenamic Acid, Etofenamate, Flufenamic Acid, Isonixin, Meclofenamic Acid, Mefanamic Acid, Niflumic Acid, Talniflumate, Terofenamate and Tolfenamic Acid;

Arylacetic acid derivatives such as Acemetacin, Alclofenac, Amfenac, Bufexamac, Cinmetacin, Clopirac, Diclofenac Sodium, Etodolac, Felbinac, Fenclofenac, Fenclorac, Fenclozic Acid, Fentiazac, Glucametacin, Ibufenac, Indomethacin, Isofezolac, Isoxepac, Lonazolac, Metiazinic Acid, Oxametacine, Proglumetacin, Sulindac, Tiaramide, Tolmetin and Zomepirac;

Arylbutyric acid derivatives such as Bumadizon, Butibufen, Fenbufen and Xenbucin;

Arylcarboxylic acids such as Clidanac, Ketorolac and Tinoridine;

Arylpropionic acid derivatives such as Alminoprofen, Benoxaprofen, Bucloxic Acid, Carprofen, Fenoprofen, Flunoxaprofen, Flurbiprofen, Ibuprofen, Ibuproxam, Indoprofen, Ketoprofen, Loxoprofen, Miroprofen, Naproxen, Oxaprozin, Piketoprofen, Pirprofen, Pranoprofen, Protizinic Acid, Suprofen and Tiaprofenic Acid;

Pyrazoles such as Difenamizole and Epirizole;

Pyrazolones such as Apazone, Benzpiperylon, Feprazone, Mofebutazone, Morazone, Oxyphenbutazone, Phenybutazone, Pipebuzone, Propyphenazone, Ramifenazone, Suxibuzone and Thiazolinobutazone;

Salicylic acid derivatives such as Acetaminosalol, Aspirin, Benorylate, Bromosaligenin, Calcium Acetylsalicylate, Diflunisal, Etersalate, Fendosal, Gentisic Acid, Glycol Salicylate, Imidazole Salicylate, Lysine Acetylsalicylate, Mesalamine, Morpholine Salicylate, 1-Naphthyl Salicylate, Olsalazine, Parsalmide, Phenyl Acetylsalicylate, Phenyl Salicylate, Salacetamide, Salicylamine O-Acetic Acid, Salicylsulfuric Acid, Salsalate and Sulfasalazine;

Thiazinecarboxamides such as Droxicam, Isoxicam, Piroxicam and Tenoxicam; and

others such as ∉-Acetamidocaproic Acid, S-Adenosylmethionine, 3-Amino-4-hydroxybutyric Acid, Amixetrine, Bendazac, Benzydamine, Bucolome, Difenpiramide, Ditazol, Emorfazone, Guaiazulene, Nabumetone, Nimesulide, Orgotein, Oxaceprol, Paranyline, Perisoxal, Pifoxime, Proquazone, Proxazole and Tenidap;

Antimalarial drugs such as Acedapsone, Amodiaquin, Arteether, Artemether, Artemisinin, Artesunate, Bebeerine, Berberine, Chirata, Chlorguanide, Chloroquine, Chlorproguanil, Cinchona, Cinchonidine, Cinchonine, Cycloguanil, Gentiopicrin, Halofantrine, Hydroxychloroquine, Mefloquine Hydrochloride, 3-Methylarsacetin, Pamaquine, Plasmocid, Primaquine, Pyrimethamine, Quinacrine, Quinine, Quinine Bisulfate, Quinine Carbonate, Quinine Dihydrobromide, Quinine Dihydrochloride, Quinine Ethylcarbonate, Quinine Formate, Quinine Gluconate, Quinine Hydriodide, Quinine Hydrochloride, Quinine Salicylate, Quinine Sulfate, Quinine Tannate, Quinine Urea Hydrochloride, Quinocide, Quinoline and Sodium Arsenate Diabasic;

Antimigraine drugs such as Alpiropride, Dihydroergotamine, Eletriptan, Ergocornine, Ergocorninine, Ergocryptine, Ergot, Ergotamine, Flumedroxone acetate, Fonazine, Lisuride, Methysergid(e), Naratriptan, Oxetorone, Pizotyline, Rizatriptan and Sumatriptan;

Antinauseant drugs such as Acetyll eucine Monoethanolamine, Alizapride, Benzquinamide, Bietanautine, Bromopride, Buclizine, Chlorpromazine, Clebopride, Cyclizine, Dimenhydrinate, Dipheniodol, Domperidone, Granisetron, Meclizine, Methalltal, Metoclopramide, Metopimazine, Nabilone, Ondansteron, Oxypendyl, Pipamazine, Piprinhydrinate, Prochlorperazine, Scopolamine, Tetrahydrocannabinols, Thiethylperazine, Thioproperzaine and Trimethobenzamide;

Antineoplastic drugs, including: Alkylating agents, such as Alkyl sulfonates such as Busulfan, Improsulfan and Piposulfan;

Aziridines such as Benzodepa, Carboquone, Meturedepa and Uredepa;

Ethylenimines and methylmelamines such as Altretamine, Triethylenemelamine, Triethylenephosphoramide, Triethylenethiophosphoramide and Trimethylolomelamine;

Nitrogen mustards such as Chlorambucil, Chlornaphazine, Chclophosphamide, Estramustine, Ifosfamide, Mechlorethamine, Mechlorethamine Oxide Hydrochloride, Melphalan, Novembichin, Phenesterine, Prednimustine, Trofosfamide and Uracil Mustard;

Nitrosoureas such as Carmustine, Chlorozotocin, Fotemustine, Lomustine, Nimustine and Ranimustine; and others such as Camptothecin, Dacarbazine, Mannomustine, Mitobronitol, Mitolactol and Pipobroman;

Antibiotics such as Aclacinomycins, Actinomycin F₁, Anthramycin, Azaserine, Bleomycins, Cactinomycin, Carubicin, Carzinophilin, Chromomycins, Dactinomycin, Daunorubicin, 6-Diazo-5-oxo-L-norleucine, Doxorubicin, Epirubicin, Mitomycins, Mycophenolic Acid, Nogalamycin, Olivomycins, Peplomycin, Plicamycin, Porfiromycin, Puromycin, Streptonigrin, Streptozocin, Tubercidin, Ubenimex, Zinostatin and Zorubicin;

Antimetabolites, including: Folic acid analogs such as Denopterin, Methotrexate, Pteropterin and Trimetrexate;

Purine analogs such as Fludarabine, 6-Mercaptopurine, Thiamiprine and Thioguanaine; and

Pyrimidine analogs such as Ancitabine, Azacitidine, 6-Azauridine, Carmofur, Cytarabine, Doxifluridine, Enocitabine, Floxuridine Fluoroouracil and Tegafur;

Enzymes such as L-Asparaginase; and

others such as Aceglatone, Amsacrine, Bestrabucil, Bisantrene, Bryostatin 1, Carboplatin, Cisplatin, Defofamide, Demecolcine, Diaziquone, Elformithine, Elliptinium Acetate, Etoglucid, Etoposide, Gallium Nitrate, Hydroxyurea, Interferon-α, Interferon-β, Interferon-γ, Interleukine-2, Lentinan, Letrozole, Lonidamine, Mitoguazone, Mitoxantrone, Mopidamol, Nitracrine, Pentostatin, Phenamet, Pirarubicin, Podophyllinicc Acid, 2-Ethylhydrazide, Polynitrocubanes, Procarbazine, PSK7, Razoxane, Sizofuran, Spirogermanium, Taxol, Teniposide, Tenuazonic Acid, Triaziquone, 2.2′.2″-Trichlorotriethylamine, Urethan, Vinblastine, Vincristine, Vindesine and Vinorelbine;

Antineoplastic (hormonal) drugs, including: Androgens such as Calusterone, Dromostanolone Propionate, Epitiostanol, Mepitiostane and Testolactone;

Antiadrenals such as Aminoglutethimide, Mitotane and Trilostane;

Antiandrogens such as Flutamide and Nilutamide; and

Antiestrogens such as Tamoxifen and Toremifene;

Antineoplastic adjuncts including folic acid replenishers such as Frolinic Acid;

Antiparkinsonian drugs such as Amantadine, Benserazide, Bietanautine, Biperiden, Bromocriptine, Budipine, Cabergoline, Carbidopa, Deprenyl (a/k/a L-deprenyl, L-deprenil, L-deprenaline and selegiline), Dexetimide, Diethazine, Diphenhydramine, Droxidopa, Ethopropazine, Ethylbenzhydramine, Levodopa, Naxagolide, Pergolide, Piroheptine, Pramipexole, Pridinol, Prodipine, Quinpirole, Remacemide, Ropinirole, Terguride, Tigloidine and Trihexyphenidyl Hydrochloride;

Antipheochromocytoma drugs such as Metyrosine, Phenoxybenzamine and Phentolamine;

Antipneumocystis drugs such as Efformithine, Pentamidine and Sulfamethoxazole;

Antiprostatic hypertrophydrugs such as Gestonorone Caproate, Mepartricin, Oxendolone and Proscar7;

Antiprotozoal drugs (Leshmania) such as Antimony Sodium Gluconate, Ethylstibamine, Hydroxystilbamidine, N-Methylglucamine, Pentamidine, Stilbamidine and Urea Stibamine;

Antiprotozoal drugs (Trichomonas) such as Acetarsone, Aminitrozole, Anisomycin, Azanidazole, Forminitrazole, Furazolidone, Hachimycin, Lauroguadine, Mepartricin, Metronidazole, Nifuratel, Nifuroxime, Nimorazole, Secnidazole, Silver Picrate, Tenonitrozole and Timidazole;

Antiprotozoal drugs (Trypanosma) such as Benznidazole, Eflornithine, Melarsoprol, Nifurtimox, Oxophenarsine, Hydrochloride, Pentamidine, Propamidine, Puromycin, Quinapyramine, Stilbamidine, Suramin Sodium, Trypan Red and Tryparasmide;

Antipuritics such as Camphor, Cyproheptadine, Dichlorisone, Glycine, Halometasone, 3-Hydroxycamphor, Menthol, Mesulphen, Methdilazine, Phenol, Polidocanol, Risocaine, Spirit of Camphor, Thenaldine, Tolpropamine and Trimeprazine;

Antipsoriatic drugs such as Acitretin, Ammonium Salicylate, Anthralin, 6-Azauridine, Bergapten(e), Chrysarobin, Etretinate and Pyrogallol;

Antipsychotic drugs, including: Butyrophenones such as Benperidol, Bromperidol, properidol, Fluanisone, Haloperidol, Melperone, Moperone, Pipamperone, Sniperone, Timiperone and Trifluperidol;

Phenothiazines such as Acetophenazine, Butaperazine, Carphenazine, Chlorproethazine, Chlorpromazine, Clospirazine, Cyamemazine, Dixyrazine, Fluphenazine, Imiclopazine, Mepazine, Mesoridazine, Methoxypromazine, Metofenazate, Oxaflumazine, Perazine, Pericyazine, Perimethazine, Perphenazine, Piperacetazine, Pipotiazine, Prochlorperazine, Promazine, Sulforidazine, Thiopropazate, Thioridazine, Trifluoperazine and Triflupromazine;

Thioxanthenes such as Chlorprothixene, Clopenthixol, Flupentixol and Thiothixene;

other tricyclics such as Benzquinamide, Carpipramine, Clocapramine, Clomacran, Clothiapine, Clozapine, Opipramol, Prothipendyl, Tetrabenazine, and Zotepine; and

others such as Alizapride, Amisulpride, Buramate, Fluspirilene, Molindone, Penfluridol, Pimozide, Spirilene and Sulpiride;

Antipyretics such as Acetaminophen, Acetaminosalol, Acetanilide,

Aconine, Aconite, Aconitine, Alclofenac, Aluminum Bis(acetylsalicylate), Aminochlorthenoxazin, Aminopyrine, Aspirin, Benorylate, Benzydamine, Berberine, p-Bromoacetanilide, Bufexamac, Bumadizon, Calcium Acetysalicylate, Chlorthenoxazin(e), Choline Salicylate, Clidanac, Dihydroxyaluminum Acetylsalicylate, Dipyrocetyl, Dipyrone, Epirizole, Etersalate, Imidazole Salicylate, Indomethacin, Isofezolac, p-Lactophenetide, Lysine Acetylsalicylate, Magnesium Acetylsalicylate, Meclofenamic Acid, Morazone, Morpholine Salicylate, Naproxen, Nifenazone, 51-Nitro-2′-propoxyacetanilide, Phenacetin, Phenicarbazide, Phenocoll, Phenopyrazone, Phenyl Acetylsalicylate, Phenyl Salicylate, Pipebuzone, Propacetamol, Propyphenazone, Ramifenazone, Salacetamide, Salicylamide O-Acetic Acid, Sodium Salicylate, Sulfamipyrine, Tetrandrine and Tinoridine;

Antirickettsial drugs such as p-Aminobenzoic Acid, Chloramphenicol, Chloramphenicol Palmitate, Chloramphenicol Pantothenate and Tetracycline;

Antiseborrheic drugs such as Chloroxine, 3-O-Lauroylpyridoxol Diacetate, Piroctone, Pyrithione, Resorcinol, Selenium Sulfides and Tioxolone;

Antiseptics, including: Guanidines such as Alexidine, Ambazone, Chlorhexidine and Picloxydine;

Halogens and halogen compounds such as Bismuth Iodide Oxide, Bismuth Iodosubgallate, Bismuth Tribromophenate, Bornyl Chloride, Calcium Iodate, Chlorinated Lime, Cloflucarban, Fluorosalan, Iodic Acid, Iodine, Iodine Monochloride, Iodine Trichloride, Iodoform, Methenamine Tetraiodine, Oxychlorosene, Povidone-Iodine, Sodium Hypochlorite, Sodium Iodate, Symclosene, Thymol Iodide, Triclocarban, Triclosan and Troclosene Potassium;

Mercurial compounds such as Hydragaphen, Meralein Sodium, Merbromin, Mercuric Chloride, Mercuric Chloride, Ammoniated, Mercuric Sodium p-Phenolsulfonate, Mercuric Succinimide, Mercuric Sulfide, Red, Mercurophen, Mercurous Acetate, Mercurous Chloride, Mercurous Iodide, Nitromersol, Potassium Tetraiodomercurate(II), Potassium Triiodomercurate (II) Solution, Thimerfonate Sodium and Thimerosal;

Nitrofurans such as Furazolidone, 2-(Methoxymethyl)-5-nitrofuran, Nidroxyzone, Nifuroxime, Nifurzide and Nitrofurazone;

Phenols such as Acetomeroctol, Bithionol, Cadmium Salicylate, Carvacrol, Chloroxylenol, Clorophene, Cresote, Cresol(s), p-Cresol, Fenticlor, Hexachlorophene, 1-Napthyl Salicylate, 2-Napthyl Salicylate, 2,4,6-Tribromo-m-cresol, and 3′,4′,5′-Trichlorosalicylanilide;

Quinolines such as Aminoquinuride, Benzoxiquine, Broxyquinoline, Chloroxine, Chlorquinaldol, Cloxyquin, Ethylhydrocupreine, Euprocin, Halquinol, Hydrastine, 8-Hydroxquinoline, 8-Hydroxquinoline Sulfate and Iodochlorhydroxyquin; and

others such as Aluminum Acetate Solution, Aluminum Subacetate Solution, Aluminum Sulfate, 3-Amino-4-hydroxybutyric Acid, Boric Acid, Chlorhexidine, Chloroazodin, m-Cresyl Acetate, Cupric Sulfate, Dibromopropamidine, Ichthammol, Negatol7, Noxytiolin, Ornidazole, Propiolactone, α-Terpineol;

Antispasmodic drugs such as Alibendol, Ambucetamide, Aminopromazine, Apoatropine, Bevonium Methyl Sulfate, Bietamiverine, Butaverine, Butropium Bromide, N-Butylscopolammonium Bromide, Caroverine, Cimetropium Bromide, Cinnamedrine, Clebopride, Coniine Hydrobromide, Coniine Hydrochloride, Cyclonium Iodide, Difemerine, Diisopromine, Dioxaphetyl Butyrate, Diponium Bromide, Drofenine, Emepronium Bromide, Ethaverine, Feclemine, Fenalamide, Fenoverine, Fenpiprane, Fenpiverinium Brcmide, Fentonium Bromide, Flavoxate, Flopropione, Gluconic Acid, Guaiactamine, Hydramitrazine, Hymecromone, Leiopyrrole, Mebeverine, Moxaverine, Nafiverine, Octamylamine, Octaverine, Pentapiperide, Phenamacide Hydrochloride, Phloroglucinol, Pinaverium Bromide, Piperilate, Pipoxolan Hydrochloride, Pramiverin, Prifinium Bromide, Properidine, Propivane, Propyromazine, Prozapine, Racefemine, Rociverine, Spasmolytol, Stilonium Iodide, Sultroponium, Tiemonium Iodide, Tiquizium Bromide, Tiropramide, Trepibutone, Tricromyl, Trifolium, Trimebutine, N,N-ITrimethyl-3,3-diphenyl-propylamine, Tropenzile, Trospium Chloride and Xenylropium Bromide;

Antithrombotic drugs such as Anagrelide, Argatroban, Cilostazol, Chrysoptin, Daltroban, Defibrotide, Enoxaparin, Fraxiparine-7, Indobufen, Lamoparan, Ozagrel, Picotamide, Plafibride, Reviparin, Tedelparin, Ticlopidine, Triflusal and Warfarin;

Antitussive drugs such as Allocamide, Amicibone, Benproperine, Benzonatate, Bibenzonium Bromide, Bromoform, Butamirate, Butethamate, Caramiphen Ethanedisulfonate, Carbetapentane, Chlophedianol, Clobutinol, Cloperastine, Codeine, Codeine Methyl Bromide, Codeine N-Oxide, Codeine Phosphate, Codeine Sulfate, Cyclexanone, Dextromethorphan, Dibunate Sodium, Dihydrocodeine, Dihydrocodeinone Enol Acetate, Dimemorfan, Dimethoxanate, α,α-Diphenyl-2-piperidinepropanol, propropizine, Drotebanol, Eprazinone, Ethyl Dibunate, Ethylmorphine, Fominoben, Guiaiapate, Hydrocodone, Isoaminile, Levopropoxyphene, Morciofone, Narceine, Normethadone, Noscapine, Oxeladin, Oxolamine, Pholcodine, Picoperine, Pipazethate, Piperidione, Prenoxdiazine Hydrochloride, Racemethorphan, Taziprinone Hydrochloride, Tipepidine and Zipeprol;

Antiulcerative drugs such as Aceglutamide Aluminum Complex, e-Acetamidocaproic Acid Zinc Salt, Acetoxolone, Arbaprostil, Benexate Hydrochloride, Bismuth Subcitrate Sol (Dried), Carbenoxolone, Cetraxate, Cimetidine, Enprostil, Esaprazole, Famotidine, Ftaxilide, Gefarnate, Guaiazulene, Irsogladine, Misoprostol, Nizatidine, Omeprazole, Ornoprostil, γ-Oryzanol, Pifamine, Pirenzepine, Plaunotol, Ranitidine, Rioprostil, Rosaprostol, Rotraxate, Roxatidine Acetate, Sofalcone, Spizofurone, Sucralfate, Teprenone, Trimoprostil, Thrithiozine, Troxipide and Zolimidine;

Antiurolithic drugs such as Acetohydroxamic Acid, Allopurinol, Potassium Citrate and Succinimide;

Antivenin drugs such as Lyovac7 Antivenin;

Antiviral drugs, including: Purines and pyrimidinones such as Acyclovir, Cytarabine, Dideoxyadenosine, Dideoxycytidine, Dideoxyinosine, Edoxudine, Floxuridine, Ganciclovir, Idoxuridine, Inosine Pranobex, MADU, Penciclovir, Trifluridine, Vidrarbine and Zidovudiine; and

others such as Acetylleucine Monoethanolamine, Amantadine, Amidinomycin, Cosalane, Cuminaldehyde Thiosemicarbzone, Foscarnet Sodium, Imiquimod, Interferon-α, Interferon-β, Interferon-γ, Kethoxal, Lysozyme, Methisazone, Moroxydine, Podophyllotoxin, Ribavirin, Rimantadine, Stallimycin, Statolon, Tromantadine, Xenazoic Acid, and the anti-influenza drugs zanamivir and oseltamivir phosphate;

Anxiolytic drugs, including: Arylpiperazines such as Buspirone, Gepirone, Ipsapirone and Tondospirone;

Benzodiazepine derivatives such as Alprazolam, Bromazepam, Camazepam, Chlordiazepoxide, Clobazam, Clorazepate, Chotiazepam, Cloxazolam, Diazepam, Ethyl Loflazepate, Etizolam, Fluidazepam, Flutazolam, Flutoprazepam, Halazepam, Ketazolam, Lorazepam, Loxapine, Medazepam, Metaclazepam, Mexazolam, Nordazepam, Oxazepam, Oxazolam, Pinazepam, Prazepam and Tofisopam;

Carbamates such as Cyclarbamate, Emylcamate, Hydroxyphenamate, Meprobamate, Phenprobamate and Tybamate; and

others such as Alpidem, Benzoctamine, Captodiamine, Chlormezanone, Etifoxine, Flesinoxan, Fluoresone, Glutamic Acid, Hydroxyzine, Lesopitron, Mecloralurea, Mephenoxalone, Mirtazepine, Oxanamide, Phenaglycodol, Suriclone and Zatosetron;

Benzodiazepine antagonists such as Flumazenil;

Bronchodilators, including: Ephedrine derivatives such as Albuterol, Bambuterol, Bitolterol, Carbuterol, Clenbuterol, Clorprenaline, Dioxethedrine, Ephedrine, Epiniphrine, Eprozinol, Etafedrine, Ethylnorepinephrine, Fenoterol, Hexoprenaline, Isoetharine, Isoproterenol, Mabuterol, Metaproterenol, N-Methylephedrine, Pirbuterol, Procaterol, Protokylol, Reproterol, Rimiterol, Salmeterol, Soterenol, Terbutaline and Tulobuterol;

Quaternary ammonium compounds such as Bevonium Methyl Sulfate, Clutropium Bromide, Ipratropium Bromide and Oxitropium Bromide;

Xanthine derivatives such as Acefylline, Acefylline piperazine, Ambuphylline, Aminophylline, Bamifylline, choline Theophyllinate, Doxofylline, Dyphylline, Enprofylline, Etamiphyllin, Etofylline, Guaithylline, Proxyphylline, Theobromine, 1-Theobromineacetic Acid and Theophylline; and

others such as Fenspiride, Medibazine, Montekulast, Methoxyphenanime, Tretoquinol and Zafirkulast;

Calcium channel blockers, including: Arylalkylamines such as Bepridil, Ditiazem, Fendiline, Gallopanil, Prenylamine, Terodiline and Verapamil;

Dihydropyridine derivatives such as Felodipine, Isradipine, Nicardipine, Nifedipine, Nilvadipine, Nimodipine, Nisoldipine and Nitrendipine;

Piperazine derivatives such as Cinnarizine, Flunarisine and Lidoflazine; and

others such as Bencyclane, Etafenone and Perhexyline;

Calcium regulators such as Calcifediol, Calcitonin, Calcitriol, Clodronic Acid, Dihydrotachysterol, Elcatonin, Etidronic Acid, Ipriflavone, Pamidronic Acid, Parathyroid Hormone and Teriparatide Acetate;

Cardiotonics such as Acefylline, Acetyldigititoxins, 2-Amino-4-picoline, Amrinone, Benfurodil Hemisuccinate, Buclasdesine, Cerberoside, Camphotamide, Convallatoxin, Cymarin, Denopamine, Deslanoside, Ditalin, Digitalis, Digitoxin, Digoxin, Dobutamine, Dopamine, Dopexamine, Enoximone, Erythrophleine, Fenalcomine, Gitalin, Gitoxin, Glycocyamine, Heptaminol, Hydrastinine, lbopamine, Lanotodises, Metamivam, Milrinone, Neriifolin, Oleandrin, Ouabain, Oxyfedrine, Prenalterol, Proscillaridin, Resibufogenin, Scillaren, Scillarenin, Strophanthin, Sulmazole, Theobromine and Xamoterol;

Chelating agents such as Deferozmine, Ditiocarb Sodium, Edetate Calcium Disodium, Edetate Disodium, Edeate Sodium, Edetate Trisodium, Penicillamine, Pentetate Calcium Trisodium, Pentectic Acid, Succimer and Trientine;

Cholecystokinin antagonists such as Proglumide;

Cholelitholytic agents such as Chenodiol, Methyl tert-Butyl Ether, Monooctanoin and Ursodiol;

Choleretics such as Alibendol, Anethole Trithion, Azintamide, Cholic Acid, Cicrotoic Acid, Clanobutin, Cyclobutyrol, Cyclovalone, Cynarin(e), Dehydrocholic Acid, Deoxycholic Acid, Dimecrotic Acid, α-Ethylbenzyl Alcohol, Exiproben, Feguprol, Fencibutirol, Fenipentol, Florantyrone, Hymecromone, Menbutone, 3-(o-Methoxyphenyl)-2-phenylacrylic Acid, Metochalcone, Moquizone, Osalmid, Ox Bile Extract, 4.4′-Oxydi-2-butanol, Piprozolin, Prozapine, 4-Salicyloylmorpholine, Sincalide, Taurocholic Acid, Timonacic, Tocamphyl, Trepibutone and Vanitiolide;

Cholinergic agents such as Aceclidine, Acetylcholine Bromide, Acetylcholide Chloride, Aclatonium Napadisilate, Benzpyrinium Bromide, Bethanechol chloride, Carbachol, Carpronium chloride, Demecarium Bromide, Dexpanthenol, Diisopropyl Paraoxon, Echothiophate Iodide, Edrophomium chloride, Eseridine, Furtrethonium, Isofluorophate, Methacholine chloride, Muscarine, Neostigmine, Oxapropanium Iodide, Physostigmine and Pyridostigmine Bromide;

Cholinesterase inhibitors such as Ambenonium Chloride, Distigmine Bromide and Galanthamine;

Cholinesterase reactivators such as Obidoximine Chloride and Pralidoxime Chloride;

Central nervous system stimulants and agents such as Amineptine, Amphetimine, Amphetaminil, Bemegride, Benzphetamine, Brucine, Caffeine, Chlorphentermine, Clofenciclan, Clortermine, Coca, Demanyl Phosphate, Dexoxadrol, Dextroamphetamine Sulfate, Diethlpropion, N-Ethylamphetamine, Ethamivan, Etifelmin, Etryptamine, Fencamfamine, Fenethylline, Fenosolone, Fluorothyl, Galanthamine, Hexacyclonate Sodium, Homocamfin, Mazindol, Megexamide, Methamphetamine, Methylphenidate, Nikethamide, Pemoline, Pentylenetetrazole, Phenidimetrazine, Phenmetrazine, Phentermine, Picrotoxin, Pipradrol, Prolintane and Pyrovalerone;

Decongestants such as Amidephrine, Cafaminol, Cyclopentamine, Ephedrine, Epinephrine, Fenoxazoline, Indanazoline, Metizoline, Naphazoline, Nordefrin Hydrochloride, Octodrine, oxymetazoline, Phenylephrine Hydrochloride, Phenylpropanolamine Hydrochloride, Phenylpropylmethylamine, Propylhexedrine, Pseudoephedrine, Tetrahydrozoline, Tymazoline and Xylometazoline;

Dental agents, including: Bisphosphonates (anti-periodontal disease and bone resorption) such as Alendronate, Clodronate, Etidronate, Pamidronate and Tiludronate; Carries Prophylactics such as Arginine and Sodium Fluoride;

Desensitizing Agents such as Potassium Nitrate and Citrate Oxalate;

Depigmentors such as Hydroquinine, Hydroquinone and Monobenzone;

Diuretics, including: Organomercurials such as Chlormerodrin, Meralluride, Mercamphamide, Mercaptomerin Sodium, Mercumallylic Acid, Mercumatilin Sodium, Mercurous Chloride and Mersalyl;

Pteridines such as Furterene and Triamterene;

Purines such as Acefylline, 7-Morpholinomethyltheophylline, Pamabrom, Protheobromine and Theobromine;

Steroids such as Canrenone, Oleandrin and Spironolactone;

Sulfonamide derivatives such as Acetazolamide, Ambuside, Azosemide, Bumetanide, Butazolamide, Chloraminophenamide, Clofenamide, Clopamide, Clorexolene, Diphenylmethane-4.4′-disulfonamide, Disulfamide, Ethbxzolamide, Furosemide, Indapamide, Mefruside, Methazolamide, Piretanide, Quinethazone, Torasemide, Tripamide and Xipamide;

Uracils such as Aminometradine and Amisometradine;

others such as Amanozine, Amiloride, Arbutin, Chlorazanil, Ethacrynic Acid, Etozolin, Hydracarbazine, Isosorbide, Mannitol, Metochalcone, Muzolimine, Perhexyline, Ticrynafen and Urea;

Dopamine receptor agonists such as Bromocriptine, Dopexamine, Fenoldopam, lbopamine, Lisuride, Naxagolide and Pergolide;

Ectoparasiticides such as Amitraz, Benzyl Benzoate, Carbaryl, Crotamiton, DDT, Dixanthogen, Isobornyl Thiocyanoacetate—Technical, Lime Sulfurated Solution, Lindane, Malathion, Mercuric Oleate, Mesulphen and Sulphur—Pharmaceutical;

Enzymes, including: Digestive enzymes such as α-Amylase (Swine Pancreas), Lipase, Pancrelipase, Pepsin and Rennin;

Mucolytic enzymes such as Lysozyme;

Penicillin inactivating enzymes such as Penicillinase; and

Proteolytic enzymes such as Collagenase, Chymopapain, Chymotrypsins, Papain and Trypsin;

Enzyme inducers (hepatic) such as Flumecinol;

Estrogens, including: Nonsteroidal estrogens such as Benzestrol, Broparoestrol, Chlorotrianisene, Dienestrol, Diethylstilbestrol, Diethylstilbestrol Diproprionate, Dimestrol, Fosfestrol, Hexestrol, Methallenestril and Methestrol; and

Steroidal estrogens such as Colpormon, Conjugated Estrogenic Hormones, Equilenin, Equilin, Estradiol, Estradiol Benzoate, Estradio17β-Cypionate, Estriol, Estrone, Ethinyl Estradiol, Mestranol, Moxestrol, Mytatrienediol, Quinestradiol and Quinestrol;

Gastric secretion inhibitors such as Enterogastrone and Octreotide;

Glucocorticoids such as 21-Acetoxyprefnenolone, Aalclometasone, Algestone, Amicinonide, Beclomethasone, Betamethasone, Budesonide, Chloroprednisone, Clobetasol, Blovetasone, Clocortolone, Cloprednol, Corticosterone, Cortisone, Cortivazol, Deflazacort, Desonide, Desoximetasone, Dexamethasone, Diflorasone, Diflucortolone, Difluprednate, Enoxolone, Fluazacort, Flucloronide, Flumehtasone, Flunisolide, Fluocinolone Acetonide, Fluocinonide, Fluocortin Butyl, Fluocortolone, Fluorometholone, Fluperolone Acetate, Fluprednidene Acetate, Fluprednisolone, Flurandrenolide, Formocortal, Halcinonide, Halometasone, Halopredone Acetate, Hydrocortamate, Hydrocortisone, Hydrocortisone Acetate, ydrocortisone Phosphate, Hydrocortisone 21-Sodium Succinate, Hydrocortisone Tebutate, Mazipredone, Medrysone, Meprednisone, Methyolprednisolone, Mometasone Furoate, Paramethasone, Prednicarbate, Prednisolone, Prednisolone 21-Diethylaminoacetate, Prednisone Sodium Phosphate, Prednisolone Sodium Succinate, Prednisolone Sodium 21-m-Sulfobenzoate, Prednisolone 21-Stearoylglycolate, Prednisolone Tebutate, Prednisolone 21-Trimethylacetate, Prednisone, Prednival, Prednylidene, Prednylidene 21-Diethylaminoacetate, Tixocortal, Triamcinolone, Triamcinolone Acetonide, Triamcinolone Benetonide and Triamcinolone Hexacetonide;

Gonad-Stimulating principles such as Buserelin, Clomiphene, Cyclofenil, Epimestrol, FSH, HCG and LH-RH;

Gonadotropic hormones such as LH and PMSG;

Growth hormone inhibitors such as Octreotide and Somatostatin;

Growth hormone releasing factors such as Semorelin;

Growth stimulants such as Somatotropin;

Hemolytic agents such as Phenylhydrazine and Phenylhydrazine Hydrochloride;

Heparin antagonists such as Hexadimethrine Bromide and Protamines;

Hepatoprotectants such as S-Adenosylmethionine, Betaine, Catechin, Citolone, Malotilate, Orazamide, Phosphorylcholine, Protoporphyrin IX, Silymarin-Group, Thiotic Acid and Tiopronin;

Immunomodulators such as Amiprilose, Bucillamine, Ditiocarb Sodium, Inosine Pranobex, Interferon-y, Interleukin-2, Lentinan, Muroctasin, Platonin, Procodazole, Tetramisole, Thymomodulin, Thymopentin and Ubenimex;

Immunosuppressants such as Azathioprine, Cyclosporins and Mizoribine;

Ion exchange resins such as Carbacrylic Resins, Cholestyramine Resin, Colestipol, Polidexide, Resodec and Sodium Polystyrene Sulfonate;

Lactation stimulating hormone such as Prolactin;

LH-RH agonists such as Buserelin, Goserelin, Leuprolide, Nafarelin, and Triptorelin;

Lipotropic agents such as N-Acetylmethionine, Choline Chloride, Choline Dehydrocholate, Choline Dihydrogen Citrate, Inositol, Lecithin and Methionine;

Lupus erythematosus suppressants such as Bismuth Sodium Triglycollamate, Bismuth Subsalicylate, Chloroquine and Hydroxychloroquine;

Mineralcorticoids such as Aldosterone, Deoxycorticosterone, Deoxycorticosterone Acetate and Fludrocortisone;

Miotic drugs such as Carbachol, Physostigmine, Pilocarpine and Pilocarpus;

Monoamine oxidase inhibitors such as Deprenyl, Iproclozide, Iproniazid, Isocarboxazid, Moclobemide, Octomoxin, Pargyline, Phenelzine, Phenoxypropazine, Pivalylbenzhydrazine, Prodipine, Toloxatone and Tranylcypromine;

Mucolytic agents such as Acetylcysteine, Bromhexine, Carbocysteine, Domiodol, Letosteine, Lysozyme, Mecysteine Hydrochloride, Mesna, Sobrerol, Stepronin, Tiopronin and Tyloxapol;

Muscle relaxants (skeletal) such as Afloqualone, Alcuronium, Atracurium Besylate, Baclofen, Benzoctamine, Benzoquinonium Chloride, C-Calebassine, Carisoprodol, Chlormezanone, Chlorphenesin Carbamate, Chlorproethazine, Chlozoxazone, Curare, Cyclarbamate, Cyclobenzaprine, Dantrolene, Decamethonium Bromide, Diazepam, Eperisone, Fazadinium Bromide, Flumetramide, Gallamine Triethiodide, Hexacarbacholine Bromide, Hexafluorenium Bromide, Idrocilamide, Lauexium Methyl Sulfate, Leptodactyline, Memantine, Mephenesin, Mephenoxalone, Metaxalone, Methocarbamol, Metocurine Iodide, Nimetazepam, Orphenadrine, Pancuronium Bromide, Phenprobamate, Phenyramidol, Pipecurium Bromide, Promoxolane, Quinine Sulfate, Styramate, Succinylcholine Bromide, Succinylcholine Chloride, Succinylcholine Iodine, Suxethonium Bromide, Tetrazepam, Thiocolchicoside, Tizanidine, Tolperisone, Tubocurarine Chloride, Vecuronium Bromide and Zoxolamine;

Narcotic antagonists such as Amiphenazole, Cyclazocine, Levallorphan, Nadide, Nalmfene, Nalorphine, Nalorphine Dinicotinate, Naloxone and Naltrexone;

Neuroprotective agents such as Dizocilpine;

Nootropic agents such as Aceglutamide, Acetylcarnitine, Aniracetam, Bifematlane, Exifone, Fipexide, Idebenone, Indeloxazune Hydrochloride, Nizofenone, Oxiracetam, Piracetam, Propentofylline, Pyritinol and Tacrine;

Ophthalmic agents such as 15-ketoprostaglandins;

Ovarian hormone such as Relaxin;

Oxytocic drugs such as Carboprost, Cargutocin, Deaminooxytocin, Ergonovine, Gemeprost, Methylergonovine, Oxytocin, Pituitary (Posterior), Prostaglandin E₂, Prostaglandin F₂ₐ and Sparteine;

Pepsin inhibitors such as Sodium Amylosulfate;

Peristaltic stimulants such as Cisapride;

Progestogens such as Allylestrenol, Anagestone, Chlormadinone

Acetate, Delmadinone Acetate, Demegestone, Desogestrel, Dimethisterone, Dydrogesterone, Ethisterone, Ethynodiol, Fluorogestone Acetate, Gestodene, Gestonorone Caproate, Haloprogesterone, 17-Hydroxy-16-methylene—progesterone, 17α-Hydroxyprogesterone, 17α-Hydroxygesterone Caproate, Lynestrenol, Medrogestone, Medroxyprogesterone, Megestrol Acetate, Melengestrol, Norethindrone, Norethynodrel, Norgesterone, Norgestimate, Norgestrel, Norgestrienone, Norvinisterone, Pentagestrone, Progesterone, Promegestone, Quingestrone and Trengestone;

Prolactin inhibitors such as Metergoline;

Prostaglandins and prostaglandin analogs such as Arbaprostil, Carboprost, Enprostil, Bemeprost, Limaprost, Misoprostol, Ornoprostil, Prostacyclin, Prostaglandin E₁, Prostaglandin E₂, Prostagland in F₂ₐ, Rioprostil, Rosaprostol, Sulprostone and Trimoprostil;

Protease inhibitors such as Aprotinin, Camostat, Gabexate and Nafamostat;

Respiratory stimulants such as Almitrine, Bemegride, Carbon Dioxide, Cropropamide, Crotethamide, Dimefline, Dimorpholamine, Doxapram, Ethamivan, Fominoben, Lobeline, Mepixanox, Metamivam, Nikethamide, Picrotoxin, Pimeclone, Pyridofylline, Sodium Succinate and Tacrine;

Sclerosing agents such as Ethanolamine, Ethylamine, 2-Hexyldecanoic Acid, Polidocanol, Quinine Bisulfate, Quinine Urea Hydrochloride, Sodium Ricinoleate, Sodium Tetradecyl Sulfate and Tribenoside;

Sedatives and hypnotics, including: Acyclic ureides such as Acecarbromal, Apronalide, Bomisovalum, Capuride, Carbromal and Ectylurea;

Alcohols such as Chlorhexadol, Ethchlorvynol, Meparfynol, 4-Methyl-5-thiazoleethanol, tert-Pentyl Alcohol and 2,2,2-Trichloroethanol;

Amides such as Butoctamide, Diethylbromoacetamide, Ibrotamide, Isovaleryl Diethylamide, Niaprazine, Tricetamide, Trimetozine, Zolpidem and Zopiclone;

Barbituric acid derivatives such as Allobarbital, Amobarbital, Aprobarbital, Barbital, Brallabarbital, Butabarbital Sodium, Butalbital, Butallylonal, Butethal, Carbubarb, Cyclobarbital, Cyclopentobarbital, Enallyipropymal, 5-Ethyl-5-(1-piperidyl) barbituric Acid, 5-Furfuryl-5-isopropylbarbituric Acid, Heptabarbital, Hexethal Sodium, Hexobarbital, Mephobarbital, Methitural, Narcobarbital, Nealbarbital, Pentobarbital Sodium, Phenallymal, Phenobarbital, Phenobarbital Sodium, Phenylmethylbarbituric Acid, Probarbital, Propallylonal, Proxibarbal, Reposal, Secobarbital Sodium, Talbutal, Tetrabarbital, Vinbarbital Sodium and Vinylbital;

Benzodiazepine derivatives such as Brotizolam, Doxefazepam, Estazolam, Flunitrazepam, Flurazepam, Haloxazolam, Loprazolam, Lormetazepam, Nitrazepam, Quazepam, Temazepam and Triazolam;

Bromides such as Ammonium Bromide, Calcium Bromide, Calcium Bromolactobionate, Lithium Bromide, Magnesium Bromide, Potassium Bromide and Sodium Bromide;

Carbamates such as Amyl Carbamate—Tertiary, Ethinamate, Hexaprpymate, Meparfynol Carbamate, Novonal and Tricholorourethan;

Chloral derivatives such as Carbocloral, Chloral Betaine, Chloral Formamide, Chloral Hydrate, Chloralantipyrine, Dichloralphenazone, Pentaerythritol Chloral and Triclofos;

Piperidinediones such as Glutehimide, Methyprylon, Piperidione, Pyrithyldione, Taglutimide and Thalidomide;

Quinazolone derivatives such as Etaqualone, Mecloqualone and Methaqualone; and

others such as Acetal, Acetophenone, Aldol, Ammonium Valerate, Amphenidone, d-Bornyl α-Bromoisovalerate, d-Bornyl Isovalerate, Bromoform, Calcium 2-Ethylbutanoate, Carfinate, α-Chlorolose, Clomethiazole, Cypripedium, Doxylamine, Etodroxizine, Etomidate, Fenadiazole, Homofenazine, Hydrobromic Acid, Mecloxamine, Menthyl Valerate, Opium, Paraldehyde, Perlapine, Propiomazine, Rilmazaf one, Sodium Oxybate, Sulfonethylmethane and Sulfonmethane;

Thrombolytic agents such as APSAC, Plasmin, Pro-Urokinase, Streptokinase, Tissue Plasminogen Activator and Urokinase;

Thyrotropic hormones such as TRH and TSH;

Uricosurics such as Benzbromarone, Ethebenecid, Orotic Acid, Oxycinchophen, Probenecid, Sulfinpyrazone, Ticrynafen and Zoxazolamine;

Vasodilators (cerebral) such as Bencyclane, Cinnarizine, Citicoline, Cyclandelate, Ciclonicate, Diisopropylamine Dichloractetate, Eburnamorine, Fenoxedil, Flunarizine, Ibudilast, Ifenprodil, Nafronyl, Nicametate, Nicergoline, Nimodipine, Papaverine, Pentifylline, Tinofedrine, Vincamine, Vinpocetine and Viquidil;

Vasodilators (coronary) such as Amotriphene, Bendazol, Benfurodil Hemisuccinate, Benziodarone, Chloacizine, Chromonar, Clobenfurol, Clonitrate, Dilazep, Dipyridamole, proprenilamine, Efloxate, Erythritol, Erythrityl Tetranitrate, Etafenone, Fendiline, Floredil, Ganglefene, Hexestrol Bis(β-diethylaminoethyl ether), Hexobendine, Itramin Tosylate, Khellin, Lidoflazine, Mannitol Hexanitrate, Medibazine, Nicorandil, Nitroglycerin, Pentaerythritol Tetranitrate, Pentrinitrol, Perhexyline, Pimethylline, Prenylamine, Propatyl Nitrate, Pyridofylline, Trapidil, Tricromyl, Trimetazidine, TroInitrate Phosphate and Visnadine;

Vasodilators (peripheral) such as Aluminum Nicotinate, Bamethan, Bencyclane, Betahistine, Bradykinin, Brovincamine, Bufoniode, Buflomedil, Butalamine, Cetiedil, Ciclonicate, Cinepazide, Cinnarizine, Cyclandelate, Diisopropylamine Dichloracetate, Eledoisin, Fenoxidil, Flunarisine, Heronicate, Ifenprodil, Inositol Niacinate, lsoxsuprine, Kallidin, Kallikrein, Moxisylyte, Nafronyl, Nicametate, Nicergoline, Nicofuranose, Nicotinyl Alcohol, Nylidrin, Pentifylline, Pentoxifylline, Piribedil, Protaglandin E₁, Suloctidil and Xanthinal Niacinate;

Vasoprotectants such as Benzarone, Bioflavonoids, Chromocarb, Clobeoside, Diosmin, Dobesilate Calcium, Escin, Rolescutol, Leucocyanidin, Metescufylline, Quercetin, Rutin and Troxerutin;

Vitamins, vitamin sources, and vitamin extracts such as Vitamins A, B, C, D, E, and K and derivatives thereof, Calciferols, Glycyrrhiza and Mecobalamin;

Vulnerary agents such as Acetylcysteine, Allantoin, Asiaticoside, Cadexomer Iodine, Chitin, Dextranomer and Oxaceprol;

Anticoagulants such as heparin;

Miscellaneous such as Erythropoietin (Hematinic), Filgrastim, Finasterlde (Benign Prostate Hypertrophy) and Interferon β1-α (Multiple Sclerosis).

Nucleic acid based-therapeutics, such as antisense nucleic acids and siRNA, or genes for gene therapy.

Gene delivery vehicles for gene therapy, such as viruses, virus particles and viroids.

Chemotherapeutic agents, including Alkylating agents such as Cyclophosphamide, Mechlorethamine, Chlorambucil and Melphalan; Anthracyclines such as Daunorubicin, Doxorubicin, Epirubicin, Idarubicin, Mitoxantrone, Valrubicin; Cytoskeletal disruptors such as Paclitaxel and Docetaxel, and other taxanes; Epothilones; Inhibitors of topoisomerase II such as Etoposide, Teniposide and Tafluposide; Nucleotide analogs and precursor analogs such as Azacitidine, Azathioprine, Capecitabine, Cytarabine, Doxifluridine, Fluorouracil, Gemcitabine, Mercaptopurine, Methotrexate and Tioguanine (formerly Thioguanine); Peptide antibiotics such as Bleomycin; Platinum-based agents such as Carboplatin, Cisplatin and Oxaliplatin; Retinoids such as All-trans retinoic acid; and Vinca alkaloids and derivatives such as Vinblastine, Vincristine, Vindesine and Vinorelbine.

In certain embodiments, the agent to be delivered is one or more proteins, hormones, vitamins or minerals. In certain embodiments, the agent to be delivered is selected from insulin, IGF-1, testosterone, vinpocetin, hexarelin, GHRP-6 or calcium. In certain embodiments, the compositions contain two or more agents.

The above list of active agents is based upon those categories and species of drugs set forth on pages THER-1 to THER-28 of The Merck Index, 12th Edition, Merck & Co. Rahway, N.J. (1996). This reference is incorporated by reference herein in its entirety.

The macromolecules and small molecules can be characterized by their ability to interact with the counterion and antisolvent, such as citrate (counterion) and isopropanol (solvent), to form intact, discrete microspheres containing a high content of the macromolecule or small molecule. The content of the macromolecule or small molecule in the microspheres can vary from about or at 5%, 10%, 15%, 20%, 25%, 30%, 40%, 45%, 50%, 55%, 60%, 65%, 70%, 75%, 80%, 85%, 90%, 91%, 92%, 93%, 94%, 95%, 96%, 97%, 98%, 99% or greater weight/weight (w/w) of the microspheres. In some embodiments, the macromolecule or small molecule content of the microsphere is substantially the same as the amount of macromolecule or small molecule initially in solution, prior to forming the microspheres.

The macromolecules used to prepare microspheres by the methods provided herein can include peptides, such as somatostatins and leuprolides, polypeptides and proteins, glycopeptides such as vancomycin, carbohydrates, including lipids, fatty acids, polysaccharides and nucleic acids (DNA, RNA or PNA, siRNA, tRNA), viruses, such as tobacco mosaic virus, virus particles, viroids and prions. In some embodiments, the macromolecules are proteins, including therapeutic proteins such as DAS181 (the sialidase fusion protein having the sequence of amino acid residues set forth in SEQ ID NO:17), alpha1-antitrypsin, PI8, eglin c, Ecotin, aprotinin, recombinant human DNase, insulin, interferons, recombinant human DNAse (rhDNAse, useful, for example, in the treatment of cystic fibrosis as an inhalation therapeutic (Genentech); see also Shak et al., Proc. Natl. Acad. Sci. USA, 87:9188-9192 (1990)), human serum albumin, human growth hormone, parathyroid hormone and calcitonin. In some embodiments, the protein is DAS181, the counterion is sodium sulfate or sodium citrate, and the antisolvent is isopropanol. In other embodiments, the macromolecule is a nucleic acid, e.g., siRNA, the counterion is polyethyleneimine (PEI) and the antisolvent is isopropanol. In yet other embodiments, the macromolecule is a virus, e.g., tobacco mosaic virus, the counterion is Na-sulfate/Na-acetate, and the antisolvent is isopropanol. In further embodiments, the macromolecule is a peptide, e.g., leuprolide or somatostatin, the counterion is sodium glutamate, and the antisolvent is isopropanol.

The small molecules used to prepare microspheres by the methods provided herein can include antibiotics, such as the aminoglycosides tobramycin and kanamycin, penicillins and tetracyclines, sterols, steroid hormones, prostaglandins, chemotherapeutic agents, such as paclitaxel, or any other small molecule of interest. For example, in one embodiment, the small molecule is tetracycline, the counterion is arginine, and the antisolvent is isopropanol. In another embodiment, the small molecule is kanamycin or tobramycin, the counterion is itaconic acid, and the antisolvent is isopropanol. In yet another embodiment, the small molecule is paclitaxel, the solvent is t-butanol, the antisolvent is water (in which sodium citrate is dissolved to form a citrate buffer), and the counterion is sodium citrate.

The methods provided herein can avoid the use of conditions, such as heat, that can compromise the activity of the compound, e.g., melting of a small molecule compound or denaturation of a protein, and reduce its activity. The microspheres provided according to the methods provided herein therefore can be used to prepare vaccines or other therapeutic medications that require compounds to retain their activity, e.g., proteins or peptides to be present in their native conformation.

The concentration of the compound in solution, used during precipitation of the microspheres, can be between about or at 0.1 mg/ml to about or at 0.2, 05, 0.8, 1.0, 2.0, 5.0, 10.0, 12.0, 15.0, 20.0, 25.0, 30.0, 35.0, 40.0, 45.0, 50.0, 60.0, 70.0, 80.0, 90.0, 100, or 200 mg/ml. In some embodiments, the concentration is between about or at 1 mg/ml and about or at 20 mg/ml. Depending on the characteristics of the molecule (pl, hydrophobicity, solubility, stability, etc.) and other process parameters, the concentration of molecule can empirically be determined to achieve formation of microspheres of a desired size. In general, molecules with lower solubility in the solvent prior to adding counterion and organic solvent can be used at lower concentrations (0.1-5 mg/ml) to form microspheres according to the methods herein, while molecules with higher solubility can be used at 1-20 mg/ml or higher. If the formation of amorphous aggregates or aggregated microspheres is observed, the concentration of the molecule generally should be decreased to reduce or prevent such aggregation.

Nature and Concentration of Counterion

The counterion can be any compound capable of neutralizing one or more oppositely charged groups on the molecule at the pH at which the method is performed. Depending on the characteristics of the molecule (pK, pl, nature and quantity of charged groups, distribution of charge groups on the surface, solubility and structural stability under different pH conditions), the pH can empirically be determined for microsphere formation. In general, for a macromolecule such as a protein, if precipitation is performed at a pH below the pK of the macromolecule, anionic counterions can be used. In general, if precipitation is performed at a pH above the pK of the macromolecule, cationic counterions can be used. The counterion can empirically be selected based on its suitability to initiate microsphere formation. In some embodiments, the counterion can have a molecular weight of 60 Daltons or greater, or about 75 Daltons or greater. The counterion can be a polymer, such as polyethylene glycol (PEG) or polyethyleneimine (PEI).

The counterions can be anionic, cationic or zwitterionic. Anionic counterions can be inorganic (phosphate, sulphate, thiocyanate, thiosulfate, hypochlorate, nitrate, bromine, iodine, etc.) or organic compounds that carry charge-polarizable groups including enol, hydroxy, —SH, carboxylic, carboxymethyl, sulfopropyl, sulfonic, and phosphoric. Organic compounds carrying other anionic groups or having negative charge due to other molecular characteristics also can be used. Compounds that can be used as anionic counterions also include, but are not limited to, the following: oxaloacetate, malate, maleate, oxalate, piruvate, citrate, succinate, fumarate, ketoglutarate, butanetricarboxylic acid, hydromuconic acid, cyclobutanedicarboxylic acid, dimethyl maleate, deoxyribonucleic acid, polyglutamic acid, folic acid, lactic acid, ascorbic acid, carminic acid, sorbic acid, malonic acid, EDTA, MOPS, TES, MES, PIPES, pyridine, tricine, betaine, sulfuric acid, thiosulfuric acid, phosphoric acid, adenosine triphosphate, nitric acid, itaconic acid, pivalic acid, dimethylmalonic acid, and perchloric acid. In some embodiments, itaconic, pivalic, dimethylmalonic, and succinic acids are used as counterions in the methods provided herein.

Cationic counterions can be inorganic (ammonium, phosphonium, sulfonium, cesium, rubidium, etc.) or organic compounds that carry groups known as amine, amide, imine, imide, guanidine, imidazole, dioxane, aniline. Organic compounds carrying other cationic groups or have positive charge polarizability due to other molecular characteristics also can be used. Compounds that can be used as cationic counterions also include, but are not limited to, the following: Tris, Bis-Tris, Bis-Tris propane, diaminopropane, piperazine, piperadine, pentylamine, diaminobutane, propylamine, trimethylamine, triethylamine, spermine, spermidine, putrescine, cadaverine, ethanolamine, diethanolamine, triethanolamine, imidazole, tetramethylammonium, trimethylammonium, ammonium, cesium, rubidium, imidazole, polyethileneimine (PEI), DEAE, TEAE, QAE.

Zwitterionic counterions possessing any charged groups in any combination can also be used. Compounds that can be used as zwitterionic counterions include, but are not limited to, the following: HEPES, BICINE, glycine, glycylglycine, 6-aminohexanoic acid, piperidic acid, natural and non-natural amino acids (e.g., histidine, glutamine, arginine, lysine).

The counterions can be used as acids (e.g. sulfuric acid) or bases (e.g. imidazole) or their salts (e.g. sodium sulfate or imidazole-HCl). Counterions that can be used in the methods provided herein include those listed by the National Formulary, United States Pharmacopeia, Japanese Pharmacopeia, or European Pharmacopeia, the clinical safety of which has been demonstrated (citric acid, malic acid, amino acids, sulfate, etc.). In some embodiments, counterions used in the methods provided herein include ones for which safety has been established or as falling into the GRAS (generally regarded as safe) category. The counterions (or their salts) can be solid at room temperature (about 25° C.), or at the intended temperature of use and storage). Combinations of two or more counterions also can be used. Volatile and liquid counterions also can be used in the methods provided herein.

The concentration of counterion generally is maintained between about or at 0 mM and about or at 0.1, 0.2, 0.5, 0.8, 1.0, 2.0, 3.0, 5.0, 7.0, 10.0, 15.0, 20.0, 30.0, 40.0, 50.0, 60.0, 70.0, 80.0, 90.0 and 100.0 mM. In some embodiments, the concentration of the counterion is between about or at 0.5 mM and about or at 20 mM. Depending on the characteristics of the macromolecule or small molecule (pl (e.g., for proteins or peptides), hydrophobicity, solubility, stability, etc.) and other process parameters, the concentration of the counterion can empirically be determined using, for example, a high-throughput format as provided herein. In general, the formation of oversized microspheres, amorphous aggregates or aggregated microspheres indicates that the concentration of counterion should be decreased, while failure to form microspheres (broken glass-like crystals or flakes) or formation of microspheres below the desired size indicates that the concentration of counterion should be increased.

Counterions that Produce Microspheres in the Absence of Added Compound

In the course of screening conditions for microsphere formation, including empirical variation of the type and nature of solvent, antisolvent, solvent/antisolvent system and counterions (sometimes in a buffer, in other embodiments present without a buffer) for each compound of interest, it was found that several control reactions containing no added compound produced microspheres of counterion/buffer. For example, a solution of 15 mM unbuffered arginine with 25% isopropanol produced microspheres with a rating of 7, with some crystallinity present. A solution containing 2 mM Na-sulfate with 0.2 mM Na-Acetate buffer at either pH 4 or pH 6 in 15% isopropanol, resulted in microspheres with a rating of 7, at both conditions. Although some clumping was present, many small, well-separated, discrete microspheres also were observed. Itaconic acid also showed a propensity to form microspheres independently, with no added compound. When a 2 mM solution of itaconic acid was buffered with sodium hydroxide at pH 4 in the presence of 15% isopropanol, microspheres were formed. A similar cocktail containing 2 mM itaconic acid buffered to pH 7 with 5% isopropanol, produced hygroscopic microspheres. Similarly, pivalic acid also was found to make microspheres independent of an additional compound. For example, when a 2 mM solution of pivalic acid was titrated to pH 5 with sodium hydroxide in the presence of 15% isopropanol, microspheres of pivalic acid were produced that had a rating of 6.

The above types of counterions can be useful as a tool for catalyzing microsphere formation in molecules that otherwise might not form micro particles.

Solvent/Antisolvent System

A solvent/antisolvent system suitable for use in the methods of microsphere formation provided herein can be based on the relative solubilities of the compound of interest in the solvent and in the antisolvent, as known and available to those of skill in the art. Alternately, the solubilities of the compound of interest in the solvent and/or the antisolvent can be determined empirically, by varying the types and concentrations of various solvents, antisolvents and counterions in a high-throughput format, as provided herein, or by other methods known to those of skill in the art including, but not limited to, dissolution saturation testing.

In general, the compound of interest that is used to form the microspheres is soluble in the selected solvent (from about or at 1 mg/ml to about or at 100 mg/ml). The antisolvent can be selected from among a group of solvents in which the compound of interest has limited or no solubility. The solvent and antisolvent generally are selected such that they are miscible, or partially miscible, at the temperatures used for dissolution to prepare the cocktail solution. In some embodiments it is possible, however, that the solvent and antisolvent can have different freezing points; therefore, lowering the temperature can cause one of the components to freeze, thereby increasing the concentration of the antisolvent, thereby inducing precipitation (e.g., in some preparations of microspheres of DAS181, using 5% isopropanol as the antisolvent). In general, it is desirable to select a solvent/antisolvent system that does not facilitate precipitation of components other than the compound of interest (e.g., counterion and excipients). The solvent and antisolvent can be a combination of an aqueous liquid and a non-aqueous and/or organic liquid, or both can be non-aqueous and/or organic liquids.

Nature and Concentration of Solvent

A number of macromolecules and small molecules, among the microparticle-forming compounds of interest, are soluble in water and aqueous solutions; hence, the solvent for such molecules generally is aqueous. For compounds that are not soluble in aqueous solvents, the solvent used in the methods provided herein generally can be water miscible and is selected from among alcohols (methanol, ethanol, 1-propanol, isopropanol, butanol, tert-butyl alcohol), chloroform, dimethyl chloride, polyhydric sugar alcohols (glycerin, erythritol, arabitol, xylitol, sorbitol, mannitol), aromatic hydrocarbons, aldehydes, ketones, esters, ethers (di-ethyl ether), alkanes (hexane, cyclohexane, petroleum ether), alkenes, conjugated dienes, toluene, dichloromethane, acetonitrile, ethyl acetate, polyols, polyimids, polyesters, polyaldehydes, dimethyl formamide (DMF), dimethyl sulfoxide (DMSO), carbon tetrachloride, and mixtures thereof. In some embodiments, the solvent can be volatile. In other embodiments, when incorporation of the solvent into the microspheres is desired, non-volatile solvents can be used that provide, for example, novel characteristics to the microspheres (e.g., sustained release or added mechanical strength). The concentration of the solvent generally can be maintained between about or at 0.1%, to about or at 0.5%, 1%, 2%, 5%, 10%, 15%, 20%, 25%, 30%, 40% or 50%, volume/volume (v/v). In some embodiments, the concentration of the solvent is between about or at 1% to about or at 30%, v/v. Organic compounds that are partially miscible or completely immiscible with water also can be used as solvents for water-insoluble compounds.

Organic solvents that can be used in the methods provided herein include alcohols and others listed as Class 3 and 2 solvents in International Conference on Harmonisation (ICH) Harmonised Tripartite Guideline (Impurities: Guideline for Residual Solvents), safe handling of which has been established in pharmaceutical and food industries.

Depending on the characteristics of the molecule (hydrophobicity, solubility, stability, etc.) and other process parameters, the choice and concentration of the solvent can be optimized, for example, using high-throughput screening on microtiter plates or similar chips or other device. In general, uncontrolled precipitation before the initiation of cooling, the formation of oversized microspheres, amorphous aggregates, aggregated microspheres or sticky aggregates indicates that solvent that affords higher solubility of the drug should be used, while failure to form microspheres (broken glass-like crystals or flakes) or formation of microspheres below the desired size indicates that use of solvent with lower drug solubility may be beneficial.

Nature and Concentration of Antisolvent

In general, if the compound of interest is water-soluble and in an aqueous solution, the antisolvent is an organic solvent. On the other hand, if the compound of interest is water-insoluble, the antisolvent is an aqueous solvent. The solvent and the antisolvent can, however, both be organic solvents. Under conditions of mixing of the cocktail reagents and/or precipitation by chilling to initiate microsphere formation, the antisolvent generally is miscible or partially miscible with the solvent in which the compound forming the microparticle is dissolved. Such solvents include, for example, water and other aqueous solutions, such as buffers, alcohols (methanol, ethanol, 1-propanol, isopropanol, butanol, tert-butyl alcohol), chloroform, polyhydric sugar alcohols (glycerin, erythritol, arabitol, xylitol, sorbitol, mannitol), aromatic hydrocarbons, aldehydes, ketones, esters, ethers (di-ethyl ether), alkanes (hexane, cyclohexane, petroleum ether), alkenes, conjugated dienes, toluene, dichloromethane, carbon tetrachloride, dimethylformamide (DMF), dimethyl sulf oxide (DMSO), acetonitrile, ethyl acetate, polyols, polyimides, polyesters, polyaldehydes, and mixtures thereof.

In some embodiments, the organic solvent can be volatile. In other embodiments, when incorporation of the organic solvent into the microspheres is desired, non-volatile organic solvents can be used that provide, for example, novel characteristics to the microspheres (e.g., sustained release or added mechanical strength). The concentration of the organic solvent generally can be maintained between about or at 0.1%, to about or at 0.5%, 1%, 2%, 5%, 10%, 15%, 20%, 25%, 30%, 40% or 50%, volume/volume (v/v). In some embodiments, the concentration of the organic solvent is between about or at 1% to about or at 30%, v/v. Organic compounds that are partially miscible or completely immiscible with water also can be used.

Organic solvents that can be used in the methods provided herein include alcohols and others listed as Class 3 and 2 solvents in International Conference on Harmonisation (ICH) Harmonised Tripartite Guideline (Impurities: Guideline for Residual Solvents), safe handling of which has been established in pharmaceutical and food industries.

Depending on the characteristics of the molecule (hydrophobicity, solubility, stability, etc.) and other process parameters, the choice and concentration of the anti-solvent can be optimized, for example, using high-throughput screening on microtiter plates or similar chips or other device. In general, uncontrolled precipitation before the initiation of cooling, the formation of oversized microspheres, amorphous aggregates, aggregated microspheres or sticky aggregates indicates that the concentration of anti-solvent should be decreased, while failure to form microspheres (broken glass-like crystals or flakes) or formation of microspheres below the desired size indicates that the concentration of the anti-solvent should be increased.

pH

In addition to initiating microsphere formation, the counterion also can serve as a buffer. Alternately, in some embodiments, a buffering compound can be used to obtain the desired pH. In some embodiments, the buffering compound is 60 Da or larger. Depending on the characteristics of the molecule (pl, hydrophobicity, solubility and stability at a specific pH, etc.) and other process parameters, the optimal pH can empirically be adjusted to achieve formation of microspheres of desired dimensions and preserve the activity of the molecule. In general, failure to form microspheres (broken glass-like crystals or flakes) indicates that the molecule may be too soluble under the conditions used. Formation of amorphous aggregates can indicate that precipitation is not well controlled and the molecule, such as a protein, may not be stable or soluble at the pH used.

It has been observed that certain compound/counterion combinations can cause immediate and uncontrolled precipitation at certain pH values. The high-throughout screening methods provided herein can be used to empirically determine the appropriate combination of protein, pH and counterion to form microspheres of desired dimensions. For example, empirical determinations including changing the pH of the cocktail, using a different counterion or decreasing the concentration of the compound in the cocktail, can conveniently and rapidly be performed in semi high-throughput or high throughput format. In general, for forming protein or polypeptide-based microspheres, a pH value that is below the pl of the protein provides optimal microsphere formation. Such empirical optimization methods are applicable to other macromolecules and small molecules as provided and exemplified herein.

Ionic Strength

The ionic strength of the cocktail solution can be modulated by adjusting the concentration of the counterion or other salts, such as chlorides or acetates. In some embodiments, no additional salt is required to produce microspheres. In certain embodiments, the ionic strength can be adjusted to preserve the structural integrity and activity of the molecule. Examples of other applications where the presence of specific salts can be beneficial include formulations of parenteral and other drugs, or foods where specific tonicity or buffering capacity may be required upon reconstitution of microspheres.

Cooling Ramp

The cocktail containing a molecule, a counterion and a suitable solvent/antisolvent system initially is prepared, prior to cooling, at a temperature at which the molecule is soluble, generally about −15° C. to about 30° C. In some embodiments, the initial temperature, prior to cooling is at ambient temperature (18° C. to 25-30° C.). In other embodiments, for example, with small molecules, the compound can be dissolved in the solvent and/or antisolvent system at much higher temperatures, for example, about or at 50° C., 60° C., 65° C., 70° C., 75° C., 80° C., 85° C., 90° C., 95° C., 100° C., 125° C., 150° C., 175° C., 200° C. or greater, then cooled to a temperature of, for example, about or at 190° C., 170° C., 150° C., 125° C., 100° C., 80° C., 75° C., 60° C., 50° C., 40° C., 30° C., 20° C., 15° C. or lower, at which the microspheres are formed. The microspheres are formed by a process such as precipitation, phase separation or colloid formation upon gradual cooling to a temperature below the temperature at which the macromolecule is dissolved and in solution. The rate at which cooling is performed can control the formation and other characteristics such as size of the microspheres. In general, when the molecule is a protein, flash-freezing in liquid nitrogen does not generate microspheres.

The rate at which cooling and freezing of the cocktail (cooling ramp) is performed can determine the final size of the microspheres. In general, a faster cooling ramp yields smaller microspheres whereas a slower cooling ramp yields larger microspheres. Without being bound by any theory, the cooling rate can determine the rate of: (1) nucleation that produces initial smaller microspheres and (2) a fusion process in which the initial microspheres coalesce (aggregate) and anneal into larger microspheres. Fusion of the smaller particles into larger ones is a time dependent process that can be determined, for example, by the duration for which liquid suspension of microspheres exists prior to freezing. Due to the reversible nature of the bonds between molecules, in the microsphere compositions provided herein, smaller microspheres annealing into larger particles can generate microspheres with smooth surfaces. Depending on the size of microparticles desired, the cooling rate can be from about 0.01° C./min or 0.01° C./min to about 20° C./min or 20° C./min; from about or at 0.05° C./min or about or at 0.1° C./min to about or at 10° C./min or about or at 15° C./min, from about or at 0.2° C./min to about or at 5° C./min, from about or at 0.5° C./min to about or at 2° C./min, or about or at 1° C./min. In some embodiments, the cooling ramp can be between 0.1° C. per minute and about 40° C. per minute. In other embodiments, a cooling ramp can be between about 0.5° C. per minute and 15° C. per minute.

Depending on the specific needs, in some embodiments it can be desirable to adapt the production process to the specific equipment. In some embodiments, a lyophilizer with temperature-controlled shelves can be used for the cooling. In other embodiments, endothermic reactions can be used for the cooling. If the microspheres produced are larger than desired, other parameters of the process including concentration of the molecule, antisolvent, counterion, ionic strength and/or pH can be modified to achieve the desired reduction in size of the microspheres.

For a faster cooling ramp (smaller particle size), the cocktail solution can be passed through a heat exchanger, such as that used in a continuous mode. If the size of microspheres needs to be increased, increased concentrations of one of the cocktail ingredients (molecule, antisolvent, counterion) can provide the desired increase in the size of microspheres.

In general, the cooling should be performed uniformly and at a steady rate to prevent the formation of aggregates and crystals or glass-like particulates. Depending on the concentration of the antisolvent, the precipitation of the molecule into microspheres can occur in several ways. At higher concentrations of antisolvent (about 5%-40%, dependent on the actual components used) the microspheres generally can form when the cocktail solution is still in liquid form. At lower concentrations of antisolvent (2-25%, dependent on the actual components used) ice crystals can form first, following which the expelled molecules and antisolvent reach can reach a critical local concentration and precipitate. A further decrease of temperature in the near-bottom layer of the lyophilizer tray can lead to complete solidification of the liquid suspension and further expulsion of the antisolvent into the top layer. An excess of antisolvent in the top layer can cause uncontrolled precipitation of the molecule and aggregation of microspheres. This effect usually can be alleviated by selecting appropriate ratios of the components—molecule, counterion, antisolvent, salts, etc. in the cocktail. In addition, maintaining a thin layer of cocktail in the lyophilization tray or mixing of the cocktail while being chilled can prevent formation of aggregates and crystals and yield uniform microspheres. For example, if a relatively low concentration of Isopropanol (e.g. 2-6%) is used, and a thin layer of cocktail (10-20 mm) is filled into the tray, and the tray is placed on a pre-chilled shelf (generally, −30° C. to −75° C.), uniform microspheres can be obtained.

The methods provided herein can, under some conditions, lead to substantially all or all the molecule being incorporated from the solution into the microspheres

High-Throughout Screening of Microparticle Formation Conditions and Optimization of Particle Formation

Depending on the characteristics of the molecule, the composition of the cocktail solution used to prepare the microspheres according to the methods provided herein can be optimized. The optimization can rapidly be performed in a medium or high throughput format using, for example microtiter plate(s) or chips where tens to hundreds to thousands to tens of thousands of cocktails can be screened simultaneously. In some embodiments, a number of pH values in conjunction with cationic, anionic or zwitterionic counterions and antisolvents at various concentrations can be screened. For example, the screening can be performed using several identical microtiter plates, to each of which the molecule of interest is added at various concentrations. Each set of test conditions can be screened in duplicate. In some embodiments, microplates with flat-bottom wells can be used with the skirt of the microtiter plate broken off to permit good heat transfer between the lyophilizer shelf and the bottoms of the wells. The microplates can be placed on the shelves of the lyophilizer and cooled to form microspheres and to subsequently solidify the suspensions. Upon freezing of the contents of the wells, a vacuum can be applied. At the end of lyophilization, one of the duplicate plates can be reconstituted with water or a buffer of choice to observe if certain conditions rendered the molecule insoluble or reduced its activity. Conditions that resulted in material that can readily be resolubilized or provide microspheres with desirable characteristics can be subjected to further analysis by spectroscopic, chromatographic, enzymatic or other assays to confirm that native structure and activity are preserved. Lyophilized material in a duplicate plate can be used for microscopy to determine whether microspheres are formed. Conditions that produced microspheres can further be modified and fine-tuned to produce microspheres of desirable size and characteristics.

Kits for performing high-throughput screens can be provided and can contain all the ingredients used in the methods provided herein including one or more of a molecule, buffers, pre-dispensed cocktail of known composition (antisolvent, counterion) and/or salts. Kits can contain 3, 4, 5, 10, 15, 20, 30, 40, 50, 100 or more (in some embodiments, 96 or more) buffers with predetermined pH, counterion, ionic strength and antisolvent in each microtiter plate. The microtiter plate supplied with the kit can be modified so that the bottoms of the wells are in direct contact with the shelf of the lyophilizer.

### C. Large-Scale Manufacture of Microparticles

The methods provided herein can be scaled for the manufacture of large quantities of microspheres. For example, the Batch Process described herein is suitable for the manufacture of high quality dry powder microspheres in an amount ranging from, for example, milligrams of to about a kilogram, based on the capacity of the mixing tank and/or lyophilizer shelf space. An alternative “continuous” process described herein can be used to manufacture amounts ranging from, for example, hundreds of grams to hundred or more kilograms (100 grams to 100 kg and above). An additional advantage of the continuous process is better control over the chilling of the cocktail.

The large scale manufacture by a batch process or by a continuous process can follow, for example, one or more of the steps described below in any combination:
Precipitation of the molecule into microspheres. This step can be performed in a batch mode by placing the cocktail solution containing the desired concentration of molecule, organic solvent and counterion in lyophilization tray(s) and placing the tray(s) onto lyophilizer shelves. Alternatively, trays can be chilled and frozen on a chilled platform or other type of equipment (e.g., a freezer) and stored for a period of time frozen and lyophilized later. Alternatively, the microspheres can be formed by precipitation in a vessel with stirring, wherein the vessel is placed onto a cold surface or a cooling coil is immersed into liquid or while the cocktail is being recirculated through a heat exchanger using a peristaltic pump. Alternatively, the microspheres can be formed by precipitation in a continuous mode, by passing the cocktail solution through a heat exchanger(s) once using a peristaltic pump.
Removal of bulk liquid. The suspension of the microspheres can be concentrated using standard centrifugation, continuous flow centrifugation (e.g., CARR ViaFuge Pilot), or filtration (e.g., on glass fiber, sintered glass, polymer filters, hollow fiber cartridges (e.g., those manufactured by GE Healthcare) or tangential flow filtration cassettes (TFF cassettes, such as those manufactured by Millipore or Sartorius)). The removal of bulk liquid (50% or greater) can result in a faster drying cycle and higher efficiency and throughput.
Drying the microspheres. The recovered microspheres formed by any mode, can be dried by conventional lyophilization. Alternatively, the microspheres can be dried under ambient temperature and atmospheric pressure, eliminating the use of lyophilizer.

### D. Microparticle Compositions

The molecules contained in the microparticle compositions obtained by the methods provided herein are substantially structurally and chemically unchanged by the methods. For example, when the molecule is a macromolecule such as Green Fluorescent Protein or Red Fluorescent Protein, their fluorescence and native conformation and activity of the proteins are retained in the microparticles. The dry microspheres, obtained by volatilizing substantially all of the solvents and/or moisture except for the solvent and other components associated with the microspheres, can be stored and their activity can substantially be recovered upon reconstitution. The relatively low moisture content of the microparticles provided herein, for example, between about or at 0.01% to about or at 0.05%, 0.1%, 0.2%, 0.3%, 0.5%, 1.0%, 2.0%. 3.0%. 4.0%, 5.0%, 5.5%, 6.0%, 6.5%. 7.0%. 7.5%, 8.0%, 8.5%, 9.0%, 9.5%, 10.0%, 10.5%, 11.0%, 11.5%, 12.0%, 12.5%, 14%, 15%, 16%, 17%, 18% 19%, or 20%, can provide improved stability. The microspheres obtained by the methods provided herein also are homogeneous in size and shape, and can be obtained reproducibly with the desired characteristics. Other techniques traditionally used for preparation of dry formulations (salt precipitation, alcohol or acetone precipitation, lyophilization, e.g.) can result in complete or partial inactivation of the molecule, e.g., denaturation of a protein. In addition, the microspheres prepared by the methods provided herein avoid the need for complex or specialized spray drying, spray freeze-drying, supercritical fluid anti-solvent based processes or milling processes (See, for example, Laube B L. The expanding role of aerosols in systemic drug delivery, gene therapy, and vaccination. Respir Care 2005; 50(9):1161-1176; Taylor G, Gumbleton M. Aerosols for Macromolecule Delivery: Design Challenges and Solutions. American Journal of Drug Delivery 2004; 2(3):143-155; Smyth H D C, Hickey A J. Carriers in Drug Powder Delivery. Implications for Inhalation System Design. American Journal of Drug Delivery 2005; 3(2):117-132; Cryan S A. Carrier-based strategies for targeting protein and peptide drugs to the lungs. AAPS J 2005; 7(1):E20-E41; LiCalsi C, Maniaci M J, Christensen T, Phillips E, Ward G H, Witham C. A powder formulation of measles vaccine for aerosol delivery. Vaccine 2001; 19(17-19):2629-2636; Maa Y F, Prestrelski S J. Biopharmaceutical powders: particle formation and formulation considerations. Curr Pharm Biotechnol 2000; 1(3):283-302; Maa Y F, Nguyen P A, Hsu S W. Spray-drying of air-liquid interface sensitive recombinant human growth hormone. J Pharm Sci 1998; 87(2):152-159; Vanbever R, Mintzes J D, Wang J et al. Formulation and physical characterization of large porous particles for inhalation. Pharm Res 1999; 16(11):1735-1742; Bot A I, Tarara T E, Smith D J, Bot S R, Woods C M, Weers J G. Novel lipid-based hollow-porous microparticles as a platform for immunoglobulin delivery to the respiratory tract. Pharm Res 2000; 17(3):275-283; Maa Y F, Nguyen P A, Sweeney T, Shire S J, Hsu C C. Protein inhalation powders: spray drying vs spray freeze drying. Pharm Res 1999; 16(2):249-254; Sellers S P, Clark G S, Sievers R E, Carpenter J F. Dry powders of stable protein formulations from aqueous solutions prepared using supercritical CO(2)-assisted aerosolization. Pharm Sci 2001; 90(6):785-797; Garcia-Contreras L, Morcol T, Bell S J, Hickey A J. Evaluation of novel particles as pulmonary delivery systems for insulin in rats. AAPS PharmSci 2003; 5(2):E9; Pfutzner A, Flacke F, Pohl R et al. Pilot study with technosphere/PTH(1-34)—a new approach for effective pulmonary delivery of parathyroid hormone (1-34). Horm Metab Res 2003; 35(5):319-323; Alcock R, Blair J A, O'Mahony D J, Raoof A, Quirk A V. Modifying the release of leuprolide from spray dried OED microparticles. J Control Release 2002; 82(2-3):429-440; Grenha A, Seijo B, Remunan-Lopez C. Microencapsulated chitosan nanoparticles for lung protein delivery. Eur J Pharm Sci 2005; 25(4-5):427-437; Edwards D A, Hanes J, Caponetti G et al. Large porous particles for pulmonary drug delivery. Science 1997; 276(5320):1868-1871; McKenna B J, Birkedal H, Bartl M H, Deming T J, Stucky G D. Micrometer-sized spherical assemblies of polypeptides and small molecules by acid-base chemistry. Angew Chem Int Ed Engl 2004; 43(42):5652-5655; Oh M, Mirkin C A. Chemically tailorable colloidal particles from infinite coordination polymers. Nature 2005; 438(7068):651-654; U.S. Pat. No. 5,981,719; U.S. Pat. No. 5,849,884 and U.S. Pat. No. 6,090,925; U.S. Patent application No. 20050234114; U.S. Pat. No. 6,051,256).

The microparticles obtained by the methods provided herein can be of any shape—a regular geometric shape including, but not limited to, spherical, elliptical, square, triangular and polyhedral, or an irregular shape. The microparticles can have sizes (mean width or diameters) in the range of from about or at 0.001 micron to about or at 0.002, 0.005, 0.01, 0.02, 0.03, 0.05, 0.1, 0.02, 0.03, 0.5, 1.0, 2.0, 2.5, 3.0, 3.5, 4.0, 4.5, 5.0, 5.5, 6.0, 6.5, 7.0, 7.5, 8.0, 8.5, 9.0, 9.5, 10.0, 15.0, 20.0, 25.0, 30.0, 35.0, 40.0, 45.0, or 50.0 or greater microns. For pulmonary administration to the alveoli, depending on the application, the size can be from about 0.1 micron or less to about or at 0.5 micron or greater, up to about or at 0.6, 0.7, 0.8, 0.9, 1.0, 1.5, 2.0 or 5.0 microns or greater. For administration by inhalation to the throat, trachea and bronchi, the size can be from about or at 0.5 microns to about or at 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 9.5, 10.0 10.0, 15.0 or 20.0 microns or greater, or in some embodiments from about or at 1.0 micron to about or at 2.0 microns. In some embodiments, the microparticles are substantially spherical in shape.

The molecules that can be used to form microparticles according to the methods provided herein can include preventative agents, prophylactic agents, therapeutic and diagnostic agents, processed foods, dietary supplements, nutritional supplements, cosmetic compounds and polymers. In some embodiments, cross-linking agents, salts, or other compounds can be included in the formulation cocktail to modify solubility of the microspheres and/or enhance their mechanical strength. In some embodiments, microspheres that are insoluble in most aqueous or organic solvents can be used to manufacture particles such as chromatographic resins and dispersible abrasives. In other embodiments, microspheres with partial solubility in solvents such as pharmaceutical vehicles for delivery can be useful in the manufacture of sustained release active agent or therapeutic formulations.

In some embodiments, the microparticles provided herein can be used in combination with an inhalation device to deliver a therapeutic dose of microspheres to the respiratory airways and lungs of a subject. For example, when the molecule is the DAS181 protein (sequence set forth in SEQ ID NO: 17), microspheres of about 0.5 micron to about 8 microns, or about 1 micron to about 5 micron can be obtained by the methods provided herein, using sodium sulfate as the counterion and isopropanol as the organic solvent. For DAS181 microspheres, which are administered to prevent or treat viral infections that initiate in the respiratory tract, such as influenza, it can be desirable to deposit the microspheres in the throat, trachea or bronchi. The DAS181 fusion protein formulated as microspheres can act by degrading the receptor sialic acids in the throat/trachea/bronchi, thus preventing viral binding and infection at these sites. For optimal delivery of the DAS181 microspheres to sites where respiratory viral infection can be initiated, i.e., in the throat, trachea or bronchi, the microspheres must not be (a) so big that they are trapped at the front end in the mouth (i.e., microspheres are too big, about 8 microns or greater); or (b) so small that they are absorbed deep in the lungs and absorbed systemically into the blood stream through the alveoli where they are not active and/or can be toxic (i.e., 0.5 micron or smaller). For delivery of the DAS181 microspheres to the throat, trachea and bronchi, a size range of about 1 micron to about 5.5-6 microns generally can be suitable. Similar behavior is observed with microparticles of a much smaller exemplary molecule, vancomycin, prepared by the methods provided herein.

The inhaler can be used to treat any medical condition in which the protein or other molecule can be administered by inhalation therapy. Typical inhalation devices can include dry powder inhalers, metered dose inhalers, and electrostatic delivery devices. Typical applications of inhalation delivery devices include the deep lung delivery of insulin and other therapeutic proteins, and vancomycin.

In some embodiments, the microspheres obtained by the methods provided herein also can be delivered by oral ingestion, intranasally, intravenously, intramuscularly, subcutaneously, transdermally, topically and by other delivery methods suitable for the delivery of therapeutic, diagnostic, nutritional or cosmetic molecules. The microsphere formulations for pulmonary delivery generally can be in a size range of about 0.5 micron to about 5-6 microns, while those designed for other types of delivery, such as subcutaneous delivery, parenteral delivery or intramuscular delivery can be in a range of from about or at 10 micron to about or at 30, 40 or 50 microns.

In some embodiments, the microspheres provided herein have no direct therapeutic effect but can serve as micro-carriers for other therapeutic agent(s). Examples of molecules useful for preparation of such microspheres include but are not limited to polysaccharides, glycans, proteins, peptides, nucleic acids, polymers or combinations thereof, or certain small molecules such as amino acids, sodium acetate, sodium sulfate, sodium citrate or combinations thereof. Therapeutic agents or other active agents can be added at the time of microsphere formation or added to the suspension of formed microspheres. Alternatively, therapeutic agents can be blended with the dry microsphere compositions by mixing, tumbling or other techniques practiced in pharmaceutical and food industries.

Polymers that can serve as micro-carriers for other therapeutic agent(s) in the microspheres provided herein can be any of those defined herein including, but not limited to, nucleic acids such as deoxyribonucleic acid (DNA), ribonucleic acid (RNA), and mixed DNA or RNA derivatives, or peptide nucleic acids (PNA), polyacrylamides, polystyrenes, polyalkyl-substituted styrenes, polyacrylates, polymethacrylates, polyacrylic acid, polymethacrylic acid, polyvinyl chloride, polyvinyl acetate, polybutadiene, polyisoprene, polyethylene glycol and polyethyleneimine. Other exemplary organic or inorganic polymers, natural and synthetic polymers, include, but are not limited to, agarose, cellulose, nitrocellulose, cellulose acetate, other cellulose derivatives, dextran, dextran-derivatives and dextran co-polymers, other polysaccharides, glass, silica gels, proteins such as gelatin, polyethylene glycols, polyethyleneimines, polyethyleneimides, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and the like.

In some embodiments, the micro-carriers can be materials that are capable of forming hydrogels. Hydrogels are water-swellable polymeric matrices that can absorb water to form elastic gels. Hydrogels and hydrogel microspheres have been tested as drug delivery systems for topical and systemic delivery to a variety of target tissues, including eye and bone. The manufacture of hydrogel microspheres has previously been accomplished using complex methods, such as the oil/water emulsion method. The methods provided herein facilitate the simple manufacture of hydrogel microspheres.

Examples of materials capable of forming hydrogels include but are not limited to various natural, genetically engineered, derivatized, and synthetic polymers such as proteins (collagen, gelatin, silk) and polysaccharides (chitosan, dextran, gellan gum, agarose). Examples 22 and 23 demonstrate that materials capable of forming hydrogels (gelatin, dextran) can be incorporated into microsphere formulations prepared by the methods provided herein, resulting in microspheres capable of forming hydrogels. The therapeutic agent or active agent of interest can be added to the cocktail formulation containing the hydrogel-forming material at any time and in any sequence during the steps leading to the formation of microspheres according to the methods provided herein. Alternatively, the therapeutic agent or active agent can be added to the solution used to hydrate/swell the microspheres or can be added to the suspension of swollen microspheres and allowed to diffuse into the particles.

The hydrogel microspheres can be crosslinked to decrease their solubility/erosion and to provide a more sustained release. Cross-linking can be performed using a variety of cross-linking functionalities known to those of skill in the art including, but not limited to, carboxyl, amino, hydroxyl, phosphate, and/or sulfhydryl groups using natural condensation or agents that mediate cross-linking, such as EDC (1-ethyl-3-(3-dimethylaminopropyl) carbodiimide hydrochloride) or other compounds employing carbodiimide and non-carbodiimide chemistries.

In some embodiments, cross-linking agents, polymers, lipophilic substances, salts such as those with poor solubility in aqueous solvents, or combinations thereof or other compounds can be included in the formulation cocktail solution to modify the solubility of the microspheres and/or enhance their mechanical strength. Slow dissolution of the microspheres can be useful in sustained release of therapeutics delivered by oral ingestion, inhalation, intranasally, intravenously, intramuscularly, transdermally, topically, subcutaneously, and by other delivery methods suitable for the delivery or application of therapeutic, diagnostic, nutritional or cosmetic molecules. In some embodiments, the microspheres can be delivered by oral ingestion in a form of a pill or capsule with an enteric coating, endocytosed from the duodenum, and the molecule released into the blood stream or other site of action.

In some embodiments, for example when the molecule is a protein or other macromolecule, the microspheres can be rendered insoluble by partial denaturation of the macromolecule, which upon delivery becomes renatured and bioavailable.

In other embodiments, the microspheres are substantially spherical in shape, and can have mean diameters within the range of from about 0.1 microns to 30.0 microns. In yet other embodiments, the mean diameter of the microspheres can be within the range of from about 0.5 microns to 5.0 microns, or from about 1.0 microns to 2.0 microns.

In yet another aspect, provided herein are devices and methods for delivering the microspheres to a subject, such as an animal or human patient in need of medical treatment. Suitable delivery routes can include parenteral, such as i.m., i.v. and s.c., and non-parenteral, such as oral, buccal, intrathecal, nasal, pulmonary, transdermal, transmucosal, and the like delivery routes. Delivery devices can include syringes, both needleless and needle containing, and inhalers.

The delivery devices can contain a single dose of the microspheres for treating a condition that is treatable by rapid or sustained release of the macromolecule in vivo, or they can contain multiple doses of microspheres, or can be multi-chambered and deliver more than one type of compound formulated as microspheres. The number of microspheres present in the single dose is dependent on the type and activity of the molecule. The single dose can be selected to achieve sustained release over a period of time that has been optimized for treating the particular medical condition. For example, when the molecule is a macromolecule, such as, for example, the DAS181 fusion protein (SEQ ID NO:17), the delivery dosage of microsphere compositions containing DAS181 can be from between about or at 0.5 mg protein per dose to about or at 100 mg protein per dose, or about or at 0.75 mg, 1 mg, 1.5 mg, 2 mg, 3 mg, 5 mg, 10 mg, 15 mg, 20 mg, 30 mg, 40 mg, 45 mg, 50 mg, 55 mg or 60 mg protein per dose. When the molecule is a small molecule, the delivery dosage can be from between about or at 0.1 mg compound per dose to about or at 1000 mg compound per dose, or about or at 0.2 mg, 0.5 mg, 1 mg, 1.5 mg, 2 mg, 3 mg, 5 mg, 10 mg, 15 mg, 20 mg, 30 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 250 mg, 300 mg, 350 mg, 400 mg, 500 mg, 600 mg, 700 mg, 800 mg, 900 mg or 1000 mg compound per dose.

The molecule component of the microsphere can be any molecule capable of forming microspheres according to the methods provided herein. For example, small molecule compounds such as those understood by those of skill in the art and provided herein, including therapeutics, diagnostic molecules, nutritional supplements and cosmetics, are contemplated for the preparation of microparticles according to the methods provided herein. Exemplified herein are small molecules belonging to a variety of classes of small molecule compounds, including the aminoglycosides tobramycin and kanamycin, the penicillin compound ampicillin, and tetracycline. Other small molecule compounds can include, but are not limited to, sterols such as cholesterol and lanosterol, steroids such as estrogen, testosterone, canrenone, oleandrin and spironolactone, sulfonamide derivatives such as Acetazolamide, Ambuside, Azosemide, Bumetanide, Butazolamide, Diphenylmethane-4.4′-disulfonamide, Disulfamide, Furosemide, uracils such as Aminometradine and Amisometradine, and the like, and prostaglandins. An organic or inorganic natural or synthetic pharmaceutical compound or drug can be incorporated into the microspheres by attaching the drug to the small molecule, and then forming the microspheres from the molecule-drug complex or conjugate.

In other embodiments, the molecule is a macromolecule including a protein, including enzymes and recombinant proteins, peptides such as somatostatins and leuprolides, glycopeptides such as vancomycin, carbohydrates, lipids, fatty acids, polysaccharides, carbohydrate- or polysaccharide-protein conjugates, nucleic acids such as DNA, PNA, RNA, siRNA, tRNA, virus, virus particles, viroids, prions, conjugates of small molecules (such as a hapten) and proteins, or mixtures thereof. In some embodiments, an organic or inorganic natural or synthetic pharmaceutical compound or drug can be incorporated into the microspheres by attaching the drug to a macromolecule, such as a protein, and then forming the microspheres from the macromolecule-drug complex or conjugate. It will be understood by those of skill in the art that the macromolecule can be a portion of a molecule such as, for example, a peptide, a single-stranded segment of a double-stranded nucleic acid molecule, or a virus particle, or other macromolecule having a tertiary and/or quaternary structure.

In some embodiments, the macromolecule is a therapeutic protein including, but not limited to, a sialidase, a sialidase fusion protein, a fusion protein containing a sialidase catalytic domain fused to a GAG-binding domain, a protease, a protease inhibitor, insulin, interferons, human growth hormone, calcitonin, rhDNase or parathyroid hormone, and the protein content of the microspheres can be from about or at 50% to about or at 60%, 65%, 70%, 75%, 80%, 85%, 90%, 95%, 96%, 97%, 98%, 99% or greater. For pulmonary administration, the microspheres can have an average size in the range of from about or at 0.5 microns to about or at 5.0 microns, and in some embodiments, between about or at 1 micron and about or at 2 microns.

Other proteins and peptides that can be used to form microspheres by the methods provided herein can include, but are not limited to, therapeutic proteins including DAS181 (DAS181; SEQ ID NO:17), al-antitrypsin, Ecotin, eglin c, serpin, Pulmozyme (rhDNase), Betaxolol™, Diclofenac™, doxorubicin, acetyl cysteine, leuprolide acetate, luteinizing hormone releasing hormone (LHRH), (D-Tryp6)-LHRH, nafarelin acetate, insulin, sodium insulin, zinc insulin, protamine, lysozyme, alpha-lactalbumin, basic fibroblast growth factor (bFGF), beta-lactoglobulin, Trypsin, calcitonin, parathyroid hormone, carbonic anhydrase, ovalbumin, bovine serum albumin (BSA), human serum albumin (HSA), phosphorylase b, alkaline phosphatase, beta-galactosidase, IgG, fibrinogen, poly-L-lysine, IgM, DNA, desmopressin acetate, growth hormone releasing factor (GHRF), somatostatin, leuprolide, antide, Factor VIII, G-CSF/GM-CSF, human growth hormone (hGH), beta interferon, antithrombin III, alpha interferon, alpha interferon 2b.

The term “macromolecule” or “small molecule” also can include a plurality of different macromolecules and/or small molecules and includes combinations such as a combination of a pharmaceutical compound and an affinity molecule for targeting the pharmaceutical compound to a tissue, organ or tumor requiring treatment. An affinity molecule can be, for example, a ligand or a receptor. Examples of ligands can include viruses, bacteria, polysaccharides, or toxins that can act as antigens to generate an immune response when administered to an animal and cause the production of antibodies. The microspheres provided herein also can be prepared from combinations or mixtures of macromolecules and small molecules

An inhaler device can be used to deliver a therapeutic compound or diagnostic compound, such as those listed above, to the respiratory airways and lungs of a subject. For example, protein microspheres, or antibiotic microspheres, such as vancomycin microspheres, can be prepared, for example by contacting an aqueous solution of the protein or vancomycin with a carboxylic acid such as citrate, or sulfate or other counterion and an organic solvent such as isopropanol, and cooling the solution to form the microspheres. The protein can be a therapeutic protein, such as a sialidase, a protease inhibitor, insulin, human growth hormone, calcitonin, rhDNase or parathyroid hormone, and the protein or vancomycin content of the microspheres can be about or at 70% to about or at 90% or more, 95% or more, or at least about 99% or more. For pulmonary administration, the microspheres, for example DAS181 microspheres or vancomycin microspheres, can be sized to have a mean diameter in the range of from about 0.5 microns to 5.0 microns, or between about 1 micron to about 2 microns.

Incubation conditions for forming the microspheres can be optimized to incorporate at least about 70%, 75%, 80%, 85%, 90%, 91%, 92%, 93%, 94%, 95%, 96%, 97%, 98%, or 99% or greater of the total amount of the molecule present in the solution prior to formation of the microspheres, by adjusting parameters including pH, temperature, concentration of molecule, or duration of reaction or incubation.

In some embodiments, a molecule or compound that does not produce microspheres of desirable characteristics, can be incorporated into microspheres having desirable characteristics, e.g., of size, delivery profile, mechanical strength, by incorporation or coupling of the compound with a carrier molecule that can form microspheres with desirable characteristics. In some embodiments, the carrier macromolecule is a protein, and the molecule or compound is bound inside and/or on the surface of the microsphere. In some embodiments, the molecule or compound also can serve as the counterion and initiate and/or facilitate the formation of microspheres.

When preparing microspheres containing a protein, a protein stabilizer such as glycerol, fatty acids, sugars such as sucrose, ions such as zinc, sodium chloride, or any other protein stabilizers known to those skilled in the art can be added prior to cooling the cocktail during microsphere formation, to minimize protein denaturation. Such stabilizers also can be added to microspheres formulated from other macromolecules or small molecules.

In some embodiments the microspheres can further be coated on the surface with suitable molecules and/or coating agents, such as those that lend resistance to acids, such as digestive acids, or proteases. In other embodiments, the microspheres can be non-covalently coated with compounds such as fatty acids or lipids. The coating can be applied to the microspheres by immersion in the solubilized coating substance, then spraying the microspheres with the substance, or by using other methods known to those of skill in the art. In some embodiments, the fatty acids or lipids are added directly to the microsphere-forming cocktail solution.

Formation of the microspheres by decreasing temperature can be performed by a multitude of conventional methods in batch or continuous modes. Microsphere formation can further be triggered by other methods including, but not limited to, modulating atmospheric pressure, g-force or surface expansion, including seeding. Microsphere formation can occur immediately upon exposure to these conditions or can require an extended period of time as provided herein.

### D. Exemplary Compounds

A. Peptides

Exemplary peptides that can be used to form microparticles by the methods provided herein are described below

Somatostatins

Somatostatin (also known as growth hormone inhibiting hormone (GHIH) or somatotropin release-inhibiting hormone (SRIF)) is a peptide hormone that regulates the endocrine system and affects neurotransmission and cell proliferation via interaction with G-protein-coupled somatostatin receptors and inhibition of the release of numerous secondary hormones. Somatostatin has two active forms produced by alternative cleavage of a single preproprotein: one of 14 amino acids, the other of 28 amino acids. Exemplary sequences corresponding to preprosomatostatin (contains signal sequence and propeptide), presomatostatin (contains propeptide), somatostatin 28 (SS-28, the 28 amino acid peptide) and somatostatin 14 (SS-14, the 14 amino acid peptide) are set forth in SEQ ID NOS: 18-21, respectively.

Somatostatin is primarily produced by neuroendocrine neurons of the periventricular nucleus of the hypothalamus, and is secreted in several locations in the digestive system, including the stomach, intestine and delta cells of the pancreas.

Somastatin is of therapeutic significance, for example, in the treatment of neuroendocrine disorders and tumors, due to its various biological actions, including inhibiting the release of growth hormone (GH), thus opposing the effects of Growth Hormone-Releasing Hormone (GHRH); inhibiting the release of thyroid-stimulating hormone (TSH); and suppressing the release of gastrointestinal hormones such as Gastrin, Cholecystokinin (CCK), Secretin Motilin, Vasoactive intestinal peptide (VIP), Gastric inhibitory polypeptide (GIP), Enteroglucagon (GIP); and pancreatic hormones, glucagon and insulin.

Leuprolide

Leuprorelin (INN) or leuprolide acetate (USAN) is a gonadotropin-releasing hormone agonist (GnRH agonist). By causing constant stimulation of the pituitary GnRH receptors, it initially causes stimulation (flare), but thereafter decreases pituitary secretion (downregulation) of gonadotropins luteinizing hormone (LH) and follicle-stimulating hormone (FSH). Like other GnRH agonists, leuprolide may be used in the treatment of hormone-responsive cancers such as prostate cancer or breast cancer, estrogen-dependent conditions (such as endometriosis or uterine fibroids), to treat precocious puberty, and to control ovarian stimulation in IVF. It also is considered a possible treatment for paraphilias. An exemplary sequence of leuprolide is set forth in SEQ ID NO: 22.

B. Antibiotics

An antibiotic includes any compound that inhibits or abolishes the growth of microorganisms, such as bacteria, fungi, or protozoans. Exemplary antibiotics that can be used to form microparticles by the methods provided herein are described below.

Aminoglycosides

Aminoglycosides are a group of antibiotics that are effective against certain types of bacteria. They include amikacin, gentamicin, kanamycin, neomycin, netilmicin, paromomycin, streptomycin, tobramycin and apramycin. Aminoglycosides are believed to work by binding to the bacterial 30S ribosomal subunit (some work by binding to the 50s subunit), inhibiting the translocation of the peptidyl-tRNA from the A-site to the P-site and also causing misreading of mRNA, leaving the bacterium unable to synthesize proteins vital to its growth.

Glycopeptides

Glycopeptide antibiotics are a class of antibiotic drugs. They contain a glycosylated cyclic or polycyclic nonribosomal peptide. Exemplary glycopeptide antibiotics include vancomycin, teicoplanin, ramoplanin, and decaplanin. This class of drugs inhibit the synthesis of cell walls in susceptible microbes by inhibiting peptidoglycan synthesis, thus inhibiting microbial growth.

Penicillins

Penicillins are a class of β-lactam antibiotics that include compounds such as Ampicillin, Azlocillin, Carbenicillin, Cloxacillin, Dicloxacillin, Flucloxacillin, Mezlocillin, Nafcillin, Penicillin, Piperacillin and Ticarcillin. β-lactamantibiotics work by inhibiting the formation of peptidoglycan cross links in the bacterial cell wall. The β-lactam moiety of penicillin binds to the enzyme (transpeptidase) that links the peptidoglycan molecules in bacteria, and this weakens the cell wall of the bacterium (in other words, the antibiotic causes cytolysis or death).

Tetracyclines

Tetracyclines are a class of natural and synthetic broad-spectrum antibiotics whose members include, for example, Tetracycline, Chlortetracycline, Oxytetracycline, Demeclocycline, Semi-synthetic Doxycycline, Lymecycline, Meclocycline, Methacycline, Minocycline and Rolitetracycline. Tetracyclines inhibit cell growth by inhibiting translation. The tetracyclines bind to the 16S part of the 30S ribosomal subunit and prevent the amino-acyl tRNA from binding to the A site of the ribosome.

C. Chemotherapeutic Agents Pharmacologic agents that are useful in the treatment of cancer fall under the general umbrella of chemotherapeutic agents, and are contemplated for preparation in the form of microspheres according to the methods provided herein. An exemplary chemotherapeutic agent is paclitaxel.

Paclitaxel

Paclitaxel is a mitotic inhibitor drug used in the treatment of cancer. Paclitaxel is an effective drug for the treatment of a variety of cancers, including lung, ovarian, breast cancer, and advanced forms of Kaposi's sarcoma. Together with docetaxel, it forms the drug category of the taxanes. Paclitaxel also is used for the prevention of restenosis (recurrent narrowing) of coronary stents; locally delivered to the wall of the coronary artery, a paclitaxel coating limits the growth of neointima (scar tissue) within stents.

D. Nucleic Acids

Nucleic acids, including those of therapeutic significance, are contemplated for the preparation of microspheres as provided herein. An exemplary therapeutic nucleic acid is siRNA.

siRNA

Small interfering RNA (siRNA), sometimes known as short interfering RNA or silencing RNA, are a class of 20-25 nucleotide-long double-stranded RNA molecules that play a variety of roles in biological systems. siRNA is involved in the RNA interference (RNAi) pathway, where the siRNA interferes with the expression of a specific gene. In addition to their role in the RNAi pathway, siRNAs also can act in RNAi-related pathways, e.g. as an antiviral mechanism or in shaping the chromatin structure of a genome. Given their potential ability to knock down essentially any gene of interest, RNAi, via siRNAs has generated a great deal of interest in possible therapeutic applications, such as influenza treatment. There are an increasing number of large-scale RNAi screens that are designed to identify the important genes in various biological pathways. Because disease processes also depend on the activity of multiple genes, it is expected that in some situations turning off the activity of a gene with a siRNA could produce a therapeutic benefit. For example, as described in Qing et al., (2003) Proc. Nat. Acad. ScL USA, 100:2718-2723, some siRNAs have been shown to inhibit PR8 and WSN influenza production in MDCK cells. The sequences of these siRNAs (sense and antisense strands) are set forth in SEQ ID NOS: 23-26.

E. Prostaglandins

A prostaglandin is any member of a group of lipid compounds that are derived enzymatically from fatty acids, are hormone or hormone-like, and have important and pleiotropic functions and effects in the animal body. Every prostaglandin contains 20 carbon atoms, including a 5-carbon ring. They are mediators and have a variety of physiological effects. The prostaglandins together with the thromboxanes and prostacyclins form the prostanoid class of fatty acid derivatives; the prostanoid class is a subclass of eicosanoids.

Due to their pleiotropic effects, prostaglandins have a variety of clinical applications, including:

To induce childbirth, parturition or abortion (PGE₂ or PGF₂, with or without mifepristone, a progesterone antagonist);

To prevent closure of patent ductus arteriosus in newborns with particular cyanotic heart defects (PGE₁);

To prevent and treat peptic ulcers (PGE);

As a vasodilator in severe Raynaud's phenomenon or ischemia of a limb;

In pulmonary hypertension;

In treatment of glaucoma; and

To treat erectile dysfunction or in penile rehabilitation following surgery.

F. Viruses

Viruses have a variety of applications as carriers or vectors, including in gene therapy and as inactivated viruses in vaccines. Microparticles of viruses, including derivative forms of the viruses such as virus particles or inactivated viruses, including, but not limited to, animal viruses, plant viruses, phages, influenza virus, parainfluenza virus, adenoviruses, retroviruses, respiratory syncytial virus, DNA-based viruses, coronavirus and rotavirus are contemplated for preparation according to the methods provided herein. Exemplified herein is the tobacco mosaic virus (TMV)

Tobacco Mosaic Virus (TMV)

Tobacco mosaic virus (TMV) is an RNA virus that infects plants, especially tobacco and other members of the family Solanaceae. The virus has a rod-like appearance. Its capsid is made from 2130 molecules of coat protein and one molecule of genomic RNA, 6390 bases long. The coat protein self assembles into the rod like helical structure around the RNA, which forms a hairpin loop structure. The protein monomer constains 158 amino acids that are assembled into four main alpha-helices, which are joined by a prominent loop proximal to the axis of the virion.

In addition to its impact on crop losses, the highly detailed knowledge regarding the structure of TMV, and the fact that it does not infect animals, makes it a valuable tool for investigations in areas including structural molecular biology, X-ray diffraction, and virus assembly and disassembly.

G. Proteins

Exemplary proteins that can be used to form microparticles by the methods provided herein are described below

Sialidases

Sialidases, also referred to as neuraminidases and N-acylneuraminosylglycohydrolases, are a family exoglycosidases that catalyze the removal of terminal sialic acid residues from sialo-glycoconjugates. Sialic acids are a family of a keto acids with 9-carbon backbones that are usually found at the outermost positions of the oligosaccharide chains attached to glycoproteins and glycolipids. These molecules are involved in a variety of biological functions and processes, such as the regulation of innate immunity, cell adhesion, and the interaction between inflammatory cells and target cells, possibly mediated through the binding of various lectins (Varki et al. (1992) Curr Opin Cell Biol 4:257-266). Sialic acids also are excellent sources of carbon, nitrogen, energy, and precursors of cell wall biosynthesis. Further still, sialic acids on eukaryotic cells can be used as receptors or coreceptors for pathogenic microorganisms, including, but not limited to, influenza virus, parainfluenza virus, some coronavirus and rotavirus Haemophilus influenzae, Streptococcus pneumonia, Mycoplasma pneumoniae, Moaxella catarrhalis, Helicobacter pylori and Pseudomonas aeruginosa. The most prominent member of the sialic acid family is N-acetylneuraminic acid (Neu5Ac), which is the biosynthetic precursor for most of the other types. Two major linkages between Neu5Ac and the penultimate galactose residues of carbohydrate side chains are found in nature, Neu5Ac α(2,3)-Gal and Neu5Ac α(2,6)-Gal. Both Neu5Ac α(2,3)-Gal and Neu5Ac α(2,6)-Gal molecules can be recognized by influenza viruses and used as the receptor through which the virus binds and initiates infection. Human influenza viruses, however, seem to prefer Neu5Ac α(2,6)-Gal, while avian and equine influenza viruses predominantly recognize Neu5Ac α(2,3)-Gal (Ito et al. (2000) Microobiol Immunol 44:423-730). The human respiratory epithelium expresses both forms of sialic acids, but α(2,6)-linked sialic acid is more abundant than α(2,3)-linked sialic acid. The low abundance of α(2,3)-linked sialic acid is most likely the basis for the species barrier for avian viruses, and indicates that reducing the level of a receptor sialic acid expressed on the airway epithelium would likely reduce the infectivity of an influenza virus. Thus, sialidases, which remove terminal sialic acid residues from sialo-glycoconjugates, present themselves as potential influenza virus therapeutic agents that function to reduce the levels of receptor sialic acids. Sialidases also can act as therapeutic agents for any other pathogen that utilizes sialic acids in the infection process including, but not limited to, M. pneumoniae, M. catarrhalis, H. pylori, H. influenzae, S. pneumonia, P. aeruginosa, parainfluenza viruses and some coronaviruses and rotaviruses.

Sialidases tend to be highly substrate specific. They can target particular types of complex molecules, such as glycoproteins or glycolipids; specific sugar linkages (e.g. 2-3, 2-6, or 2-8); or can be sensitive to the nature of the linkage sugar itself (e.g. D-galactose, N-acetyl-D-galactosamine). Substrate molecules include, but are not limited to, oligosaccharides, polysaccharides, glycoproteins, gangliosides, and synthetic molecules. For example, a sialidase can cleave bonds having α(2,3)-Gal, α(2,6)-Gal, or α(2,8)-Gal linkages between a sialic acid residue and the remainder of a substrate molecule. A sialidase also can cleave any or all of the linkages between the sialic acid residue and the remainder of the substrate molecule. Many sialidase proteins have been purified from microbes and higher eukaryotes and of these, several have been shown to catalyze the removal of terminal sialic acid residues than can serve as receptors for pathogenic microorganisms. For example, among the large bacterial sialidases are those that that can degrade the influenza receptor sialic acids Neu5Ac α(2,6)-Gal and Neu5Ac α(2,3)-Gal, including sialidases from Clostridium perfringens, Actinomyces viscosus, Arthrobacter ureafaciens, and Micromonospora viridifaciens. Other sialidases that can serve as therapeutic agents include the human sialidases, such as those encoded by the genes NEU2 and NEU4.

Sialidase-GAG Fusion Proteins

Sialidase-GAG fusion proteins are proteins that are made up of a sialidase protein, or catalytically active portion thereof, fused to a glycosaminoglycan (GAG)-binding sequence. As such, these proteins effectively contain an anchoring domain (the GAG-binding sequence) and a therapeutic domain (the sialidase protein, or catalytically active portion thereof). The sialidase-GAG fusion proteins are designed to bind to the epithelium and remove the surrounding sialic acids, and can therefore be used as a therapeutic agent against pathogens that utilize sialic acids in the infection process. The ability of the fusion protein to bind to the epithelium increases its retention when the fusion protein is administered, for example, as an inhalant to treat influenza infection. The GAG-binding sequence acts as an epithelium-anchoring domain that tethers the sialidase to the respiratory epithelium and increases its retention and potency.

Heparan sulfate, closely related to heparin, is a type of glycosaminoglycan (GAG) that is ubiquitously present on cell membranes, including the surface of respiratory epithelium. Many proteins specifically bind to heparin/heparan sulfate, and the GAG-binding sequences in these proteins have been identified. For example, the GAG-binding sequences of human platelet factor 4 (PF4) (SEQ ID NO:3), human interleukin 8 (IL8) (SEQ ID NO:4), human antithrombin III (AT III) (SEQ ID NO:5), human apoprotein E (ApoE) (SEQ ID NO:6), human angio-associated migratory cell protein (AAMP) (SEQ ID NO:7), or human amphiregulin (SEQ ID NO:8) have been shown to exhibit high affinity for heparin (Lee et al. (1991) PNAS 88:2768-2772; Goger et al. (2002) Biochem. 41:1640-1646; Witt et al. (1994) Curr Bio 4:394-400; Weisgraber et al. (1986) J Bio Chem 261:2068-2076). The GAG-binding sequences of these proteins are distinct from their receptor-binding sequences, so they do not induce the biological activities associated with the full-length proteins or the receptor-binding domains. These sequences, or other sequences that can bind heparin/heparan sulfate, can be used as epithelium-anchoring-domains in sialidase-GAG fusion proteins.

In the context of a sialidase-GAG fusion protein, the sialidase can include the entire sialidase protein, or a catalytically active portion thereof. For example, sialidase-GAG fusion protein can contain the 901 amino acid sialidase protein from A. viscosus set forth in SEQ ID NO:1. In another example, the sialidase-GAG fusion protein can contain the 394 amino acid catalytically active portion of a sialidase protein from A. viscosus set forth in SEQ ID NO:2. The GAG-binding sequence can be linked to the sialidase by recombinant methods. In some examples, the fusion protein can include an amino acid linker, such as four glycine residues. Furthermore, linkage can be via the N- or C-terminus of the GAG-binding sequence, or the N- or C-terminus of the sialidase. Exemplary examples of sialidase-GAG fusion proteins include those polypeptides set forth in SEQ ID NOS: 9-13, and 17. In a further example, the sialidase and GAG-binding sequence components can be linked using chemical or peptide linkers, by any method known in the art.

Proteinase Inhibitor 8

Proteinase inhibitor 8 (PI8), also known as Serpin B8, is a serine protease inhibitor (serpin) Serpins are a large superfamily of structurally related proteins that are expressed in viruses, insects, plants and higher organisms, but not in bacteria or yeast. Serpins regulate the activity of proteases involved in many biological process, including coagulation, fibrinolysis, inflammation, cell migration, and tumorigenesis. They contain a surface-exposed reactive site loop (RSL), which acts as a “bait” for proteases by mimicking a protease substrate sequence. On binding of the target protease to the serpin, the RSL is cleaved, after which the protease is covalently linked to the serpin. The protease in the newly formed serpin-protease complex is inactive (Huntington et al. (2000) Nature 407:923-926).

PI8 is a member of a subfamily of serpins of which chicken ovalbumin is the archtype. Like other serpins that belong to this family, PI8 lacks a typical cleavable N-terminal signal sequence, resulting in a 374 amino acid protein (SEQ ID NO:14) that resides mainly intracellularly. Other members of this human ovalbumin-like subfamily include plasminogen activator inhibitor type 2 (PAI-2), monocyte neutrophil elastase inhibitor (MNEI), squamous cell carcinoma antigen (SCCA)-1, leupin (SCCA-2) maspin (PI5), protease inhibitor 6 (PI6), protease inhibitor (PI9) and bomapin (PI10). Within this family the serpins P16, PI8, and PI9 show the highest structural homology (up to 68% amino acid identity) (Sprecher et al. (1995) J Biol Chem 270:29854-29861). PI-8 has been shown to inhibit trypsin, thrombin, factor Xa, subtilisin A, furin, and also chymotrypsin in vitro. It is released by platelets and appears to be involved in the regulation of furin activity and, therefore, platelet aggregation (LeBlond et al. (2006) Thromb Haemost 95:243-252).

In addition to their role in the regulation of endogenous biological processes, such as coagulation, serine protease inhibitors also can function to inhibit the biological activities of exogenous microorganisms. For example, a number of serine protease inhibitors have been shown to reduce influenza virus activation in cultured cells, chicken embryos and in the lungs of infected mice. The serpins bind to hemagglutinin (HA) molecules on the surface of the influenza virus and inhibit its activity, thus reducing the infectivity of the virus. For example trypsin inhibitors, such as: aprotinin (Zhimov et al. (2002) J Virol 76:8682-8689), leupeptin (Zhimov et al. (2002) J Virol 76:8682-8689; Tashiro et al. (1987) J Gen Virol 68:2039-2043), soybean protease inhibitor (Barbey-Morel et al. (1987) J Infect Dis 155:667-672), e-aminocaproic acid (Zhimov et al. 1982. Arch Virol 73:263-272) and n-p-tosyl-L-lysine chloromethylketone (TLCK) (Barbey-Morel et al. (1987) J Infect Dis 155:667-672) have all been shown to inhibit influenza virus infection, and are candidate therapeutic agents for use in the treatment of influenza virus infection. Thus, as a related trypsin inhibitor, PI8 also can be used as a therapeutic agent in the treatment of influenza virus infection.

Surface Active Agents

The compositions provided herein can contain one or more surface active agents that are added in an amount sufficient to stabilize the cocktail solutions and/or the microspheres. The selection of an appropriate amount of surface active agent is a function of the nature of the compound, solvent and antisolvent.

In certain embodiments, the surface active agent can be selected from sodium lauryl sulfate; sorbitan laurate, sorbitan palmitate, sorbitan stearate (available under the tradename Span® 20-40-60 etc.); polysorbates such as polyoxyethylene (20) sorbitan monolaurate, polyoxyethylene (20) sorbitan monopalmitate, polyoxyethylene (20) sorbitan monostearate (available under the tradename TWEENS® 20-40-60 etc.); benzalkonium chloride, mixed chain phospholipids, cationic lipids, oligolipids, phospholipids, carnitines, sphingosines, sphingomyelins, ceramides, glycolipids, lipoproteins, apoproteins, amphiphilic proteins, amphiphilic peptides, amphiphilic synthetic polymers, and combinations thereof. Other exemplary surface active agents for use herein include, but are not limited to

i) Natural lipids, i.e. Cholesterol, Sphingosine and Derivatives, Gangliosides, Sphingosine derivatives (Soy Bean), Phytosphingosine and derivatives (Yeast), Choline (Phosphatidylcholine), Ethanolamine (Phosphatidylethanolamine), Glycerol (Phosphatidyl-DL-glycerol), Inositol (Phosphatidylinositol), Serine (Phosphatidylserine (Sodium Salt)), Cardiolipin, Phosphatidic Acid, Egg Derived, Lyso (Mono Acyl) Derivatives (Lysophosphatides), Hydrogenated Phospholipids, Lipid Tissue Extracts,

ii) Synthetic lipids, i.e. Asymmetric Fatty Acid, Symmetric Fatty Acid—Saturated Series, Symmetric Fatty Acid—Unsaturated Series, Acyl Coenzyme A (Acetoyl Coenzyme A, Butanoyl Coenzyme A, Crotanoyl Coenzyme A, Hexanoyl Coenzyme A, Octanoyl Coenzyme A, Decanoyl Coenzyme A, Lauroyl Coenzyme A, Myristoyl Coenzyme A, Palmitoyl Coenzyme A, Stearoyl Coenzyme A, Oleoyl Coenzyme A, Arachidoyl Coenzyme A, Arachidonoyl Coenzyme A, Behenoyl Coenzyme A, Tricosanoyl Coenzyme A, Lignoceroyl Coenzyme A, Nervonoyl Coenzyme A, Hexacosanoyl Coenzyme A,

iii) Sphingolipids, i.e. D-erythro (C-18) Derivatives (Sphingosine, such as: D-erythro Sphingosine (synthetic), Sphingosine-1-Phosphate, N,N Dimethylsphingosine, N,N,N-Trimethylsphingosine, Sphingosylphosphorylcholine, Sphingomyelin and Glycosylated Sphingosine), Ceramide Derivatives (Ceramides, D-erythro Ceramide-1-Phosphate, Glycosulated Ceramides), Sphinganine (Dihydrosphingosine) (Sphinganine-1-Phosphate, Sphinganine (C20), D-erythro Sphinganine, N-Acyl-Sphinganine C2, N-Acyl-Sphinganine C8, N-acyl-Sphinganine C16, N-Acyl-Sphinganine C18, N-Acyl-Sphinganine C24, N-Acyl-Sphinganine C24:1), Glycosylated (C18) Sphingosine and Phospholipid Derivatives (Glycosylated-Sphingosine) (Sphingosine, βD-Glucosyl, Sphingosine, βD-Galactosyl, Sphingosine, βD-Lactosyl), Glycosylated-Ceramide (D-Glucosyl-β1-1′ Ceramide (C8), D-Galactosyl-β1-1′ Ceramide (C8), D-Lactosyl-β1-1′ Ceramide (C8), D-Glucosyl-β1-1′ Ceramide (C12), D-Galactosyl-β1-1′Ceramide (C12), D-Lactosyl-β1-1′ Ceramide (C12)), Glycosylated-Phosphatidylethanolamine (1,2-Dioleoyl-sn-Glycero-3-Phosphoethanolamine-N-Lactose), D-erythro (C17) Derivatives (D-erythro Sphingosine, D-erythro Sphingosine-1-phosphate), D-erythro (C20) Derivatives (D-erythro Sphingosine), L-threo (C18) Derivatives (L-threo Sphingosine, Safingol (L-threo Dihydrosphingosine)), Sphingosine Derivatives (Egg, Brain & Milk) (D-erythro-Sphingosine, Sphingomyelin, Ceramides, Cerebrosides, Brain Sulfatides), Gangliosides (Gangliosides Structures, Gangliosides—Ovine Brain, Gangliosides—Porcine Brain), Sphingosine Derivatives (Soy Bean) (Glucosylceramide), Phytosphingosine Derivatives (Yeast) (Phytosphingosine, D-ribo-Phytosphingosine-1-Phosphate, N-Acyl Phytosphingosine C2, N-Acyl Phytosphingosine C8, N-Acyl Phytosphingosine C18,

iv) Acyl coenzyme A, i.e. Acetoyl Coenzyme A (Ammonium Salt), Butanoyl Coenzyme A (Ammonium Salt), Crotanoyl Coenzyme A (Ammonium Salt), Hexanoyl Coenzyme A (Ammonium Salt), Octanoyl Coenzyme A (Ammonium Salt), Decanoyl Coenzyme A (Ammonium Salt), Lauroyl Coenzyme A (Ammonium Salt), Myristoyl Coenzyme A (Ammonium Salt), Palmitoyl Coenzyme A (Ammonium Salt), Stearoyl Coenzyme A (Ammonium Salt), Oleoyl Coenzyme A (Ammonium Salt), Arachidoyl Coenzyme A (Ammonium Salt), Arachidonoyl Coenzyme A (Ammonium Salt), Behenoyl Coenzyme A (Ammonium Salt), Tricosanoyl Coenzyme A (Ammonium Salt), Lignoceroyl Coenzyme A (Ammonium Salt), Nervonoyl Coenzyme A (Ammonium Salt), Hexacosanoyl Coenzyme A (Ammonium Salt), Docosahexaenoyl Coenzyme A (Ammonium Salt),

v) Oxidized lipids, i.e. 1-Palmitoyl-2-Azelaoyl-sn-Glycero-3-Phosphocholine, 1-O-Hexadecyl-2-Azelaoyl-sn-Glycero-3-Phosphocholine, 1-Palmitoyl-2-Glutaroyl-sn-Glycero-3-Phosphocholine (PGPC), 1-Palmitoyl-2-(9′-oxo-Nonanoyl)-sn-Glycero-3-Phosphocholine, 1-Palmitoyl-2-(5′-oxo-Valeroyl)-sn-Glycero-3-Phosphocholine,

vi) Ether lipids, i.e.: Diether Lipids (Dialkyl Phosphatidylcholine, Diphytanyl Ether Lipids), Alkyl Phosphocholine (Dodedylphosphocholine), O-Alkyl diacylphosphatidylcholinium (1,2-Diacyl-sn-Glycero-3-Ethylphosphocholine), Synthetic PAF & Derivatives (1-Alkyl-2-Acyl-Glycero-3-Phosphocholine & Derivatives),

vii) Fluorescent lipids, i.e.: Glycerol Based (Phosphatidylcholine (NBD), Phosphatidic Acid (NBD), Phosphatidylethanolamine (NBD), Phosphatidylglycerol (NBD), Phosphatidylserine (NBD)), Sphingosine Based (Ceramide (NBD), Sphingomyelin (NBD), Phytosphingosine (NBD), Galactosyl Cerebroside (NBD)), Headgroup Labeled Lipids (Glycerol Based) (Phosphatidylethanolamine (NBD), Phosphatidylethanolamine (Lissamine Rhodamine B), Dioleoyl Phosphatidylethanolamine (Dansyl, Pyrene, Fluorescein), Phosphatidylserine (NBD), Phosphatidylserine (Dansyl)), 25-NBD-Cholesterol,

viii) Other lipids including, but not limited to Lecithin, Ultralec-P (ADM), Soy powder,

ix) Surfactants including, but not limited to polyethylene glycol 400; sodium lauryl sulfate; sorbitan laurate, sorbitan palmitate, sorbitan stearate (available under the tradename Span® 20-40-60 etc.); polysorbates such as polyoxyethylene (20) sorbitan monolaurate, polyoxyethylene (20) sorbitan monopalmitate, polyoxyethylene (20) sorbitan monostearate (available under the tradename TWEENS® 20-40-60 etc.); benzalkonium chloride.

In certain embodiments, the phospholipids for use are phosphatidylcholines, phosphatidylethanolamines, phosphatidylserines, phosphatidylglycerols, phosphatidylinositols, phosphatidic acids, mixed chain phospholipids, lysophospholipids, hydrogenated phospholipids, partially hydrogenated phospholipids, and mixtures thereof.

In certain embodiments, the surface active agent is selected from polysorbate-80, lecithin and phosphatidylcholine. The surface active agents are present in an amount sufficient to stabilize the cocktail solution and/or the resulting microspheres.

The amount of surface active agent can be empirically determined and is a function of the agent selected, and the desired form of the resulting microsphere composition. The amount included can be from less than 0.1% by weight up to 35% or more. In certain embodiments, the surface active agent is present at a concentration of about 1%, 2%, 3%, 4%, 5%, 6%, 7%, 8%, 9%, 10%, 15%, 20%, 25% by weight up to about 30% by weight of the total weight of the composition. In certain embodiments, the surface active agent is present at a concentration of about 1 weight % up to about 20 weight % of the total weight of the composition. In certain embodiments, the surface active agent is present at a concentration of about 1 weight % up to about 15 weight % of the total weight of the composition. In other embodiments, the surface active agent is present at a concentration of about 1 weight % up to about 10 weight % of the total weight of the composition. In other embodiments, the surface active agent is present at a concentration of about 1 weight % up to about 8 weight % of the total weight of the composition. In other embodiments, the surface active agent is present at a concentration of about 1 weight % up to about 6 weight % of the total weight of the composition. In other embodiments, the surface active agent is present at a concentration of about 1 weight % up to about 4 weight % of the total weight of the composition. In other embodiments, the surface active agent is present at a concentration of about 20 weight % of the total weight of the composition. In other embodiments, the surface active agent is present at a concentration of about 15 weight % of the total weight of the composition. In other embodiments, the surface active agent is present at a concentration of about 13 weight % of the total weight of the composition. In other embodiments, the surface active agent is present at a concentration of about 11 weight % of the total weight of the composition. In other embodiments, the surface active agent is present at a concentration of about 8 weight % of the total weight of the composition. In other embodiments, the surface active agent is present at a concentration of about 6 weight % of the total weight of the composition. In other embodiments, the surface active agent is present at a concentration of about 4 weight % of the total weight of the composition. In other embodiments, the surface active agent is present at a concentration of about 2 weight % of the total weight of the composition. In other embodiments, the surface active agent is present at a concentration of about 1 weight % of the total weight of the composition.

Optional additional Agents

The compositions provided herein can optionally, in addition to the compound of interest, contain one or more pharmaceutical or nutraceutical or diagnostic or cosmetic or other such active agent for administering to a subject. Generally the agents are those that have a function in a host, e.g., immune regulation, regulation of biochemical processes, or enzymatic activity. Any agent that can be formulated as described herein can be administered in the compositions provided herein. Where the agent is a therapeutic, the compositions contain a therapeutically effective amount of an agent to be delivered. The particular amount of active agent in a dosage will vary widely according to the nature of the active agent, the nature of the condition being treated, the age and size of the subject, and other parameters. In addition, the compound forming the microsphere can itself also be an active agent.

Generally, the amount of additional active agent or nutrient besides the compound in the composition will vary from less than about 0.01% by weight to about 20% by weight of the composition, or more and typically are formulated for single dosage administration. A single dosage can vary from about 0.01 μg to 10 mg of an agent per kilogram of body weight of the host, with dosages from about 0.1 μg to 1 mg/kg being commonly employed. These concentrations, however, are general guidelines only and particular amounts and dosages may be selected based on the active agent being administered, the condition being treated, and the treatment regimen being employed means an amount of a drug or an active agent that is sufficient to provide the desired local or systemic effect and performance at a reasonable benefit/risk ratio to a subject attending any medical treatment.

Agents can be selected from inorganic and organic drugs including, but not limited to drugs that act on the peripheral nerves, adrenergic receptors, cholinergic receptors, nervous system, skeletal muscles, cardiovascular system, smooth muscles, blood circulatory system, synaptic sites, neuro-effector junctional sites, endocrine system, hormone systems, immunological system, reproductive system, skeletal system, autocoid systems, alimentary and excretory systems, histamine systems, and the like. The active agents that can be delivered using the compositions provided herein include, but are not limited to, anticonvulsants, analgesics, antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids, sympathomimetics, parasympathomimetics, tranquilizers, urinary tract drugs, vaccines, vaginal drugs, vitamins, minerals, nonsteroidal anti-inflammatory drugs, angiotensin converting enzymes, polynucleotides, polypeptides, polysaccharides, and nutritional supplements including herbal supplements.

The level of agent to be delivered is from about 0.01% up to about 50%, from about 0.1% up to about 40%, from about 0.1% up to about 30%, from about 0.1% up to about 20%, from about 0.1% up to about 10%, from about 0.1% up to about 9%, from about 0.1% up to about 8%, from about 0.1% up to about 7%, from about 0.1% up to about 6%, from about 0.1% up to about 5%, from about 0.1% up to about 4%, from about 0.1% up to about 3%, from about 0.1% up to about 2%, from about 0.1% up to about 1% by weight of the composition. The agent to be delivered can be water soluble, slightly water soluble, or soluble in an organic solvent or an oil. In certain embodiments, the agent to be delivered is selected from among antibiotics, chemotherapeutics, antivirals, anticonvulsants, analgesics, antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, non denatured whey protein, ophthalmics, psychic energizers, sedatives, steroids, sympathomimetics, parasympathomimetics, tranquilizers, urinary tract drugs, vaccines, vaginal drugs, vitamins, minerals, nonsteroidal anti-inflammatory drugs, angiotensin converting enzymes, polynucleotides, polypeptides, polysaccharides, and nutritional supplements including herbal supplements.

Exemplary active agents are the same as the classes of compounds listed as being suitable compounds for preparing microparticles, and they are set forth in the “Macromolecules and Small Molecules” section herein as “Exemplary Active Agent Categories for Macromolecules and Small Molecules.”

### E. Uses of the Compositions

Therapeutic and diagnostic applications of the microspheres include drug delivery, vaccination, gene therapy, and in vivo tissue or tumor imaging. Routes of administration include oral or parenteral administration; mucosal administration; ophthalmic administration; intravenous, subcutaneous, intra articular, or intramuscular injection; inhalation administration; and topical administration.

The diseases and disorders can include, but are not limited to neural disorders, respiratory disorders, immune system disorders, muscular disorders, reproductive disorders, gastrointestinal disorders, pulmonary disorders, digestive disorders, metabolic disorders, cardiovascular disorders, renal disorders, proliferative disorders, cancerous diseases and inflammation.

The microparticles provided herein can be used to treat Infectious diseases, such as arboviral infections, botulism, brucellosis, candidiasis, campylobacteriosis, chickenpox, chlamydia, cholera, coronovirus infections, staphylococcus infections, coxsackie virus infections, Creutzfeldt-Jakob disease, cryptosporidiosis, cyclospora infection, cytomegalovirus infections, Epstein-Barr virus infection, dengue fever, diphtheria, ear infections, encephalitis, influenza virus infections, parainfluenza virus infections giardiasis, gonorrhea, Haemophilus influenzae infections, hantavirus infections, viral hepatitis, herpes simplex virus infections, HIV/AIDS, helicobacter infection, human papillomavirus (HPV) infections, infectious mononucleosis, legionellosis, leprosy, leptospirosis, listeriosis, lyme disease, lymphocytic choriomeningitis, malaria, measles, marburg hemorrhagic fever, meningitis, monkeypox, mumps, mycobacteria infection, mycoplasma infection, norwalk virus infection, pertussis, pinworm infection, pneumococcal disease, Streptococcus pneumonia infection, Mycoplasma pneumoniae infection, Moraxella catarrhalis infection, Pseudomonas aeruginosa infection, rotavirus infection, psittacosis, rabies, respiratory syncytial virus infection, (RSV), ringworm, rocky mountain spotted fever, rubella, salmonellosis, SARS, scabies, sexually transmitted diseases, shigellosis, shingles, sporotrichosis, streptococcal infections, syphilis, tetanus, trichinosis, tuberculosis, tularemia, typhoid fever, viral meningitis, bacterial meningitis, west nile virus infection, yellow fever, adenovirus-mediated infections and diseases, retrovirus-mediated infectious diseases, yersiniosis zoonoses, and any other infectious respiratory, pulmonary, dermatological, gastrointestinal and urinary tract diseases.

Other diseases and conditions, including arthritis, asthma, allergic conditions, Alzheimer's disease, cancers, cardiovascular disease, multiple sclerosis (MS), Parkinson's disease, cystic fibrosis (CF), diabetes, non-viral hepatitis, hemophilia, bleeding disorders, blood disorders, genetic disorders, hormonal disorders, kidney disease, liver disease, neurological disorders, metabolic diseases, skin conditions, thyroid disease, osteoporosis, obesity, stroke, anemia, inflammatory diseases and autoimmune diseases.

### F. Combinations, Kits, Articles of Manufacture

Combinations and kits containing the combinations provided herein, including microparticles or ingredients for forming the microparticles such as a small molecule or a macromolecule of interest, counterions, solvents, buffers, or salts and optionally including instructions for administration are provided. The combinations include, for example, the compositions as provided herein and reagents or solutions for diluting the compositions to a desired concentration for administration to a host subject, including human beings. The combinations also can include the compositions as provided herein and additional nutritional and/or therapeutic agents, including drugs, as provided herein.

Additionally provided herein are kits containing the above-described combinations and optionally instructions for administration by oral, subcutaneous, transdermal, intravenous, intramuscular, ophthalmic or other routes, depending on the protein and optional additional agent(s) to be delivered.

The compositions provided herein can be packaged as articles of manufacture containing packaging material, a composition provided herein, and a label that indicates that the composition, e.g., a DAS181 formulation or a vancomycin formulation, is formulated for oral, pulmonary or other delivery.

The articles of manufacture provided herein can contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, bottles, and any packaging material suitable for a selected formulation and intended mode of administration and treatment.

The following examples are included for illustrative purposes only and are not intended to limit the scope of the invention.

### Example 1

### Preparation of Microspheres of the Sialidase Fusion Protein, DAS181

A. Purification of DAS181

DAS181 is a fusion protein containing the heparin (glycosaminoglycan, or GAG) binding domain from human amphiregulin fused via its N-terminus to the C-terminus of a catalytic domain of Actinomyces Viscosus (sequence of amino acids set forth in SEQ ID NO:17). The DAS181 protein was purified as described in Malakhov et al., Antimicrob. Agents Chemother., 1470-1479, 2006, which is incorporated in its entirety by reference herein. Briefly, the DNA fragment coding for DAS181 was cloned into the plasmid vector pTrc99a (Pharmacia; SEQ ID NO:16) under the control of a IPTG (isopropyl-β-D-thiogalactopyranoside)-inducible promoter. The resulting construct was expressed in the BL21 strain of Escherichia Coli (E. Coli).

The E. Coli cells containing the expressed construct were lysed by sonication in 50 mM phosphate buffer, pH 8.0; 0.3 M NaCl and 10% glycerol. The clarified lysate was passed through an SP-Sepharose column. Proteins were eluted from the column with lysis buffer that contained 0.8 M NaCl. The fraction eluted from SP-Sepharose was adjusted to 1.9 M ammonium sulfate ((NH₄)₂SO₄), clarified by centrifugation, and loaded onto a butyl-Sepharose column. The column was washed with two volumes of 1.3 M (NH₄)₂SO₄, and the DAS181 fusion protein was eluted with 0.65 M (NH₄)₂SO₄.

For the final step, size exclusion chromatography was performed on Sephacryl S-200 equilibrated with phosphate-buffered saline (PBS). The protein purity was determined to be greater than 98% as assessed by sodium dodecyl sulfate-polyacrylamide gel electrophoresis, reversed-phase high-pressure liquid chromatography, and enzyme-linked immunosorbent assay with antibodies generated against E. Coli cell proteins. The purified DAS181, molecular weight 44,800 Da, was dialyzed against 2 mM sodium acetate buffer, pH 5.0.

B. Activity of DAS181

The sialidase activity of DAS181 was measured using the fluorogenic substrate 4-methylumbelliferyl-N-acetyl-α-D-neuraminic acid (4-MU-NANA; Sigma). One unit of sialidase is defined as the amount of enzyme that releases 10 nmol of MU from 4-MU-NANA in 10 minutes at 37° C. (50 mM CH₃COOH-NaOH buffer, pH 5.5) in a reaction that contains 20 nmol of 4-MU-NANA in a 0.2 ml volume (Potier et al., Anal. Biochem., 94:287-296, 1979). The specific activity of DAS181 was determined to be 1,300 U/mg protein (0.77 μg DAS181 protein per unit of activity).

C. Preparation of Microspheres Using Purified DAS181

DAS181 (10 mg/ml), purified and prepared as described under Section A above, was used to form 200 μl cocktails as shown below. The cocktails contained either glycine or citrate as counterions, and isopropanol as organic solvent, as follows:

1) DAS181+5 mM glycine, pH 5.0;

2) DAS181+5 mM glycine, pH 5.0+10% isopropanol;

3) DAS181+5 mM sodium citrate, pH 5.0;

4) DAS181+5 mM sodium citrate, pH 5.0+10% isopropanol;

Plastic microcentrifuge tubes containing the cocktails with ingredients as described in 1)-4) above were gradually cooled from:

(a) ambient temperature (about 25° C.) to 4° C. by placing the cocktails in a refrigerator, followed by:

(b) cooling to −20° C. by placing the resulting cocktail from (a) in a freezer, followed by:

(c) freezing to −80° C. by placing the resulting cocktail from b) in a freezer.

Under optimal conditions, microspheres would be expected to form between about 4° C. to about −20° C. (generally in the range of about −2° C. to about −15° C.). Freezing to −80° C. is carried out to remove ingredients from the cocktail other than the microspheres (e.g., solvent, etc.) by freeze-drying. Cocktail 4) was prepared in triplicate, two aliquots in plastic tubes and one in a glass tube. One aliquot (in a plastic tube) was cooled as described above, while the two other aliquots (one in a plastic tube and one in a glass tube) were subjected to snap cooling/freezing by dipping the tubes into liquid nitrogen.

Upon freezing, all tubes were placed into the lyophilizer and the volatiles (water and isopropanol) were removed by sublimation, leaving the dry pellets.

Results: The dry pellets recovered from the cocktails treated as described above, were tested for the presence of microspheres. Of the above samples, microspheres with good dispersivity characteristics, about 2 microns (μm) in size, were observed only with cocktail 4) containing citrate counterion and isopropanol and subjected to gradual cooling. The counterion glycine did not prove to be optimal for the DAS181 protein (cocktail 2)), showing a mixture of glass-like crystals and agglomerates with only a few microspheres. When no organic solvent was present, a glass-like mass of lyophilized DAS181 protein was obtained and no microspheres were observed (cocktails 1) and 3)). Snap-freezing of cocktail 4) in a glass tube produced glass-like crystals and no microspheres, while snap-freezing of cocktail 4) in a plastic tube (cooling rate is slightly slower due to slower diffusion of heat through plastic than through glass) produced agglomerated microspheres.

This example demonstrates that microspheres with narrow size distribution and good dispersivity (minimal agglomeration) can be produced by a combination of appropriate protein, counterion, organic solvent and gradual cooling, using the methods provided herein.

### Example 2

### Size of DAS181 Microspheres as a Function of Organic Solvent Concentration

DAS181 was purified and used to prepare microspheres as described above in Example 1 (see cocktail 4)), using a combination of DAS181 protein (10 mg/ml), citrate counterion (sodium citrate, 5 mM) and isopropanol organic solvent (10%, 20% or 30%). The resulting cocktail solutions were cooled from ambient temperature (about 25° C.) to 4° C., followed by cooling to −20° C., followed by freezing to −80° C., as described in Example 1. Upon freezing to 80° C., the tubes are placed in a lyophilizer and the volatiles (water and isopropanol) were removed by sublimation, leaving the dry powder containing microspheres.

Results: Microsphere formation was observed with all three concentrations: 10%, 20%, or 30%, of the organic solvent isopropanol. The dimensions of the microspheres however varied, depending on the concentration of the organic solvent. The sizes of the microspheres as determined by comparing the particles to a grid on a hemocytometer were estimated to be 2 microns using 10% isopropanol, 4 microns using 20% isopropanol, and 5-6 microns using 30% isopropanol. These results demonstrate that the size of the microparticles can be engineered as desired using an appropriate concentration of organic solvent.

### Example 3

### Size of DAS181 Microspheres as a Function of Protein Concentration

DAS181 was purified and used to prepare microspheres as described above in Example 1 (see cocktail 4)), using a combination of DAS181 protein (5 mg/ml or 10 mg/ml), citrate counterion (sodium citrate, 5 mM) and isopropanol (5% or 20%). The resulting cocktail solutions were cooled from ambient temperature (about 25° C.) to 4° C., followed by cooling to −20° C., followed by freezing to −80° C., as described in Example 1. Upon freezing to −80° C., the tubes were placed in a lyophilizer and the volatiles (water and isopropanol) were removed by sublimation, leaving the dry powder containing microspheres.

Results: Microsphere formation was observed with both concentrations of protein (5 mg/ml and 10 mg/ml), and both concentrations of organic solvent (5% or 20%). The dimensions of the microspheres however varied. Cocktails containing 5 mg/ml or 10 mg/ml protein and 5% isopropanol produced microspheres estimated to be about 1.5 micron in size. The cocktail containing 5 mg/ml protein and 20% isopropanol produced microspheres of an estimated size of about 3 microns, while the cocktail containing 10 mg/ml protein and 20% isopropanol produced microspheres of an estimated size of about 4 microns. These results demonstrate that the size of the microparticles can be engineered as desired using an appropriate concentration of protein, or an appropriate combination of concentration of organic solvent and concentration of protein.

### Example 4

### Size of DAS181 Microspheres as a Function of Counterion Concentration

DAS181 was purified and used to prepare microspheres as described above in Example 1 (see cocktail 4)), using a combination of DAS181 protein (10 mg/ml), citrate counterion (sodium citrate; 2 mM, 3 mM or 6 mM) and isopropanol (20%). The cocktail solutions were mixed in glass vials and cooled from +20° C. to −40° C. at a freeze ramp of 1° C. per minute in a Millrock Lab Series lyophilizer. Volatiles (water and isopropanol) were removed by sublimation at 100 mTorr with primary drying at −30° C. for 12 hours and secondary drying at 30° C. for 3 hours, leaving the dry powder containing microspheres.

Results: Microsphere formation was observed at all three tested concentrations of citrate counterion. The size of the microspheres increased from 1 micron at 2 mM citrate, to 3 microns at 3 mM citrate, to 5 microns at 6 mM citrate. Addition of 1 mM sodium acetate or 1 mM sodium chloride to the cocktail containing 2 mM citrate did not affect formation of the microspheres triggered by the citrate counterion. These results demonstrate that the size of the microparticles can be engineered as desired using an appropriate concentration of counterion.

### Example 5

### DAS181 Microspheres Formed in the Presence of Surfactants

The addition of surfactants to macromolecular (e.g., protein) microspheres often can improve characteristics of the microspheres that render them suitable for administration to a subject, such as flowability, dispersivity and disposition for a particular route of administration, such as intranasal or oral inhalation. To test whether surfactants can be incorporated into the methods of manufacturing microspheres as provided herein, the production of DAS181 microspheres was undertaken as described in Example 1 above, except that in addition, a surfactant was added to the solution.

To a cocktail solution containing 5 mg/ml DAS181, 5 mM sodium citrate, and 20% isopropanol, was added a surfactant (3.5% w/w lecithin, 0.7% w/w Span-85® (sorbitan trioleate), or 3.5% w/w oleic acid). The microspheres were formed by cooling the solutions to 4° C., followed by cooling to −20° C., followed by freezing to −80° C. for lyophilization as described above in Example 1. Upon freezing, the tubes were placed into a lyophilizer and the volatiles (water and isopropanol) were removed by sublimation, leaving the dry powder containing microspheres.

Results: The microspheres resulting from treatment of each of the above cocktails as described above were spread on glass slides using cover slips rubbed in a circular motion. Efficient microsphere formation was observed in all cases. When the samples containing surfactant were compared to the sample containing all the remaining ingredients but no added surfactant, it was noted that the microspheres formed in the presence of surfactant had improved dispersivity (lesser agglomeration or aggregation).

### Example 6

### Preparation of Microspheres of Bovine Serum Albumin (BSA) by Selection of Suitable Types and Concentrations of Organic Solvents and Counterions

As described herein, the methods provided herein can empirically be optimized in high-throughput format to obtain microspheres having desired characteristics including size, flowability and dispersivity. The purpose of this experiment was to demonstrate that by varying types and concentrations of organic solvents and counterions, as well as pH of the cocktail, size and quality of microspheres of a protein of interest, in this case bovine serum albumin (BSA), can be adjusted.

Cocktail solutions containing 5 mg/ml of BSA and various organic solvents and counterions at indicated pH and concentrations (see Table 1) were placed in a microtiter plate (final volume per well of 0.1 ml). Cocktails were cooled from +20° C. to −40° C. at a freeze ramp of 1° C. per minute in a Millrock Lab Series lyophilizer. Volatiles were removed by sublimation at 100 mTorr, with a primary drying at −30° C. for 12 hours and secondary drying at 30° C. for 3 hours.

Results: The results are shown in Table 1 below. For the BSA protein, combinations (of counterion and organic solvent, respectively) that produced the most uniform microspheres with minimal crystallization or aggregation include:

(1) citrate+isopropanol
(2) citrate+acetone
(3) itaconic acid+1-propanol
(4) glycine+dioxane
(5) glycine+1-propanol
(6) rubidium+1-propanol
(7) perchlorate+1-propanol

TABLE 1
High-throughput screening of BSA microspheres
formed under different conditions

| Counterion | pH | Organic Solvent | Product description |
|---|---|---|---|
| 5 mM pivalic | 4.0 | 5% Cyclohexanol | 0.5-1 micron microspheres |
| acid | | | with occasional crystals |
| 5 mM pivalic | 4.0 | 5% 1-propanol | 0.5-1 micron microspheres |
| acid | | | with some aggregates |
| 5 mM pivalic | 4.0 | 5% butyl alcohol | Aggregated microspheres |
| acid | | | |
| 5 mM pivalic | 4.0 | 5% p-Dioxane | Aggregated microspheres |
| acid | | | |
| 5 mM rubidium | 9.0 | 5% Cyclohexanol | 0.5-1 micron microspheres. |
| chloride | | | Aggregates and occasional |
| | | | crystals |
| 5 mM rubidium | 9.0 | 5% 1-propanol | 0.5-1 micron microspheres |
| chloride | | | |
| 5 mM rubidium | 9.0 | 5% butyl alcohol | Few microspheres (0.5-1 |
| chloride | | | micron). Mostly aggregates |
| | | | and crystals |
| 5 mM rubidium | 9.0 | 5% p-Dioxane | 1-2 microns microspheres |
| chloride | | | with some aggregates |
| 5 mM sodium | 4.0 | 5% Cyclohexanol | 1-2 microns microspheres |
| bromide | | | with some aggregates |
| 5 mM sodium | 4.0 | 5% 1-propanol | Few microspheres (0.5-2 |
| bromide | | | micron). Mostly aggregates |
| | | | and crystals |
| 5 mM sodium | 4.0 | 5% butyl alcohol | Few microspheres (0.5-1 |
| bromide | | | micron). Mostly aggregates |
| | | | and crystals |
| 5 mM sodium | 4.0 | 5% p-Dioxane | 1-2 microns microspheres |
| bromide | | | with some aggregates |
| 5 mM sodium | 4.0 | 5% Cyclohexanol | 0.5-2 microns microspheres |
| perchlorate | | | with some crystals and |
| | | | aggregates |
| 5 mM sodium | 4.0 | 5% 1-propanol | 0.5-1 micron microspheres |
| perchlorate | | | |
| 5 mM sodium | 4.0 | 5% butyl alcohol | Few 1-2 microns microspheres. |
| perchlorate | | | Mostly crystals and aggregates |
| 5 mM sodium | 4.0 | 5% p-Dioxane | Aggregated microspheres |
| perchlorate | | | |
| 5 mM calcium | 4.0 | 5% Cyclohexanol | Few 1-2 microns microspheres, |
| phosphate | | | mostly aggregates |
| 5 mM calcium | 4.0 | 5% 1-propanol | 1-2 microns microspheres |
| phosphate | | | with some aggregates |
| 5 mM calcium | 4.0 | 5% butyl alcohol | Few 1-2 micron microspheres. |
| phosphate | | | Mostly crystals and aggregates |
| 5 mM calcium | 4.0 | 5% p-Dioxane | Aggregated microspheres |
| phosphate | | | |
| 5 mM trieth- | 9.0 | 5% Cyclohexanol | 0.5-1 micron microspheres |
| ylamine | | | with some crystals and |
| | | | aggregates |
| 5 mM trieth- | 9.0 | 5% 1-propanol | 1-2 micron microspheres |
| ylamine | | | with some aggregates |
| 5 mM trieth- | 9.0 | 5% butyl alcohol | Few 1-2 micron microspheres. |
| ylamine | | | Mostly crystals and aggregates |
| 5 mM trieth- | 9.0 | 5% p-Dioxane | Aggregated microspheres |
| ylamine | | | |
| 5 mM glycine | 9.0 | 5% Cyclohexanol | 0.5-1 micron microspheres |
| | | | with some crystals and |
| | | | aggregates |
| 5 mM glycine | 9.0 | 5% 1-propanol | 0.5-2 micron microspheres |
| | | | with occasional aggregates |
| 5 mM glycine | 9.0 | 5% butyl alcohol | Few 1-2 micron microspheres. |
| | | | Mostly crystals and aggregates |
| 5 mM glycine | 9.0 | 5% p-Dioxane | 1-2 micron microspheres |
| 5 mM sodium | 4.0 | 15% isopropanol | 1-2 micron microspheres |
| citrate | | | |
| 5 mM sodium | 4.0 | 15% acetone | 0.5-1 micron microspheres |
| citrate | | | |
| 5 mM itaconic | 4.0 | 15% 1-propanol | 1-2 micron microspheres |
| acid | | | |

These results demonstrate that, for each protein, multiple formulations can readily be screened for the best microsphere formation (desired dimensions, uniformity, dispersivity, minimal aggregation and crystal formation, etc.) in high-throughput format. The combinations of reagents and conditions (counterion, organic solvent, pH, concentrations) selected from the initial screen can then further be fine-tuned as desired.

### Example 7

### Preparation of Microspheres Using a Variety of Proteins

The methods provided herein can be used to prepare microspheres using a variety of proteins. In addition to DAS181 and BSA exemplified above, the methods were used to prepare microspheres from trypsin, hemoglobin, DNase I, lysozyme, ovalbumin, RNAse A, hexahistidine-tagged human proteinase inhibitor 8 (PI8, having the sequence of amino acids set forth in SEQ ID NO:15), red fluorescent protein (RFP) and green fluorescent protein (GFP).

DNase I, trypsin and hemoglobin were purchased from Worthington. Lysozyme, ovalbumin, and RNAse A were purchased from Sigma. Purification of 6×His tagged PI8, GFP and RFP: 6×His tagged PI8, GFP and RFP were expressed and purified essentially as described for DAS181 in Example 1 above, with the following modifications:

Purification of 6×His tagged GFP and 6×His tagged RFP: Constructs encoding Red Fluorescent protein and Green Fluorescent protein with N-terminal His₆ tags were expressed in E. Coli as 6×His-tagged proteins. Expression of Red Fluorescent protein was allowed to proceed overnight in LB medium with 1 mM IPTG. Green Fluorescent protein was induced for 3 hour in TB medium with 1 mM IPTG. Cell lysates from 4 liters of induced cultures were clarified by centrifugation and the proteins were purified by metal chelate affinity chromatography on Fast-Flow Chelating resin (GE Healthcare) charged with Nickel and packed into C-10 columns (GE Healthcare).

The proteins were further purified by Gel Filtration Chromatography on a 0.5 cm×70 cm Sephacryl 200 column equilibrated with phosphate buffered saline. The proteins were dialyzed against 2 mM sodium acetate buffer, pH 5.0, and concentrated on a Centriprep (Amicon).

Purification of 6×His tagged PI8: A construct encoding PI8 with an N-terminal His₆ tag was expressed in E. Coli as 6×His-tagged PI8. Purification was performed as described for 6×His RFP and 6×His GFP above, with the exception that all buffers used in the various chromatographic purification steps contained 1 mM TCEP (Tris(2-carboxyethyl)phosphine hydrochloride).

Preparation of microspheres: Cocktail solutions containing 5 mg/ml of protein and various counterions, organic solvents and pH as listed below were prepared in a microtiter plate as described above in Example 6.

TABLE 2
Combinations Used to Produce Microspheres of Different Proteins

| | | | | Microsphere |
|---|---|---|---|---|
| | | | Organic | Size |
| Protein | Counterion | pH | Solvent | (microns) |
| Trypsin | 5 mM arginine | 8.0 | 5% isopropanol | 0.5-1 |
| Lysozyme | 5 mM citrate | 8.0 | 5% isopropanol | 4-5 |
| PIN 168 (PI8) | 5 mM citrate | 5.0 | 7% isopropanol | 2-5 |
| DNase I | 5 mM citrate | 4.0 | 5% isopropanol | 0.4-1 |
| RNase A | 5 mM citrate | 4.0 | 5% isopropanol | 0.4-1 |
| Hemoglobin | 5 mM glycine | 5.0 | 10% isopropanol | 0.4-0.7 |
| Ovalbumin | 5 mM pivalic | 4.0 | 10% isopropanol | 0.5-1 |
| | acid | | | |
| Red fluores- | 5 mM pivalic | 7.0 | 10% 1-propanol | 1-4 (occasional |
| cent protein | acid | | | aggregates) |
| Green fluores- | 5 mM pivalic | 7.0 | 10% 1-propanol | 0.5-1.5 |
| cent protein | acid | | | |

The microtiter plate was cooled from +20° C. to −40° C. at a freeze ramp of 1° C. per minute in a Millrock Lab Series lyophilizer. Volatiles (water and isopropanol) were removed by sublimation at 100 mTorr with primary drying at −30° C. for 12 hours and secondary drying at 30° C. for 3 hours, leaving the dry powder containing microspheres.

The dry powders were spread on glass slides and microphotography was performed through either 32× or 100× objective. All the combinations listed in Table 2 above produced microspheres of good quality (uniform size distribution, dispersivity, with few aggregates and/or crystals). The microspheres varied in size from about 0.4-1 micron (RNAse A, DNAse I) to about 2-5 microns (6×His PI8, lysozyme), depending on the protein. This example demonstrates that the methods provided herein can be used to produce microspheres from a wide variety of proteins.

### Example 8

Aerodynamic Particle Size Distribution of DAS181 Microspheres for Inhalation: a Comparison of the Method Provided Herein with Spray-Drying

As described herein, the methods provided herein can be used to produce microspheres in any desired size range, including a range of about 0.5 micron to about 6-8 microns for delivery via inhalation.

A. Preparation of Microspheres

To test the aerodynamic particle size distribution of DAS181 dry powder (microspheres) formulated for delivery by inhalation, DAS181 microspheres were prepared using two methods as follows:

(a) A DAS181 aqueous solution containing 14 mg/ml DAS181, 5 mM sodium citrate, pH 5.0 was spray dried into an air stream at 55° C., to produce microspheres.
(b) Alternately, DAS181 microspheres were produced according to the methods provided herein. To a DAS181 aqueous solution containing 14 mg/ml DAS181, 5 mM sodium citrate, pH 5.0, was added 5% isopropanol as organic solvent. The resulting solution was cooled from +20° C. to −40° C. at a freeze ramp of 1° C. per minute in a Milirock Lab Series lyophilizer. Volatiles (water and isopropanol) were removed by sublimation at 100 mTorr with primary drying at −30° C. for 12 hours and secondary drying at 30° C. for 3 hours, leaving the dry powder containing microspheres.

B. Aerodynamic Particle Size Distribution of Microspheres

The microspheres prepared as described in Example 8A were tested by Andersen Cascade Impaction. The deposition of pharmaceuticals in the respiratory tract can be predicted by the aerodynamic behavior of particles (microspheres) on the stages/collection plates of the cascade impactor.

The cascade impaction experiment was performed using DAS181 microspheres prepared by one of the two alternate methods described in section A above, i.e., either by spray-drying or by the methods provided herein. The microspheres (10 mg) were loaded into gelatin capsules. The gelatin capsules were placed into a CycloHaler (PharmaChemie) dry powder inhaler and subjected to cascade impaction. An 8-stage, non-viable Andersen Cascade Impactor (Thermo Electron, Boston) modified for use at 90 liters per minute of air flow and equipped with a USP throat, induction cone and no preseparator, was used. The collection plates of the impactor representing various areas/stages of deposition post-inhalation (trachea, primary and secondary bronchi, terminal bronchi, alveoli, etc.) were coated with silicon spray to prevent bouncing of the microspheres. The microspheres from the stages and collection plates were recovered into a phosphate buffered saline containing 0.1% Tween, and the amount of deposited DAS181 recovered from each stage and collection plate was quantified by measuring absorbance at 280 nm.

Results: The geometric size of microspheres produced by the two methods was assessed by light microscopy and found to be essentially identical (range of 1.5-3.0 microns) for both methods. As shown in Table 3 below, however, the aerodynamic particle size distribution of the two preparations differs significantly between the two methods. For the microspheres produced according to a method as provided herein (Le., method (b) as set forth in section A above), less than 25% remained trapped in the mouth (throat/cone of the impactor assembly), while greater than 70% of the microspheres were delivered to the trachea and lungs (with greater than 40% in the terminal bronchi and alveoli). In comparison, less than 50% of the DAS181 microspheres formed by spray-drying (method (a) as set forth in section A above) was delivered to the trachea and lungs (less than 20% in the terminal bronchi and alveoli). The results demonstrate that methods provided herein can produce microspheres for delivery into deep lungs, and that the microspheres produced by methods provided herein have superior disagglomeration and flowability properties (provide a higher delivered dose) compared to microspheres produced by a spray-drying method.

TABLE 3
Results of Cascade Impaction Analyses of DAS181 Microspheres

| | | | Percent Deposition of DAS181 | |
|---|---|---|---|---|
| Component of the | Corresponding | Expected Deposition | Microspheres Produced | Microspheres |
| Andersen Cascade | Size Cut-Off | in Respiratory | by Method (a) (i.e., | Produced by |
| Impactor | (microns) | Airways | Spray Drying) | Method (b) |
| Throat + Cone | >10 | oral cavity | 42.9 | 16.6 |
| −2 (S + P) | 8.0-10 | oral cavity | 3.7 | 4.9 |
| −1 (S + P) | 6.5-8.0 | oropharynx | 5.9 | 5.5 |
| −0 | 5.2-6.5 | pharynx | 5.8 | 4.0 |
| 1 | 3.5-5.2 | trachea/bronchi | 12.5 | 9.3 |
| 2 | 2.6-3.5 | secondary bronchi | 11.6 | 12.6 |
| 3 | 1.7-2.6 | terminal bronchi | 11.0 | 24.0 |
| 4 | 1.0-1.7 | alveoli | 4.5 | 19.2 |
| 5 | 0.43-1.0 | Alveoli | 1.4 | 3.5 |

### Example 9

### Large Scale Manufacture of Microspheres

This example demonstrates that the methods provided herein can be scaled for the manufacture of large quantities of DAS181. The Batch Process described herein is suitable for the manufacture of high quality dry powder microspheres in an amount ranging from, for example, milligrams to about a kilogram and is limited by the capacity of the mixing tank and/or lyophilizer shelf space. An alternative “continuous” process described herein can be used to manufacture amounts ranging from, for example, hundreds of grams to hundred or more kilograms (100 grams to 100 kg and above). Additional advantage of continuous process is a better control over the chilling of the cocktail.

The large scale manufacture by a batch process or by a continuous process can follow, for example, one or more of the steps described below in any combination of steps or specific alternative methods:
Precipitation of protein into microspheres. This step can be performed in a batch mode by placing the cocktail solution containing the desired concentration of protein, organic solvent and counterion in lyophilization tray(s) and placing the tray(s) onto lyophilizer shelves. Alternatively, trays can be chilled and frozen on a chilled platform or other type of equipment (e.g., a freezer) and stored for a period of time frozen and lyophilized later. Alternatively, the microspheres can be formed by precipitation in a vessel with stirring, wherein the vessel is placed onto a cold surface or a cooling coil is immersed into liquid or while the cocktail is being recirculated through a heat exchanger using a peristaltic pump. Alternatively, the microspheres can be formed by precipitation in a continuous mode, by passing the cocktail solution through a heat exchanger(s) once using a peristaltic pump.
Removal of bulk liquid. The suspension of the microspheres can be concentrated using standard centrifugation, continuous flow centrifugation (e.g., CARR ViaFuge Pilot), or filtration (e.g., on glass fiber, sintered glass, polymer filters, hollow fiber cartridges (e.g., those manufactured by GE Healthcare) or tangential flow filtration cassettes (TFF cassettes, such as those manufactured by Millipore or Sartorius)). The removal of bulk liquid (50% or greater) can result in a faster drying cycle and higher efficiency and throughput.
Drying the microspheres. The recovered microspheres formed by any mode, can be dried by conventional lyophilization. Alternatively, the microspheres can be dried under ambient temperature and atmospheric pressure, eliminating the use of lyophilizer.

Results: DAS181 protein was successfully processed into dry powder (microspheres) by a continuous mode as described herein. Cocktail containing 10 mg/ml DAS181, 20% isopropanol, 2 mM sodium sulfate was passed through 35 SERIES heat exchanger (Exergy, Garden City, NY) coupled with a NESLAB circulating cryostat using a peristaltic pump so that during the passage the cocktail was cooled from about 25° C. to about −12° C. The resulting suspension of microspheres exiting the heat exchanger was pumped into a prechilled lyophilization tray (−40° C.), frozen and lyophilized or, alternatively, pumped directly into liquid nitrogen and then lyophilized. The resulting microspheres, which were analyzed by microscopy and cascade impaction, showed uniform microspheres with minimal aggregation and good dispersivity and were similar in dimensions and aerodynamic particle size distribution to the microspheres produced by batch mode. When the formulated DAS181 cocktail solution was not chilled (not passed through heat exchanger, thus no precipitation of microspheres was induced) and poured directly into liquid nitrogen, no microspheres were observed and, instead, glass-like crystals were observed after lyophilization.

### Example 10

Batch Mode Process and Formulation of DAS181 Microspheres for Delivery to Upper and Central Respiratory Airways

This example describes formulation and a process for manufacture of DAS181 microspheres. The contents of the DAS181 cocktail solution and their relative amounts are shown in Table 4 below.

TABLE 4
Batch Manufacturing Formula for DAS181 Microspheres.

| | | | | | Final | | |
|---|---|---|---|---|---|---|---|
| | Amount for one batch⁽¹⁾ | | | | concentration | | |
| | Stock solution | | Amount | | in formulated | | |
| Ingredient | concentration | | added | | cocktail | | Function |
| DAS181 protein | 19.55 | g/L | 3.306 L, API | | 12 | g/L | Active ingredient |
| Sodium acetate⁽²⁾ | 1.12 | mM | solution | | 0.688 | mM | pH buffer |
| Acetic acid⁽²⁾ | 0.63 | mM | | | 0.0387 | mM | pH buffer |
| Sodium Sulfate | 500 | mM | 0.0215 | L | 2 | mM | Microparticle |
| | | | | | | | formation agent |
| | | | | | | | (counterion) |
| Isopropanol | 100% | v/v | 0.269 | L | 5% | v/v | Microparticle |
| | | | | | | | formation agent |
| Calcium chloride | 500 | mM | 0.0028 | L | 0.268 | mM | Stability enhancing |
| | | | | | | | agent |

| | | | | | |
|---|---|---|---|---|---|
| Water for | neat | 1.79 | L | NA | Diluent |
| irrigation | | | | | |

⁽¹⁾Batch size: final volume of formulated cocktail 5.38 L. Theoretical yield 74 g of
bulk DAS181 Dry Powder.
⁽²⁾Components of the DAS181 protein (API) stock solution.

A. Production of Bulk Drug Substance

The terms Drug Substance, Active Pharmaceutical Ingredient, and API are used interchangeably in this example and refer to the DAS181 protein. Production of DAS181 protein in bulk was conducted as follows. First, bulk amounts of DAS181 were expressed in E. coli (BL21 strain) essentially as described in Example 1. The E. coli cells expressing the DAS181 protein were washed by diafiltration in a fermentation harvest wash step using Toyopearl buffer 1, UFP-500-E55 hollow fiber cartridge (GE Healthcare) and a Watson-Marlow peristaltic pump.

The recombinant DAS181 protein was then purified in bulk from the cells. The detailed specifications of the components and buffers used in the bulk purification of DAS181 are provided in Tables 5 and 6 below. The harvested and washed cells were lysed in a homogenization step by passing the cells twice through using Niro-Soave Panda cell disruptor. The homogenate thus obtained was clarified by microfiltration using the Toyopearl buffer 1, Hydrosart 0.2 micron TFF cassette and a Watson Marlow pump. The clarified homogenate was then concentrated by allowing the lysate to recirculate without fresh buffer feed. Next, DAS181 protein was captured from the clarified homogenate on a Toyopearl SP-550C resin which was washed in a series of buffers (see Table 5) before the DAS181 protein was eluted from the resin. The sodium chloride concentration of the eluate was adjusted to 1.0 M in a final buffer of 50 mM phosphate at pH 8.0. The DAS181-containing eluate was then passed through a Toyopearl Hexyl-650C resin for further purification using a Toyopearl Buffer 4. The resin eluate containing DAS181 protein was then buffer-exchanged into 5 mM sodium acetate in a diafiltration step (see step 8 in Table 5). The concentrated protein was next passed through a Sartorius Q SingleSep Filter in order to remove DNA in a flow-through mode. Isopropanol was added to the Q SingleSep filtrate to a final concentration of 20% v/v. The DAS181 protein in the buffer was passed through an Amberchrome CG300M resin equilibrated with an Amberchrom buffer (see step 11 in Table 5). The purified bulk DAS181 protein was then buffer-exchanged into formulation buffer and concentrated by diafiltration (see step 12 of Table 5).

TABLE 5
Purification of bulk DAS181 drug substance

| 1 | Purpose | Fermentation Harvest Wash | |
|---|---|---|---|
| | Cartridge | GE UFP-500-E55 | Specifications |
| | Activity | Buffer Name | Inlet PSI |
| | Diafiltration | Toyopearl Buffer 1 | 25-35 |
| 2 | Purpose | Homogenization | |
| | Activity | Step | Buffer Name |
| | Equilibration | Equilibration | Harvest Buffer |
| | Homogenization 1st Pass | | Sample Load |
| | Homogenization 2nd Pass | | Sample Load |
| 3 | Purpose | Homogenate | |
| | | Clarification (Diafiltration) | |
| | TFF Cartridge | HydroSart 10K 0.6 m² | Specifications |
| | Activity | Buffer Name | Inlet PSI |
| | Recirculation | Sample Load | 40 |
| | Diafiltration | Toyopearl Buffer 1 | <50 |
| 4 | Purpose | Permeate Concentration | |
| | TFF Cartridge | HydroSart 10K 0.6 m² | Specifications |
| | Activity | Buffer Name | Inlet PSI |
| | Recirculation | Sample Load | NS |
| | Concentration | Sample Load | <50 |
| 5 | Purpose | DAS181 capture performed in bind and elute mode | |
| | Resin | Toyopearl SP-550C | |
| | Activity | Step | Buffer Name |
| | Loading | Sample Load | Clar. Homogenate |
| | Wash | SP Wash 1 | Toyopearl Buffer 1 |
| | | SP Wash 2 | Toyopearl Buffer 2 |
| | | SP Wash 3 | Toyopearl Buffer 3 |
| | | SP Wash 4 | Toyopearl Buffer 2 |
| | | SP Wash 5 | Toyopearl Buffer 1 |
| | Elution | Elution | Toyopearl Buffer 4 |
| 6 | Purpose | Adjust NaCl Concentration | |
| | Method | Add NaCl to 1.0M | |
| | Final Buffer | 50 mM phosphate, 1.0M NaCl, pH 8.0 | |
| 7 | Purpose | DAS181 purification in flow-through mode | |
| | Resin | Toyopearl Hexyl-650C | |
| | Activity | Step | Buffer Name |
| | Loading | Sample Load | Cond. Hexyl Load |
| 8 | Purpose | Concentration & Diafiltration | |
| | TFF Cartridge | HydroSart 10K 0.6 m² | Specifications |
| | Activity | Buffer Name | Recirc. L/min* |
| | Recirculation | Toyopearl Buffer 6 | 15-16 |
| | Concentration | Hexyl Product Pool | 15-16 |
| | Diafiltration | Toyopearl Buffer 6 | 15-16 |
| | Recirculation | Toyopearl Buffer 6 | NS |
| 9 | Purpose | Remove DNA in flow-through mode | |
| | Resin | Sartorius Q SingleSep Filter | |
| | Activity | Step | Buffer Name |
| | Loading | Sample Load | |
| 10 | Purpose | Buffer Adjustment | |
| | Method | Add Isopropanol to 20% | |
| | Final Buffer | 5 mM Acetate, 20% Isopropanol, pH 5.0 | |
| 11 | Purpose | DAS181 polishing in flow-through mode | |
| | Resin | Amberchrome CG300M | |
| | Activity | Step | Buffer Name |
| | Loading | Sample Load | Amberchrom Load |
| 12 | Purpose | Concentration & Diafiltration | |
| | TFF Cartridge | HydroSart 10K 0.6 m² | Specifications |
| | Activity | Buffer Name | Recirc. L/min* |
| | Recirculation | Formulation Buffer | 15-16 |
| | Concentration | Amberchrom Product Pool | 15-16 |
| | Diafiltration | Formulation Buffer | 15-16 |

*Volumes in liters, except 4x denotes multiples of the retentate volume
CV = Column Volumes
NR = Not Recorded
NS = Not Specified

TABLE 6
Buffers used during the DAS181 purification process

| Buffer Name | Buffer Composition |
|---|---|
| Toyopearl Buffer 1 | 50 mM potassium phosphate, 0.3M NaCl, pH 8.0 |
| Toyopearl Buffer 2 | 1.1 mM potassium phosphate, 2.9 mM |
| | sodium phosphate, 154 mM NaCl, pH 7.4 |
| Toyopearl Buffer 3 | 1.1 mM potassium phosphate, 2.9 mM |
| | sodium phosphate, 154 mM NaCl, 1% Triton |
| | X-100, 0.1% SDS, 0.5% sodium deoxycholate, |
| | pH 7.4 |
| Toyopearl Buffer 4 | 50 mM potassium phosphate, 1.0M NaCl, pH 8.0 |
| Toyopearl Buffer 5 | 50 mM potassium phosphate, 0.5M NaCl, pH 8.0 |
| Toyopearl Buffer 6 | 5 mM sodium acetate, pH 5.0 |
| Toyopearl Buffer 7 | 5 mM sodium acetate, 60% isopropanol, pH 5.0 |
| Formulation Buffer | 1.75 mM sodium acetate, pH 5.0 |
| 3% Isoproyl Alcohol | 3% isopropanol |
| Amberchrom Buffer | 5 mM sodium acetate, 20% isopropanol, |
| | pH 5.0 adjusted with acetic acid |
| 1.0N NaOH 3% | 1.0N NaOH, 3% isopropanol |
| Isopropanol | |
| 1.0N NaOH | 1.0N NaOH |
| 0.5N NaOH | 0.5N NaOH |
| 0.1N NaOH | 0.1N NaOH |
| 70% Isopropyl | 70% isopropanol |
| Alcohol | |
| 20% EtOH | 20% ethyl alcohol |

B. Batch Manufacturing Process

The ingredients set forth in Table 4 above were combined to form DAS181 microspheres in a large scale batch process as described below.

### Step I: Thawing of Bulk Drug Substance

Frozen 0.2 μm-filtered bulk Drug Substance in plastic bottles was thawed overnight at ambient temperature (25±3° C.).

### Step II: Weighing of the Excipients and Preparation of Solutions

35.51 g of Sodium Sulfate anhydrous powder was weighed and Q.S. to 500 mL with Water For Irrigation, then stirred to obtain a clear solution. 18.38 g of Calcium Chloride dihydrate powder was weighed and Q.S. to 250 mL with Water For Irrigation, then stirred to obtain a clear solution.

### Step III: Preparation of the DAS181 Cocktail Solution

To 3.3 L of concentrated Drug Substance (19.55 g/L), 1.79 L of Water For Irrigation was added slowly with stirring, followed by 0.0215 L of Sodium Sulfate solution, 0.0028 L of Calcium Chloride solution and 0.269 L of isopropanol. The solution was stirred to ensure complete mixing of components.

### Step IV: Filtration of Formulated Cocktail Solution Through 0.2 μm Filter

The formulated cocktail solution of Step III was filtered through a 0.2 μm filter into sterile media bags to control particulates and bioburden.

Step V: Filling into Lyophilization Trays

The formulated filtered solution was dispensed into autoclaved Lyoguard lyophilization trays. To ensure even cooling of the solution and formation of high quality microspheres, 6 trays were each filled with 0.9 L or less of cocktail solution.

### Step V1: Freezing and Lyophilization

The trays were placed onto lyophilizer (Hull 120FSX200) shelves pre-chilled to −45±5° C. and the solution was allowed to chill and freeze. Formation of microspheres occurred while the solution was being frozen. The freezing is allowed to proceed for 1-2 h to ensure complete solidification. The product temperature was verified by reading the thermocouples attached to two of the six trays.

The lyophilization cycle steps are as follows:
a) Set vacuum to 160 microns and allow to evacuate to 100-200 microns;
b) Ramp shelf temperature to +10° C. over 3 h;
c) Hold shelf temperature at +10° C. for 36 h (primary drying);
d) Thermocouple traces examined to verify that primary drying phase is completed and the product temperature has stabilized at +10° C.±5° C. for 15-30 h.
e) Ramp shelf temperature to +30° C. over 1 h and hold for 3-5 h (secondary drying).
   Step VII: Transfer of Bulk DAS181 Microspheres into Container and Mixing

A section on the bottom film of each Lyoguard lyophilization tray was cleaned using sanitizing wipes and a 3×3 cm opening was made with a scalpel. The dry microspheres were transferred into a plastic bottle. The bottle was capped and tumbled forty times, changing directions with each inversion. The tumbling was to ensure uniformity of bottle content. Samples for analytical testing were taken and the bottle was recapped and sealed into plastic bags for storage.

In the DAS181 microsphere bulk manufacturing process as described above, sulfate was demonstrated to be a safe substance for use as a counterion, and reproducibly produced microspheres with a narrow size distribution. Further, the organic solvent isopropanol was a good solvent of choice because (1) a class 3 solvent, (2) it can produce microspheres in a wide range (2-30%, v/v) of concentrations, and (3) it has a relatively high freezing point so its vapors can efficiently be trapped during lyophilization.

The protein concentration in the final formulation could be varied (10-14 mg/ml), as could the concentration of counterion (1-5 mM) and isopropanol (2-30% v/v), without substantial impact on the physical properties of the microspheres or the activity of the DAS181 protein in the microspheres. At higher concentrations of isopropanol (15-30%), the microspheres formed while the cocktail was still fully liquid. At lower concentrations (2-15%), ice crystals began to form first, followed by precipitation to form microspheres.

C. Yield of DAS181 in the Microspheres

The theoretical yield of DAS181 in the dry microspheres is calculated according to the following formula:

Theoretical yield=DAS181 protein, g÷protein fraction in Dry Powder (microspheres)

The protein fraction value (0.866) was established empirically by analysis of several manufactured batches of DAS181 microspheres. The theoretical yield for the amounts as set forth in Table 2 is 64.56 g÷0.866=74.55 g. The actual yield of DAS181 Dry Powder was found to be 64 g.

Results: The suitability of the microspheres prepared as described in section B above for administration by oral inhalation was tested by Andersen Cascade Impaction. The results are summarized in Table 7 below. The deposition of pharmaceuticals in the respiratory tract can be predicted by deposition of particles (microspheres) on the stages/collection plates of the cascade impactor. For a pharmaceutical, e.g., DAS181 microspheres, that is administered to prevent or treat viral infections that initiate in the respiratory tract, such as influenza, it is desirable to deposit the pharmaceutical in the throat, trachea, bronchi (upper and central respiratory airways). The DAS181 fusion protein delivered to upper and central respiratory airways cleaves off the receptor sialic acids from mucous membranes, thus preventing viral binding and infection at these sites. For optimal delivery of the DAS181 microspheres to sites where respiratory viral infection can be initiated, i.e., in the throat, trachea or bronchi, the microspheres must not be (a) so big that they are trapped at the front end in the mouth (i.e., microspheres are too big, about 8 microns or greater); or (b) so small that they are deposited in deep lungs and absorbed systemically into the blood stream (i.e., 0.5 microns or smaller). For delivery of the DAS181 microspheres to the throat, trachea and bronchi, a size range of about 1 micron to about 5.5-6 microns generally is suitable.

DAS181 microspheres manufactured as described above were characterized by Andersen cascade impaction and found to be suitable for delivery to upper and central respiratory airways with sufficiently low percentage (<5%) deposited in the alveoli.

TABLE 7
Aerodynamic Particle Size Distribution of DAS181 dry powder at 60
liters per minute.

| | | | | Percent of |
|---|---|---|---|---|
| | | | DAS181 | total |
| Component of | | | protein | DAS181 |
| Andersen Cascade | Corresponding size | Expected deposition in | deposited (in | protein |
| Impactor | cut-off, microns | respiratory airways | mg) | recovered |
| Inhaler (Cyclohaler) | | | 1.57 ± 0.11 | 20.13% |
| Throat/Cone | >10 | Oral cavity | 0.93 ± 0.19 | 11.92% |
| −1 (Stage + Plate) | 8.6-10 | Oral cavity | 0.50 ± 0.10 | 6.41% |
| −0 (Stage + Plate) | 6.5-8.6 | oropharynx | 0.40 ± 0.03 | 5.13% |
| 1 (Stage + Plate) | 4.4-6.5 | pharynx | 0.58 ± 0.03 | 7.44% |
| 2 (Stage + Plate) | 3.3-4.4 | trachea/bronchi | 0.83 ± 0.07 | 10.64% |
| 3 (Stage + Plate) | 2.0-3.3 | Secondary bronchi | 1.80 ± 0.09 | 23.08% |
| 4 (Stage + Plate) | 1.1-2.0 | Terminal bronchi | 0.82 ± 0.08 | 10.51% |
| 5 (Stage + Plate) | 0.54-1.1 | alveoli | 0.23 ± 0.03 | 2.95% |
| 6 (Stage + Plate) | 0.25-0.54 | alveoli | 0.14 ± 0.03 | 1.79% |
| ΣACI (Emitted) | | | 6.24 ± 0.10 | 80.00% |

10 ± 1.0 mg of DAS181 Dry Powder (8.5 mg ± 10% DAS181 protein) was filled into HPMC capsule
ΣACI (Emitted) fraction is the sum of all material recovered from USP Throat, Induction Cone and stages −1 to 6.

DAS181 microspheres were further characterized by laser diffraction, which demonstrated, consistent with the cascade impaction results, that the majority of the microspheres produced by the method described in this Example are within a size range of between 1 micron and 5 microns in size. Scanning Electron Microscopy (FEI Quanta 200 Scanning Electron Microscope, Everhart Thornley (ET) detector) of the DAS181 microspheres prepared according to the method described in this Example revealed that the microspheres are present as agglomerates of hundreds and thousands of individual particles approximately 0.5-3 micron in size. The agglomerates however are easily dissipated by air turbulence produced during the actuation through dry powder inhaler (as demonstrated by Andersen Cascade Impaction or laser diffraction). Light microscopy of microspheres dispersed in a liquid surfactant (e.g. Triton X-100 or Tween 20) or non-polar solvent (e.g., alcohol, acetone, or acetonitrile) that does not dissolve the microspheres, confirmed that aggregates are easily dissipated into individual uniform microspheres.

### Example 11

### Preparation of DAS181 Microspheres Using Sulfates Other than the Sodium Salt

Studies have shown that in certain instances, e.g., in some asthmatics, the presence of sodium in formulations for pulmonary administration could carry a risk of inducing airway hyperresponsiveness (Agrawal et al., Lung, 183:375-387 (2005)). This example therefore tested alternate salts, such as salts of other metals such as potassium, magnesium and calcium.

DAS181 microspheres were manufactured as described above in Example 1. Cocktail solutions containing 12 mg/mL DAS181 and 5% (v/v) isopropanol contained as counterions the indicated sulfates at 2 mM concentration, pH 4.5-5.0. The microspheres were formed by cooling the solutions from +25° C. to −45° C. Upon freezing, the volatiles (water and isopropanol) were removed by sublimation, leaving the dry powder containing microspheres.

The aerodynamic particle size distribution of the dry powder was assessed by Andersen Cascade Impaction, and the amount of DAS181 per stage was determined by UV measurement at 226 nm (A₂₂₆). The results are shown below in Table 8. The results demonstrate that sulfate salts other than the sodium salt can be used as counterion to obtain DAS181 microspheres of a size range such that the majority are delivered to the throat, trachea and bronchi, in an amount that is comparable to the amount delivered when sodium sulfate is used as the counterion.

TABLE 8
Aerodynamic Particle Size Distribution of DAS181
microspheres formulated with or without sodium

| | | Expected | | | | |
|---|---|---|---|---|---|---|
| | Corresponding | deposition in | Percent DAS181 per stage | | | |
| | size cut-off, | respiratory | Sodium | Potassium | Magnesium | Calcium |
| | microns | airways | Sulfate | Sulfate | Sulfate | Sulfate |
| Inhaler | | | 19.86% | 28.58% | 21.41% | 16.71% |
| Capsule | | | 2.07% | 2.30% | 1.88% | 0.00% |
| Throat + Cone | >10 | Oral cavity | 11.67% | 9.00% | 12.91% | 16.79% |
| −1(S + P) | 8.6-10 | Oral cavity | 10.00% | 3.43% | 7.86% | 14.87% |
| −0(S + P) | 6.5-8.6 | oropharynx | 5.30% | 3.08% | 4.71% | 7.77% |
| 1(S + P) | 4.4-6.5 | pharynx | 6.97% | 5.86% | 6.58% | 7.54% |
| 2(S + P) | 3.3-4.4 | trachea/bronchi | 7.55% | 8.24% | 6.90% | 6.43% |
| 3(S + P) | 2.0-3.3 | Secondary bronchi | 19.57% | 20.21% | 17.01% | 12.65% |
| 4(S + P) | 1.1-2.0 | Terminal bronchi | 12.39% | 14.00% | 13.00% | 10.39% |
| 5(S + P) | 0.54-1.1 | alveoli | 2.80% | 2.99% | 4.31% | 4.69% |
| 6(S + P) | 0.25-0.54 | alveoli | 1.82% | 2.31% | 3.44% | 2.16% |

The dry powders also were incubated at +37° C. or +53° C. for a duration as indicated in Table 9 and tested for sialidase activity using the 4-MU-NANA assay as described in Example 1 and incorporated by reference herein. The relative activity compared to non-lyophilized DAS181 microspheres stored at −80° C. is shown in Table 9. The results show that the stability of the microspheres prepared using the various metal sulfates as counterions were comparable to that of sodium sulfate, with retention of almost all or all the activity for over 2 months at 37° C. and retention of almost all (sodium and potassium sulfates) or over 85% (magnesium and zinc sulfates) of the activity for over 10 days at 53° C. This experiment demonstrates that various non-sodium containing counterions can produce microspheres with desirable characteristics.

TABLE 9
Sialidase activity of DAS181 microsphere formulations:
accelerated stability studies.

| | Percent Activity Remaining | | | |
|---|---|---|---|---|
| | Temperature | | | |
| | 37° C. | | 53° C. | |
| | Incubation Days | | | |
| | 42 Days | 69 Days | 11 Days | 39 Days |
| 2 mM Sodium Sulfate + | 107.14% | 105.62% | 110.66% | 23.66% |
| 0.268 mM CaCl₂ | | | | |
| 2 mM Potassium Sulfate + | 97.37% | 104.00% | 101.54% | 52.76% |
| 0.268 mM CaCl₂ | | | | |
| 2 mM Magnesium Sulfate + | 123.81% | 107.29% | 85.93% | 60.00% |
| 0.268 mM CaCl₂ | | | | |
| 13.34 mM Calcium/ | 116.67% | 93.20% | 87.12% | 40.48% |
| 2 mM Sulfate | | | | |

### Example 12

### Stability of DAS181 Microspheres

The stability of the DAS181 protein in the microspheres was assessed by measuring sialidase activity over time using the 4-MU-NANA activity assay as described above in Example 1 and as incorporated by reference herein. The production of dry DAS181 microspheres was undertaken in a cocktail solution containing 10 mg/mL DAS181, 2 mM sodium sulfate, 5% v/v isopropanol. To some solutions, 0.01% w/v sugar (sorbitol, mannitol, trehalose or sucrose) was added. The microspheres were formed by cooling the solutions from ±25° C. to −45° C. Upon freezing, the volatiles (water and isopropanol) were removed by sublimation, leaving the dry powders containing microspheres.

A. Stability of DAS181 Microspheres without Sugars

The DAS181 dry powder microspheres formulated without sugars were stored at room temperature (25° C.) in a container next to Drierite desiccant (Hammond Drierite, Xenia, Ohio). The dry powder retained its original potency (as measured by sialidase activity using 4-MU-NANA according to Examplel and as incorporated by reference herein; results shown in Table 10) and aerodynamic particle size distribution (as measured by Andersen Cascade impaction; Table 11) for at least 8 months.

TABLE 10
Specific activity of DAS181 dry powder.

| Test | Time 0 | 3 months | 8 months |
|---|---|---|---|
| Sialidase Activity with | 100% | 102.0% | 99.9% |
| reference to time 0 | | | |

TABLE 11
Aerodynamic particle size distribution of DAS181 dry powder

| | | Expected | | | |
|---|---|---|---|---|---|
| | Corresponding | deposition in | | | |
| ACI | size cut-off, | respiratory | | | |
| Component | microns | airways | Time 0 | 3 Months | 8 Months |
| Throat + | >10 | Oral cavity | 19.57 ± 2.43 | 26.00 ± 0.30 | 18.57 ± 4.14 |
| Cone | | | | | |
| Stage −1 | 8.6-10 | Oral cavity | 17.87 ± 0.51 | 12.87 ± 1.56 | 15.13 ± 2.41 |
| Stage −0 | 6.5-8.6 | oropharynx | 10.27 ± 0.93 | 7.07 ± 0.32 | 9.80 ± 1.80 |
| Stage 1 | 4.4-6.5 | pharynx | 8.57 ± 0.49 | 8.80 ± 0.26 | 7.73 ± 0.57 |
| Stage 2 | 3.3-4.4 | trachea/bronchi | 10.67 ± 0.23 | 10.70 ± 0.35 | 9.30 ± 0.82 |
| Stage 3 | 2.0-3.3 | Secondary | 21.10 ± 0.75 | 21.80 ± 0.52 | 21.90 ± 0.87 |
| | | bronchi | | | |
| Stage 4 | 1.1-2.0 | Terminal | 10.10 ± 0.75 | 10.63 ± 0.80 | 14.50 ± 3.22 |
| | | bronchi | | | |
| Stage 5 | 0.54-1.1 | alveoli | 1.47 ± 0.23 | 1.73 ± 0.06 | 2.37 ± 0.06 |
| Stage 6 | 0.25-0.54 | alveoli | 0.33 ± 0.06 | 0.40 ± 0.10 | 0.73 ± 0.06 |

Table 11:
Aerodynamic particle distribution was assessed by Andersen Cascade Impaction and expressed as % of total DAS181 protein recovered.
Capsules were filled with 10 mg of DAS181 dry powder and actuated using Cyclohaler dry powder inhaler as delivery device. Air flow rate was 60 Liters per minute.
Assays were performed in triplicate, mean and standard deviation are shown.

B. Stability of DAS181 Microspheres Formulated with Sugars

The sialidase activity of DAS181 in the dry powder microsphere formulations containing sugars and in the unlyophilized microsphere formulations stored at −80° C., were measured using the fluorescent substrate 4-MU-NANA as described in Example 1 and as incorporated by reference herein. The dry powder formulations containing no sugar or various sugars as indicated below in Table 12 were stored at +42° C. for 4 weeks (forced degradation). The results are shown in Table 12. Relative to unlyophilized formulations stored at −80° C., the formulation containing no sugar retained almost 80% of its activity. The addition of various sugars increase the stability so that about 88-98% of the activity is retained, depending on the sugar.

TABLE 12

| | Percent Sialidase Activity |
|---|---|
| Sugar | Remaining after 4 weeks at 42° C. |
| No Sugar | 79.82 |
| Sorbitol | 91.23 |
| Mannitol | 89.47 |
| Trehalose | 97.37 |
| Sucrose | 88.60 |

The stabilizing effect of higher percentages of sugars on the DAS181 microspheres was investigated. The presence of glycine in the reaction cocktails served to prevent crystallization of the sugars during the manufacture of the DAS181 microspheres. The study showed that up to 15% of Trehalose, Sucrose, Sorbitol, or Mannitol, when combined with 5% glycine, can be incorporated into the DAS181 microsphere-forming reactions without forming crystals during lyophilization. The microspheres were indistinguishable from the ones produced without sugars, based on their appearance under light microscopy and scanning electron microscopy (SEM). The aerodynamic particle size distribution of the microspheres also remained unaffected.

5 mg of the resulting DAS181 dry powder was placed in clear size 3 HPMC capsules and stored at 37° C. The percentage amount of high molecular weight DAS181 oligomers (degradation products) was quantitatively analyzed by size exclusion HPLC. The results in Table 12A demonstrate the protective effect of the sugars, with Trehalose and Sucrose providing the best protection. Results are expressed as % of oligomers at 0 months, 1 month, 2 months or 3 months.

TABLE 12A

% of oligomers

| | 0 months | 1 month | 2 months | 3 months |
|---|---|---|---|---|
| Mannitol | 0.0 | 6.01 | 9.79 | 12.59 |
| Trehalose | 0.0 | 3.75 | 5.82 | 6.79 |
| Sucrose | 0.0 | 3.92 | 5.67 | 8.16 |
| Sorbitol | 0.0 | 4.88 | 7.35 | 9.59 |

The stabilizing effect of Sucrose and Trehalose on DAS181 microsphere formulations was further confirmed by the following experiments. Formulations containing the sugars and a sugar-free control were produced and either left as bulk powders (unencapsulated) or placed into clear size 3 HPMC capsules. The samples were stored at 37° C. The percentage of high molecular weight DAS181 oligomers (degradation products) was quantitated by size exclusion HPLC. The results in Table 12B (below) again demonstrate that Trehalose and Sucrose sugars provided significant protection. Results are expressed as % of oligomers at 0 months, 1 month, 2 months or 8 months.

TABLE 12 B

| | % of oligomers | | | | | | |
|---|---|---|---|---|---|---|---|
| | | HPMC Capsules | | | Unencapsulated | | |
| Sugar | t = 0 | t = 1 | t = 2 | t = 8 | t = 1 | t = 2 | t = 8 |
| Sucrose | 0.0 | 4.2 | 8.9 | 15.0 | 0.0 | 1.1 | 0.9 |
| Trehalose | 0.0 | 4.8 | 8.6 | 14.3 | 0.0 | 1.0 | 1.0 |
| None | 0.0 | 5.0 | 9.4 | 18.6 | 0.9 | 2.4 | 3.5 |

Formulations contained 15% w/w Sugar, 5% w/w Glycine, 2 mM Acetate pH 6.0, and 2 mM MgSO₄

### Example 13

### Preparation of Microspheres Using a Variety of Classes of Compounds

The methods provided herein can be used to prepare microspheres using a variety of classes of compounds. In addition to proteins such as DAS181, BSA, trypsin, hemoglobin, DNase I, lysozyme, ovalbumin, RNAse A, hexahistidine-tagged human proteinase inhibitor 8 (PI8, having the sequence of amino acids set forth in SEQ ID NO:15), red fluorescent protein (RFP) and green fluorescent protein (GFP), as described in the above Examples, this Example demonstrates that the method can be used to prepare microspheres of:

A. The antibiotics—Vancomycin, Tobramycin, Kanamycin and Ampicillin

B. A nucleic acid—siRNA

C. A virus—Tobacco Mosaic Virus

D. The peptides—leuprolide and somatostatin

The microspheres prepared from A-D above were compared to those of the protein, DAS181.

Preparation of microspheres: for each of the compounds listed in A-D above, and for DAS181, cocktail solutions containing 2 mg/ml of compound dissolved in an aqueous solvent and various counterions, antisolvents and pH, as listed below, were prepared in a 96-well microtiter plate (0.1 ml cocktail/well) at room temperature. Control solutions contained either solvent or antisolvent alone, with or without the counterion. Cocktails were cooled by placing in a freezer. The chilled plates were transferred onto pre-chilled (−45° C.) shelves of a Millrock Lab Series Lyophilizer, and the vacuum was applied. The frozen cocktail solutions were allowed to lyophilize for 16 hours.

The lyophilized powders from the bottoms of the wells were transferred onto glass slides and analyzed by light microscopy for appearance. The quality of the product microspheres was scored based on the uniformity of the microspheres, the absence of undesirable non-microsphere particles (glass-like crystalline forms), and the absence of aggregates. Table 13 below provides an exemplary scoring system, based on appearance.

TABLE 13
Scoring System for Assessing Quality of Microspheres

| | | Presence of |
|---|---|---|
| | | Glass like |
| | Presence/Quality of | crystals and/or |
| Score | Microspheres | Aggregates |
| 0 | Absent | Exclusive |
| 1 | Almost absent | Almost exclusive |
| 2 | scarce | Highly dominant |
| 3 | observable | Dominant |
| 4 | Present in large quantities | Present in smaller |
| | relative to glass-like crystals or | quantities than |
| | aggregates | the microspheres |
| 5 | Dominant | Scarce |
| 6 | Almost uniform | Observable, but |
| | | minimal |
| 7 | Uniform | Observable, but |
| | | very minimal |
| 8 | Uniform | Very few |
| 9 | Very uniform | Almost absent |
| 10 | Perfect and uniform | Absent |

Table 14 below shows the various combinations of compound, solvent, antisolvent and counterion that were used to generate microspheres, and the aualitv of the resultina microspheres.

TABLE 14
Combinations Used to Produce Microspheres of Different
Compounds
A. Antibiotics

| | | | |
|---|---|---|---|
| | | | Microsphere |
| Counterion | Antisolvent | pH | Quality |

Compound: Tobramycin

| | | | |
|---|---|---|---|
| 2 mM Na-Citrate | 5% | 5.0 | 4 |
| | isopropanol | | |
| 2 mM Na- | 5% | 4.0 | 2 |
| Glutamate | isopropanol | | |
| 2 mM Arginine | 5% | 7.0 | 5 |
| HCl/NaOH | isopropanol | | |
| 2 mM Itaconic Acid- | 5% | 4.0 | 9 |
| Na | isopropanol | | |
| 2 mM Na-Citrate | 15% | 5.0 | 3 |
| | isopropanol | | |
| 2 mM Na- | 15% | 4.0 | 2 |
| Glutamate | isopropanol | | |
| 2 mM Arginine | 15% | 7.0 | 3 |
| HCl/NaOH | isopropanol | | |
| 2 mM Arginine | 15% | 9.0 | 5 |
| HCl/NaOH | isopropanol | | |
| 2 mM Itaconic Acid- | 15% | 4.0 | 8 |
| Na | isopropanol | | |
| 2 mM Itaconic Acid- | 15% n- | 4.0 | 8 |
| Na | propanol | | |

Compound: Kanamycin

| | | | |
|---|---|---|---|
| 2 mM Na-Citrate | 5% | 5.0 | 8 |
| | isopropanol | | |
| 2 mM Na- | 5% | 4.0 | 3 |
| Glutamate | isopropanol | | |
| 2 mM Itaconic Acid- | 5% | 4.0 | 8 |
| Na | isopropanol | | |
| 2 mM Itaconic Acid- | 5% | 7.0 | 8 |
| Na | isopropanol | | |
| 2 mM Na-Citrate | 15% | 5.0 | 9 |
| | isopropanol | | |
| 2 mM Na- | 15% | 4.0 | 4 |
| Glutamate | isopropanol | | |
| 2 mM Na- | 15% | 7.0 | 4 |
| Glutamate | isopropanol | | |
| 2 mM Arginine | 15% | 7.0 | 4 |
| HCl/NaOH | isopropanol | | |
| 2 mM Arginine | 15% | 9.0 | 0 |
| HCl/NaOH | isopropanol | | |
| 2 mM Itaconic Acid- | 15% | 4.0 | 9 |
| Na | isopropanol | | |
| 2 mM Itaconic Acid- | 15% | 7.0 | 7 |
| Na | isopropanol | | |
| 2 mM Na-Citrate | 5% n- | 5.0 | 7 |
| | propanol | | |
| 2 mM Na- | 5% n- | 4.0 | 5 |
| Glutamate | propanol | | |
| 2 mM Itaconic Acid- | 5% n- | 7.0 | 9 |
| Na | propanol | | |
| 2 mM Itaconic Acid- | 15% n- | 4.0 | 8 |
| Na | propanol | | |

Compound: Ampicillin

| | | | |
|---|---|---|---|
| 2 mM Na-Citrate | 5% | 5.0 | 5 |
| | isopropanol | | |
| 2 mM Na- | 5% | 4.0 | 4 |
| Glutamate | isopropanol | | |
| 2 mM Itaconic Acid- | 5% | 4.0 | 3 |
| Na | isopropanol | | |
| 2 mM Na-Citrate | 15% | 5.0 | 2 |
| | isopropanol | | |
| 2 mM Na- | 15% | 4.0 | 3 |
| Glutamate | isopropanol | | |
| 2 mM Itaconic Acid- | 15% | 4.0 | 7 |
| Na | isopropanol | | |
| 2 mM Na-Citrate | 5% n- | 5.0 | 3/4 |
| | propanol | | |
| 2 mM Na- | 5% n- | 4.0 | 3-5 |
| Glutamate | propanol | | |
| 2 mM Na- | 15% n- | 4.0 | 4 |
| Glutamate | propanol | | |
| 2 mM Itaconic Acid- | 15% n- | 4.0 | 3 |
| Na | propanol | | |
| 2 mM Itaconic Acid- | 15% n- | 7.0 | 7 |
| Na | propanol | | |

Compound: Vancomycin

| | | | |
|---|---|---|---|
| 2 mM Na-Citrate | 5% | 5.0 | 4 |
| | isopropanol | | |
| 2 mM Na- | 5% | 4.0 | 4 |
| Glutamate | isopropanol | | |
| 2 mM Na- | 5% | 7.0 | 7 |
| Glutamate | isopropanol | | |
| 2 mM Arginine | 5% | 7.0 | 7 |
| HCl/NaOH | isopropanol | | |
| 2 mM Arginine | 5% | 9.0 | 6 |
| HCl/NaOH | isopropanol | | |
| 2 mM Itaconic Acid- | 5% | 4.0 | 8 |
| Na | isopropanol | | |
| 2 mM Itaconic Acid- | 5% | 7.0 | 8 |
| Na | isopropanol | | |
| 2 mM Na-Citrate | 15% | 5.0 | 4 |
| | isopropanol | | |
| 2 mM Na- | 15% | 4.0 | 4 |
| Glutamate | isopropanol | | |
| 2 mM Na- | 15% | 7.0 | 3 |
| Glutamate | isopropanol | | |
| 2 mM Arginine | 15% | 7.0 | 4 |
| HCl/NaOH | isopropanol | | |
| 2 mM Arginine | 15% | 9.0 | 4 |
| HCl/NaOH | isopropanol | | |
| 2 mM Itaconic Acid- | 15% | 4.0 | 7 |
| Na | isopropanol | | |
| 2 mM Itaconic Acid- | 15% | 7.0 | 3 |
| Na | isopropanol | | |
| 2 mM Na-Citrate | 5% n- | 5.0 | 9 |
| | propanol | | |
| 2 mM Na- | 5% n- | 4.0 | 6 |
| Glutamate | propanol | | |
| 2 mM Na- | 5% n- | 7.0 | 8 |
| Glutamate | propanol | | |
| 2 mM Arginine | 5% n- | 7.0 | 3 |
| HCl/NaOH | propanol | | |
| 2 mM Arginine | 5% n- | 9.0 | 4 |
| HCl/NaOH | propanol | | |
| 2 mM Itaconic Acid- | 5% n- | 4.0 | 5 |
| Na | propanol | | |
| 2 mM Itaconic Acid- | 5% n- | 7.0 | 2 |
| Na | propanol | | |
| 2 mM Na-Citrate | 15% n- | 5.0 | 9 |
| | propanol | | |
| 2 mM Na- | 15% n- | 4.0 | 7 |
| Glutamate | propanol | | |
| 2 mM Na- | 15% n- | 7.0 | 4 |
| Glutamate | propanol | | |
| 2 mM Arginine | 15% n- | 9.0 | 4 |
| HCl/NaOH | propanol | | |
| 2 mM Itaconic Acid- | 5% n- | 7.0 | 5 |
| Na | propanol | | |

The experiments with Vancomycin were also performed on a larger scale (20 mg Vancomycin), namely, at 2 mg/ml Vancomycin in a 5 ml total volume; and at 10 mg/ml Vancomycin in a 2 ml total volume. All cocktail mixes tested produced very high quality microspheres of Vancomycin as described below:

20 mg Vancomycin

| | | | Microsphere |
|---|---|---|---|
| Counterion | Antisolvent | pH | Quality |
| 5 mM Na-Citrate | 15% n- | 5.0 | 9 |
| | propanol | | |
| 5 mM Na-glutamate | 5% n- | 7.0 | 9 |
| | propanol | | |
| 5 mM Na-Citrate | 15% n- | 5.0 | 10 |
| | propanol | | |
| 5 mM Na-glutamate | 5% n- | 7.0 | 9 |
| | propanol | | |

B. Nucleic Acid - siRNA

| | | | |
|---|---|---|---|
| | | | Microsphere |
| Counterion | Antisolvent | pH | Quality |

Compound: siRNA

| | | | |
|---|---|---|---|
| 2 mM Na-Citrate | 5% | 4.0 | 2 |
| | isopropanol | | |
| 2 mM Na-Citrate | 5% | 5.0 | 2 |
| | isopropanol | | |
| 2 mM Na-Citrate | 5% | 7.0 | 2 |
| | isopropanol | | |
| 2 mM Na- | 5% | 4.0 | 3 |
| Glutamate | isopropanol | | |
| 2 mM Na- | 5% | 7.0 | 1 |
| Glutamate | isopropanol | | |
| 2 mM Arginine | 5% | 7.0 | 1 |
| HCl/NaOH | isopropanol | | |
| 2 mM Arginine | 5% | 9.0 | 1 |
| HCl/NaOH | isopropanol | | |
| 2 mM Itaconic Acid- | 5% | 4.0 | 3 |
| Na | isopropanol | | |
| 2 mM Itaconic Acid- | 5% | 7.0 | 0 |
| Na | isopropanol | | |
| 2 mM Pivalic Acid | 5% | 4.0 | 1 |
| | isopropanol | | |
| 2 mM Pivalic Acid | 5% | 5.0 | 2 |
| | isopropanol | | |
| 2 mM PEI 750000 | 5% | | 7 |
| | isopropanol | | |
| 2 mM PEI 25000 | 5% | | 7 |
| | isopropanol | | |
| 2 mM PEI 2000 | 5% | | 3 |
| | isopropanol | | |
| 2 mM Na- | 5% | 4.0 | 1 |
| Sulfate/Na-Acetate | isopropanol | | |
| 2 mM Na- | 5% | 6.0 | 1 |
| Sulfate/Na-Acetate | isopropanol | | |
| 2 mM Na-Citrate | 15% | 4.0 | 2 |
| | isopropanol | | |
| 2 mM Na-Citrate | 15% | 5.0 | 1 |
| | isopropanol | | |
| 2 mM Na-Citrate | 15% | 7.0 | 0 |
| | isopropanol | | |
| 2 mM Na- | 15% | 7.0 | 1 |
| Glutamate | isopropanol | | |
| 2 mM Arginine | 15% | 7.0 | 4/5 |
| HCl/NaOH | isopropanol | | |
| 2 mM Arginine | 15% | 9.0 | 4 |
| HCl/NaOH | isopropanol | | |
| 2 mM Itaconic Acid- | 15% | 4.0 | 4 |
| Na | isopropanol | | |
| 2 mM Pivalic Acid | 15% | 4.0 | 3 |
| | isopropanol | | |
| 2 mM Pivalic Acid | 15% | 5.0 | 3 |
| | isopropanol | | |
| 2 mM PEI 750000 | 15% | | 6 |
| | isopropanol | | |
| 2 mM PEI 25000 | 15% | | 6 |
| | isopropanol | | |
| 2 mM Na- | 15% | 4.0 | 1 |
| Sulfate/Na-Acetate | isopropanol | | |
| 2 mM Na- | 15% | 6.0 | 3 |
| Sulfate/Na-Acetate | isopropanol | | |

Compound: siRNA (2 mg/ml)

| | | | |
|---|---|---|---|
| None | 15% | | 10 |
| | isopropanol | | |
| 2 mM Arginine | 15% | 7.0 | 7/8 |
| HCl/NaOH | isopropanol | | |
| 2 mM Arginine | 15% | 9.0 | 4 |
| HCl/NaOH | isopropanol | | |
| 2 mM Itaconic Acid- | 15% | 4.0 | 3 |
| Na | isopropanol | | |
| 2 mM PEI 25000 | 15% | | 8 |
| | isopropanol | | |
| 1 mM PEI 25000 | 15% | | 7 |
| | isopropanol | | |
| 0.5 mM PEI 25000 | 15% | | 7 |
| | isopropanol | | |
| 0.1 mM PEI 25000 | 15% | | 7 |
| | isopropanol | | |
| 2 mM Arginine | 30% | 7.0 | 5 |
| HCl/NaOH | isopropanol | | |
| 2 mM Arginine | 30% | 9.0 | 5/6 |
| HCl/NaOH | isopropanol | | |
| 2 mM Itaconic Acid- | 30% | 4.0 | 8 |
| Na | isopropanol | | |
| 2 mM PEI 25000 | 30% | | 7 |
| | isopropanol | | |
| 1 mM PEI 25000 | 30% | | 8 |
| | isopropanol | | |
| 0.5 mM PEI 25000 | 30% | | 9 |
| | isopropanol | | |
| 0.1 mM PEI 25000 | 30% | | 6 |
| | isopropanol | | |

Compound: siRNA (0.25 mg/ml)

| | | | |
|---|---|---|---|
| 2 mM Arginine | 15% | 7.0 | 0 |
| HCl/NaOH | isopropanol | | |
| 2 mM Arginine | 15% | 9.0 | 0 |
| HCl/NaOH | isopropanol | | |
| 2 mM Arginine | 30% | 7.0 | 6 |
| HCl/NaOH | isopropanol | | |
| 2 mM Arginine | 30% | 9.0 | 3 |
| HCl/NaOH | isopropanol | | |

Compound: siRNA (5 mg/ml)

| | | | |
|---|---|---|---|
| 2 mM Arginine | 15% | 7.0 | 3 |
| HCl/NaOH | isopropanol | | |
| 2 mM Itaconic acid- | 15% | 4.0 | 5 |
| Na | isopropanol | | |
| 2 mM Arginine | 30% | 7.0 | 4 |
| HCl/NaOH | isopropanol | | |
| 2 mM Arginine | 30% | 4.0 | 4 |
| HCl/NaOH | isopropanol | | |

C. Virus - Tobacco Mosaic Virus
Compound: Tobacco Mosaic Virus
(0.5 mg/ml)

| | | | Microsphere |
|---|---|---|---|
| Counterion | Antisolvent | pH | Quality |
| None | 5% | | 8 |
| | isopropanol | | |
| 2 mM Na-Citrate | 5% | 4.0 | 9 |
| | isopropanol | | |
| 2 mM Na-Citrate | 5% | 5.0 | 4 |
| | isopropanol | | |
| 2 mM Pivalic Acid- | 5% | 5.0 | 6 |
| Na | isopropanol | | |
| 2 mM Na- | 5% | 7.0 | 7 |
| Glutamate | isopropanol | | |
| 2 mM Arginine | 5% | 7.0 | 8 |
| HCl/NaOH | isopropanol | | |
| 2 mM Arginine | 5% | 9.0 | 8 |
| HCl/NaOH | isopropanol | | |
| 2 mM Na- | 5% | 4.0 | 10 |
| sulfate/Na-acetate | isopropanol | | |
| 2 mM Na- | 5% | 6.0 | 7 |
| sulfate/Na-acetate | isopropanol | | |
| 2 mM Na-Citrate | None** | 4.0 | 5 |
| 2 mM Na-Citrate | None | 5.0 | 2 |
| 2 mM Pivalic Acid- | None | 5.0 | 8 |
| Na | | | |
| 2 mM Na- | None | 7.0 | 3 |
| Glutamate | | | |
| 2 mM Arginine | None | 7.0 | 6 |
| HCl/NaOH | | | |
| 2 mM Arginine | None | 9.0 | 2 |
| HCl/NaOH | | | |
| 2 mM Na- | None | 4.0 | 4 |
| sulfate/Na-acetate | | | |
| 2 mM Na- | None | 6.0 | 6 |
| sulfate/Na-acetate | | | |

**As noted above, microspheres of good quality could be formed with tobacco mosaic virus, even in the absence of antisolvent. Some crystallinity was observed, but depending on the choice of counterion (e.g., pivalic acid), uniform microspheres could be obtained without the addition of antisolvent.

D. Peptides—Somatostatin and Leuprolide

| | | | |
|---|---|---|---|
| | | | Microsphere |
| Counterion | Antisolvent | pH | Quality |

Compound: Somatostatin

| | | | |
|---|---|---|---|
| 2 mM Na-citrate | 5% | 4.0 | 5 |
| | isopropanol | | |
| 2 mM Na-citrate | 5% | 5.0 | 5 |
| | isopropanol | | |
| 2 mM Na-citrate | 5% | 7.0 | 5 |
| | isopropanol | | |
| 2 mM Arginine | 5% | 7.0 | 5 |
| HCl/NaOH | isopropanol | | |
| 2 mM Arginine | 5% | 9.0 | 5 |
| HCl/NaOH | isopropanol | | |
| 2 mM Itaconic Acid- | 5% | 4.0 | 5 |
| Na | isopropanol | | |
| 2 mM Itaconic Acid- | 5% | 7.0 | 3 |
| Na | isopropanol | | |
| 2 mM Pivalic Acid- | 5% | 4.0 | 5 |
| Na | isopropanol | | |
| 2 mM Pivalic Acid- | 5% | 7.0 | 6 |
| Na | isopropanol | | |
| 2 mM Na- | 5% | 4.0 | 6 |
| Glutamate | isopropanol | | |
| 2 mM Na- | 5% | 7.0 | 4 |
| Glutamate | isopropanol | | |
| 2 mM PEI 750000 | | | 4 |
| 2 mM PEI 25000 | | | 4 |
| 2 mM PEI 2000 | | | 3 |
| 2 mM Na- | 5% | 4.0 | 8 |
| sulfate/Na-acetate | isopropanol | | |
| 2 mM Na- | 5% | 6.0 | 5 |
| sulfate/Na-acetate | isopropanol | | |
| 2 mM Na-citrate | 15% | 4.0 | 5 |
| | isopropanol | | |
| 2 mM Na-citrate | 15% | 5.0 | 5 |
| | isopropanol | | |
| 2 mM Na-citrate | 15% | 7.0 | 4 |
| | isopropanol | | |
| 2 mM Arginine | 15% | 7.0 | 5 |
| HCl/NaOH | isopropanol | | |
| 2 mM Arginine | 15% | 9.0 | 7 |
| HCl/NaOH | isopropanol | | |
| 2 mM Itaconic Acid- | 15% | 4.0 | 5 |
| Na | isopropanol | | |
| 2 mM Itaconic Acid- | 15% | 7.0 | 6 |
| Na | isopropanol | | |
| 2 mM Pivalic Acid- | 15% | 4.0 | 6 |
| Na | isopropanol | | |
| 2 mM Pivalic Acid- | 15% | 7.0 | 6 |
| Na | isopropanol | | |
| 2 mM Na- | 15% | 4.0 | 7 |
| Glutamate | isopropanol | | |
| 2 mM Na- | 15% | 7.0 | 3 |
| Glutamate | isopropanol | | |
| 2 mM PEI 750000 | | | 3 |
| 2 mM PEI 25000 | | | 4 |
| 2 mM PEI 2000 | | | 4 |
| 2 mM Na- | 15% | 4.0 | 4 |
| sulfate/Na-acetate | isopropanol | | |
| 2 mM Na- | 15% | 6.0 | 6 |
| sulfate/Na-acetate | isopropanol | | |

Compound: Leuprolide

| | | | |
|---|---|---|---|
| 2 mM Na-citrate | 5% | 4.0 | 7 |
| | isopropanol | | |
| 2 mM Na-citrate | 5% | 5.0 | 7 |
| | isopropanol | | |
| 2 mM Na-citrate | 5% | 7.0 | 7 |
| | isopropanol | | |
| 2 mM Arginine | 5% | 7.0 | 5 |
| HCl/NaOH | isopropanol | | |
| 2 mM Arginine | 5% | 9.0 | 6 |
| HCl/NaOH | isopropanol | | |
| 2 mM Itaconic Acid- | 5% | 4.0 | 5 |
| Na | isopropanol | | |
| 2 mM Itaconic Acid- | 5% | 7.0 | 5 |
| Na | isopropanol | | |
| 2 mM Pivalic Acid- | 5% | 4.0 | 7 |
| Na | isopropanol | | |
| 2 mM Pivalic Acid- | 5% | 7.0 | 6 |
| Na | isopropanol | | |
| 2 mM Na- | 5% | 4.0 | 7 |
| Glutamate | isopropanol | | |
| 2 mM Na- | 5% | 7.0 | 4 |
| Glutamate | isopropanol | | |
| 2 mM PEI 750000 | | | 4 |
| 2 mM PEI 25000 | | | 6 |
| 2 mM PEI 2000 | | | 3 |
| 2 mM Na- | 5% | 4.0 | 8 |
| sulfate/Na-acetate | isopropanol | | |
| 2 mM Na- | 5% | 6.0 | 5 |
| sulfate/Na-acetate | isopropanol | | |
| 2 mM Na-citrate | 15% | 4.0 | 5/6 |
| | isopropanol | | |
| 2 mM Na-citrate | 15% | 5.0 | 7 |
| | isopropanol | | |
| 2 mM Na-citrate | 15% | 7.0 | 4 |
| | isopropanol | | |
| 2 mM Arginine | 15% | 7.0 | 4 |
| HCl/NaOH | isopropanol | | |
| 2 mM Arginine | 15% | 9.0 | 5 |
| HCl/NaOH | isopropanol | | |
| 2 mM Itaconic Acid- | 15% | 4.0 | 5 |
| Na | isopropanol | | |
| 2 mM Itaconic Acid- | 15% | 7.0 | 5 |
| Na | isopropanol | | |
| 2 mM Pivalic Acid- | 15% | 4.0 | 7 |
| Na | isopropanol | | |
| 2 mM Pivalic Acid- | 15% | 7.0 | 7 |
| Na | isopropanol | | |
| 2 mM Na- | 15% | 4.0 | 7 |
| Glutamate | isopropanol | | |
| 2 mM Na- | 15% | 7.0 | 8 |
| Glutamate | isopropanol | | |
| 2 mM PEI 750000 | | | 8 |
| 2 mM PEI 25000 | | | 4 |
| 2 mM PEI 2000 | | | 4 |
| 2 mM Na- | 15% | 4.0 | 7 |
| sulfate/Na-acetate | isopropanol | | |
| 2 mM Na- | 15% | 6.0 | 5 |
| sulfate/Na-acetate | isopropanol | | |

E. DAS181 Protein
Compound: DAS181

| | | | Microsphere |
|---|---|---|---|
| Counterion | Antisolvent | pH | Quality |
| 2 mM Na-citrate | 5% | 4.0 | 7 |
| | isopropanol | | |
| 2 mM Na-citrate | 5% | 5.0 | 7 |
| | isopropanol | | |
| 2 mM Na-citrate | 5% | 7.0 | 5 |
| | isopropanol | | |
| 2 mM Arginine | 5% | 7.0 | 4 |
| HCl/NaOH | isopropanol | | |
| 2 mM Arginine | 5% | 9.0 | 5 |
| HCl/NaOH | isopropanol | | |
| 2 mM Itaconic Acid- | 5% | 4.0 | 5 |
| Na | isopropanol | | |
| 2 mM Itaconic Acid- | 5% | 7.0 | 5 |
| Na | isopropanol | | |
| 2 mM Pivalic Acid- | 5% | 4.0 | 5 |
| Na | isopropanol | | |
| 2 mM Pivalic Acid- | 5% | 7.0 | 7 |
| Na | isopropanol | | |
| 2 mM Na- | 5% | 4.0 | 6 |
| Glutamate | isopropanol | | |
| 2 mM Na- | 5% | 7.0 | 6 |
| Glutamate | isopropanol | | |
| 2 mM PEI 750000 | | | 8 |
| 2 mM PEI 25000 | | | 10 |
| 2 mM PEI 2000 | | | 7 |
| 2 mM Na- | 5% | 4.0 | 8 |
| sulfate/Na-acetate | isopropanol | | |
| 2 mM Na- | 5% | 6.0 | 7 |
| sulfate/Na-acetate | isopropanol | | |
| 2 mM Na-citrate | 15% | 4.0 | 5/6 |
| | isopropanol | | |
| 2 mM Na-citrate | 15% | 5.0 | 7 |
| | isopropanol | | |
| 2 mM Na-citrate | 15% | 7.0 | 6 |
| | isopropanol | | |
| 2 mM Arginine | 15% | 7.0 | 4 |
| HCl/NaOH | isopropanol | | |
| 2 mM Arginine | 15% | 9.0 | 7 |
| HCl/NaOH | isopropanol | | |
| 2 mM Itaconic Acid- | 15% | 4.0 | 7 |
| Na | isopropanol | | |
| 2 mM Itaconic Acid- | 15% | 7.0 | 7 |
| Na | isopropanol | | |
| 2 mM Pivalic Acid- | 15% | 4.0 | 6 |
| Na | isopropanol | | |
| 2 mM Pivalic Acid- | 15% | 7.0 | 7 |
| Na | isopropanol | | |
| 2 mM Na- | 15% | 4.0 | 6 |
| Glutamate | isopropanol | | |
| 2 mM Na- | 15% | 7.0 | 5 |
| Glutamate | isopropanol | | |
| 2 mM PEI 750000 | | | 8 |
| 2 mM PEI 25000 | | | 9 |
| 2 mM PEI 2000 | | | 6 |
| 2 mM Na- | 15% | 4.0 | 7 |
| sulfate/Na-acetate | isopropanol | | |
| 2 mM Na- | 15% | 6.0 | 8 |
| sulfate/Na-acetate | isopropanol | | |
| 2 mM Na-citrate | 5% n- | 5.0 | 10 |
| | propanol | | |
| 2 mM Arginine | 5% n- | 7.0 | 3 |
| HCl/NaOH | propanol | | |
| 2 mM Arginine | 5% n- | 9.0 | 7 |
| HCl/NaOH | propanol | | |
| 2 mM Itaconic Acid- | 5% n- | 4.0 | 5 |
| Na | propanol | | |
| 2 mM Itaconic Acid- | 5% n- | 7.0 | 2 |
| Na | propanol | | |
| 2 mM Na- | 5% n- | 4.0 | 6 |
| Glutamate | propanol | | |
| 2 mM Na- | 5% n- | 7.0 | 4 |
| Glutamate | propanol | | |
| 2 mM Na-citrate | 15% n- | 5.0 | 8/9 |
| | propanol | | |
| 2 mM Arginine | 15% n- | 9.0 | 6 |
| HCl/NaOH | propanol | | |
| 2 mM Itaconic Acid- | 15% n- | 4.0 | 4 |
| Na | propanol | | |
| 2 mM Itaconic Acid- | 15% n- | 7.0 | 2 |
| Na | propanol | | |
| 2 mM Na- | 15% n- | 4.0 | 6/7 |
| Glutamate | propanol | | |
| 2 mM Na- | 15% n- | 7.0 | 4 |
| Glutamate | propanol | | |

Results: These experiments demonstrate that by selecting the appropriate combination of: (a) type and (b) concentration of compound, counterion and antisolvent, microspheres of good quality (at least 6, as high as 10) can be obtained using a wide variety of macromolecules and small molecules. Depending on the particular combination of compound, counterion and antisolvent, the quality of the microspheres often was as good or better than the quality of microspheres obtained using the sialidase fusion protein, DAS181, under comparable conditions.

In control cocktail reactions containing no compounds, it was noted that some counterions, such as polyethyleneimine (PEI) and Na-acetate/sulfate, could form microspheres under certain conditions. Without being bound by any theory, depending on the compound of interest, such counterions potentially could act as “primers” or “carriers” that help to nucleate and/or facilitate the formation of higher quality microspheres, relative to those obtained with “non-nucleating” counterions. For example, formation of a compound (see, for example, in Table 14 above, microspheres formed from siRNA and DAS181, using PEI as counterion).

The results further demonstrate that under certain conditions, microspheres can be formed in the absence of counterion and/or antisolvent. For example, in the case of siRNA, very high quality (scale of 10) microspheres were obtained when no counterion was added. Similarly, tobacco mosaic virus formed microspheres in the absence of counterion and, in some instances, in the absence of antisolvent.

### Example 14

### Size and Quality of Microspheres as a Function of Concentration of the Cocktail Components (Compound, Counterion, Antisolvent)

This Example demonstrates that the size and quality of the microspheres of small molecule compounds, like those of macromolecules (see Examples 2-4), can readily be optimized by varying parameters, such as the concentrations of compound, counterion, and/or antisolvent, in a variety of permutations in high-throughput format. By conducting these reactions in high-throughput format, conditions that are optimal for microsphere formation of any compound can rapidly be identified.

96-well plates containing cocktail solutions of Tetracycline, Kanamycin or Ampicillin under various concentration conditions were set up as described in Example 1. Arginine was used as counterion and isopropanol was used as antisolvent. Concentrations of each of the cocktail components—the compound, the counterion and the antisolvent—were varied as shown below in Table 15, and the effect on microsphere quality assessed.

TABLE 15

| | Arginine | | |
|---|---|---|---|
| | Concentration | Isopropanol | Microsphere |
| | (mg/ml) | (%) | Quality |
| Tetracycline | | | |
| Concentration | | | |
| (mg/ml) | | | |
| 25 | 60 | 0 | 1 |
| 25 | 60 | 10 | 3 |
| 25 | 60 | 20 | 4 |
| 25 | 60 | 30 | 7 |
| 25 | 60 | 40 | 5 |
| 25 | 60 | 50 | 6 |
| 20 | 48 | 60 | 6 |
| 15 | 36 | 70 | 1 |
| 25 | 60 | 50 | 5 |
| 25 | 50 | 50 | 9 |
| 25 | 40 | 50 | 9 |
| 25 | 30 | 50 | 9 |
| 25 | 20 | 50 | 7 |
| 25 | 10 | 50 | 7 |
| 25 | 5 | 50 | 7 |
| 25 | 0 | 50 | 9 |
| 0 | 15 | 25 | 7 |
| 5 | 15 | 25 | 9 |
| 10 | 15 | 25 | 8/9 |
| 15 | 15 | 25 | 8/9 |
| 20 | 15 | 25 | 7 |
| 25 | 15 | 25 | 8 |
| 30 | 15 | 25 | 9 |
| 31.25 | 15 | 25 | 7 |
| Kanamycin | | | |
| Concentration | | | |
| (mg/ml) | | | |
| 25 | 60 | 0 | 0 |
| 25 | 60 | 10 | 0 |
| 25 | 60 | 20 | 0 |
| 25 | 60 | 30 | 3 |
| 25 | 60 | 40 | 0 |
| 25 | 60 | 50 | 0 |
| 20 | 48 | 60 | 6 |
| 25 | 60 | 50 | 0 |
| 25 | 50 | 50 | 0 |
| 25 | 40 | 50 | 2 |
| 25 | 30 | 50 | 4 |
| 25 | 20 | 50 | 7 |
| 25 | 10 | 50 | 10 |
| 25 | 5 | 50 | 7 |
| 25 | 0 | 50 | 8 |
| 0 | 15 | 25 | 0 |
| 5 | 15 | 25 | 4 |
| 10 | 15 | 25 | 8 |
| 15 | 15 | 25 | 7 |
| 20 | 15 | 25 | 7 |
| 25 | 15 | 25 | 8 |
| 30 | 15 | 25 | 7 |
| 31.25 | 15 | 25 | 9 |
| Ampicillin | | | |
| Concentration | | | |
| (mg/ml) | | | |
| 25 | 60 | 0 | 0 |
| 25 | 60 | 10 | 3 |
| 25 | 60 | 20 | 0 |
| 25 | 60 | 30 | 0 |
| 25 | 60 | 40 | 0 |
| 25 | 60 | 50 | 6 |
| 15 | 36 | 70 | 0 |
| 25 | 60 | 50 | 2 |
| 25 | 20 | 50 | 0 |
| 25 | 10 | 50 | 1 |
| 25 | 5 | 50 | 1 |
| 25 | 0 | 50 | 2 |

### Results:

Tetracycline: With tetracycline, the absence of antisolvent resulted in few, if any, microspheres being formed. As the concentration of antisolvent was increased, the quality of the microspheres increased, reaching a maximum at about 30% isopropanol. Increasing the isopropanol concentration beyond 30% resulted in a decrease in overall microsphere quality and the formation of larger, chunky agglomerations of microspheres and crystalline solids. At isopropanol concentrations of 60% and 70%, the cocktail mixture was found to precipitate before freezing, resulting in large amounts of aggregates and crystals. A few microspheres were formed at the highest isopropanol concentrations, but they were not of uniform size.

Microsphere quality was then assessed as a function of counterion (arginine) concentration. It was found that at constant antisolvent and tertracycline concentration, the microsphere size distribution decreases and its overall quality increases as the concentration of arginine is decreased, with 30 mg/ml arginine giving the smallest size distribution as visualized by light microscopy. It was interesting to note that in the absence of counterion, small microspheres with very little size variation were observed. Their overall quality was high, although some amount of aggregation was present.

Microsphere quality was also assessed by increasing tetracycline concentration at constant antisolvent and arginine concentrations. In the absence of tetracycline, hygroscopic arginine microspheres were formed. As the tetracycline concentration was increased, the microsphere size was found to increase to a maximum at a concentration of 25-30 mg/ml tetracycline, then decreased again as the concentration of tetracycline was further increased. At 25-30 mg/ml tetracycline, aggregation also was minimal; the aggregation increased as the tetracycline concentration was further increased.

Kanamycin: The formation of microspheres from kanamycin required at least 25-30% antisolvent (isopropanol); below this concentration, hygroscopic crystals were formed. At 60% isopropanol, better results were obtained, although there was precipitation prior to freezing, which compromised microsphere quality.

When the kanamycin microspheres were studied as a function of varying arginine concentration, it was found that decreasing the arginine concentration resulted in higher quality, smaller microspheres. At less than 10 mg/ml arginine, however, the microspheres became larger and tended to agglomerate more. In the absence of arginine, high quality microspheres were again obtained, although some aggregation was present.

When varying the concentration of kanamycin, it was found that as the concentration of kanamycin was increased, the quality of the microspheres increased and the size decreased.

Ampicillin: Although microspheres were obtained using ampicillin, their hygroscopic nature made it difficult to unambiguously assign their quality. In general, several conditions produced distinct microspheres, with the best quality microspheres being observed at high (50%) antisolvent concentration.

This example demonstrates that a variety of small molecule antibiotics can produce microspheres by the methods provided herein. The example also demonstrates that under certain conditions, the addition of a counterion may not be necessary for microparticle formation. This was observed for all three antibiotics tested. Without being bound by theory, it is possible that the compounds themselves function as counterions, or that the preparations used in the experiments contained excipients/impurities/bulking agents that served as counterions.

### Example 15

### Preparation of Microspheres from Water-Insoluble Molecules: Paclitaxel

The chemotherapeutic agent, Paclitaxel, has a log P value that is higher than 3 (log of the octanol/water partition coefficient) (Bombuwala et. al., Beilstein J. Org. Chem. 2006, 2:13), which is a value that is representative of a large portion of the small molecule drugs that are currently on the market. Hence, the identification of conditions for Paclitaxel microsphere formation should be applicable to a large number of therapeutically relevant compounds.

Paclitaxel, being water-insoluble, was dissolved in one of the following organic solvents: Isopropanol, t-Butyl Alcohol or DMSO. A 20 mg/ml stock solution of paclitaxel in each of the organic solvents was used to generate cocktail solutions in 96-well plates, where the net concentration of paclitaxel in each well (Le, reaction) was 2 mg/ml paclitaxel. With ispropanol, a 20 mg/ml slurry was obtained and used as the stock solution, because the solubility of paclitaxel in isopropanol is lower than in t-Butyl Alcohol and DMSO. 2 mM Na-citrate buffer, pH 5.0, was used as the antisolvent and counterion. The various experimental conditions are listed below in Table 16. The plates containing the cocktail solutions with varying concentrations of antisolvent/buffer relative to the concentration of organic solvent, were then placed in a −80° C. freezer for lyophilization.

TABLE 16
Compound: Paclitaxel (2 mg/ml)

| Citrate Buffer | |
|---|---|
| (mM) | Solvent (%) |
| 2 | 90% isopropanol |
| 2 | 75% isopropanol |
| 2 | 50% isopropanol |
| 2 | 25% isopropanol |
| 2 | 10% isopropanol |
| 0 | 100% isopropanol |
| 0 | 50% isopropanol |
| 2 | 90% t-butanol |
| 2 | 75% t-butanol |
| 2 | 50% t-butanol |
| 2 | 25% t-butanol |
| 2 | 10% t-butanol |
| 0 | 100% t-butanol |
| 0 | 50% t-butanol |
| 2 | 90% DMSO |
| 2 | 75% DMSO |
| 2 | 50% DMSO |
| 2 | 25% DMSO |
| 2 | 10% DMSO |
| 0 | 100% DMSO |
| 0 | 50% DMSO |

### Results:

With all three organic solvents, it was found that paclitaxel precipitated if the solvent concentration was 25% or less. Optical microscopy of the lyophilized samples showed that with the organic solvent isopropanol, the microparticles increased in quality as the concentration of isopropanol was lowered from 90%, with the best microspheres being formed in 50% isopropanol. When the concentration of isopropanol was lowered below 50%, crystals were observed, potentially due to precipitation of paclitaxel prior to freezing. When 50% isopropanol was used with no citrate counterion, the microspheres showed a higher tendency to aggregate than in the presence of 2 mM citrate. With 100% isopropanol and no citrate counterion, the samples appeared to have high crystallinity and were aggregated, although many small microparticles were also observed.

With t-butanol, the optimum solvent concentration was higher than isopropanol, with best results being observed at 90% t-butanol and aggregation and rod-like formations increasing as the concentration of solvent was decreased. At 25% and 10% t-butanol, significant crystallinity was observed, likely due to precipitation of paclitaxel before freezing. In 100% t-butanol with no citrate counterion, the microspheres were almost as high in quality as the best quality microspheres observed at 90% t-butanol and 2 mM citrate, as discussed above. When the concentration of t-butanol was lowered to 50% in the absence of citrate counterion, high quality microspheres were still present, although the aggregation increased.

The results obtained with the organic solvent DMSO showed higher amounts of crystallinity in general, along with aggregated microspheres of lower quality, relative to isopropanol and t-butanol. This could be due to the high boiling point of DMSO, as the water in the solutions likely evaporated/sublimed first upon lyophilization, leaving nearly pure DMSO solution from which the paclitaxel crystallized. Optical microscopy data did however reveal the presence of microspheres, with the best microspheres being observed when 50% DMSO was used.

### Example 16

### Effect of Drug, Antisolvent and Counterion Ratios on the Quality of Microspheres

Experiments were performed to evaluate the effect of antisolvent and counterion concentration variation on the formation of microspheres. The peptides leuprolide and somatostatin, and the antibiotics vancomycin and tobramycin, were tested under a variety of conditions for forming microspheres. Table 17 describes the conditions under which the reactions were performed. Samples were analyzed in 96-well plates as described in the previous Examples.

TABLE 17
Compound: Leuprolide (2 mg/ml)

| Na-glutamate, pH | Isopropanol | Microsphere |
|---|---|---|
| 7.0 (mM) | (%) | Quality |
| 2 | 50 | 9 |
| 2 | 40 | 7 |
| 2 | 30 | 8 |
| 2 | 20 | 9 |
| 2 | 10 | 5 |
| 2 | 5 | 7 |
| 2 | 2.5 | 8 |
| 2 | 0 | 3 |
| 17 | 5 | 5 |
| 15 | 5 | 5 |
| 12.5 | 5 | 7 |
| 10 | 5 | 7 |
| 7.5 | 5 | 7 |
| 5 | 5 | 6 |
| 2.5 | 5 | 7 |
| 0 | 5 | 6 |

Compound: Somatostatin (2 mg/ml)

| Na-Sulfate/Na- | | |
|---|---|---|
| Acetate, pH 4.0 | Isopropanol | Microsphere |
| (mM) | (%) | Quality |
| 2 | 50 | 9 |
| 2 | 40 | 8 |
| 2 | 30 | 9 |
| 2 | 20 | 7 |
| 2 | 10 | 6/7 |
| 2 | 5 | 6 |
| 2 | 2.5 | 3/4 |
| 2 | 0 | 3 |
| 17 | 5 | 6 |
| 15 | 5 | 8 |
| 12.5 | 5 | 9 |
| 10 | 5 | 7 |
| 7.5 | 5 | 7 |
| 5 | 5 | 6/7 |
| 2.5 | 5 | 6 |
| 0 | 5 | 7 |

Compound: Vancomycin (2 mg/ml)

| Na-citrate, pH 5.0 | Isopropanol | Microsphere |
|---|---|---|
| (mM) | (%) | Quality |
| 2 | 50 | 7 |
| 2 | 40 | 9 |
| 2 | 30 | 8 |
| 2 | 20 | 10 |
| 2 | 10 | 7 |
| 2 | 5 | 9 |
| 2 | 2.5 | 7 |
| 2 | 0 | 7 |
| 17 | 5 | 6 |
| 15 | 5 | 7 |
| 12.5 | 5 | 7 |
| 10 | 5 | 8 |
| 7.5 | 5 | 9/8 |
| 5 | 5 | 9 |
| 2.5 | 5 | 9/8 |
| 0 | 5 | 7 |

Compound: Tobramycin (2 mg/ml)

| Itaconic acid-Na, | Isopropanol | Microsphere |
|---|---|---|
| pH 4.0 (mM) | (%) | Quality |
| 2 | 50 | 7 |
| 2 | 40 | 6/7 |
| 2 | 30 | 8/9 |
| 2 | 20 | 8 |
| 2 | 10 | 9 |
| 2 | 5 | 7/8 |
| 2 | 2.5 | 7/8 |
| 2 | 0 | 4/5 |
| 17 | 5 | Crystals |
| 15 | 5 | Crystals |
| 12.5 | 5 | Crystals |
| 10 | 5 | Crystals |
| 7.5 | 5 | Crystals |
| 5 | 5 | Crystals |
| 2.5 | 5 | 7 |
| 0 | 5 | 8 |

Results:

In the leuprolide group, decreasing antisolvent concentration reduced the aggregation of microspheres with an optimum at 10% isopropanol, and the aggregation increased again as the isopropanol concentration was further reduced down to 0%. When the counterion concentration was varied at constant antisolvent concentration (5%), 17 mM counterion showed a high degree of crystal formation. The crystal formation decreased as the counterion (buffer) concentration was decreased, until at 10 mM, the microspheres are evenly sized and well separated. As the buffer concentration was further decreased beyond 10 mmM, the aggregation began to increase again, with a moderate degree of crystallinity being observed at 0 mM glutamate.

In the case of somatostatin, even-sized well-separated microspheres were observed at 50% isopropanol. The level of aggregation increased as the concentration of antisolvent was decreased, to an optimum of 10% isopropanol. Below 10% isopropanol, crystals began to appear and continued to increase as the antisolvent concentration was decreased to 0% isopropanol, where a majority of the sample was crystalline with only a few aggregated microspheres. When the counterion concentration was varied at constant antisolvent concentration (5%), at 17 mM Sulfate/Acetate and 5% isopropanol, microspheres were present, but a high degree of crystallinity also was observed. As the concentration of counterion was decreased, the amount of crystals present decreased until well separated microspheres were detected at 12.5 mM counterion concentration. As the sulfate/acetate concentration was further decreased, aggregation increased again and microsphere size decreased. Somatostatin was also found to form microspheres in the absence of counterion, but they were aggregated and of varying (not uniform) size.

In the case of vancomycin, with changing antisolvent concentration, small but well defined microspheres were produced from 50% down to 2.5%. When the solvent concentration was further dropped down to 0% isopropanol, a high degree of crystallinity was present, with a few aggregated microspheres. When counterion concentration was varied, the microspheres were found to be highly aggregated at 17 mM citrate, and the amount of aggregation decreased as the counterion concentration was decreased. The best microspheres formed below 7.5 mM citrate, but as the counterion concentration was further dropped down to zero, the amount of aggregation again increased.

In the case of tobramycin, as the antisolvent concentration was varied, with 50% isopropanol there was a significant amount of crystallinity and aggregation, although microspheres were also detected. As the antisolvent concentration was reduced from 40% to 10%, the microspheres formed were found to be well-separated and of high quality. When the antisolvent concentration was further decreased from 5% to 0%, the amount of aggregation again increased and at 0% there was a high degree of crystallinity along with significant numbers of aggregated microspheres.

### Example 17

### Aerodynamic Particle Size Distribution of Vancomycin Microspheres for Inhalation

As described herein, the methods provided herein can be used to produce microspheres in any desired size range, including a range of about 0.5 micron to about 6-8 microns for delivery via inhalation.

A. Preparation of Microspheres Vancomycin was dissolved in aqueous buffer at a final concentration of 10 mg/ml. The cocktail contained 5 mM sodium citrate pH 5.0 as counterion and 15% v/v n-propanol as anti-solvent. A 2 ml aliquot of cocktail was frozen in a 10-ml lyophilization vial placed in a −80° C. freezer for 1 hour. The frozen vial was transferred onto a −45° C. lyophilizer shelf and freeze dried for 36 hours.

B. Aerodynamic Particle Size Distribution of Microspheres

The microspheres prepared as described in Example 5 were tested by Cascade Impaction using a New Generation Impactor. The deposition of pharmaceuticals in the respiratory tract can be predicted by the aerodynamic behavior of particles (microspheres) on the stages/collection plates of the cascade impactor.

The microspheres (10 mg) were loaded into HPMC (hydroxypropyl methylcellulose) capsule. The capsule was placed into a CycloHaler (PharmaChemie) dry powder inhaler and subjected to cascade impaction. The collection plates of the impactor representing various areas/stages of deposition post-inhalation (trachea, primary and secondary bronchi, terminal bronchi, alveoli, etc.) were coated with silicon spray to prevent bouncing of the microspheres. The microspheres from the stages and collection plates were recovered into a phosphate buffered saline containing 0.1% Tween, and the amount of deposited vancomycin recovered from each stage and collection plate was quantified by measuring absorbance at 280 nm.

Results: The geometric size of microspheres was assessed by light microscopy and found to be in the range of 1.0-3.0 microns. As shown in Table 18 below, the aerodynamic particle size was consistent with the observed geometric size. The results demonstrate that methods provided herein can produce microspheres for delivery into deep lungs, and that the microspheres produced by methods provided herein have good disagglomeration and flowability properties (provide a higher delivered dose).

TABLE 18
Results of Cascade Impaction Analyses of Vancomycin Microspheres

| Component of | Corresponding | Expected Deposition | Percent |
|---|---|---|---|
| the Cascade | Size Cut-Off | in Respiratory | Deposition of |
| Impactor | (microns) | Airways | Vancomycin |
| Capsule + | NA | NA | 37.57 |
| device | | | |
| Throat | >10 | oral cavity | 10.48 |
| 1 | >8.06 | Oral cavity/pharynx | 3.33 |
| 2 | 4.46-8.06 | pharynx | 7.71 |
| 3 | 2.82-4.46 | trachea/bronchi | 15.47 |
| 4 | 1.66-2.82 | secondary bronchi | 16.69 |
| 5 | 0.94-1.66 | terminal bronchi/ | 6.38 |
| | | alveoli | |
| 6 | 0.55-0.94 | alveoli | 1.59 |
| 7 | 0.34-0.55 | alveoli | 0.51 |
| 8 | <0.34 | alveoli | 0.27 |

### Example 18

### Preparation of Microspheres Using Prostaglandin

Prostaglandins are a group of hormone-like compounds that are implicated in numerous physiological processes and, therefore, have clinical applications. One of the prostaglandins, the prostacyclin PGI₂, is a drug that is currently marketed for pulmonary hypertension. The API half life of this drug at physiological pH is on the order of minutes, requiring the drug to be administered through continuous infusion in order to have an appreciable effect. Therefore, it is desirable to create a PGI₂ formulation that is inhalable and works directly at the target site of action in the lungs, avoiding the pharmacokinetic effects associated with clearance rates and stability in the bloodstream. This example demonstrates that the methods provided herein can be used to prepare high quality, inhalable microspheres of prostaglandins.

The experiments were performed using PGI₂ and an analog of PGI₂, Ciprostene, at a concentration of 2 mg/ml. Cocktail solutions were mixed at room temperature, then cooled by placing in a freezer. The chilled plates were transferred onto pre-chilled (−45° C.) shelves of a Millrock Lab Series Lyophilizer, and the vacuum was applied. The frozen cocktail solutions were allowed to lyophilize for 16 hours.

Because the resulting prostaglandin microspheres are hygroscopic, upon microsphere initiation, the humidity was maintained at low levels during the experiments, using a nitrogen gas tank attached to the backfill system on the lyophilizer. Each of the reaction tubes was flushed with N₂. In addition, the backfill valve on the lyophilizer was left open to continually flush a low humidity atmosphere over the samples. The microscope used to visualize the resulting microspheres was contained within a plastic bag that was continually purged with dry N₂. Sample tubes were placed under the bag to equilibrate for approximately 30 seconds, before opening and spreading onto the glass slide.

Prostaglandin is unstable at pH values lower than 8; therefore, the following basic buffers were used in this experiment: Polyethyleneimine (PEI), Triethylamine (TEA) and Arginine. Ciprostene is not highly soluble in aqueous solutions, therefore n-propanol was added to the buffer, in amounts that rendered the compound soluble. The solvent/antisolvent system for the prostaglandins was water/n-propanol/t-butanol (water/aqueous buffer being more of the “antisolvent” component for Ciprostene, which has poor solubility in water, and n-propanol/Tert-Butyl alcohol (t-butanol, tBA) being more of the “antisolvent” component for PGI2, which has higher solubility in water). The results are summarized in Table 19 below:

TABLE 19

| | | | | |
|---|---|---|---|---|
| Buffer/ | | | | Microsphere |
| Counterion | pH | (% n-propanol) | (% t-butanol) | Quality |

Compound: Prostaglandin I₂

| | | | | |
|---|---|---|---|---|
| 2 mM Arginine | 9 | 20 | 0 | 7 |
| 2 mM Arginine | 9 | 30 | 0 | 5 |
| 2 mM Arginine | 9 | 20 | 5 | 7/8 |
| 2 mM Arginine | 9 | 20 | 30 | 5 |
| 2 mM Arginine | 9 | 20 | 55 | 5/6 |
| 2 mM Arginine | 9 | 20 | 70 | 4 |
| 2 mM TEA | 11 | 20 | 0 | 0 |
| 2 mM TEA | 11 | 30 | 0 | 2/3 |
| 2 mM TEA | 11 | 20 | 5 | 7/8 |
| 2 mM TEA | 11 | 20 | 30 | 8 |
| 2 mM TEA | 11 | 20 | 55 | 7 |
| 2 mM TEA | 11 | 20 | 70 | 8 |
| 2 mM PEI | 10.75 | 20 | 0 | 7 |
| 2 mM PEI | 10.75 | 30 | 0 | 8 |
| 2 mM PEI | 10.75 | 20 | 5 | 7 |
| 2 mM PEI | 10.75 | 20 | 30 | 5 |
| 2 mM PEI | 10.75 | 20 | 55 | 7 |
| 2 mM PEI | 10.75 | 20 | 70 | 6 |

Compound: Ciprostene

| | | | | |
|---|---|---|---|---|
| 2 mM Arginine | 9 | 20 | 0 | 7/8 |
| 2 mM Arginine | 9 | 30 | 0 | 6 |
| 2 mM Arginine | 9 | 20 | 5 | 8 |
| 2 mM Arginine | 9 | 20 | 30 | 6/7 |
| 2 mM Arginine | 9 | 20 | 55 | 8 |
| 2 mM Arginine | 9 | 20 | 70 | 7 |
| 2 mM TEA | 11 | 20 | 0 | 4/5 |
| 2 mM TEA | 11 | 30 | 0 | 3/4 |
| 2 mM TEA | 11 | 20 | 5 | 8/7 |
| 2 mM TEA | 11 | 20 | 30 | 7 |
| 2 mM TEA | 11 | 20 | 55 | 6/7 |
| 2 mM TEA | 11 | 20 | 70 | 5/6 |
| 2 mM PEI | 10.75 | 20 | 0 | 6 |
| 2 mM PEI | 10.75 | 30 | 0 | 6/7 |
| 2 mM PEI | 10.75 | 20 | 5 | 9 |
| 2 mM PEI | 10.75 | 20 | 30 | 8 |
| 2 mM PEI | 10.75 | 20 | 55 | 7 |
| 2 mM PEI | 10.75 | 20 | 70 | 6 |

### Results:

With PGI₂, several conditions were identified for good quality microsphere formation, with several ratings above 6 and a maximum rating of 8. When the buffer/counterion was Arginine, lower concentrations of n-propanol and t-butanol favored better microsphere formation alcohol concentrations, crystal formation was observed. When the buffer/counterion was TEA, on the other hand, higher concentrations of t-butanol favored higher quality microsphere formation. When the buffer/counterion was PEI, the best quality microspheres were obtained at a higher concentration of n-propanol (30%), and in the absence of t-butanol.

Ciprostene is a more stable analog of PGI₂, and it also appeared to be less hygroscopic. With Arginine buffer, no particular concentration-dependent trend was observed, but several solvent conditions produced high quality microspheres with ratings of 8 (see, e.g 20% n-Propanol/5% t-butanol and 20% n-Propanol/55% t-butanol). With TEA, the quality of microspheres obtained with n-propanol in the absence of t-butanol was low. The quality of microspheres increased as t-butanol was added to the cocktail solution, with a maximum at about 5% t-butanol. As the concentration of t-butanol was increased even further, increasing amounts of aggregation was observed. PEI proved to be the best counterion for ciprostene, with a maximum microsphere quality rating of 9 at 20% n-propanoV5cY0 t-butanol. As the concentration of t-butanol was further increased, increasing amounts of aggregation were observed.

The results demonstrate that high quality microspheres of prostaglandin can be formed under a variety of conditions, which should facilitate a stable formulation for pulmonary delivery.

### Example 19

### Effect of Cooling Rate on the Quality of Microspheres

This example demonstrates that a controlled cooling rate, during which the cocktail solutions from which the microspheres are produced are maintained at specific temperatures for defined periods of time, as opposed to flash-freezing, produces higher quality microspheres with desired characteristics. Flash freeze experiments were conducted with five different cocktails that previously produced excellent microspheres under standard freezing conditions performed according to the methods provided herein. The compound/counterion/antisolvent conditions were as follows:

1) Paclitaxel/citrate pH 5.0/90% t-butanol (see Example 15)
2) DAS181/citrate pH 5/5% n-propanol (see Example 13)
3) Tobacco Mosaic Virus/Na sulfate-Na acetate pH 4/5% isopropanol (see Example 13)
4) Vancomycin/citrate pH 5/5% n-propanol (see Example 13)
5) Tetracycline/Arginine/25-30% isopropanol (see Example 14)

Experiments were performed with 200 μl of each of the above cocktail solutions in a 2 ml lyophilization bottle (first flash freeze condition), and 25 μl of each of the above cocktail solutions in a PCR tube (second flash freeze condition). The samples in the lyophilization bottles took approximately 15 seconds to freeze. The samples in the PCR tubes took approximately 3 seconds or less to freeze.

### Results:

Microscopic analysis of the samples showed that in most cases, the freezing rate has a significant effect on the formation of microspheres. The Paclitaxel samples were mostly crystalline in both cases after the flash freeze, although there was evidence that microspheres were beginning to form. The DAS181 cocktail showed high quality microspheres, with a rating of 9, when flash frozen in the lyophilization bottle. The quality of the DAS181 microspheres, however, was reduced to a rating of 5 in the faster-freezing PCR tube experiment; a significant amount of rod-like crystals were observed, although there were some microparticles present. With Tobacco Mosaic Virus, high quality microspheres, with a rating of 9, were formed in both flash freeze cases. It therefore appears that the formation of Tobacco Mosaic Virus microspheres, under the conditions tested, was not highly affected by the rate of freezing.

With Vancomycin, on the other hand, the quality of the microspheres decreased as the freezing rate was increased. While the Vancomycin cocktail produced a microsphere rating of 9/10 under normal freezing conditions, as described in Example 13, the 200 μl flash freeze sample provided lower quality microspheres with a rating of 7 and observed aggregation. The PCR tube flash freeze produced ever lower quality microspheres, with a rating of 5, higher amounts of aggregation and a significant amount of rod-like crystals. Thus, in the case of Vancomycin, faster freeze rates resulted in lower quality microspheres. Similarly, with Tetracycline, while microsphere ratings of 8/9 were obtained under normal freezing conditions (see Example 14), both flash freeze conditions produced lower quality microspheres of rating 5/6, with significant aggregation.

The results demonstrate that the freezing rate can have an impact on the quality of microspheres generated according to the methods provided herein. The impact, however, is dependent on the compound forming the microspheres. As shown in this example, for some compounds, such as Paclitaxel, DAS181, Vancomycin and Tetracycline, if the freezing rate is too rapid, the microspheres can get trapped in crystalline phases or aggregate before having the opportunity to grow to a reasonable size.

### Example 20

### Efficiency of Nucleic Acid Incorporation into Microspheres

To assess the process yield for nucleic acid incorporation into microspheres, the following experiment was conducted. One mg of yeast tRNA (Sigma, Type X-SA) in 0.5 ml volume (2 mg/ml final concentration in the cocktail) was combined with isopropanol (IPA; 40% final concentration) and sodium citrate (100 mM final concentration) at pH 8.0. Formation of microparticles from the resulting cocktail was induced by placing the cocktail on ice. The microspheres were fixed by the addition of 10 ml (20 volumes) of IPA, and pelleted by centrifugation at 5000 rpm for 3 min. The pellet was dried in a vacuum. Microscopic analysis confirmed the formation of high quality microspheres, 1-2 micron in size, and the absence of aggregated material or crystals.

The amount of tRNA recovered in the pellet and the supernatant was quantitated by UV absorption at 260 nm. It was found that 78% of the tRNA was packaged into the microparticles and 22% tRNA remained in the supernatant. This result demonstrated that tRNA, and likely other nucleic acids such as DNA and siRNA, can be efficiently condensed and packaged into a microsphere formulation.

### Example 21

### siRNA that is Incorporated into Microspheres Retains its Activity

Experiments were performed to assess if the method of producing microspheres as provided herein inhibits the activity of the molecules incorporated in the microspheres.

Preparation of siRNA Microspheres

The exemplary molecule used in this experiment is double stranded GAPDH siRNA (sense sequence 5′-UGGUUUACAUGUUCCAAUAUU-3′(SEQ ID. NO: 27); antisense sequence 5′-UAUUGGAACAUGUAAACCAUU-3′ (SEQ ID NO: 28); with two “UU” overhangs at each 3′-end). Microspheres containing GAPDH siRNA in various cocktail formulations, as described below, were produced:

1: 2 mM Arginine, pH 7.0, 15% IPA, 2 mg/ml siRNA

2: 2 mM PEI (25,000 mol wt, branched, Sigma), pH 10, 15% IPA, 2 mg/ml siRNA

3: 2 mM ltaconic Acid, pH 8.0, 15% IPA, 2 mg/ml siRNA

4: 10 mM (Glutamic acid, Lysine, Alanine, 3:2:5 molar ratio), 5% IPA, 1 mg/ml,

5: 10 mM (Lysine, Citric acid, 1:4 molar ratio), 15% IPA, 1 mg/ml siRNA,

6: 10 mM (Lysine, Citric acid, 1:1 molar ratio), 15% IPA, 1 mg/ml siRNA

7: 10 mM Alanine, 15% IPA, 1 mg/ml siRNA

Control formulations contained all cocktail ingredients with the exception of siRNA. A lyophilized siRNA control contained no excipients and 15% IPA.

The resulting cocktails were chilled to form microspheres and frozen in a single step by placing the vial onto the shelf of a −80° C. freezer. Lyophilization was performed overnight at shelf temperature of +10° C. and a vacuum of 150 mTorr.

Activity of siRNA in Microsphere Formulations

The siRNA microspheres isolated from the lyophilization were then reconstituted and transfected to Hep-2 cells. As a positive control, the same amount of GAPDH siRNA in the original buffer was lyophilized, reconstituted, and transfected, without formation of microspheres. At 48 hr post transfection, the level of GAPDH in the Hep-2 cells was measured using a fluorescent enzymatic assay. The results (Table 20) demonstrated that siRNAs processed into microspheres had gene-silencing activity that was equivalent to or, in some instances, even greater than that of the corresponding positive control (i.e., 100% or more gene-silencing activity). Microscopic analyses confirmed the formation of hiah Quality microspheres.

TABLE 20
siRNA Gene Silencing Activity when used alone or when incorporated
into microspheres.

| Sample | | |
|---|---|---|
| No. | Formulation Cocktail | % Activity |
| | Lyophilized siRNA positive control (no | 100 ± 20 |
| | microsphere) | |
| 1 | 2 mM Arginine, pH 7.0, 15% IPA, 2 mg/ml siRNA | 75 ± 25 |
| 2 | 2 mM PEI (25,000 mol wt, branched, Sigma), | 115 ± 15 |
| | pH 10, 15% IPA, 2 mg/ml siRNA | |
| 3 | 2 mM Itaconic Acid, pH 8.0, 15% IPA, | 110 ± 2 |
| | 2 mg/ml siRNA | |
| 1 neg | Same as 1, but no siRNA (negative control) | 0 |
| 2 neg | Same as 2, but no siRNA (negative control) | 0 |
| 3 neg | Same as 3, but no siRNA (negative control) | 0 |
| 4 | 10 mM (Glutamic acid, Lysine, Alanine, 3:2:5 | 135 ± 25 |
| | molar ratio), 5% IPA, 1 mg/ml siRNA | |
| 5 | 10 mM (Lysine, Citric acid, 1:4 molar ratio), 15% | 125 ± 25 |
| | IPA, 1 mg/ml siRNA | |
| 6 | 10 mM (Lysine, Citric acid, 1:1 molar ratio), 15% | 130 ± 20 |
| | IPA, 1 mg/ml siRNA | |
| 7 | 10 mM Alanine, 15% IPA, 1 mg/ml siRNA | 125 ± 20 |
| 4 neg | Same as 4, but no siRNA (negative control) | 3 ± 5 |
| 5 neg | Same as 5, but no siRNA (negative control) | 11 ± 12 |
| 6 neg | Same as 6, but no siRNA (negative control) | 30 ± 35 |
| #7 neg | Same as 7, but no siRNA (negative control) | 31 ± 34 |

GAPDH siRNA containing microspheres, generated by the methods described herein, were reconstituted in water to 10 uM siRNA. The negative controls are composed of each formulation without the siRNA. For the positive control, lyophilized GAPDH siRNA was reconstituted to 10 uM siRNA. Each siRNA sample was transfected into Hep-2 cells using lipid-based siPORT ™ NeoFX ™ transfection reagent (Applied Biosystems #AM4510). At 48 hr post transfection, GAPDH enzyme activity was measured using the KDalert ™ GAPDH Assay Kit (Applied Biosystems #AM1639). Fluorescence readings in the negative controls (no siRNA used in transfections) were used to set the baseline. The changes of fluorescent reading in the positive controls (siRNA not subjected to lyophilization) were set as 100% activity for siRNA.

### Example 22

### Microspheres Containing Nucleic Acids as Active Agents and Gelatin as a Carrier

This Example demonstrates that the methods provided herein can be used to prepare microspheres containing gelatin, and the gelatin can act as a carrier for other active agents in the microspheres. The gelatin-containing microspheres are stable, and they retain their stability when nucleic acids are incorporated along with the gelatin. Microspheres were prepared containing gelatin from a variety of sources as follows:

A. Gelatin from bovine skin, Type B (Sigma, G9382)

B. Gelatin from porcine skin, Type A (Sigma, G2500)

C. Gelatin from cold water fish skin (Sigma, G7041)

Preparation of microspheres containing gelatin: for each of the gelatin compounds listed in A-C above, cocktail solutions containing from 2.5 mg/ml to 25 mg/ml of gelatin dissolved in aqueous solvent, counter ions at different pH, and IPA as antisolvent at different concentrations, as listed below, were prepared in a 96-well microtiter plate (0.1 ml cocktail/well) at room temperature. The cocktails in the 96-well plates were cooled by placing in a freezer. The chilled plates were transferred onto pre-chilled (−45° C.) shelves of a Millrock Lab Series Lyophilizer, and a vacuum was applied. The frozen cocktail solutions were allowed to lyophilize for 16 hours.

The lyophilized powders from the bottoms of the wells were transferred onto glass slides and analyzed by light microscopy for appearance. The quality of the product microspheres was scored based on the uniformity of the microspheres, the absence of undesirable non-microsphere particles (glass-like crystalline forms), and the absence of aggregates. The scoring system as described in Table 13 was used.

Table 21 below shows the various combinations of compound, solvent, antisolvent and counterion that were used to generate microspheres, and the quality of the resulting microspheres.

TABLE 21
Gelatin Microspheres

| | | | | |
|---|---|---|---|---|
| Concentration of | | | | Microsphere |
| Compound | Counterion | Antisolvent | pH | Quality |

Compound: Gelatin from
bovine skin, Type B

| | | | | |
|---|---|---|---|---|
| 2.5 mg/ml | 20 mM Citric Acid | 10% | 3.5 | 2 |
| | | isopropanol | | |
| 2.5 mg/ml | 20 mM Citric Acid | 20% | 3.5 | 3 |
| | | isopropanol | | |
| 2.5 mg/ml | 20 mM Citric Acid | 30% | 3.5 | 8 |
| | | isopropanol | | |
| 10 mg/ml | 20 mM Citric Acid | 5% | 3.5 | 6 |
| | | isopropanol | | |
| 10 mg/ml | 20 mM Citric Acid | 10% | 3.5 | 5 |
| | | isopropanol | | |
| 10 mg/ml | 20 mM Citric Acid | 20% | 3.5 | 2 |
| | | isopropanol | | |
| 10 mg/ml | 20 mM Citric Acid | 30% | 3.5 | 2 |
| | | isopropanol | | |
| 25 mg/ml | 20 mM Citric Acid | 30% | 3.5 | 1 |
| | | isopropanol | | |

Compound: Gelatin from
porcine skin, Type A

| | | | | |
|---|---|---|---|---|
| 2.5 mg/ml | 20 mM Citric Acid | 5% | 3.5 | 1 |
| | | isopropanol | | |
| 2.5 mg/ml | 20 mM Citric Acid | 10% | 3.5 | 2 |
| | | isopropanol | | |
| 2.5 mg/ml | 20 mM Citric Acid | 20% | 3.5 | 2 |
| | | isopropanol | | |
| 5 mg/ml | 20 mM Citric Acid | 10% | 3.5 | 6 |
| | | isopropanol | | |
| 5 mg/ml | 20 mM Citric Acid | 20% | 3.5 | 5 |
| | | isopropanol | | |
| 5 mg/ml | 20 mM Citric Acid | 30% | 3.5 | 2 |
| | | isopropanol | | |
| 10 mg/ml | 20 mM Citric Acid | 20% | 3.5 | 1 |
| | | isopropanol | | |
| 10 mg/ml | 20 mM Citric Acid | 30% | 3.5 | 1 |
| | | isopropanol | | |

Compound: Gelatin from
cold water fish skin

| | | | | |
|---|---|---|---|---|
| 2.5 mg/ml | 20 mM Citric Acid | 5% | 3.5 | 3 |
| | | isopropanol | | |
| 2.5 mg/ml | 20 mM Citric Acid | 10% | 3.5 | 2 |
| | | isopropanol | | |
| 2.5 mg/ml | 20 mM Citric Acid | 20% | 3.5 | 2 |
| | | isopropanol | | |
| 2.5 mg/ml | 20 mM Citric Acid | 30% | 3.5 | 5 |
| | | isopropanol | | |
| 5 mg/ml | 20 mM Citric Acid | 5% | 3.5 | 6 |
| | | isopropanol | | |
| 5 mg/ml | 20 mM Citric Acid | 10% | 3.5 | 6 |
| | | isopropanol | | |
| 5 mg/ml | 20 mM Citric Acid | 20% | 3.5 | 8 |
| | | isopropanol | | |
| 5 mg/ml | 20 mM Citric Acid | 30% n- | 3.5 | 9 |
| | | propanol | | |
| 10 mg/ml | 20 mM Citric Acid | 5% | 3.5 | 1 |
| | | isopropanol | | |
| 10 mg/ml | 20 mM Citric Acid | 20% | 3.5 | 7 |
| | | isopropanol | | |
| 10 mg/ml | 20 mM Citric Acid | 30% | 3.5 | 5 |
| | | isopropanol | | |
| 25 mg/ml | 20 mM Citric Acid | 20% | 3.5 | 8 |
| | | isopropanol | | |
| 25 mg/ml | 20 mM Citric Acid | 30% | 3.5 | 6 |
| | | isopropanol | | |
| 2.5 mg/ml | 20 mM Tris | 5% | 8 | 1 |
| | | isopropanol | | |
| 2.5 mg/ml | 20 mM Tris | 10% | 8 | 1 |
| | | isopropanol | | |
| 2.5 mg/ml | 20 mM Tris | 20% | 8 | 8 |
| | | isopropanol | | |
| 2.5 mg/ml | 20 mM Tris | 30% | 8 | 7 |
| | | isopropanol | | |
| 5 mg/ml | 20 mM Tris | 10% | 8 | 5 |
| | | isopropanol | | |
| 5 mg/ml | 20 mM Tris | 30% | 8 | 5 |
| | | isopropanol | | |
| 10 mg/ml | 20 mM Tris | 10% | 8 | 1 |
| | | isopropanol | | |
| 10 mg/ml | 20 mM Tris | 20% | 8 | 5 |
| | | isopropanol | | |
| 10 mg/ml | 20 mM Tris | 30% | 8 | 1 |
| | | isopropanol | | |
| 25 mg/ml | 20 mM Tris | 20% | 8 | 9 |
| | | isopropanol | | |
| 25 mg/ml | 20 mM Tris | 30% | 8 | 9 |
| | | isopropanol | | |

Preparation of microspheres containing gelatin and nucleic acids: for each of the three gelatin compounds listed in A-C above, cocktail solutions containing 15 mg/ml of gelatin and various concentrations of tRNA dissolved in aqueous solvent, with counter ions at different pH, and IPA as antisolvent at different concentrations, as listed below, were prepared in a 96-well microtiter plate (0.1 ml cocktail/well) at room temperature. tRNA used in this experiment was type X-SA, from Bakers Yeast (Sigma, R8759). The cocktail solutions were cooled by placing in a freezer. The chilled plates were transferred onto pre-chilled (−45° C.) shelves of a Millrock Lab Series Lyophilizer, and a vacuum was applied. The frozen cocktail solutions were allowed to lyophilize for 16 hours.

The lyophilized powders from the bottoms of the wells were transferred onto glass slides and analyzed by light microscopy for appearance. The quality of the product microspheres was scored based on the uniformity of the microspheres, the absence of undesirable non-microsphere particles (glass-like crystalline forms), and the absence of aggregates.

| | | | | |
|---|---|---|---|---|
| Concentration of | | | | Microsphere |
| tRNA | Counterion | Antisolvent | pH | Quality |

Compound: Gelatin from bovine
skin, Type B with tRNA

| | | | | |
|---|---|---|---|---|
| 2 mg/ml | 10 mM Citric Acid | 10% | 3.5 | 7 |
| | | isopropanol | | |
| 2 mg/ml | 10 mM Citric Acid | 20% | 3.5 | 5 |
| | | isopropanol | | |
| 2 mg/ml | 10 mM Citric Acid | 30% | 3.5 | 4 |
| | | isopropanol | | |
| 2 mg/ml | 10 mM Citric Acid | 40% | 3.5 | 5 |
| | | isopropanol | | |
| 1 mg/ml | 10 mM Citric Acid | 10% | 3.5 | 1 |
| | | isopropanol | | |
| 1 mg/ml | 10 mM Citric Acid | 20% | 3.5 | 3 |
| | | isopropanol | | |
| 1 mg/ml | 10 mM Citric Acid | 30% | 3.5 | 5 |
| | | isopropanol | | |
| 1 mg/ml | 10 mM Citric Acid | 40% | 3.5 | 5 |
| | | isopropanol | | |
| 0.5 mg/ml | 10 mM Citric Acid | 10% | 3.5 | 3 |
| | | isopropanol | | |
| 0.5 mg/ml | 10 mM Citric Acid | 20% | 3.5 | 2 |
| | | isopropanol | | |
| 0.5 mg/ml | 10 mM Citric Acid | 30% | 3.5 | 3 |
| | | isopropanol | | |
| 0.5 mg/ml | 10 mM Citric Acid | 40% | 3.5 | 3 |
| | | isopropanol | | |
| 0.1 mg/ml | 10 mM Citric Acid | 30% | 3.5 | 2 |
| | | isopropanol | | |
| 0.1 mg/ml | 10 mM Citric Acid | 40% | 3.5 | 4 |
| | | isopropanol | | |
| 2 mg/ml | 10 mM Tris | 30% | 8 | 2 |
| | | isopropanol | | |
| 2 mg/ml | 10 mM Tris | 40% | 8 | 1 |
| | | isopropanol | | |
| 1 mg/ml | 10 mM Tris | 40% | 8 | 2 |
| | | isopropanol | | |
| 0.5 mg/ml | 10 mM Tris | 40% | 8 | 1 |
| | | isopropanol | | |

Compound: Gelatin from porcine
skin, Type A with tRNA

| | | | | |
|---|---|---|---|---|
| 2 mg/ml | 10 mM Citric Acid | 10% | 3.5 | 4 |
| | | isopropanol | | |
| 2 mg/ml | 10 mM Citric Acid | 20% | 3.5 | 6 |
| | | isopropanol | | |
| 2 mg/ml | 10 mM Citric Acid | 30% | 3.5 | 2 |
| | | isopropanol | | |
| 2 mg/ml | 10 mM Citric Acid | 40% | 3.5 | 5 |
| | | isopropanol | | |
| 1 mg/ml | 10 mM Citric Acid | 10% | 3.5 | 2 |
| | | isopropanol | | |
| 1 mg/ml | 10 mM Citric Acid | 40% | 3.5 | 2 |
| | | isopropanol | | |
| 0.5 mg/ml | 10 mM Citric Acid | 10% | 3.5 | 3 |
| | | isopropanol | | |
| 0.5 mg/ml | 10 mM Citric Acid | 20% | 3.5 | 1 |
| | | isopropanol | | |
| 0.5 mg/ml | 10 mM Citric Acid | 30% | 3.5 | 5 |
| | | isopropanol | | |
| 0.5 mg/ml | 10 mM Citric Acid | 40% | 3.5 | 1 |
| | | isopropanol | | |
| 0.1 mg/ml | 10 mM Citric Acid | 30% | 3.5 | 8 |
| | | isopropanol | | |
| 0.1 mg/ml | 10 mM Citric Acid | 40% | 3.5 | 3 |
| | | isopropanol | | |
| 2 mg/ml | 10 mM Tris | 30% | 8 | 1 |
| | | isopropanol | | |
| 2 mg/ml | 10 mM Tris | 40% | 8 | 2 |
| | | isopropanol | | |
| 1 mg/ml | 10 mM Tris | 30% | 8 | 3 |
| | | isopropanol | | |
| 1 mg/ml | 10 mM Tris | 40% | 8 | 3 |
| | | isopropanol | | |
| 0.5 mg/ml | 10 mM Tris | 30% | 8 | 3 |
| | | isopropanol | | |
| 0.5 mg/ml | 10 mM Tris | 40% | 8 | 3 |
| | | isopropanol | | |
| 0.1 mg/ml | 10 mM Tris | 40% | 8 | 2 |
| | | isopropanol | | |

Compound: Gelatin from cold
water fish skin with tRNA

| | | | | |
|---|---|---|---|---|
| 2 mg/ml | 10 mM Citric Acid | 10% | 3.5 | 5 |
| | | isopropanol | | |
| 2 mg/ml | 10 mM Citric Acid | 20% | 3.5 | 5 |
| | | isopropanol | | |
| 2 mg/ml | 10 mM Citric Acid | 30% | 3.5 | 4 |
| | | isopropanol | | |
| 2 mg/ml | 10 mM Citric Acid | 40% | 3.5 | 5 |
| | | isopropanol | | |
| 1 mg/ml | 10 mM Citric Acid | 10% | 3.5 | 4 |
| | | isopropanol | | |
| 1 mg/ml | 10 mM Citric Acid | 20% | 3.5 | 4 |
| | | isopropanol | | |
| 1 mg/ml | 10 mM Citric Acid | 30% | 3.5 | 6 |
| | | isopropanol | | |
| 1 mg/ml | 10 mM Citric Acid | 40% | 3.5 | 4 |
| | | isopropanol | | |
| 0.5 mg/ml | 10 mM Citric Acid | 10% | 3.5 | 10 |
| | | isopropanol | | |
| 0.5 mg/ml | 10 mM Citric Acid | 20% | 3.5 | 8 |
| | | isopropanol | | |
| 0.5 mg/ml | 10 mM Citric Acid | 30% | 3.5 | 8 |
| | | isopropanol | | |
| 0.5 mg/ml | 10 mM Citric Acid | 40% | 3.5 | 7 |
| | | isopropanol | | |
| 0.1 mg/ml | 10 mM Citric Acid | 20% | 3.5 | 7 |
| | | isopropanol | | |
| 0.1 mg/ml | 10 mM Citric Acid | 30% | 3.5 | 7 |
| | | isopropanol | | |
| 0.1 mg/ml | 10 mM Citric Acid | 40% | 3.5 | 7 |
| | | isopropanol | | |
| 2 mg/ml | 10 mM Tris | 20% | 8 | 6 |
| | | isopropanol | | |
| 2 mg/ml | 10 mM Tris | 30% | 8 | 5 |
| | | isopropanol | | |
| 2 mg/ml | 10 mM Tris | 40% | 8 | 6 |
| | | isopropanol | | |
| 1 mg/ml | 10 mM Tris | 10% | 8 | 4 |
| | | isopropanol | | |
| 1 mg/ml | 10 mM Tris | 20% | 8 | 5 |
| | | isopropanol | | |
| 1 mg/ml | 10 mM Tris | 30% | 8 | 7 |
| | | isopropanol | | |
| 1 mg/ml | 10 mM Tris | 40% | 8 | 4 |
| | | isopropanol | | |
| 0.5 mg/ml | 10 mM Tris | 10% | 8 | 2 |
| | | isopropanol | | |
| 0.5 mg/ml | 10 mM Tris | 20% | 8 | 8 |
| | | isopropanol | | |
| 0.5 mg/ml | 10 mM Tris | 30% | 8 | 8 |
| | | isopropanol | | |
| 0.5 mg/ml | 10 mM Tris | 40% | 8 | 4 |
| | | isopropanol | | |
| 0.1 mg/ml | 10 mM Tris | 10% | 8 | 2 |
| | | isopropanol | | |
| 0.1 mg/ml | 10 mM Tris | 20% | 8 | 4 |
| | | isopropanol | | |
| 0.1 mg/ml | 10 mM Tris | 30% | 8 | 3 |
| | | isopropanol | | |
| 0.1 mg/ml | 10 mM Tris | 40% | 8 | 5 |
| | | isopropanol | | |

Results: These experiments demonstrate that by selecting the appropriate parameters, stable gelatin microspheres can be obtained. Further, active agents such as nucleic acids can be incorporated into the gelatin matrix to produce a drug product with defined potency.

### Example 23

### Preparation of Microspheres Using a Polysaccharide as a Carrier

This Example demonstrates that the methods provided herein can be used to prepare microspheres containing polysaccharides. The polysaccharides in turn can be carriers for therapeutic agents or active agents incorporated into the microspheres. The following compounds were tested:

A) Dextran Sulfate Sodium Salt (Sigma, D 6924)

B) Hydroxypropyl-B-cyclodextrin (Tokyo Chemical Industry Co, Ltd, H0979)

Preparation of microspheres: for the compounds A) and B) above, cocktail solutions containing from 0.5 mg/ml to 10 mg/ml of compound, with counter ions at different pH, and IPA as antisolvent at different concentrations, as listed below, were prepared in a 96-well microtiter plate (0.1 ml cocktail/well) at room temperature. Cocktails were cooled by placing in a freezer. The chilled plates were transferred onto pre-chilled (−45° C.) shelves of a Millrock Lab Series Lyophilizer, and a vacuum was applied. The frozen cocktail solutions were allowed to lyophilize for 16 hours.

The lyophilized powders from the bottoms of the wells were transferred onto glass slides and analyzed by light microscopy for appearance. The quality of the product microspheres was scored based on the uniformity of the microspheres, the absence of undesirable non-microsphere particles (glass-like crystalline forms), and the absence of aggregates.

| | | | | |
|---|---|---|---|---|
| Concentration | | | | Microsphere |
| of Compound | Counterion | Antisolvent | pH | Quality |

Compound: Dextran
Sulfate

| | | | | |
|---|---|---|---|---|
| 5 mg/ml | 10 mM Citric Acid | 5% | 3.5 | 4 |
| | | isopropanol | | |
| 5 mg/ml | 10 mM Citric Acid | 10% | 3.5 | 5 |
| | | isopropanol | | |
| 5 mg/ml | 10 mM Citric Acid | 20% | 3.5 | 2 |
| | | isopropanol | | |
| 5 mg/ml | 10 mM Citric Acid | 30% | 3.5 | 6 |
| | | isopropanol | | |
| 1 mg/ml | 10 mM Citric Acid | 5% | 3.5 | 2 |
| | | isopropanol | | |
| 1 mg/ml | 10 mM Citric Acid | 10% | 3.5 | 4 |
| | | isopropanol | | |
| 1 mg/ml | 10 mM Citric Acid | 20% | 3.5 | 3 |
| | | isopropanol | | |
| 5 mg/ml | 10 mM Tris | 5% | 8 | 2 |
| | | isopropanol | | |
| 5 mg/ml | 10 mM Tris | 10% | 8 | 2 |
| | | isopropanol | | |
| 5 mg/ml | 10 mM Tris | 20% | 8 | 2 |
| | | isopropanol | | |
| 10 mg/ml | 30 mM Citric Acid | 5% | 3.5 | 2 |
| | | isopropanol | | |
| 10 mg/ml | 30 mM Citric Acid | 10% | 3.5 | 3 |
| | | isopropanol | | |
| 10 mg/ml | 30 mM Citric Acid | 20% | 3.5 | 2 |
| | | isopropanol | | |
| 5 mg/ml | 30 mM Citric Acid | 5% | 3.5 | 2 |
| | | isopropanol | | |
| 5 mg/ml | 30 mM Citric Acid | 10% | 3.5 | 3 |
| | | isopropanol | | |
| 5 mg/ml | 30 mM Citric Acid | 20% | 3.5 | 2 |
| | | isopropanol | | |
| 5 mg/ml | 30 mM Citric Acid | 30% | 3.5 | 2 |
| | | isopropanol | | |
| 1 mg/ml | 30 mM Citric Acid | 5% | 3.5 | 2 |
| | | isopropanol | | |
| 1 mg/ml | 30 mM Citric Acid | 20% | 3.5 | 1 |
| | | isopropanol | | |
| 10 mg/ml | 10 mM Citric Acid | 5% | 5.2 | 1 |
| | | isopropanol | | |
| 10 mg/ml | 10 mM Citric Acid | 10% | 5.2 | 4 |
| | | isopropanol | | |
| 10 mg/ml | 10 mM Citric Acid | 20% | 5.2 | 2 |
| | | isopropanol | | |
| 10 mg/ml | 10 mM Citric Acid | 30% | 5.2 | 1 |
| | | isopropanol | | |
| 5 mg/ml | 10 mM Citric Acid | 5% | 5.2 | 2 |
| | | isopropanol | | |
| 5 mg/ml | 10 mM Citric Acid | 10% | 5.2 | 3 |
| | | isopropanol | | |
| 5 mg/ml | 10 mM Citric Acid | 20% | 5.2 | 1 |
| | | isopropanol | | |
| 10 mg/ml | 30 mM Citric Acid | 20% | 5.2 | 1 |
| | | isopropanol | | |
| 10 mg/ml | 30 mM Citric Acid | 30% | 5.2 | 1 |
| | | isopropanol | | |
| 5 mg/ml | 30 mM Citric Acid | 5% | 5.2 | 3 |
| | | isopropanol | | |
| 5 mg/ml | 30 mM Citric Acid | 10% | 5.2 | 1 |
| | | isopropanol | | |
| 5 mg/ml | 30 mM Citric Acid | 20% | 8 | 1 |
| | | isopropanol | | |
| 5 mg/ml | 30 mM Citric Acid | 30% | 5.2 | 3 |
| | | isopropanol | | |
| 10 mg/ml | 30 mM Tris | 5% | 8 | 3 |
| | | isopropanol | | |
| 10 mg/ml | 30 mM Tris | 10% | 8 | 1 |
| | | isopropanol | | |
| 10 mg/ml | 30 mM Tris | 20% | 8 | 3 |
| | | isopropanol | | |
| 10 mg/ml | 30 mM Tris | 30% | 8 | 4 |
| | | isopropanol | | |
| 5 mg/ml | 30 mM Tris | 5% | 8 | 3 |
| | | isopropanol | | |
| 5 mg/ml | 30 mM Tris | 20% | 8 | 4 |
| | | isopropanol | | |
| 5 mg/ml | 30 mM Tris | 30% | 8 | 2 |
| | | isopropanol | | |
| 10 mg/ml | 10 mM Tris | 5% | 11 | 2 |
| | | isopropanol | | |
| 10 mg/ml | 10 mM Tris | 30% | 11 | 1 |
| | | isopropanol | | |
| 5 mg/ml | 10 mM Tris | 5% | 11 | 2 |
| | | isopropanol | | |
| 5 mg/ml | 10 mM Tris | 10% | 11 | 5 |
| | | isopropanol | | |
| 5 mg/ml | 10 mM Tris | 20% | 11 | 5 |
| | | isopropanol | | |
| 5 mg/ml | 10 mM Tris | 30% | 11 | 2 |
| | | isopropanol | | |
| 1 mg/ml | 10 mM Tris | 5% | 11 | 5 |
| | | isopropanol | | |
| 1 mg/ml | 10 mM Tris | 10% | 11 | 4 |
| | | isopropanol | | |
| 1 mg/ml | 10 mM Tris | 20% | 11 | 4 |
| | | isopropanol | | |
| 1 mg/ml | 10 mM Tris | 30% | 11 | 3 |
| | | isopropanol | | |
| 10 mg/ml | 30 mM Tris | 10% | 11 | 1 |
| | | isopropanol | | |
| 10 mg/ml | 30 mM Tris | 30% | 11 | 5 |
| | | isopropanol | | |
| 5 mg/ml | 30 mM Tris | 5% | 11 | 1 |
| | | isopropanol | | |
| 5 mg/ml | 30 mM Tris | 30% | 11 | 2 |
| | | isopropanol | | |

Compound:
Hydroxypropyl-B-
cyclodextrin

| | | | | |
|---|---|---|---|---|
| 5 mg/ml | 10 mM Citric Acid | 5% | 3.5 | 1 |
| | | isopropanol | | |
| 5 mg/ml | 10 mM Citric Acid | 20% | 3.5 | 2 |
| | | isopropanol | | |
| 5 mg/ml | 10 mM Citric Acid | 30% | 3.5 | 1 |
| | | isopropanol | | |
| 1 mg/ml | 10 mM Citric Acid | 10% | 3.5 | 1 |
| | | isopropanol | | |
| 1 mg/ml | 10 mM Citric Acid | 20% | 3.5 | 4 |
| | | isopropanol | | |
| 1 mg/ml | 10 mM Citric Acid | 30% | 3.5 | 5 |
| | | isopropanol | | |
| 0.5 mg/ml | 10 mM Citric Acid | 5% | 3.5 | 2 |
| | | isopropanol | | |
| 5 mg/ml | 10 mM Tris | 20% | 8 | 1 |
| | | isopropanol | | |

Results: These experiments demonstrate that by selecting the appropriate combination of: (a) type and (b) concentration of compound, counterion and antisolvent, polysaccharide microspheres can be obtained.

### Example 24

### Amino Acid Microspheres

This Example demonstrates that the methods provided herein can be used to prepare microspheres containing various amino acids, which could be active agents or therapeutic agents themselves, or serve as carriers for other active agents and therapeutic agents. Microspheres of the following amino acids were prepared:

A. Alanine

B. Glutamic Acid

C. Tryptophan

D. Methionine

E. Phenylalanine

F. Glycine

G. Lycine

Preparation of amino acid microspheres: for each of the compounds listed in A-G above, cocktail solutions containing 20 mM amino acid dissolved in aqueous solvent, at different pH, and ispropanol (IPA) as antisolvent at different concentrations, as listed below, were prepared in a 96-well microtiter plate (0.1 ml cocktail/well) at room temperature. Cocktails were cooled by placing in a freezer. The chilled plates were transferred onto pre-chilled (−45° C.) shelves of a Millrock Lab Series Lyophilizer, and the vacuum was applied. The frozen cocktail solutions were allowed to lyophilize for 16 hours.

The lyophilized powders from the bottoms of the wells were transferred onto glass slides and analyzed by light microscopy for appearance. The quality of the product microspheres was scored based on the uniformity of the microspheres, the absence of undesirable non-microsphere particles (glass-like crystalline forms), and the absence of aggregates. The scoring system as described in Table 13 was used.

Table 24 below shows the various combinations of compound, solvent, and antisolvent that were used to generate microspheres, and the quality of the resulting microspheres.

TABLE 24
Amino Acid Microspheres
Amino Acids:

| | | |
|---|---|---|
| Antisolvent | pH | Microsphere Quality |

Compound: Alanine

| | | |
|---|---|---|
| 5% isopropanol | 7 | 9 |
| 10% isopropanol | 7 | 2 |
| 20% isopropanol | 7 | 8 |
| 30% isopropanol | 7 | 5 |
| 5% isopropanol | 6 | 7 |

Compound: Glutamic Acid

| | | |
|---|---|---|
| 5% isopropanol | 3.2 | 6 |
| 10% isopropanol | 3.2 | 3 |
| 20% isopropanol | 3.2 | 6 |

Compound: Tryptophan

| | | |
|---|---|---|
| 5% isopropanol | 7 | 5 |
| 10% isopropanol | 7 | 4 |
| 20% isopropanol | 7 | 7 |
| 30% isopropanol | 7 | 4 |
| 20% isopropanol | 6 | 2 |
| 30% isopropanol | 6 | 7 |

Compound: Methionine

| | | |
|---|---|---|
| 5% isopropanol | 7 | 3 |
| 10% isopropanol | 7 | 4 |
| 20% isopropanol | 7 | 1 |
| 30% isopropanol | 7 | 2 |
| 5% isopropanol | 5.7 | 2 |
| 10% isopropanol | 5.7 | 7 |
| 30% isopropanol | 5.7 | 1 |

Compound: Phenylalanine

| | | |
|---|---|---|
| 10% isopropanol | 7 | 6 |
| 20% isopropanol | 7 | 8 |
| 30% isopropanol | 7 | 5 |
| 5% isopropanol | 5.5 | 6 |
| 10% isopropanol | 5.5 | 5 |
| 20% isopropanol | 5.5 | 3 |
| 30% isopropanol | 5.5 | 7 |

Compound: Glycine

| | | |
|---|---|---|
| 5% isopropanol | 7 | 7 |
| 10% isopropanol | 7 | 6 |
| 20% isopropanol | 7 | 4 |
| 30% isopropanol | 7 | 4 |
| 5% isopropanol | 6 | 5 |
| 10% isopropanol | 6 | 5 |
| 20% isopropanol | 6 | 3 |
| 30% isopropanol | 6 | 6 |

Compound: Lysine

| | | |
|---|---|---|
| 5% isopropanol | 7 | 3 |
| 20% isopropanol | 7 | 7 |
| 30% isopropanol | 7 | 3 |
| 5% isopropanol | 5.5 | 4 |
| 10% isopropanol | 5.5 | 5 |
| 30% isopropanol | 5.5 | 7 |

Results: These experiments demonstrate that by selecting the appropriate combination of: (a) type and (b) concentration of amino acid, counterion and antisolvent, microspheres made of amino acids can be obtained.

Since modifications will be apparent to those of skill in this art, it is intended that this invention be limited only by the scope of the appended claims.

What is claimed is:

1. A method of making microparticles of a compound, comprising:
a) adding a counterion to a solution containing the compound in a solvent;
b) adding an antisolvent to the solution; and
c) gradually cooling the solution to a temperature below about 25° C., whereby a composition containing microparticles comprising the compound is formed, wherein steps a), b) and c) are performed simultaneously, sequentially, intermittently, or in any order.

2. The method of claim 1, wherein the counterion is not a polymer.

3. The method of claim 1, wherein the antisolvent is not a polymer.

4. The method of claim 1, wherein prior to step a), the compound is dissolved in the solvent at a temperature of about or at 30° C. or below.

5. The method of claim 4, wherein prior to step a), the compound is dissolved in the solvent at a temperature of about or at 25° C. or below.

6. The method of claim 1, wherein steps a) and b) are performed at ambient temperature.

7. The method of claim 1, wherein none of the solutions of steps a)-c) are heated and/or maintained at a temperature above about or at 30° C.

8. The method of claim 1, wherein the compound is not a protein or a polypeptide.

9. The method of claim 1, wherein steps a) and b) are performed simultaneously, sequentially, intermittently, or in any order, followed by step c).

10. The method of claim 1, wherein steps b) and c) are performed simultaneously, sequentially, intermittently, or in any order, preceded by step a).

11. The method of claim 1, wherein steps a) and c) are performed simultaneously.

12. The method of claim 1, wherein the counterion and the compound are identical to one another.

13. The method of claim 1, wherein the compound and the counterion are different from one another.

14. The method of claim 1, wherein the counterion and the antisolvent are identical to one another.

15. The method of claim 1, wherein the compound is a macromolecule with a molecular weight of about or at 1000 or 1000 to about or at five billion or five billion Daltons.

16. The method of claim 15, wherein the compound is a macromolecule with a molecular weight of about or at 1000 or 1000 to about or at one billion or one billion Daltons.

17. The method of claim 16, wherein the compound is a macromolecule with a molecular weight of about or at 1000 or 1000 to about or at 50 million or 50 million Daltons.

18. The method of claim 17, wherein the compound is a macromolecule with a molecular weight of about or at 1000 or 1000 to about or at 10 million or 10 million Daltons.

19. The method of claim 18, wherein the compound is a macromolecule with a molecular weight of about or at 1000 or 1000 to about or at one million or one million Daltons.

20. The method of claim 19, wherein the compound is a macromolecule with a molecular weight of about or at 1000 or 1000 to about or at 500,000 or 500,000 Daltons.

21. The method of claim 20, wherein the compound is a macromolecule with a molecular weight of about or at 1000 or 1000 to about or at 100,000 or 100,000 Daltons.

22. The method of claim 21, wherein the compound is a macromolecule with a molecular weight of about or at 1000 or 1000 to about or at 50,000 or 50,000 Daltons.

23. The method of claim 22, wherein the compound is a macromolecule with a molecular weight of about or at 1000 or 1000 to about or at 10,000 or 10,000 Daltons.

24. The method of claim 23, wherein the compound is a macromolecule with a molecular weight of about or at 1000 or 1000 to about or at 5,000 or 5,000 Daltons.

25. The method of claim 24, wherein the compound is a macromolecule with a molecular weight of about or at 1000 or 1000 to about or at 2,000 or 2000 Daltons.

26. The method of claim 1, wherein the compound is a small molecule.

27. The method of claim 15, wherein the macromolecule is selected from among a polynucleotide, a nucleic acid, a polypeptide, a glycopeptide, a protein, a carbohydrate, a lipid, a fatty acid, a polysaccharide, carbohydrate- or polysaccharide-protein conjugates, virus, virus particles, viroids, prions and mixtures thereof.

28. The method of claim 2, wherein the compound is a macromolecule, and the macromolecule is selected from among a polynucleotide, a nucleic acid, a polypeptide, a glycopeptide, a protein, a carbohydrate, a lipid, a fatty acid, a polysaccharide, carbohydrate- or polysaccharide-protein conjugates, virus, virus particles, viroids, prions and mixtures thereof.

29. The method of claim 15, wherein the macromolecule is selected from among hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, and cholesterol lowering agents.

30. The method of claim 27, wherein the macromolecule is conjugated to a small molecule.

31. The method of claim 30, wherein the small molecule is selected from among haptens, hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, and cholesterol lowering agents.

32. The method of claim 26, wherein the small molecule has a molecular weight of about or at 50 to about or at 1000 Daltons.

33. The method of claim 26, wherein the small molecule is selected from among haptens, hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrythmic agents, vasodilators, antihypertensive agents, antidiabetic agents, anticoagulants, and cholesterol lowering agents.

34. The method of claim 33, wherein the small molecule is an antibiotic.

35. The method of claim 34, wherein the antibiotic is selected from among aminoglycosides, ansamycins, carbacephem, carbapenems, cephalosporins, macrolides, penicillins, quinolones, sulfonamides and tetracyclines.

36. The method of claim 35, wherein the antibiotic is a penicillin or a tetracycline.

37. The method of claim 35, wherein the antibiotic is an aminoglycoside.

38. The method of claim 37, wherein the aminoglycoside is kanamycin or tobramycin.

39. The method of claim 33, wherein the small molecule is an antiviral agent.

40. The method of claim 39, wherein the antiviral agent is for treatment of influenza, parainfluenza or respiratory syncytial virus-mediated infections.

41. The method of claim 40, wherein the antiviral agent is zanamivir or oseltamivir phosphate.

42. The method of claim 33, wherein the small molecule is a chemotherapeutic agent.

43. The method of claim 42, wherein the chemotherapeutic agent is selected from among alkylating agents, anthracyclines, cytoskeletal disruptors, epothilones, inhibitors of topoisomerase II, nucleotide analogs, platinum-based agents, retinoids and vinca alkaloids.

44. The method of claim 43, wherein the chemotherapeutic agent is a cytoskeletal disruptor.

45. The method of claim 44, wherein the cytoskeletal disruptor is paclitaxel.

46. The method of claim 33, wherein the small molecule is a prostaglandin.

47. The method of claim 27, wherein the macromolecule is a nucleic acid.

48. The method of claim 47, wherein the nucleic acid is selected from among DNA, RNA and PNA.

49. The method of claim 48, wherein the nucleic acid is RNA.

50. The method of claim 49, wherein the RNA is selected from among siRNA, tRNA, snRNA and ribozymes.

51. The method of claim 50, wherein the RNA is siRNA.

52. The method of claim 27, wherein the macromolecule is a virus.

53. The method of claim 52, wherein the virus is tobacco mosaic virus.

54. The method of claim 27, wherein the macromolecule is a glycopeptide.

55. The method of claim 54, wherein the glycopeptide is vancomycin.

56. The method of claim 27, wherein the macromolecule is a peptide.

57. The method of claim 56, wherein the peptide is leuprolide.

58. The method of claim 56, wherein the peptide is somatostatin

59. The method of claim 1, wherein the compound is water-insoluble.

60. The method of claim 1, wherein steps a), b) and c) are performed sequentially in the order: a), then b), then c).

61. The method of claim 1, wherein the solvent is miscible or partially miscible with the antisolvent.

62. The method of claim 1, further comprising, after step c), separating the microparticles from the solution to remove components other than the microparticles.

63. The method of claim 62, wherein the composition consists essentially of the microparticles comprising the compound.

64. The method of claim 62, wherein the separation is effected by sedimentation or by filtration.

65. The method of claim 62, wherein the separation is effected by freeze-drying.

66. The method of claim 1, wherein the antisolvent is selected from among water, buffered solutions, aliphatic alcohols, aromatic alcohols, chloroform, polyhydric sugar alcohols, aromatic hydrocarbons, aldehydes, ketones, esters, ethers, dioxanes, alkanes, alkenes, conjugated dienes, dichloromethane, carbon tetrachloride, dimethylformamide (DMF), dimethyl sulfoxide (DMSO), acetonitrile, ethyl acetate, polyols, polyimides, polyimines, polyesters, polyaldehydes and mixtures thereof.

67. The method of claim 66, wherein the antisolvent is an aliphatic alcohol or an aromatic alcohol.

68. The method of claim 67, wherein the antisolvent is an aliphatic alcohol.

69. The method of claim 68, wherein the aliphatic alcohol is isopropanol.

70. The method of claim 1, wherein the counterion is selected from among an anionic compound, a cationic compound and a zwitterionic compound.

71. The method of claim 70, wherein the counterion is an anionic compound.

72. The method of claim 71, wherein the anionic compound is selected from among sodium citrate, sodium sulfate, zinc sulfate, magnesium sulfate, potassium sulfate and calcium sulfate.

73. The method of claim 72, wherein the anionic compound is sodium sulfate.

74. The method of claim 70, wherein the counterion is selected from among citric acid, itaconic acid and pivalic acid.

75. The method of claim 70, wherein the counterion is an amino acid.

76. The method of claim 75, wherein the compound is glycine or arginine.

77. The method of claim 15, wherein the counterion is a polymer, and the macromolecule is selected from among a polynucleotide, a nucleic acid, a carbohydrate, a lipid, a fatty acid, a polysaccharide, carbohydrate- or polysaccharide-protein conjugates, a virus, virus particles, viroids, prions and mixtures thereof.

78. The method of claim 77, wherein the polymer is the counterion and the antisolvent.

79. The method of claim 78, wherein the polymer is polyethylene glycol (PEG) or polyethyleneimine (PEI).

80. The method of claim 1, wherein the counterion is a polymer.

81. The method of claim 80, wherein the polymer is the counterion and the antisolvent.

82. The method of claim 81, wherein the polymer is polyethylene glycol (PEG) or polyethyleneimine (PEI).

83. The method of claim 1, wherein the microparticles are obtained by precipitation, by phase separation or by colloid formation.

84. The method of claim 1, wherein the pH of the solution is from about 4.0 or 4.0 to about 9.0 or 9.0.

85. The method of claim 1, wherein the resulting microparticle composition further comprises micro-carriers, acid-resistant coating agents, protease-resistant coating agents, enteric coating agents, bulking agents, excipients, inactive ingredients, stability enhancers, taste and/or odor modifiers or masking agents, vitamins, sugars, therapeutic agents, anti-oxidants, immuno-modulators, trans-membrane transport modifiers, anti-caking agents, chitosans or flowability enhancers.

86. The method of claim 85, wherein the resulting microparticle further comprises a micro-carrier, and the micro-carrier is selected from among amino acids, carboxylic acids, proteins, nucleic acids, polysaccharides, and materials that are capable of forming hydrogels.

87. The method of claim 86, wherein the micro-carrier is a material that is capable of forming a hydrogel.

88. The method of claim 87, wherein the micro-carrier is gelatin or dextran.

89. The method of claim 88, wherein the compound is a protein.

90. The method of claim 88, wherein the compound is a nucleic acid.

91. The method of claim 90, wherein the nucleic acid is siRNA and the micro-carrier is gelatin.

92. The method of claim 1, wherein the compound is a micro-carrier.

93. The method of claim 92, wherein the micro-carrier is selected from among amino acids, carboxylic acids, proteins, nucleic acids, polysaccharides, and materials that are capable of forming hydrogels.

94. The method of claim 93, wherein the micro-carrier is a material that is capable of forming a hydrogel.

95. The method of claim 94, wherein the micro-carrier is gelatin.

96. The method of claim 95, wherein the micro-carrier is dextran.

97. The method of claim 1, wherein the amount of compound in the microparticles relative to the total amount of compound in the solution of step a) is about 5% or 5% to greater than about 99% or 99%, w/w.

98. The method of claim 97, wherein the amount of compound in the microparticles relative to the total amount of compound in the solution of step a) is about 10% or 10% to about 85% or 85%, w/w.

99. The method of claim 98, wherein the amount of compound in the microparticles relative to the total amount of compound in the solution of step a) is about 25% or 25% to about 55% or 55%, w/w.

100. The method of claim 97, wherein the amount of compound in the microparticles relative to the total amount of compound in the solution of step a) is about 80% or 80% to greater than about 99% or 99%, w/w.

101. The method of claim 1, wherein the temperature is between about or at 4° C. to about or at −200° C.

102. The method of claim 101, wherein the temperature is between about or at 2° C. to about or at −170° C.

103. The method of claim 102, wherein the temperature is between about 0° C. or 0° C. to about −2° C. or −2° C. to from about −150° C. or −150° C. to about −165° C. or −165° C.

104. The method of claim 1, wherein the resulting composition has a shelf life of from about or at one week to about or at 1 month, from about or at 1 month to about or at six months, from about or at six months to about or at one year, from about or at 1 year to about or at 2 years, or from about or at 2 years to about or at 5 years at a temperature of about or at 55° C., 50° C., 45° C., 44° C., 42° C., 40° C., 39° C., 38° C., 37° C. or below.

105. The method of claim 1, wherein the solution and/or the resulting composition further comprises an active agent.

106. The method of claim 105, wherein the active agent is selected from among antibiotics, chemotherapeutic agents, antidiabetics, anticonvulsants, analgesics, antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids, sympathomimetics, parasympathomimetics, tranquilizers, urinary tract drugs, vaccines, vaginal drugs, nonsteroidal anti-inflammatory drugs, angiotensin converting enzymes, polynucleotides, polypeptides, polysaccharides, enzymes, hormones, vitamins, minerals, and nutritional supplements.

107. The method of claim 1, wherein the moisture content of the microparticles is adjusted whereby at least about 90% or 90% of the activity of the compound is retained after storage for about six months to about 1 year at a temperature of about 25° C.

108. The method of claim 1, wherein the moisture content of the microparticles is from about or at 0.01% to about or at 20%.

109. The method of claim 1, wherein the concentration of counterion added to the solution is from about or at 0 mM or 0 mM to about or at 100 mM or 100 mM.

110. The method of claim 109, wherein the concentration of counterion added to the solution is from about or at 0 mM or 0 mM to about or at 50 nnM or 50 mM.

111. The method of claim 110, wherein the concentration of counterion added to the solution is from about or at 1 mM or 1 mM to about or at 5 mM or 5 mM.

112. The method of claim 111, wherein the concentration of counterion added to the solution is about or at 2 mM.

113. The method of claim 1, wherein the gradual cooling is by chilling.

114. The method of claim 1, wherein the gradual cooling is by an endothermic reaction.

115. The method of claim 1, wherein the gradual cooling is at a rate of from about or at 0.01° C./min or 0.01° C./min to about or at 20° C./min or 20° C./min.

116. The method of claim 115, wherein the gradual cooling is at a rate of about or at 0.2° C./min to about or at 5° C./min.

117. The method of claim 116, wherein the gradual cooling is at a rate of about or at 0.5° C./min to about or at 2° C./min

118. The method of claim 117, wherein the gradual cooling is at a rate of about or at 1° C./min.

119. The method of claim 1, wherein the size of the microparticles is from about or at 0.001 μm or 0.001 μm to about or at 50 μm or 50 μm.

120. The method of claim 119, wherein the size of the microparticles is from about or at 0.5 μm or 0.5 μm to about or at 10 μm or 10 μm.

121. The method of claim 120, wherein the size of the microparticles is from about or at 1.0 μm to about or at 2.0, 3.0, 4.0 or 5.0 μm.

122. A composition, comprising microparticles of a compound and a counterion, wherein the compound and the counterion are different from one another.

123. The composition of claim 122, wherein the compound is a macromolecule with a molecular weight of about or at 1000 or 1000 to about or at five billion or five billion Daltons.

124. The composition of claim 123, wherein the compound is a macromolecule with a molecular weight of about or at 1000 or 1000 to about or at one billion or one billion Daltons.

125. The composition of claim 124, wherein the compound is a macromolecule with a molecular weight of about or at 1000 or 1000 to about or at 50 million or 50 million Daltons.

126. The composition of claim 125, wherein the compound is a macromolecule with a molecular weight of about or at 1000 or 1000 to about or at 5 million or 5 million Daltons.

127. The composition of claim 126, wherein the compound is a macromolecule with a molecular weight of about or at 1000 or 1000 to about or at one million or one million Daltons.

128. The composition of claim 127, wherein the compound is a macromolecule with a molecular weight of about or at 1000 or 1000 to about or at 200,000 or 200,000 Daltons.

129. The composition of claim 128, wherein the compound is a macromolecule with a molecular weight of about or at 1000 or 1000 to about or at 50,000 or 50,000 Daltons.

130. The composition of claim 129, wherein the compound is a macromolecule with a molecular weight of about or at 1000 or 1000 to about or at 10,000 or 10,000 Daltons.

131. The composition of claim 130, wherein the compound is a macromolecule with a molecular weight of about or at 1000 or 1000 to about or at 3,000 or 3000 Daltons.

132. The composition of claim 122, wherein the compound is a small molecule.

133. The composition of claim 123, wherein the macromolecule is selected from among a polynucleotide, a nucleic acid, a polypeptide, a glycopeptide, a protein, a carbohydrate, a lipid, a fatty acid, a polysaccharide, carbohydrate- or polysaccharide-protein conjugates, virus, virus particles, viroids, prions and mixtures thereof.

134. The composition of claim 123, wherein the macromolecule is selected from among hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, and cholesterol lowering agents.

135. The composition of claim 123, wherein the macromolecule is conjugated to a small molecule.

136. The composition of claim 135, wherein the small molecule is selected from among haptens, hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, and cholesterol lowering agents.

137. The composition of claim 132, wherein the small molecule has a molecular weight of about or at 50 to about or at 1000 Daltons.

138. The composition of claim 137, wherein the small molecule is selected from among haptens, hormones, prostaglandins, antibiotics, chemotherapeutic agents, hematopoietics, anti-infective agents, antiulcer agents, antiallergic agents, antipyretics, analgesics, anti-inflammatory agents, antidementia agents, antiviral agents, antitumor agents, antidepressants, psychotropic agents, cardiotonics, diuretics, antiarrhythmic agents, vasodilators, antihypertensive agents, antidiabetic agents, anticoagulants, and cholesterol lowering agents.

139. The composition of claim 138, wherein the small molecule is an antibiotic.

140. The composition of claim 139, wherein the antibiotic is selected from among aminoglycosides, ansamycins, carbacephem, carbapenems, cephalosporins, macrolides, penicillins, quinolones, sulfonamides and tetracyclines.

141. The composition of claim 138, wherein the compound is an antiviral agent.

142. The composition of claim 141, wherein the antiviral agent is for treatment of influenza, parainfluenza, or respiratory syncytial virus-mediated infections.

143. The composition of claim 142, wherein the antiviral agent is zanamivir or oseltamivir phosphate.

144. The composition of claim 138, wherein the compound is a chemotherapeutic agent.

145. The composition of claim 144, wherein the chemotherapeutic agent is paclitaxel.

146. The composition of claim 138, wherein the compound is a prostaglandin.

147. The composition of claim 133, wherein the macromolecule is a nucleic acid.

148. The composition of claim 147, wherein the nucleic acid is selected from among DNA, RNA and PNA.

149. The composition of claim 148, wherein the nucleic acid is RNA.

150. The composition of claim 149, wherein the RNA is selected from among siRNA, tRNA, snRNA and ribozymes.

151. The composition of claim 150, wherein the RNA is siRNA.

152. The composition of claim 133, wherein the macromolecule is a virus.

153. The composition of claim 133, wherein the macromolecule is a peptide.

154. The composition of claim 153, wherein the peptide is leuprolide or somatostatin.

155. The composition of claim 122, wherein the compound is water-insoluble.

156. The composition of claim 122, wherein the counterion is selected from among an anionic compound, a cationic compound and a zwitterionic compound.

157. The composition of claim 156, wherein the counterion is an anionic compound.

158. The composition of claim 157, wherein the anionic compound is selected from among sodium citrate, sodium sulfate, zinc sulfate, magnesium sulfate, potassium sulfate and calcium sulfate.

159. The composition of claim 122, wherein the counterion is selected from among citric acid, itaconic acid and pivalic acid.

160. The composition of claim 122, wherein the counterion is an amino acid.

161. The composition of claim 160, wherein the counterion is glycine or arginine.

162. The composition of claim 122, wherein the counterion is polyethylene glycol (PEG) or polyethyleneimine (PEI).

163. The composition of claim 122, wherein the resulting microparticle composition further comprises micro-carriers, acid-resistant coating agents, protease-resistant coating agents, enteric coating agents, bulking agents, excipients, inactive ingredients, stability enhancers, taste and/or odor modifiers or masking agents, vitamins, sugars, therapeutic agents, anti-oxidants, immuno-modulators, trans-membrane transport modifiers, anti-caking agents, chitosans or flowability enhancers.

164. The composition of claim 163, wherein the resulting microparticle composition further comprises a micro-carrier.

165. The composition of claim 164, wherein the micro-carrier is selected from among amino acids, carboxylic acids, proteins, nucleic acids, polysaccharides, and materials that are capable of forming hydrogels.

166. The composition of claim 165, wherein the micro-carrier is a material that is capable of forming a hydrogel.

167. The composition of claim 166, wherein the micro-carrier is gelatin or dextran.

168. The composition of claim 167, wherein the compound is a protein.

169. The composition of claim 167, wherein the compound is a nucleic acid.

170. The composition of claim 169, wherein the nucleic acid is siRNA and the micro-carrier is gelatin.

171. The composition of claim 122, wherein the compound is a micro-carrier.

172. The composition of claim 171, wherein the micro-carrier is selected from among amino acids, carboxylic acids, proteins, nucleic acids, polysaccharides, and materials that are capable of forming hydrogels.

173. The composition of claim 172, wherein the micro-carrier is a material that is capable of forming a hydrogel.

174. The composition of claim 173, wherein the micro-carrier is gelatin or dextran.

175. The composition of claim 122 that has a shelf life of from about or at one week to about or at 1 month, from about or at 1 month to about or at six months, from about or at six months to about or at one year, from about or at 1 year to about or at 2 years, or from about or at 2 years to about or at 5 years at a temperature of about or at 55° C., 50° C., 45° C., 44° C., 42° C., 40° C., 39° C., 38° C., 37° C. or below.

176. The composition of claim 122 that further comprises an active agent.

177. The composition of claim 176, wherein the active agent is selected from among antibiotics, chemotherapeutic agents, antidiabetics, anticonvulsants, analgesics, antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids, sympathomimetics, parasympathomimetics, tranquilizers, urinary tract drugs, vaccines, vaginal drugs, nonsteroidal anti-inflammatory drugs, angiotensin converting enzymes, polynucleotides, polypeptides, polysaccharides, enzymes, hormones, vitamins, minerals, and nutritional supplements.

178. The composition of claim 122, wherein the amount of compound in the microparticles is from about or at 0.1% to about or at 99% or greater, w/w.

179. The composition of claim 178, wherein the amount of compound in the microparticles is from about 90% to about 99% w/w.

180. The composition of claim 122, wherein the moisture content of the microparticles is adjusted whereby at least about 90% or 90% of the activity of the compound is retained after storage for about or at six months to about or at 1 year at a temperature of about 25° C.

181. The composition of claim 122, wherein the amount of counterion in the microparticles is from about 0.01% or 0.01% to about 60% or 60% w/w.

182. The composition of claim 181, wherein the amount of counterion in the microparticles is from about 0.1% or 0.1% to about 10% or 10% w/w.

183. The composition of claim 182, wherein the amount of counterion in the microparticles is from about 0.2% or 0.2% to about 5% or 5% w/w.

184. The composition of claim 183, wherein the amount of counterion in the microparticles is from about 6% or 6% to about 12% or 12%.

185. The composition of claim 122 that is for oral administration or ingestion.

186. The composition of claim 122 that is for intravenous, intranasal, parenteral, pulmonary, subcutaneous, ophthalmic or intramuscular administration.

187. The composition of claim 122 that is for inhalation.

188. The composition of claim 122, wherein the size of the microparticles is from about 0.001 μm or 0.001 μm to about 10 turn or 10 μm.

189. An article of manufacture, comprising the composition of claim 122, a packaging material for the composition and a label that indicates that the composition is for a therapeutic, nutraceutical or cosmetic indication.

190. The article of claim 189, wherein the composition is for a therapeutic indication.

191. The article of claim 190, wherein the therapeutic indication is influenza, parainfluenza or respiratory disorders.

192. The article of claim 191, further comprising an inhaler for pulmonary administration of the composition.

193. A method of preventing or treating an infectious disease, comprising administering a therapeutically effective amount of the composition of claim 122 to a subject.

194. The method of claim 193, wherein the infectious disease is selected from among influenza, parainfluenza, respiratory syncytial virus.

195. The method of claim 194, wherein the administering is performed orally, intravenously, intranasally, parenterally, subcutaneously, transdermally, topically, intraarticularly, intramuscularly or by inhalation.

196. A method of making microparticles of siRNA, comprising:
(a) adding an antisolvent to a solution of siRNA in an aqueous solvent; and
(b) gradually cooling the solution to a temperature below about 25° C., whereby a composition containing microparticles comprising siRNA is formed, wherein steps (a) and (b) are performed simultaneously, sequentially, intermittently, or in any order.

197. The method of claim 196, further comprising:
(c) adding a counterion, wherein steps (a), (b) and (c) are performed simultaneously, sequentially, intermittently, or in any order.

198. The method of claim 196, wherein the antisolvent is isopropanol.

199. The method of claim 196, wherein the solvent is water.

200. A composition, comprising microparticles of siRNA.

201. The composition of claim 200, further comprising a counterion.

202. A method of making microparticles of a virus, comprising:
(a) adding an antisolvent to a solution of virus in an aqueous solvent; and
(b) gradually cooling the solution to a temperature below about 25° C., whereby a composition containing microparticles comprising a virus is formed, wherein steps (a) and (b) are performed simultaneously, sequentially, intermittently, or in any order.

203. The method of claim 202, further comprising:
(c) adding a counterion, wherein steps (a), (b) and (c) are performed simultaneously, sequentially, intermittently, or in any order.

204. The method of claim 202, wherein the antisolvent is isopropanol.

205. A method of making microparticles of a virus, comprising:
(a) adding a counterion to a solution of virus in an aqueous solvent; and
(b) gradually cooling the solution to a temperature below about 25° C., whereby a composition containing microparticles comprising a virus is formed, wherein steps (a) and (b) are performed simultaneously, sequentially, intermittently, or in any order.

206. The method of claim 205, wherein the solvent is water.

207. A composition, comprising microparticles of a virus.

208. The composition of claim 207, further comprising a counterion.
